(19) United States
(12) Patent Application Publication
(10) Pub. No.: US 2010/0285559 A1
(43) Pub. Date: Nov. 11, 2010

(54) RADIATION-RESISTANT MUTANTS OF A HALOPHILIC ARCHAEON AND USES THEREOF

(76) Inventors: Shiladitya DasSarma, Ellicott City, MD (US); Linda Deveaux, Pocatello, ID (US); Douglas P. Wells, Pocatello, ID (US)

Correspondence Address: MOORE & VAN ALLEN PLLC, P.O. BOX 13706, Research Triangle Park, NC, 27709, US

(21) Appl. No.: 12/679,937

(22) PCT Filed: Sep. 25, 2008
(86) PCT No.: PCT/US08/77596

§ 371 Date: Jul. 29, 2010

Related U.S. Application Data
(60) Provisional application No. 60/974,945, filed on Sep. 25, 2007.

(51) Int. Cl. C12N 13/00; C12N 1/21; A62D 3/02

(52) U.S. Cl. 435/173.9; 435/252.3; 435/262.5

(57) ABSTRACT

The present invention relates to variants of the model halophile Halobacterium sp. NRC-1 that have been exposed to cycles of irradiation with high doses of 18-20 MeV electrons. These novel isolated halobacterium exhibit increased resistance to radiation and may be used for bioremediation of toxic and highly radioactive waste sites and/or for studying and modulating the mechanism involved in such resistance.

### CROSS-REFERENCE TO RELATED APPLICATION

The present application claims priority to U.S. Provisional Patent Application No. 60/974,945 filed on Sep. 25, 2007 the contents of which are hereby incorporated by reference herein.

### BACKGROUND OF THE INVENTION

1. Field of the Invention

The present invention relates to novel isolated and mutant Halobacterium sp strains, and more particularly, to novel variants of halophilic archaea that exhibit increased resistance to electron-beam radiation and may be used for bioremediation of highly radioactive waste sites and/or for studying and modulating the mechanism involved in such resistance.

2. Background of the Related Art

Extremely halophilic archaea are among the most extremophilic microorganisms, and they display tolerance to multiple stressors (1). Halophilic archaea are found in brines containing saturating levels of sodium chloride such as in saltern crystallizer ponds that are exposed to intense solar radiation. To survive under these conditions, they have developed remarkable resistance to UV radiation and have a highly efficient photoreactivation repair system (2). In addition, this class of microorganisms has been shown to survive prolonged exposure to desiccation and may live in brine inclusions in ancient underground salt deposits (3). Their resistance to desiccation and radiation may also be responsible for their prolonged survival in outer space, where they have been recovered after longer exposure than any other vegetative cells tested (4).

Among the extremely halophilic archaea, Halobacterium sp. NRC-1 is a well-characterized model organism with a completely sequenced genome and post-genomic tools for the study of numerous cellular pathways (5). This microbe is easily cultured in the laboratory and is amenable to genetic studies through a facile gene knockout system and whole genome DNA microarrays (6). As a result, Halobacterium sp. NRC-1 has been used for a wide range of studies, including UV and ionizing radiation tolerance (7-13). Halobacterium sp. NRC-1 is able to efficiently repair extensive double-strand DNA breaks in its genomic DNA, produced both by desiccation and by ionizing radiation, as well as cyclopyrimidine dimers and 6-4 pyrimidone photoproducts produced by UV radiation.

The genome of Halobacterium sp. NRC-1 contains a 2-Mbp chromosome and two related extrachromosomal replicons of 365 and 191 kbp (14). The genome contains homologs of both bacterial and eukaryotic DNA repair genes (15, 16). Some of the predicted proteins involved in nucleotide excision repair, such as Rad2 (FEN1/XPG), Rad3 (XPD), Eif4A (RAD1/XPF) and Rad 25 (XPB), are more closely related to eukaryotic proteins than to bacterial proteins. However, other proteins, including the UvrABC excision repair system, are more similar to the bacterial counterparts (17). The presence of homologs to bacterial excision repair genes is unusual for the archaea, and recent genetic knockout studies have established their importance in dark repair in Halobacterium sp. NRC-1 (18). Genetic studies have shown that the active photoreactivation system of Halobacterium sp. NRC-1 is mediated by a photolyase, coded by the phr2 gene (2). In a transcriptome analysis of Halobacterium sp. NRC-1 exposed to UV radiation, genes likely involved in homologous recombination, including radA1, were shown to be significantly induced, suggesting the involvement of homologous recombination in repair of DNA damage (12, 19).

Halobacterium sp. NRC-1 has recently been shown to have high resistance to γ radiation (20). The resistance was attributed to the ability to repair double-strand breaks as well as to the protective properties of specific membrane pigments to oxidative damage. The high salinity in which this organism and other halophiles grow also provides protection against damage by decreasing the oxygen content and hence levels of damaging oxygen-produced radicals (21). Given the already considerable resistance to radiation exhibited by this organism, it was unclear whether genetic changes would allow survival after higher challenging doses of radiation. The radiation resistance of some bacterial species has been increased up to 20-fold by repeatedly exposing growing cultures to doses of radiation that allow very few survivors (22, 23). In these cases, however, the natural resistance of the starting organisms to both radiation and desiccation was very low in comparison to Halobacterium. In addition, the relatively low rate of dose delivery from these radiation sources necessitated longer times for delivery of the higher doses, during which DNA repair mechanisms may have been operating. It is likely that simultaneous mutation of many genes involved in DNA repair would be required in these cases for changes in resistance.

Thus, it would be advantageous to provide variants of Halobacterium sp. NRC-1 that exhibit increased radiation-resistance that may be used in circumstances that require higher levels of radiation, such as ionizing radiation, to effect remediation of highly radioactive waste sites or sterilization activities. Further such variants can be used as a testing model to determine what genes need to be modulated for the purpose of up-regulating or down-regulating such resistance to extreme levels of radiation including gamma radiation.

### SUMMARY OF THE INVENTION

Accordingly, the present invention provides a method of isolating variants of the halophile Halobacterium sp. NRC-1 that exhibit resistance to extreme levels of radiation, the method comprising:
a) providing a culture medium comprising a NRC-1 halobacterium sp;
b) irradiating the culture with at least a dose rate of 10 kGy/s to 30 kGy/s and more preferably from about 16 kGy to 23 kGy;
c) isolating the viable halobacterium strains;
d) repeating steps b and c at least one additional time; and
e) selecting and isolating variants of Halobacterium displaying increased gene transcripts of rfa3, rfa8 and ral with at least a 1.9 to 4 fold increase relative to the wild-type and/or non-irradiated Halobacterium sp. NRC-1 strain.

Preferably, the steps of b) and c) are repeated at least three additional times to isolate organisms with increased radiation resistance. The radiation is preferably delivered by a source that provides a pulsed electron beam having the ability to deliver 18-20 MeV electrons at a peak dose ranging from about 4.0×10⁴Gy/s to 2.5×10⁵ Gy/s (1 Gy=100 rads). The pulse parameters may include a pulse width of 1 to 3 ms, with a repetition rate of from about 40 to 80 HZ.

In another aspect, the present invention relates to a mutant Halobacterium strain isolated by the irradiating NRC-1 halobacterium sp with electron beam radiation as described above, wherein the mutant strain has lost the gas vesicle production of the parent strain thereby rendering the mutant colonies translucent. Further, the mutant Halobacterium strains exhibited an LD₅₀ from about 11 kGy to about 25 kGy and additionally exhibited greater radiation resistance in the actively growing stage.

In a still further aspect of the present invention provides for mutant strains of NRC-1 halobacterium sp having been exposed to at least two cycles of electron beam radiation in a range from 10 kGy/s to 30 kGy/s, and preferably four cycles, wherein there is increased transcript levels of rfa3, rfa8 and ral genes relative to the wild type halobacterium sp. Importantly, the rfa3 (SEQ ID NO: 1), rfa8 (SEQ ID NO: 2), and ral (SEQ ID NO: 3), genes are homologs to the single-strand DNA-binding proteins RPA that exhibits DNA repair, replication and recombination relevant to DNA metabolism.

Yet another aspect of the present invention relates to a method for increasing the repair mechanisms of DNA damage through increased stabilization of exposed single-strand DNA, the method comprising, inducing increased expression levels of rfa3, rfa8 and ral genes in a Halobacterium sp strain.

In another aspect, the present invention relates to a method for decontaminating a waste site comprising radioactive materials, the method comprising;
a) introducing a mutant Halobacterium sp NRC-1 strain isolated after exposing the Halobacterium sp NRC-1 strain to at least two cycles of electron beam radiation as described above; and
b) providing sufficient nutrients for maintaining the culture for decontaminating the waste products.

In yet another aspect, the present invention provides for protecting a surface from radiation ranging from UV to gamma radiation, the method comprising,
covering the surface with a mutant Halobacterium sp NRC-1 strain or components thereof wherein the mutant Halobacterium sp NRC-1 strain or components thereof is isolated after exposing a wild type Halobacterium sp NRC-1 strain to at least one cycle of electron beam radiation and preferably at least four cycles in a range from 10 kGy/s to 30 kGy/s.

Another aspect of the present invention relates to a method for modulating resistance to high levels of radiation in an organism, the method comprising:
introducing an active agent into the organism to modulate the up-regulation or down-regulation of expression of genes encoding single stranded DNA-binding proteins wherein the single stranded DNA-binding proteins are involved in DNA repair.

In the present invention the single stranded DNA-binding proteins are expressed by genes included in the rfa3 operon in the mutant Halobacterium sp NRC-1 strains. The genes expressed by the rfa3 operon in the variants of the present invention include the rfa3, rfa8 and ral genes. Additionally the single stranded DNA-binding proteins include the replication Protein A (RPA) in eukaryotics or single-strand binding protein (SSBs) in bacteria, wherein the single stranded DNA-binding proteins bind to exposed single-strand regions to act as a signal for damage repair proteins.

Notably, the modulating active agent may include a homologous nucleotide sequence that binds to the genes of the rfa3 operon thereby inactivating such expression and down regulating radiation resistance. In the alternative the modulating agent may include an agent that induces increased expression of genes encoding single stranded DNA-binding proteins that are involved in DNA repair. Importantly, having the ability to modulate such resistance to radiation allows for inactivation of such activity by targeting molecules that are responsible for such resistance. This knowledge may be used to enhance radiation therapy of cancer cells or inactivate resistance characteristics that interfere with sterilization.

In a further aspect, the present invention provides for a biological indicator to ensure the quality control of radiation sterilization in the food industry, hospital settings or space travel, wherein the biological indicator is a mutant Halobacterium sp NRC-1 strain isolated after exposing the Halobacterium sp NRC-1 strain to at least one cycle of electron beam radiation and more preferably at least two or more cycles of such radiation as described herein.

In yet another aspect, the present invention relates to isolating a gene from a mutant Halobacterium sp NRC-1 that induces increased levels of gene transcripts of rfa3, rfa8 and ral with at least a 1.9 to 4 fold increase relative to the wild-type and/or non-irradiated Halobacterium sp. NRC-1 strain; amplifying the gene and including same in a vector system for transfecting to a bacteria host cell to provide increased survival rate of such bacteria in ionizing radiation conditions.

Still further, the present invention relates to a method for expressing a protein of choice in high radiation conditions including bombardment with high energy radiation, the method comprising:
inserting nucleotide sequences encoding for the protein of choice into a mutant Halobacterium sp NRC-1 that exhibits increased gene transcripts of rfa3, rfa8 and ral with at least a 1.9 to 4 fold increase relative to the wild-type and/or non-irradiated Halobacterium sp. NRC-1;
maintaining suitable conditions for expressing the protein of choice by the mutant Halobacterium sp NRC-1; and recovering the protein of choice.

In yet another aspect, the present invention provides for use of a variant Halobacterium sp. NRC-1 strain that exhibits resistance to extreme levels of radiation and transfecting same with an expression vector comprising nucleic acid encoding for a protein of choice including enzymes that are useful for denitrification, dehalogenation or decontamination of unwanted organic or inorganic toxic waste.

Other features and advantages of the invention will be apparent from the following detailed description, drawings and claims.

### BRIEF DESCRIPTION OF THE FIGURES

FIG. 1 shows the selection of Halobacterium strains LH5 and LH7a from parent strain NRC-1 through irradiation-growth cycles. Independent aliquots of a stationary-phase culture of Halobacterium sp. NRC-1 were irradiated at the doses indicated and allowed to recover by inoculation into fresh medium prior to subsequent irradiations.

FIG. 2 shows the survival of exponential-phase Halobacterium sp. NRC-1, LH5 and LH7a compared to Deinococcus radiodurans R1 after electron-beam irradiation. Values from multiple experiments were binned according to dose. Error bars represent the errors on the mean survival per bin. NRC-1 (), D. radiodurans (◯), LH5 (▪), and LH7a (□). Individual data points were fitted to the Boltzmann curves shown. NRC-1 (—), D. radiodurans (-•-), LH5 (- - -), and LH7a (- - - - -).

FIG. 3 shows the survival of stationary-phase Halobacterium sp. NRC-1, LH5 and LH7a compared to Deinococcus radiodurans R1 after electron-beam irradiation. Values from multiple experiments were binned according to dose. Error bars represent the errors on the mean survival per bin. NRC-1 (), D. radiodurans (◯), LH5 (▪), and LH7a (□). Individual data points were fitted to the Boltzmann curves shown. NRC-1 (—) D. radiodurans (-•-), LH5 (- - -), and LH7a (- - - - -).

FIG. 4 shows the whole-genome microarray hybridization results comparing mutant strains LH5 (panel A) and LH7a (panel B) with the wild-type strain (NRC-1). For each ORF represented on the array, the logarithm10 of the hybridization ratio of mutant cells (Cy5-labeled cDNA) to wildtype cells (Cy3-labeled cDNA) is displayed in black marks on the y axis. The location of ORFs within the entire 2.6-Mb genome maps is shown on the x axis. Expression ratios of selected genes are indicated.

FIG. 5 shows analysis of rfa3 operon in Halobacterium sp. NRC-1. Panel A: Agarose gel electrophoresis of reverse transcriptase-PCR assays with primers targeting upstream regions of the predicted transcriptional start site, −29 and +3, as well as intergenic regions of rfa3 and rfa8 and rfa8 and ral, respectively. “D” stands for genomic DNA as template, “+” for assays with RNA as template and conducted with reverse transcriptase, and “−” for assays with RNA as template conducted without reverse transcriptase. Sizes of products were as predicted (for −29, 506 bp; for +3, 541 bp; for rfa3/rfa8, 416 bp; for rfa8/ral, 268 bp). Panel B: Upstream region of rfa3 in Halobacterium sp. NRC-1. Box with solid line, TATA box; box with broken line, haloarchaeal transcriptional start site consensus [(T/C)(A/G)NG]; arrows, positions of primers −29F and +3F; start codon is in bold. Absolute nucleotide positions are given for SEQ ID NO: 7 and 8.

### DETAILED DESCRIPTION OF THE INVENTION

The present invention provides for extremely radiation-resistant mutants of Halobacterium sp. NRC-1, which are the most radiation-resistant organisms currently known, by selection for isolates displaying increased resistance to electron-beam radiation. Through transcriptome analysis, the gene expression difference has been identified in these mutants that is likely responsible for increased radiation resistance.

In order to facilitate review of the various embodiments of the present invention and provide an understanding of the various elements and constituents used in making and using the present invention, the following terms used in the invention description have the following meanings.

The term “nucleic acid” as used herein, is a composition or sequence comprising nucleotide subunits, wherein the subunits can be deoxyribonucleotides, ribonucleotides, deoxyribonucleotide analogs, ribonucleotide analogs or any combinations thereof.

Halobacterium sp. NRC-1 is irradiated with a device that has the ability to generate electrons in a range from 4 MeV to 25 MeV. Preferably, a linear accelerator is used which is capable of rapidly moving or accelerating electrons down a long (linear) tube. At the end of the tube, a beam of electrons would be emitted. An electron beam is another form of radiation. Any linear accelerators (“linacs”) which produce megavoltage X-rays or electron beams may be used. The shape and intensity of the beam produced by a linac may be modified or collimated by a variety of means. Electron beams are useful for treating bacteria because the maximum of dose deposition occurs near the surface.

The variant Halobacterium sp. NRC-1 strains of the present invention having increased resistance to high energy ionizing radiation can be used for transformation using well known techniques. Transformation, in the context of the current invention, is the process by which exogenous DNA is inserted into a bacterium, causing the bacterium to change its genotype and/or phenotype. Such a change in genotype or phenotype may be transient or otherwise. Exogenous DNA is any DNA, whether naturally occurring or otherwise, from any source that is capable of being inserted into any organism. Preferably, exogenous DNA is any DNA, whether naturally occurring or otherwise, from any source that is capable of being inserted into bacteria. Such exogenous DNA includes, without limitation, plasmid DNA, cosmid DNA, eukaryotic (particularly mammalian, and most particularly human) DNA, DNA libraries, cDNA libraries, expression vectors and phage DNA (such as bacteriophage lambda DNA).

A number of procedures exist for the preparation of competent variant Halobacterium sp. NRC-1 strains of the present invention and the introduction of DNA into same. A very simple, moderately efficient transformation procedure for use with bacteria, such as E. coli involves re-suspending log-phase bacteria in ice-cold 50 mM calcium chloride at about 10¹⁰ bacteria/ml and keeping them ice-cold for about 30 min. Plasmid DNA (0.1 mg) is then added to a small aliquot (0.2 ml) of these now competent bacteria, and the incubation on ice continued for a further 30 minutes, followed by a heat shock of 2 minutes at 42° C. The bacteria are then usually transferred to nutrient medium and incubated for some time (30 minutes to 1 hour) to allow phenotypic properties conferred by the plasmid to be expressed, e.g., antibiotic resistance commonly used as a selectable marker for plasmid-containing cells. Protocols for the production of competent bacteria have been described in Molecular Cloning, A Laboratory Manual, Cold Spring Harbor Press, Cold Spring Harbor, N.Y. (1982).

Another rapid and simple method for introducing genetic material into bacteria is electoporation. This technique is based upon the use of high-voltage electric pulses that can induce cell plasma membranes to fuse. Subsequently, it was found that when subjected to electric shock (typically a brief exposure to a voltage gradient of 4000-16000 V/cm), the bacteria take up exogenous DNA from the suspending solution, apparently through holes momentarily created in the plasma membrane. A proportion of these bacteria become stably transformed and can be selected if a suitable marker gene is carried on the transforming DNA.

The invention also relates to a method of producing a desired protein by transforming the bacteria of the present with a DNA molecule encoding said desired protein. Thus, the invention concerns a method of producing a desired protein comprising obtaining a bacterium according to the present invention, transforming said bacterium with a DNA molecule capable of expressing said desired protein, and culturing said transformed bacterium under conditions sufficient to produce said desired protein. Upon culturing the bacterium under appropriate conditions, the bacterium is capable of producing the desired protein even under high radiation or caustic conditions. The desired protein may then be isolated, and a substantially pure protein obtained by well known protein purification techniques.

The Halobacterium sp. NRC-1 of the present invention can be cultured under aerobic conditions in a nutrient medium containing one or more sources of carbon, nitrogen and optionally nutrient inorganic salts and/or trace elements. The carbon sources are, for example, starch, glucose, sucrose, dextrin, fructose, molasses, glycerol, lactose or galactose, preferably glucose. The sources of nitrogen are, for example, soybean meal, peanut meal, yeast extract, beef extract, peptone, tryptone, malt extract, corn steep liquor, gelatin or casamino acids, preferably soybean meal and corn steep liquor. As the organic trace nutrients, amino acids, vitamins, fatty acids, nucleic acids, those containing these substances such as peptone, casamino acid, yeast extract and soybean protein decomposition products are used. The nutrient inorganic salts and trace elements are, for example, sodium hydrogen phosphate, potassium hydrogen phosphate, sodium chloride, cobalt chloride, calcium chloride, calcium carbonate, potassium nitrate, ammonium sulfate or magnesium sulfate, preferably cobalt chloride and calcium carbonate. Cultivation of the culture is usually carried out at temperatures between 20-45° C. and in a medium having a pH from about 6.0 to about 8.0, and preferably, about 7.0 to about 8.0.

It is contemplated that the present invention may be practiced using either batch, fed-batch or continuous processes and that any known mode of fermentation would be suitable. A classical batch fermentation is a closed system where the composition of the medium is set at the beginning of the fermentation and not subjected to artificial alterations during the fermentation. Thus, at the beginning of the fermentation the medium is inoculated with the desired organism or organisms and fermentation is permitted to occur adding nothing to the system. Typically, however, a batch fermentation is “batch” with respect to the addition of carbon source and attempts are often made at controlling factors such as pH and oxygen concentration. In batch systems the metabolite and biomass compositions of the system change constantly up to the time the fermentation is stopped. Within batch cultures, cells moderate through a static lag phase to a high growth log phase and finally to a stationary phase where growth rate is diminished or halted. If untreated, cells in the stationary phase will eventually die.

A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the medium.

Continuous fermentation is an open system wherein a defined fermentation medium is added continuously to a bioreactor and an equal amount of conditioned medium is removed simultaneously for processing. Continuous fermentation generally maintains the cultures at a constant high density. Continuous fermentation allows for the modulation of one factor or any number of factors that affect cell growth or end product concentration. For example, one method will maintain a limiting nutrient such as the carbon source or nitrogen source at low concentration and allow all other parameters to be in excess. In other systems a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the fermentation.

The variant Halobacterium sp. NRC-1 strains of the present invention are particularly useful in methods of monitoring the effectiveness of sterilization or decontamination procedures that utilize ionizing radiation. The various aspects of the present invention find particular use in, for example, monitoring sterilization and decontamination of water, food supply, hospital surface although the present invention is not limited thereto. In particular, the bacterium may be used as an indicator microorganism for such sterilization procedures. That is, since the present mutant bacterium is more resistant to ionizing radiation than other microorganisms that are possible contaminants in, for example, water supply, hospital setting, the determination that the possible bacteria contaminant has been completely removed from such sterilizing system, the radiation resistant bacterium of the present invention is a reliable indicator that all contaminating microorganisms have been eliminated.

The radiation resistant bacterium of the present invention may be used for treating mixed waste in the presence of high levels of radiation, high temperature, high pH, and high saline environments. Heretofore, no organism having these combined abilities has been available for such remediation efforts. As such, the radiation resistant bacterium of the present invention may be used in bioreactors, biofilters, rotating biological contactors, and other gaseous and/or liquid bioreactors to treat liquid and gaseous waste while being exposed to high radiation levels.

The ability of the radiation resistant bacteria of the present invention to survive and grow during exposure to high radiation levels increases the innate ability of the bacteria to effectively bioremediate any contaminants as well as accumulate various metals and radionuclides. Remediation in the presence of high radiation fields is key for the mixed hazardous wastes. Conventional organisms useful for bioremediation of organic waste would not tolerate the harsh conditions of radiation, pH, and salinity. The current isolate is able to survive under such conditions and may, therefore, provide effective treatment and bioremediation of contaminants and metals within the waste.

Having now generally described the invention, the same will be more readily understood through reference to the following examples which are provided by way of illustration, and are not intended to be limiting of the present invention unless specified. All patents, patent applications and publications mentioned herein are incorporated by reference in their entirety.

### EXAMPLES

### Materials and Methods

Strains and Growth Conditions

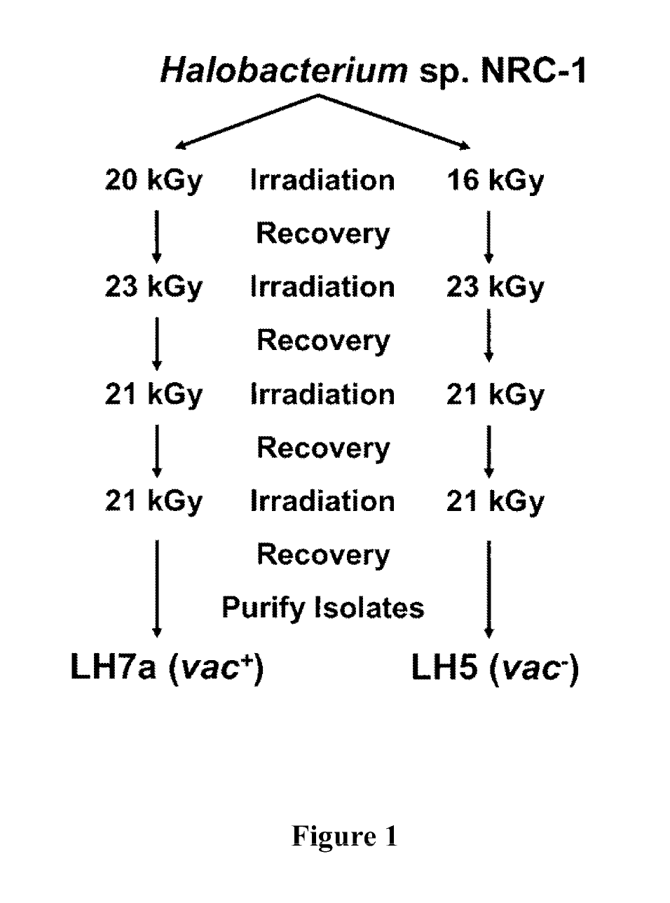

The strains of Halobacterium used were derivatives f strain NRC-1 (FIG. 1). Deinococcus radiodurans R1 (ATCC 13939) was a gift from John Battista at Louisiana State University. Halobacterium cultures were grown in CM+ medium (24) at 42° C. with shaking, without supplemental illumination. D. radiodurans was grown in TGY (0.5% tryptone, 0.3% yeast extract, 0.1% glucose) at 30° C. with aeration (25). For solid medium, 2% agar was added. Halobacterium plates were incubated at 42° C. for 7 days, then removed and allowed to continue to grow at room temperature until no new colonies appeared. D. radiodurans plates were incubated at 30° C. for 3-5 days. Liquid cultures for irradiation were inoculated from purified isolates on solid medium. Stock cultures were maintained in glycerol at −80° C. For short-term use, purified cultures were maintained on stock plates at 4° C. Growth rates were monitored by measuring the optical density at 600 nm (OD600) of cultures in liquid medium over time. Samples of growing cultures were diluted and plated at various times to ensure that vastly different cell sizes, the presence of nonviable cells, or differences in gas vesicle production did not skew the OD600 readings. For determination of survival, irradiated liquid cultures were immediately diluted and spread on plates. Survival after irradiation was determined by comparing surviving cells to an un-irradiated control.

Irradiation and Dosimetry

The 20 MeV pulsed electron LINAC delivered 18-20 MeV electrons at a peak current of 80 mA, with a peak dose rate of 2.5×10⁵ Gy/s and an average dose rate of 30 Gy/s. The pulse width was 2 ms, and the repetition rate was 60 Hz. Samples were irradiated at room temperature in 0.2-ml thin-walled PCR tubes held 2.5 m from the beam port. At this distance, the uniformity of the beam was within 10% of the peak dose over a circular area of 10 cm diameter, which encompassed the sample holder size. Samples were located on isocontours of dose with sample dose variations less than 1%. Beam location was determined before and after irradiations with PIN diodes, and the sample doses delivered were measured using a GEX corporation thin-film dosimetry system and GEX B3 radiochromic film (26).

Irradiation-Growth Cycles

A stationary-phase culture (4×10⁸ cells/ml) of Halobacterium NRC-1 in 0.1-ml aliquots was exposed to electrons from a 20 MeV LINAC at doses of 16.4±1.6 and 19.5±2 kGy. These aliquots were diluted into 2 ml of CM+ medium and incubated with shaking for 2-5 days at 42° C. until growth was visible. Glycerol was added to 15%, and 1 ml of each suspension was frozen at −80° C. Prior to the next irradiation, the frozen stocks were used to inoculate 2 ml of CM+ medium and grown for 2 clays at 42° C. Aliquots of these cultures were irradiated at 23±2.3 kGy. Each was then diluted into 2 ml broth and incubated until growth was visible. Once again, 0.1-ml aliquots of these cultures were irradiated at 20.6±2.1 kGy. Fifty microliters of each irradiated sample was diluted into 2 ml CM+ medium and grown for 7 days, until growth was visible. These cultures were irradiated in 0.1-ml aliquots at 21±2.1 kGy. Fifty microliters of each aliquot was diluted into 1 ml CM+ medium until growth was visible, approximately 7 days. These two cultures were streaked on solid medium and individual isolates were purified four times.

LH5 was derived from the cultures originally irradiated at 16.4 kGy and LH7a from the culture irradiated at 19.5 kGy. This procedure is summarized in FIG. 1.

Cell Irradiation and Survival Measurement

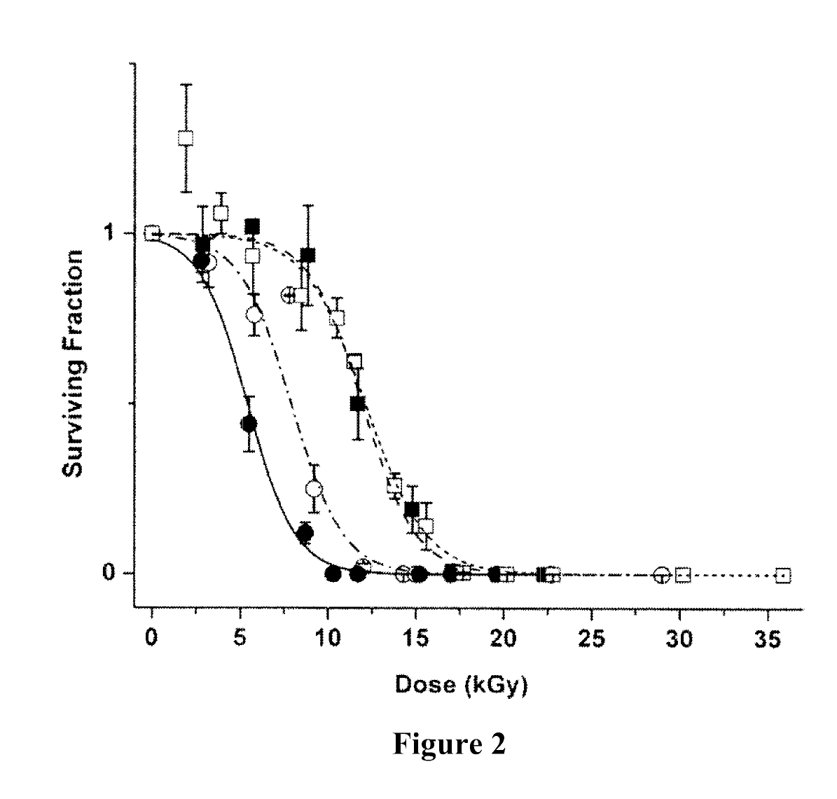

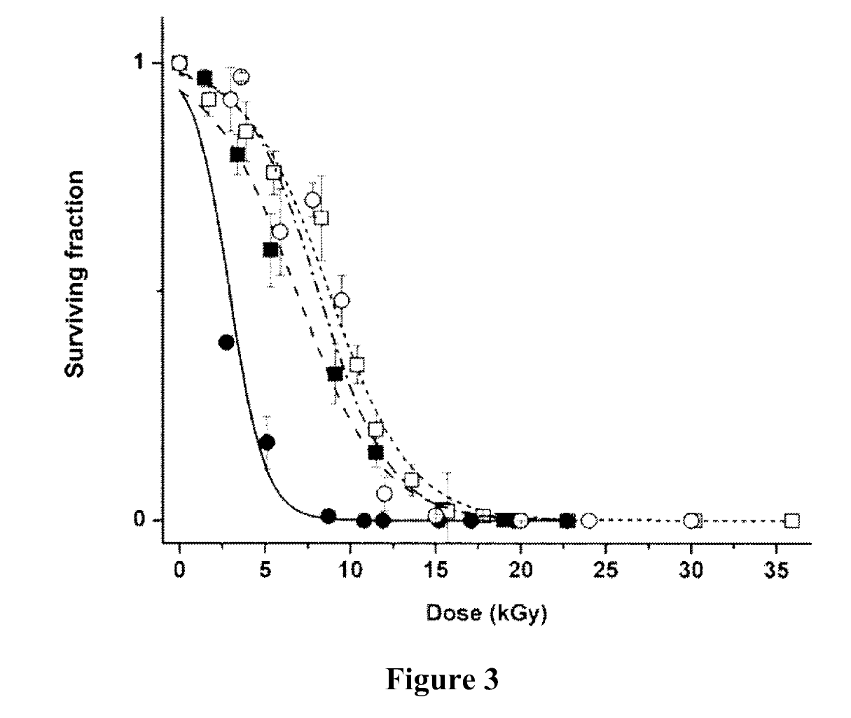

Each strain was grown in liquid culture to the appropriate density and culture state (exponential or stationary phase). In an individual experiment, a culture was divided into aliquots and subjected to a predetermined set of doses, including no radiation. The aliquots were diluted in growth medium immediately after irradiation and plated in duplicate or triplicate. In a given experiment, the multiple platings of the un-irradiated (0 Gy) aliquot were counted and averaged to give the initial cell density in colony-forming units per milliliter (cfu/ml). This value represented 100% survival and was used as the basis of comparison for all irradiated aliquots of that culture. The duplicate or triplicate platings of each irradiated aliquot were averaged to determine cfu/ml at that dose. The surviving fraction for that dose was determined by dividing the cfu/ml by the cfu/ml representing 100% survival (0 Gy, or initial cell density) of that culture. For each strain, multiple experiments (independent cultures) were performed on at least two different days, and the surviving fractions were combined (FIGS. 2 and 3).

Survival Curve Fitting

For each strain, surviving fractions from multiple irradiations were combined and plotted in OriginPro 7 (OriginLab Corporation, Northampton, Mass.). The resulting data were fitted to a sigmoidal Boltzmann function,

$$[ \frac{( {A}_{1} + {A}_{2} )}{1 + {e}^{( X - {X}_{0} ) / dx}} ] + ( {A}_{2} ) ,$$

with two parameters fixed at A₂=0 and A₁=1. Here X₀ is the LD₅₀ and dx is the width of the transition from surviving fraction=1 to surviving fraction=0 (see FIGS. 2 and 3). Individual values from multiple irradiations (ranging in number from 30 to 67 as shown in Table 1 which shows the electron beam doses delivered and mean survival of Halobacterium strains and Deinococcus radiodurans for each strain were combined and used for curve fitting. Bins were selected for clarity of presentation, with each bin (with one exception; see Table 1) containing a minimum of two data points and an average of five. The binned values were used for y-error determination in FIGS. 2 and 3, which is calculated as the error on the mean surviving fraction of each bin. For clarity, the binned data points and standard error are presented in FIGS. 2 and 3; however, the curve in these figures was generated from the fit of the unbinned data points.

Microarray Analysis

Halobacterium sp. strain NRC-1 and derivatives LH5 and LH7a were grown under aerobic conditions to early exponential growth phase (OD600 of 0.15 to 0.3). Cells were harvested by chilling the incubation vessels in an ethanol-dry ice bath for 1 min followed by centrifugation of the culture (8,000 g, 5 min, 2° C.) in chilled beakers. Total RNA was isolated immediately after harvest at 25° C. using an Agilent Total RNA isolation kit (Agilent Technologies, Palo Alto, Calif.), and DNA was hydrolyzed using amplification grade DNase (Sigma-Aldrich, St. Louis, Mo.). Lysis of cells was carried out directly in lysis buffer. Two treatments with DNase (Invitrogen, Carlsbad, Calif.) were carried out to remove contaminating DNA from the RNA preparations. To minimize biological noise, RNA preparations from three 50-ml cultures of each strain grown under identical conditions were pooled to equal parts for cDNA synthesis. cDNA was prepared from 7 ug total RNA with Super Script III reverse transcriptase (Invitrogen) and Cy3- or Cy5-dCTP (Amersham Biosciences, Piscataway, N.J.), and purified after alkaline hydrolysis of RNA on Qiagen mini-elute columns (Qiagen, Valencia, Calif.). The labeled cDNA targets were mixed with hybridization buffer and control targets and hybridized to microarray slides assembled into a hybridization chamber (Agilent) for 17 h at 60° C. in the dark. After hybridization, the slides were washed as described in the Agilent protocol and scanned for the Cy3 and Cy5 fluorescent signals with an Agilent DNA-microarray scanner (Model no. G2565BA). Transcriptome profiling of cells was carried out in duplicate. Relative mRNA levels were determined by parallel two-color hybridization to oligonucleotide (60-mer) microarrays representing 2474 open reading frames (ORFs) representing 97% of unique Halobacterium sp. NRC-1 ORFs according to Muller and DasSarma (10). Previous results showed that differences in the relative intensity of the channels could be adjusted for by intensity-dependent LOWESS normalization (10, 27). Image processing and statistical analysis were carried out using Feature Extraction Software Version 7.1 (Agilent). Log ratios for each feature were calculated and the significance of the log ratio was assessed by calculating the most conservative log ratio error and significance value (P value) using a standard error propagation algorithm (Agilent) and a universal error model (Rosetta Biosoftware, Seattle, Wash.).

Reverse-Transcriptase PCR(RT-PCR)

Total RNA was isolated as described above. DNA was removed from the RNA by three treatments with RNase-free DNase I (Qiagen). cDNA was synthesized from 0.2 to 0.8 ug RNA and 2 pmol of specific primer with SuperScript II RNase H⁻ Reverse Transcriptase (Invitrogen) as described by the supplier. The PCR amplification mixtures contained 6 ul cDNA, 200 nM of each primer, 200 uM dNTP, 1 U Taq polymerase in PCR buffer with 1.5 mM MgCl2. For amplification of parts of rfa3-rfa8 and rfa8-ral, and to map the transcriptional start site of rfa3, the following primer pairs were used (positions in the NRC-1 chromosome of primers in parentheses): −29F (1594051-1594069)/−29R (1594539-1594556); +3F (1594082-1594103)/+3R (1594603-1594622); rfa3-rfa8F (1594829-1594848)/rfa3-rfa8R (1595225-1595244); rfa8-ralF (1595510-1595534)/rfa8-ralR (1595754-1595777). PCR parameters were as follows: 3 min at 92° C., 30 cycles of 0.45 min at 94° C., 1 min at 50° C. for the primer pair −29F/−29R, at for +3F/+3R, and at 60° C. for the other two pairs, 1 min at 72° C., followed by 7 min at 72° C.

Isolation of Extremely Radiation-Resistant Mutants of Halobacterium sp. NRC-1

Using a series of irradiation-growth enhancements based on a procedure used by Davies and Sinskey on Salmonella typhimurium (22), a culture of Halobacterium sp. NRC-1 was enriched for individuals with increased tolerance to high doses of electron-beam radiation. The two isolates chosen for further study, LH5 and LH7a, were derived from independently irradiated and derived cultures (FIG. 1). These strains were also phenotypically distinct. Strain LH5 had lost the gas vesicle production capability of the parent NRC-1, due to an IS element insertion into the gvpJ gene (data not shown) rendering colonies translucent, whereas strain LH7a retained the gas vesicle-positive phenotype (opaque colonies) of the parent strain, NRC-1.

Characterization of Radiation Resistance in Halobacterium sp. NRC-1 and Radiation-Resistant Mutants LH5 and LH7a

To determine the extent of the radiation resistance acquired by the two mutants, exponential-phase cultures (OD₆₀₀<0.2) of these strains and the parent strain NRC-1 were irradiated with 18-20 MeV electrons from a standard medical LINAC (linear accelerator at the Idaho Accelerator Centerover a range of doses. For comparison, the survival of D. radiodurans R1 was also measured, one of the most radiation-resistant organisms known (28). Exponentially growing Halobacterium wild-type parent strain, NRC-1, had an LD₅₀ of 5.4 kGy (±0.2). The LD₅₀ of D. radiodurans, irradiated under the same conditions, was 7.9 kGy (±0.2). In contrast, the LD₅₀ for the Halobacterium isolate LH5 was 11.9 kGy (±0.4) and that of LH7a was 12.1 kGy (±0.3). Moreover, survivors of LH5 and LH7a were reproducibly detected at doses as high as 25 kGy, where there were few or no survivors of D. radiodurans. The graph showing surviving fractions and fitting of the data for the exponentially growing cultures is presented in FIG. 2. Since microorganisms generally display greater resistance in stationary phase (29-31), each strain was also tested in stationary phase (FIG. 3). It was confirmed (20) that Halobacterium sp. NRC-1, unlike most other microbes, shows greater resistance when actively growing and tested the response of LH5 and LH7a at this growth density. As expected, the LD₅₀ of NRC-1 in the stationary phase was significantly less (2.9 kGy±0.2) than the value for the exponential-phase cultures (5.4 kGy). Both radiation-resistant isolates also showed decreased resistance when in the stationary phase. The LD50 of LH5 decreased to 6.8 kGy (±0.3) and that of LH7a decreased to 8.7 kGy (±0.2). One simple explanation for the increased radiation resistance of strains LH5 and LH7a would be reduced growth rate. Reduction in growth rate has been shown to increase survival of D. radiodurans after irradiation (29, 30). Given the radiation doses received by these Halobacterium cells, it is highly likely that each individual contains numerous mutations, some of which may affect the growth rate. To rule out the latter possibility, it was verified that the doubling times of LH5 and LH7a were the same as that of the parent strain NRC-1 by measuring the OD600 of cultures grown under standard conditions (data not shown).

Transcriptional Analysis of Strains LH5 and LH7a

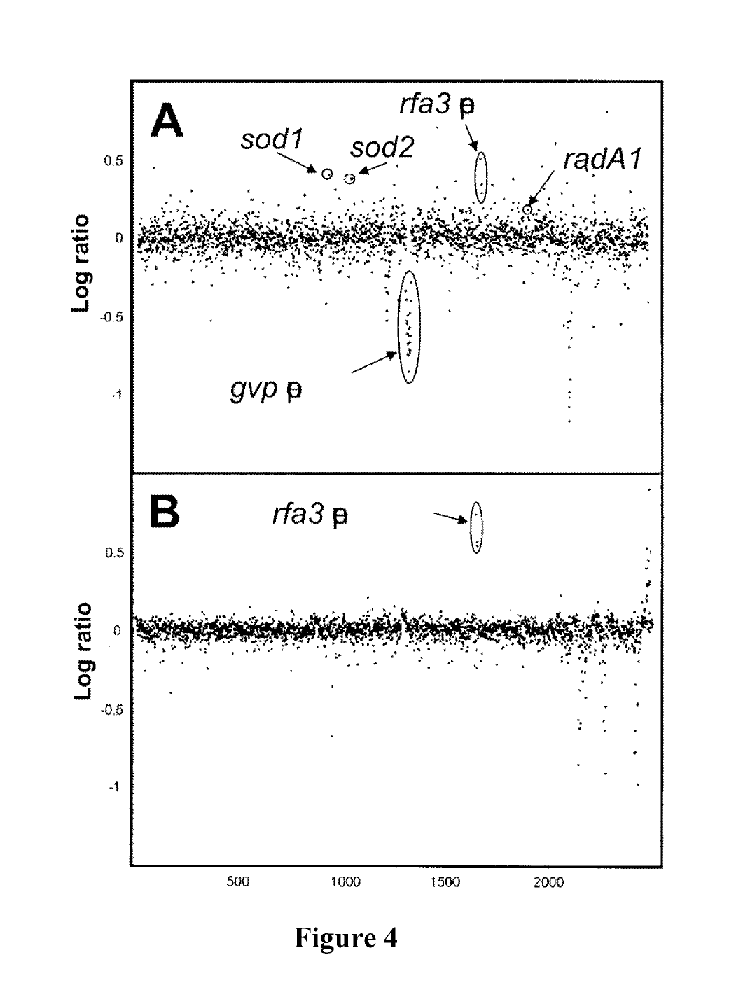

In principle, the molecular basis for increased radiation resistance of strains LH5 and LH7a may be attributed to one or more mutations in the genome. These mutations may result in increased activity of proteins involved in protection against and/or repair of radiation-induced damage. Alternatively, regulatory elements that govern expression of genes involved in protection or repair may be affected. To test for the latter scenario on a genome-wide scale, previously described in situ synthesized oligonucleotide arrays was used (10) to compare the transcriptomes of the mutant strains LH5 and LH7a to that of the wild-type strain NRC-1 under our standard conditions (see the Materials and Methods). In strain LH5, 47 and 72 genes out of 2474 ORFs (97% genome coverage) displayed more than a 1.5-fold (P<0.01) increase or reduction, respectively, in transcript level compared to the wild type (FIG. 4; Table 2). Most interestingly, genes involved in homologous recombination, e.g., radA1 (archaeal homolog of rad51/recA) and rfa3, rfa8, ral (single-stranded DNA-binding protein complex), and protection against reactive oxygen species, e.g., sod1 and sod2 (Mn-dependent superoxide dismutases), were induced in LH5. As expected from the gas vesicle-deficient phenotype of LH5, the gvp (gas vesicle protein) genes (7) had much lower expression levels in that strain. In LH7a, 11 and 36 genes had transcript levels that were significantly increased or decreased, respectively (FIG. 4; Table 2 in Supplementary Information). Among those, rfa3, rfa8 and ral were the only genes that had induced transcript levels in both LH5 and LH7a (2.1- to 3.7-fold and 3.4- to 5.5-fold, respectively; Table 2). Table 2 provides for Log ratios and p-values for gene expression comparisons between two mutant Halobacterium strains (LH5 and LH7a), and the parent strain NRC-1. Gene id reflects relative position of each potential ORF; Gene name is the genetic designation for each gene. Those of unknown function have the genetic designation “vng”. For each comparison, the log₁₀ ratio for each feature is presented, as well as the relative change (fold change) and the significance value (p-value). The functional category describes the known or anticipated cellular function of each ORF.

Transcription and Promoter Analysis of the rfa3 Operon

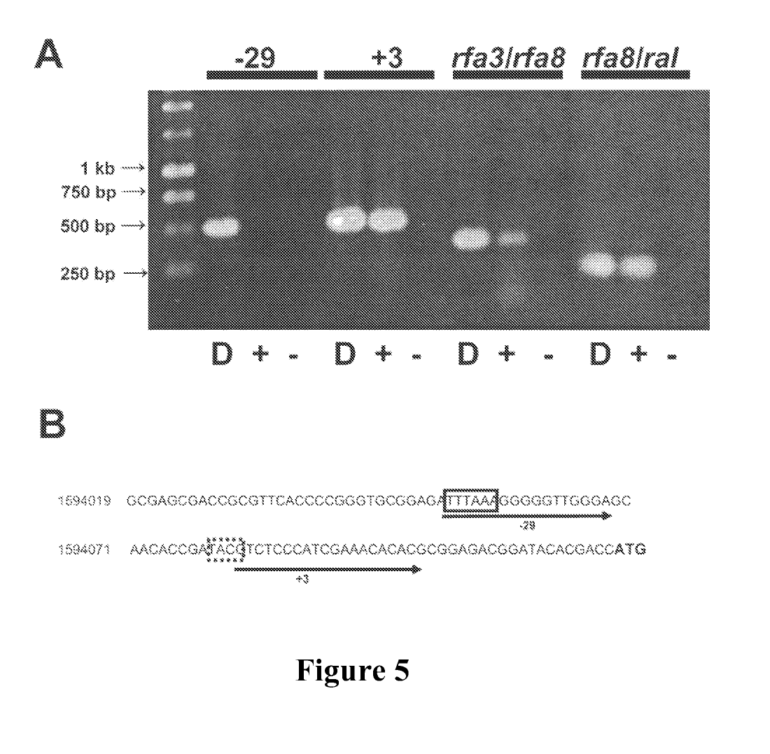

To gain further insight into the transcriptional organization and regulation of the rfa3, rfa8 and ral gene region, RT-PCR analysis was used first to determine whether rfa3 and rfa8 and rfa8 and ral are transcriptionally linked and likely form an operon. Primer pairs flanking the two intergenic regions produced amplification products (FIG. 5A), confirming that transcription proceeds from rfa3 through rfa8 to ral, resulting in a polycistronic transcript. To determine the transcriptional start site, two primer pairs were designed, with one set (−29F and R) having one end 29 by upstream of the predicted transcription start point and the other within the coding region of the gene, while the second set (+3F and R) had one end 3 bp within the predicted transcription start point and the other within the coding region of the gene (see FIG. 5B for the upstream primer positions). The results of RT-PCR showed that the second set (+3F and R) produced a PCR product, whereas the first set did not (−29F and R) (FIG. 5A). These results are consistent with the location of the transcription start point at ˜40 bp[IAC1] upstream of the ATG start codon. Although this codon corresponds to the second ATG in the predicted rfa3 ORF from genome annotation (13), subsequent alignment of rfa3 orthologs (data not shown) in other archaea showed it to be consistent with the translation start. A classical TATA-box motif was found centered about 25 by upstream of the putative transcriptional start site mapped (FIG. 5B).

### Discussion

The ability of the model haloarchaeon Halobacterium sp. NRC-1 and its mutants to tolerate ionizing radiation was addressed and the mechanism of the increased radiation resistance of two derivatives, LH5 and LH7a, using DNA microarrays was also addressed. The results discussed herein indicate that these organisms are extremely resistant to ionizing radiation, most likely reflecting their natural environment, where frequent exposure to intense solar radiation and prolonged exposure to desiccation would have similar DNA-damaging effects. Although the degree of resistance of the wild-type Halobacterium sp. NRC-1 is slightly lower than that of some of the most radiation-resistant microorganisms known, such as D. radiodurans, its LH5 and LH7a mutant derivatives are even more highly resistant than any known natural isolates, including the thermophilic species Rubrobacter radiotoleran (32). The mechanism of acquisition of increased radiation resistance was apparent from transcriptomic analysis of the LH5 and LH7a mutants compared to the NRC-1 wild-type parent. Coincidence of induction of the rfa3 operon in two independent radiation-resistant mutants strongly suggested that the single-stranded DNA-binding protein RPA is critical to the phenotype. The naturally high radiation resistance of Halobacterium may reflect the high salinity of the growth medium. The standard growth medium for D. radiodurans lacks the protective salt concentrations necessary for growth of the halophile Halobacterium (33). Comparisons in identical medium were impossible, because the two microorganisms cannot coexist in a single medium. Interestingly, many of the high-dose survivors of the Halobacterium isolates displayed a significant delay in growth (not shown) and required extended incubation times to reach macroscopic colony formation size. Upon re-isolation, however, these survivors formed colonies at the same rate as un-irradiated controls, and there was no evidence of long-term post-irradiation growth delay. This phenomenon was not observed for the irradiated D. radiodurans cultures.

Microorganisms generally display increased sensitivity to radiation when actively growing. The lower level of resistance during exponential phase has been attributed to saturation of the repair machinery during rapid growth (29). Although most microbial species tested, including D. radiodurans, have been reported to show higher resistance during stationary phase, there are examples of the reverse situation (30, 31). Recently, Halobacterium NRC-1 has been reported to show maximum resistance to γ radiation when in the exponential phase (20), however, the same effect was not shown as in the present invention wherein the increase in transcripts of genes rfa3, rfa8 and ral was unexpectedly high.

Although reduction in growth rate has been shown to increase survival of D. radiodurans after irradiation (29, 30), this explanation was ruled out for the increased radiation resistance of strains LH5 and LH7a by verifying doubling times. Moreover, as shown, greater resistance is observed in exponentially growing Halobacterium cells, both wildtype and mutants, than in stationary-phase cells.

The LD₅₀ for stationary-phase Halobacterium NRC-1 (2.9 kGy) was slightly lower than that estimated from the data of Kotteman et al. (20) who used a ⁶⁰Co γ-radiation source (approximately 3.3 kGy). This may reflect the differences in radiation types (electron beam and γ rays), dose rates (30 Gy/s and 121 Gy/s), radiation delivery (pulsed and continuous), uncertainty in dosimetry, and culture conditions (such as the oxygenation state of the saturated cultures).

Comparison of the transcriptome of wild-type strain NRC-1 to the LH5 and LH7a mutants using DNA microarrays and further characterization by RT-PCR showed that an operon containing three genes, rfa3, rfa8 and ral, was up-regulated in both mutant strains. The predicted proteins encoded by the rfa3 and rfa8 genes (34 kDa and 21 kDa) are homologs to the family of single-strand DNA-binding proteins (COG1599 and 3390). The encoded proteins may form, together with the ral gene product (15 kDa), the large, medium and small subunits of a protein complex similar to the eukaryotic single-strand DNA-binding protein RPA (which contains 70-, 32- and 14-kDa subunits) (34). Given the wide-ranging roles played by RPA in DNA repair (nucleotide excision, base excision, mismatch and doublestrand break repair), as well as in DNA replication and recombination, induction of these genes may be highly relevant to almost all aspects of DNA metabolism in our isolated mutants (35, 36).

The closely related archaeal and eukaryotic proteins are designated replication protein A (RPA) while the bacterial proteins are termed SSB (for single-strand DNA-binding protein) (35). The homologous SSB protein from D. radiodurans has recently been crystallized and its three-dimensional structure determined (37). This protein is present under nonstressed conditions at levels approximately tenfold higher than the homologous protein in E. coli. Exposure to ionizing radiation increases the level almost fourfold. This increase correlates with our observation of increased inherent levels of expression from the rfa3 operon in LH5 and LH7a and suggests a common mechanism for resistance to high levels of DNA damage through increased stabilization of exposed single-strand DNA. The genome of D. radiodurans has recently been shown to reassemble after radiation damage through a mechanism involving DNA replication and homologous recombination, processes that generate extensive regions of single-strand DNA requiring protection by SSB (38). Regulation of levels of these ubiquitous proteins may be a general survival mechanism in cells from diverse organisms.

Structural analyses have shown that all RPAs and SSBs contain at least one conserved nonspecific single-strand DNA-binding domain, called an oligonucleotide/oligosaccharide-binding (OB) fold (34-37). The Rfa3 protein of Halobacterium contains one OB fold (positioned at ⁸⁴VDITSI¹⁵⁷) and also one nucleic acid-binding Zn-finger motif (¹⁸⁹RCP-HGE²⁰⁹). The Rfa8 protein contains one OB fold (⁴⁸VFV-ITM¹³⁰), and the Ral protein contains a coiled-coil region (⁷⁴SKS-TDS¹¹¹). Orthologous genes (66-70% amino acid sequence identity) were found in the genomes of the related haloarchaea Haloarcula marismortui and Haloferax volcanii. Similar operons are also present in members of the euryarchaeal families Thermococcales and Methanosarcinales (39, 40). However, there is no significant sequence similarity of the small subunit proteins from members of the three families.

In a previous study, the rfa3 operon was also found to be induced after irradiation of the Halobacterium sp. NRC-1 wild-type strain with UVC light (12) but at a much lower level and rfa8 was not induced. This observation, combined with the striking up-regulation of this key protein reported here in two independently isolated mutant strains in the absence of inducing doses of radiation, indicates that increased transcript levels of the rfa3 operon are advantageous during repair of both non-ionizing and ionizing radiation-induced DNA damage in Halobacterium. The proteins encoded by this operon may therefore be involved in the recognition or resolution of a common damage repair intermediate.

The role of the RPA protein in archaea DNA repair is of significant interest. The present findings suggest that regulation of these proteins is important for DNA repair in archaea (41). However, previous work describing mutants with increased radiation resistance in E. coli and Salmonella did not show similar effects in these bacteria (22, 23). In Saccharomyces cerevisiae, of the numerous mutations involved in recombination, replication and repair, including many in the RPA genes, none has been shown to increase the inherent resistance in this organism. Given the similarity between the archaeal and eukaryotic DNA repair systems, this information is relevant to the understanding of human cell response to high doses of radiation, such as occurs during cancer therapy, and allow intervention strategies to be developed.

### REFERENCES

The references discussed herein are hereby incorporated by reference herein for all purposes.
1. S. DasSarma, Extreme halophiles are models for astrobiology. Microbe 1, 120-126 (2006).
2. S. McCready and L. Marcello, Repair of UV damage in Halobacterium salinarum. Biochem. Soc. Trans. 31, 694-698 (2003).
3. S. A. Fish, T. J. Shepherd, T. J. McGenity and W. D. Grant, Recovery of 16S ribosomal RNA gene fragments from ancient halite. Nature 417, 432-436 (2002).
4. R. L. Mancinelli, M. R. White and L. J. Rothschild, Biopan-survival I: exposure of the osmophiles synechococcus sp. (Nageli) and haloarcula sp. to the space environment. Adv. Space Res. 22, 327-334 (1998).
5. S. DasSarma, Genome sequence of an extremely halophilic archaeon. In Microbial Genomes (C. Fraser, T. Read and K. Nelson, Eds.), pp. 383-399. Humana Press, Totowa, N.J., 2004.
6. B. Berquist. J. Muller, P. DasSarma and S. DasSarma, Genetic systems for halophilic Archaea. In Methods in Microbiology (A. Oren and F. Rainey, Eds.), pp. 637-668. Elsevier/Academic Press, Dordrecht, 2005.
7. H. D. Shukla and S. DasSarma, Complexity of gas vesicle biogenesis in Halobacterium sp. strain NRC-1: Identification of five new proteins. J. Bacteriol. 186, 3182-3186 (2004).
8. N. S. Baliga, S. P. Kennedy, W. V. Ng, L. Hood and S. DasSarma, Genomic and genetic dissection of an archaeal regulon. Proc. Natl. Acad. Sci. USA 98, 2521-2525 (2001).
9. G. Wang, S. P. Kennedy, S. Fasiludeen, C. Rensing and S. DasSarma. Arsenic resistance in Halobacterium sp. strain NRC-1 examined by using an improved gene knockout system. J. Bacteriol. 186, 3187-3194 (2004).
10. J. A. Muller and S. DasSarma, Genomic analysis of anaerobic respiration in the archaeon Halobacterium sp. strain NRC-1: Dimethyl sulfoxide and trimethylamine N-oxide as terminal electron acceptors. J. Bacteriol. 187, 1659-1667 (2005).
11. B. R. Berquist and S. DasSarma, An archaeal chromosomal autonomously replicating sequence element from an extreme halophile, Halobacterium sp. strain NRC-1. J. Bacterial. 185, 5959-5966 (2003).
12. S. McCready, J. A. Muller, I. Boubriak, B. R. Berquist, W. L. Ng and S. DasSarma, UV irradiation induces homologous recombination genes in the model archaeon, Halobacterium sp. NRC-1. Saline Syst. 1, 3 (2005).
13. K. Whitehead, A. Kish, M. Pan, A. Kaur, D. J. Reiss, N. King, L. Hohmann, J. DiRuggiero and N. S. Baliga, An integrated systems approach for understanding cellular responses to gamma radiation. Mol. Syst. Biol. 2, 47 (2006).
14. W. V. Ng. S. P. Kennedy, G. G. Mahairas. B. Berquist. M. Pan, H. D. Shukla. S. R. Lasky, N. S. Baliga, V. Thorsson and S. Das-Sarma, Genome sequence of Halobacterium species NRC-1. Proc. Natl. Acad. Sci. USA 97, 12176-12181 (2000).
15. R. F. Peck, C. Echavarri-Erasun, E. A. Johnson, W. V. Ng, S. P. Kennedy, L. Hood, S. DasSarma and M. P. Krebs, brp and blh are required for synthesis of the retinal cofactor of bacteriorhodopsin in Halobacterium salinarum. J. Biol. Chem. 276, 5739-5744 (2001).
16. S. P. Kennedy, W. V. Ng, S. L. Salzberg, L. Hood and S. DasSarma, Understanding the adaptation of Halobacterium species NRC-1 to its extreme environment through computational analysis of its genome sequence. Genome Res. 11, 1641-1650 (2001).
17. M. F. White, Archaeal DNA repair: Paradigms and puzzles. Biochem. Soc. Trans. 31, 690-693 (2003).
18. D. J. Crowley, I. Boubriak, B. R. Berquist, M. Clark, E. Richard, L. Sullivan, S. DasSarma and S. McCready, The uvrA, uvrB and uvrC genes are required for repair of ultraviolet light induced DNA photoproducts in Halobacterium sp. NRC-1. Saline Syst. 2, 11 (2006).
19. N. S. Baliga, S. J. Bjork, R. Bonneau, M. Pan, C. Iloanusi, M. C. Kottemann, L. Hood and J. DiRuggiero, Systems level insights intothe stress response to UV radiation in the halophilic archaeon Halobacterium NRC-1. Genome Res. 14, 1025-1035 (2004).
20. M. Kottemann, A. Kish, C. Iloanusi, S. Bjork and J. DiRuggiero, Physiological responses of the halophilic archaeon Halobacterium sp. strain NRC1 to desiccation and gamma irradiation. Extremophiles 9, 219-227 (2005).
21. R. M. Quint, S. Solar and H. Stan-Lotter, Radiation sensitivity of N. pharaonis in comparison with E. coli K12 strains. Radiat. Environ. Biophys. 41, 145-148 (2002).
22. R. Davies and A. J. Sinskey, Radiation-resistant mutants of Salmonella typhimurium LT2: development and characterization. J. Bacterial. 113, 133-144 (1973).
23. D. Ewing, Production of radiation-resistant E. coli strains by daily X-irradiation. Int. J. Radiat. Biol. 71, 253-258 (1997).
24. S. DasSarma and E. Fleischmann, Halophiles. Cold Spring Harbor Laboratory Press, Plainview, N.Y., 1995.
25. A. M. Earl, S. K. Rankin, K. P. Kim, O. N. Lamendola and J. R. Battista, Genetic evidence that the uvsE gene product of Deinococcus radiodurans R1 is a UV damage endonuclease. J. Bacterial. 184, 1003-1009 (2002).
26. L. C. DeVeaux, D. P. Wells, A. Hunt, T. Webb, W. Beezhold and J. F. Harmon, Accelerator-based radiation sources for next-generation radiobiological research. Nucl. Instrum. Methods Phys. Res. A 562, 981-984 (2006).
27. W. Cleveland, Robust locally weighted regression and smoothing scatterplots. J. Am. Stat. Assoc. 74, 829-836 (1979).
28. M. M. Cox and J. R. Battista, Deinococcus radiodurans—the consummate survivor. Nat. Rev. Microbial. 3, 882-892 (2005).
29. M. L. Freedman and A. K. Bruce, The relationship of radioresistance to balanced growth-rate in Micrococcus radiodurans. Int. J. Radiat. Biol. Relat. Stud. Phys. Chem. Med. 19, 111-121 (1971).
30. L. C. Keller and R. B. Maxcy, Effect of physiological age on radiation resistance of some bacteria that are highly radiation resistant. Appl. Environ. Microbial. 47, 915-918 (1984).
31. J. W. Hastings, W. H. Holzapfel and J. G. Niemand, Radiation resistance of lactobacilli isolated from radurized meat relative to growth and environment. Appl. Environ. Microbial. 52, 898-901 (1986).
32. A. C. Ferreira, M. F. Nobre, E. Moore, F. A. Rainey, J. R. Battista and M. S. da Costa, Characterization and radiation resistance of new isolates of Rubrobacter radiotolerans and Rubrobacter xylanophilus. Extremophiles 3, 235-238 (1999).
33. A. M. Earl, M. M. Mohundro, I. S. Mian and J. R. Battista, The IrrE protein of Deinococcus radiodurans R1 is a novel regulator of recA expression. J. Bacterial. 184, 6216-6224 (2002).
34. Y. H. Chen, S. A. Kocherginskaya, Y. Lin, B. Sriratana, A. M. Lagunas, J. B. Robbins, R. I. Mackie and I. K. Cann, Biochemical and mutational analyses of a unique clamp loader complex in the archaeon Methanosarcina acetivorans. J. Biol. Chem. 280, 41852-41863 (2005).
35. F. Chedin, E. M. Seitz and S. C. Kowalczykowski, Novel homologs of replication protein A in archaea: implications for the evolution of ssDNA-binding proteins. Trends Biochem. Sci. 23, 273-277 (1998).
36. Y. Zou, Y. Liu, X. Wu and S. M. Shell, Functions of human replication protein A (RPA): from DNA replication to DNA damage and stress responses. J. Cell Physiol. 208, 267-273 (2006).
37. D. A. Bernstein, J. M. Eggington, M. P. Killoran, A. M. Misic, M. M. Cox and J. L. Keck, Crystal structure of the Deinococcus radiodurans single-stranded DNA-binding protein suggests a mechanism for coping with DNA damage. Proc. Natl. Acad. Sci. USA 101, 8575-8580 (2004).
38. K. Zahradka, D. Slade, A. Bailone, S. Sommer, D. Averbeck, M. Petranovic, A. B. Lindner and M. Radman, Reassembly of shattered chromosomes in Deinococcus radiodurans. Nature 443, 569-573 (2006).
39. K. Komori and Y. Ishino, Replication protein A in Pyrococcus furiosus is involved in homologous DNA recombination. J. Biol. Chem. 276, 25654-25660 (2001).
40. T. J. Kelly, P. Simancek and G. S. Brush, Identification and characterization of a single-stranded DNA-binding protein from the archaeon Methanococcus jannaschii. Proc. Natl. Acad. Sci. USA 95, 14634-14639 (1998).
41. L. Cubeddu and M. F. White, DNA damage detection by an archaeal single-stranded DNA-binding protein. J. Mol. Biol. 353, 507-516 (2005).

TABLE 1

| | Mean | Max/Min | # Data | Mean | |
|---|---|---|---|---|---|
| | Dose | Dose | points per | Fraction | |
| Strain tested^{a} | (kGy) | (kGy) | bin | surviving | Std. Error^{b} |
| Halobacterium NRC-1 (S) | 0^{c} | n/a | 5 | 1^{e} | n/a |
| | 2.8 | 2.8^{d} | 1 | 3.9E−1 | n/a |
| | 5.1 | 4.9, 5.6 | 3 | 1.7E−1 | 5.8E−2 |
| | 8.7 | 8.4, 9.1 | 2 | 9.1E−3 | 7.7E−3 |
| | 11 | 10.6, 11.3 | 3 | 6.2E−7 | 6.2E−7 |
| | 12 | 11.9, 11.9 | 2 | 2.0E−5 | 3.2E−6 |
| | 15 | 15.1, 15.3 | 4 | 2.0E−6 | 1.2E−6 |
| | 19 | 17.1, 19.7 | 6 | 2.6E−6 | 2.6E−6 |
| | 23 | 22.0, 23.3 | 4 | 0 | n/a^{f} |
| Halobacterium NRC-1 (E) | 0 | n/a | 10 | 1 | n/a |
| | 2.8 | 2.7, 2.9 | 4 | 9.2E−1 | 3.5E−2 |
| | 5.4 | 5.3, 5.6 | 4 | 4.4E−1 | 8.1E−2 |
| | 8.7 | 8.0, 9.1 | 10 | 1.2E−1 | 3.1E−2 |
| | 10 | 10.1, 10.5 | 2 | 5.5E−5 | 4.4E−5 |
| | 12 | 11.3, 12.1 | 8 | 2.4E−4 | 1.2E−4 |
| | 15 | 14.8, 15.5 | 4 | 6.6E−7 | 6.6E−7 |
| | 17 | 16.4, 17.6 | 5 | 9.2E−8 | 9.2E−8 |
| | 20 | 19.0, 20.3 | 6 | 0 | n/a |
| | 23 | 22.0, 23.0 | 3 | 0 | n/a |
| Halobacterium LH5 (S) | 0 | n/a | 5 | 1 | n/a |
| | 1.5 | 1.0, 1.9 | 2 | 9.7E−1 | 2.0E−2 |
| | 3.4 | 2.8, 4.0 | 2 | 8.0E−1 | 4.4E−2 |
| | 5.3 | 4.9, 5.9 | 4 | 5.9E−1 | 8.0E−2 |
| | 9.1 | 8.4, 9.9 | 3 | 3.2E−1 | 6.6E−2 |
| | 12 | 10.6, 13.1 | 5 | 1.5E−1 | 3.2E−2 |
| | 15 | 15.1, 16.0 | 4 | 2.3E−2 | 1.0E−2 |
| | 19 | 17.1, 19.7 | 5 | 1.2E−3 | 1.0E−3 |
| | 23 | 22.0, 23.3 | 4 | 5.0E−6 | 3.4E−6 |
| Halobacterium LH5 (E) | 0 | n/a | 5 | 1 | n/a |
| | 2.9 | 2.8, 3.0 | 2 | 9.7E−1 | 1.1E−1 |
| | 5.7 | 5.6, 5.7 | 2 | 1.0 | 2.0E−2 |
| | 8.8 | 8.4, 9.1 | 5 | 9.3E−1 | 1.5E−1 |
| | 12 | 11.3, 12.1 | 4 | 5.0E−1 | 1.0E−1 |
| | 15 | 14.4, 15.1 | 3 | 1.9E−1 | 6.9E−2 |
| | 17 | 16.7, 17.6 | 3 | 9.0E−3 | 5.4E−3 |
| | 20 | 18.8, 20.5 | 3 | 1.2E−4 | 6.5E−5 |
| | 22 | 21.5, 23.3 | 3 | 2.1E−4 | 1.8E−4 |
| Halobacterium LH7a (S) | 0 | n/a | 6 | 1 | n/a |
| | 1.7 | 1.0, 1.9 | 5 | 9.2E−1 | 3.6E−2 |
| | 3.9 | 3.9, 4.0 | 4 | 8.5E−1 | 6.5E−2 |
| | 5.5 | 4.9, 5.9 | 6 | 7.6E−1 | 4.8E−2 |
| | 8.3 | 8.3, 8.3 | 3 | 6.6E−1 | 9.3E−2 |
| | 10 | 10.5, 10.6 | 6 | 3.4E−1 | 4.1E−2 |
| | 12 | 11.5, 11.5 | 3 | 2.0E−1 | 3.1E−2 |
| | 14 | 13.1, 13.8 | 4 | 8.8E−2 | 3.4E−2 |
| | 16 | 15.3, 16.0 | 6 | 2.1E−2 | 8.3E−3 |
| | 18 | 17.7, 17.8 | 6 | 9.6E−3 | 1.5E−3 |
| | 20 | 19.3, 20.1 | 6 | 5.5E−4 | 3.6E−4 |
| | 23 | 22.0, 23.3 | 6 | 6.9E−6 | 3.5E−6 |
| | 30 | 30.2, 30.2 | 3 | 1.5E−8 | 8.5E−9 |
| | 36 | 35.9, 35.9 | 3 | 0 | n/a |
| Halobacterium LH7a (E) | 0 | n/a | 4 | 1 | n/a |
| | 1.9 | 1.9, 1.9 | 3 | 1.3 | 1.6E−2 |
| | 3.9 | 3.9, 3.9 | 3 | 1.1 | 6.0E−2 |
| | 5.7 | 5.7, 5.7 | 3 | 9.3E−1 | 1.1E−1 |
| | 8.5 | 8.3, 9.0 | 4 | 8.2E−1 | 1.0E−1 |
| | 11 | 10.5, 10.5 | 3 | 7.5E−1 | 5.9E−2 |
| | 12 | 11.5, 11.5 | 3 | 6.2E−1 | 1.9E−2 |
| | 14 | 13.8, 13.8 | 3 | 2.3E−1 | 3.7E−2 |
| | 16 | 14.8, 15.8 | 4 | 1.4E−1 | 5.2E−2 |
| | 18 | 17.6, 17.8 | 7 | 3.9E−3 | 2.0E−3 |
| | 20 | 20.1, 20.5 | 4 | 3.6E−5 | 3.6E−5 |
| | 23 | 22.8, 23.3 | 4 | 1.0E−7 | 1.0E−7 |
| | 30 | 30.2, 30.2 | 3 | 0 | n/a |
| | 36 | 35.9, 35.9 | 3 | 0 | n/a |
| D. radiodurans R1 (E) | 0 | n/a | 10 | 1 | n/a |
| | 3.2 | 2.8, 3.6 | 6 | 9.1E−1 | 7.3E−2 |
| | 5.8 | 5.7, 6.0 | 8 | 7.6E−1 | 6.1E−2 |
| | 7.8 | 7.8, 7.8 | 2 | 8.2E−1 | 2.0E−3 |
| | 9.2 | 9.1, 9.5 | 6 | 2.5E−1 | 7.0E−2 |
| | 12 | 11.9, 12.2 | 8 | 2.0E−2 | 1.1E−2 |
| | 14 | 14.1, 14.4 | 4 | 8.3E−5 | 4.8E−5 |
| | 15 | 14.8, 15.3 | 6 | 4.4E−4 | 2.6E−4 |
| | 29 | 29.0, 29.0 | 2 | 3.2E−6 | 3.2E−6 |
| D. radiodurans R1 (S) | 0 | n/a | 10 | 1 | n/a |
| | 3.0 | 2.8, 3.1 | 3 | 9.2E−1 | 7.0E−2 |
| | 3.6 | 3.6, 3.6 | 2 | 9.7E−1 | 8.5E−3 |
| | 5.9 | 5.7, 6.0 | 6 | 6.3E−1 | 9.5E−2 |
| | 7.8 | 7.8, 7.8 | 2 | 7.0E−1 | 3.8E−2 |
| | 9.5 | 9.4, 9.5 | 2 | 4.8E−1 | 5.5E−2 |
| | 12 | 12.0, 12.2 | 4 | 5.8E−2 | 3.7E−2 |
| | 15 | 14.1, 15.8 | 11 | 1.0E−2 | 3.1E−3 |
| | 20 | 18.8, 20.8 | 9 | 1.5E−4 | 9.6E−5 |
| | 24 | 24.2, 24.2 | 2 | 0 | n/a |
| | 30 | 29.0, 30.8 | 3 | 0 | n/a |

^{a}Each strain was tested in both the exponential phase (E) and stationary phase (S) of growth.
^{b}Standard error was the error on the mean. By definition, there is no error on the mock-irradiated value of 1. For the single bin with one data point, there is no error.
^{c}Target dose of 0 represents mock-irradiated cells. Max/min is not applicable.
^{d}There was only one data point at this dose.
^{e}Survival fraction of mock-irradiated cells is the mean of all mock-irradiated data points divided by this mean, which is, by definition, unity.
^{f}The standard error formula is not applicable to multiple measurements of 0.

TABLE 2

| | | LH5 vs. NRC-1 | | | LH7a vs. NRC-1 | | | |
|---|---|---|---|---|---|---|---|---|
| Gene | Gene | log | fold | | | fold | | |
| id | Name | ratio | change | p value | log ratio | change | p value | functional category |
| 1 | vng1 | −0.044 | 0.904 | 0.326478 | 0.077 | 1.195 | 0.150633 | unknown |
| 2 | yvrO | −0.081 | 0.829 | 0.294532 | 0.062 | 1.153 | 0.32265 | transport |
| 3 | vng3 | −0.064 | 0.864 | 0.361413 | 0.021 | 1.051 | 0.705 | cellular process |
| 5 | vng5 | 0.015 | 1.036 | 0.566339 | −0.005 | 0.989 | 0.713333 | unknown |
| 6 | glmS | −0.022 | 0.951 | 0.430965 | 0.034 | 1.080 | 0.566917 | amino acid metabolism |
| 8 | graD5 | −0.024 | 0.947 | 0.461229 | 0.052 | 1.127 | 0.332592 | nucleotide metabolism |
| 9 | graD2 | −0.016 | 0.964 | 0.514761 | 0.036 | 1.088 | 0.7115 | nucleotide metabolism |
| 11 | hsx | −0.093 | 0.808 | 0.193372 | 0.060 | 1.148 | 0.305833 | general function prediction only |
| 13 | vng13 | 0.089 | 1.227 | 0.341297 | 0.056 | 1.138 | 0.381893 | DNA metabolism |
| 14 | vng14 | −0.019 | 0.958 | 0.660281 | 0.022 | 1.053 | 0.875833 | amino acid metabolism |
| 15 | vng15 | −0.080 | 0.832 | 0.663868 | −0.009 | 0.979 | 0.86225 | unknown |
| 16 | vng16 | −0.034 | 0.924 | 0.557449 | −0.041 | 0.909 | 0.438333 | unknown |
| 17 | vng17 | −0.043 | 0.906 | 0.538786 | 0.061 | 1.150 | 0.637917 | unknown |
| 18 | vng18 | 0.125 | 1.334 | 0.081921 | 0.009 | 1.022 | 0.50905 | unknown |
| 20 | vng20 | −0.014 | 0.968 | 0.55031 | 0.022 | 1.053 | 0.93 | unknown |
| 21 | vng21 | −0.089 | 0.815 | 0.204453 | −0.040 | 0.912 | 0.78575 | DNA metabolism |
| 22 | vng22 | −0.027 | 0.939 | 0.380721 | −0.016 | 0.964 | 0.690917 | unknown |
| 23 | vng23 | 0.062 | 1.152 | 0.426539 | 0.002 | 1.005 | 0.766167 | unknown |
| 24 | vng24 | 0.055 | 1.136 | 0.237963 | 0.024 | 1.058 | 0.546733 | unknown |
| 25 | vng25 | −0.137 | 0.729 | 0.104324 | 0.062 | 1.154 | 0.378667 | unknown |
| 26 | vng26 | 0.002 | 1.004 | 0.229905 | 0.038 | 1.091 | 0.534 | DNA metabolism |
| 27 | vng27 | −0.134 | 0.734 | 0.404226 | 0.038 | 1.091 | 0.536317 | unknown |
| 28 | vng28 | −0.012 | 0.972 | 0.702273 | 0.056 | 1.138 | 0.369363 | DNA metabolism |
| 29 | vng29 | −0.020 | 0.956 | 0.310077 | 0.048 | 1.118 | 0.53475 | unknown |
| 30 | vng30 | 0.033 | 1.078 | 0.421542 | 0.031 | 1.075 | 0.60525 | unknown |
| 31 | vng31 | 0.000 | 1.001 | 0.360254 | −0.044 | 0.903 | 0.454167 | unknown |
| 32 | vng32 | 0.046 | 1.111 | 0.512991 | 0.011 | 1.026 | 0.7125 | unknown |
| 33 | vng33 | 0.029 | 1.068 | 0.229254 | 0.005 | 1.012 | 0.812333 | unknown |
| 34 | vng34 | 0.015 | 1.036 | 0.888032 | −0.010 | 0.976 | 0.682 | unknown |
| 35 | vng35 | −0.030 | 0.933 | 0.605471 | 0.022 | 1.053 | 0.755417 | DNA metabolism |
| 37 | vng37 | 0.065 | 1.162 | 0.498761 | −0.072 | 0.847 | 0.152 | unknown |
| 38 | vng38 | 0.009 | 1.021 | 0.596209 | 0.006 | 1.015 | 0.494 | unknown |
| 39 | arlR1 | −0.151 | 0.707 | 0.148702 | 0.029 | 1.068 | 0.685667 | transcription and regulation |
| 40 | ptr1 | −0.029 | 0.936 | 0.476658 | 0.004 | 1.009 | 0.466917 | transcription and regulation |
| 41 | vng41 | −0.085 | 0.823 | 0.144353 | −0.005 | 0.988 | 0.789167 | unknown |
| 42 | tnp1 | 0.058 | 1.142 | 0.42401 | −0.056 | 0.879 | 0.116863 | DNA metabolism |
| 43 | vng43 | −0.205 | 0.624 | 0.165622 | 0.010 | 1.024 | 0.59775 | DNA metabolism |
| 44 | vng44 | −0.071 | 0.849 | 0.254016 | 0.004 | 1.010 | 0.372167 | DNA metabolism |
| 45 | vng45 | −0.008 | 0.982 | 0.524912 | 0.010 | 1.023 | 0.703167 | DNA metabolism |
| 46 | ugd | 0.036 | 1.086 | 0.551393 | −0.015 | 0.967 | 0.828333 | nucleotide metabolism |
| 47 | graD6 | −0.011 | 0.975 | 0.424789 | 0.054 | 1.131 | 0.341967 | nucleotide metabolism |
| 50 | vng50 | −0.187 | 0.650 | 0.110524 | −0.025 | 0.944 | 0.48215 | unknown |
| 51 | rfbU1 | 0.021 | 1.049 | 0.330056 | −0.017 | 0.962 | 0.621 | translation |
| 53 | vng53 | −0.088 | 0.816 | 0.377444 | −0.010 | 0.977 | 0.865 | unknown |
| 54 | vng54 | 0.035 | 1.085 | 0.439189 | 0.050 | 1.122 | 0.456333 | unknown |
| 55 | vng55 | 0.000 | 1.000 | 1 | −0.262 | 0.547 | 0.781 | unknown |
| 56 | vng56 | −0.135 | 0.733 | 0.236267 | 0.046 | 1.112 | 0.668167 | DNA metabolism |
| 57 | vng57 | 0.006 | 1.013 | 0.411551 | −0.053 | 0.886 | 0.462667 | unknown |
| 58 | vng58 | −0.127 | 0.746 | 0.163981 | −0.149 | 0.710 | 0.013973 | unknown |
| 60 | lpg | −0.027 | 0.940 | 0.675291 | −0.063 | 0.866 | 0.275417 | cellular process |
| 61 | vng61 | −0.019 | 0.957 | 0.549027 | −0.036 | 0.921 | 0.523333 | unknown |
| 62 | lpb | −0.074 | 0.844 | 0.310097 | 0.010 | 1.024 | 0.480333 | cellular process |
| 63 | galE2 | −0.047 | 0.898 | 0.476802 | −0.016 | 0.965 | 0.612 | carbohydrate and lipid metabolism |
| 64 | graD3 | 0.026 | 1.063 | 0.324249 | 0.029 | 1.070 | 0.72725 | nucleotide metabolism |
| 65 | gmd | 0.042 | 1.102 | 0.352229 | −0.023 | 0.948 | 0.553167 | carbohydrate and lipid metabolism |
| 66 | vng66 | 0.011 | 1.027 | 0.187195 | −0.031 | 0.932 | 0.580417 | unknown |
| 67 | vng67 | −0.024 | 0.946 | 0.732971 | −0.053 | 0.886 | 0.457 | unknown |
| 68 | vng68 | 0.121 | 1.322 | 0.075759 | −0.015 | 0.966 | 0.891 | unknown |
| 69 | vng69 | −0.066 | 0.858 | 0.338643 | −0.082 | 0.829 | 0.107725 | unknown |
| 70 | vng70 | −0.052 | 0.886 | 0.484482 | −0.011 | 0.975 | 0.4505 | unknown |
| 72 | nbp1 | −0.022 | 0.950 | 0.652188 | −0.037 | 0.919 | 0.593667 | transcription and regulation |
| 73 | vng73 | 0.066 | 1.164 | 0.241595 | 0.016 | 1.038 | 0.836 | unknown |
| 75 | vng75 | −0.107 | 0.782 | 0.348958 | 0.012 | 1.029 | 0.8435 | unknown |
| 76 | vng76 | 0.098 | 1.252 | 0.309096 | −0.027 | 0.939 | 0.7525 | unknown |
| 77 | vng77 | −0.030 | 0.934 | 0.504992 | −0.072 | 0.848 | 0.245258 | unknown |
| 79 | vng79 | −0.030 | 0.934 | 0.597437 | 0.053 | 1.129 | 0.6675 | unknown |
| 80 | vng80 | 0.035 | 1.084 | 0.422627 | −0.065 | 0.862 | 0.509 | unknown |
| 81 | moaE | 0.001 | 1.002 | 0.639423 | −0.023 | 0.949 | 0.327633 | cofactor and secondary |
| | | | | | | | | metabolites |
| 84 | moeB | 0.071 | 1.176 | 0.354562 | −0.009 | 0.981 | 0.688417 | cofactor and secondary |
| | | | | | | | | metabolites |
| 85 | moaA | −0.124 | 0.751 | 0.376908 | −0.047 | 0.897 | 0.5349 | cofactor and secondary |
| | | | | | | | | metabolites |
| 86 | moeA2 | −0.002 | 0.995 | 0.750474 | 0.005 | 1.012 | 0.532667 | cofactor and secondary |
| | | | | | | | | metabolites |
| 89 | moeA1 | −0.080 | 0.832 | 0.230508 | −0.057 | 0.877 | 0.30922 | cofactor and secondary |
| | | | | | | | | metabolites |
| 90 | pimT1 | −0.035 | 0.922 | 0.312918 | −0.009 | 0.978 | 0.358817 | cellular process |
| 91 | vng91 | 0.024 | 1.058 | 0.618553 | 0.055 | 1.136 | 0.308 | unknown |
| 94 | vng94 | 0.065 | 1.162 | 0.422203 | −0.006 | 0.986 | 0.81725 | unknown |
| 95 | gapB | 0.444 | 2.781 | 4.68E−07 | −0.063 | 0.866 | 0.293938 | carbohydrate and lipid metabolism |
| 96 | vng96 | 0.030 | 1.072 | 0.347588 | −0.048 | 0.896 | 0.426625 | amino acid metabolism |
| 97 | hsp2 | 0.052 | 1.127 | 0.29275 | −0.084 | 0.824 | 0.1348 | cellular process |
| 98 | rimK | −0.015 | 0.967 | 0.669505 | 0.077 | 1.194 | 0.205475 | translation |
| 99 | rpl10e | 0.134 | 1.360 | 0.055751 | −0.045 | 0.901 | 0.405217 | translation |
| 101 | cspD1 | 0.111 | 1.292 | 0.496509 | 0.021 | 1.050 | 0.6785 | cellular process |
| 102 | vng102 | 0.006 | 1.014 | 0.54495 | −0.041 | 0.910 | 0.5188 | unknown |
| 104 | serA3 | −0.014 | 0.969 | 0.788012 | 0.052 | 1.128 | 0.3595 | amino acid metabolism |
| 105 | vng105 | −0.065 | 0.861 | 0.477894 | −0.081 | 0.829 | 0.524233 | unknown |
| 106 | rmeM | 0.037 | 1.088 | 0.175875 | 0.012 | 1.028 | 0.756333 | DNA metabolism |
| 107 | rmeS | −0.212 | 0.614 | 0.044526 | −0.046 | 0.899 | 0.384167 | DNA metabolism |
| 108 | rmeR | −0.185 | 0.653 | 0.098988 | 0.001 | 1.002 | 0.724167 | DNA metabolism |
| 110 | vng110 | −0.069 | 0.854 | 0.215352 | −0.043 | 0.905 | 0.613833 | unknown |
| 112 | vng112 | −0.073 | 0.846 | 0.248892 | −0.100 | 0.795 | 0.275925 | unknown |
| 113 | bolR1 | 0.096 | 1.246 | 0.452711 | −0.025 | 0.944 | 0.723667 | transcription and regulation |
| 115 | yusZ1 | 0.075 | 1.187 | 0.329874 | −0.116 | 0.765 | 0.040887 | general function prediction only |
| 116 | vng116 | −0.052 | 0.887 | 0.438563 | 0.043 | 1.103 | 0.645583 | unknown |
| 117 | vng117 | −0.070 | 0.852 | 0.425075 | 0.043 | 1.103 | 0.4863 | unknown |
| 118 | vng118 | −0.036 | 0.921 | 0.474246 | 0.032 | 1.078 | 0.475008 | unknown |
| 119 | vng119 | 0.026 | 1.061 | 0.587989 | 0.051 | 1.126 | 0.39525 | unknown |
| 120 | vng120 | 0.112 | 1.293 | 0.08796 | 0.076 | 1.190 | 0.18045 | unknown |
| 121 | vng121 | 0.027 | 1.063 | 0.838606 | 0.020 | 1.048 | 0.893833 | unknown |
| 123 | trp1 | 0.025 | 1.059 | 0.210146 | −0.046 | 0.900 | 0.553494 | transport |
| 124 | vng124 | −0.023 | 0.949 | 0.536382 | 0.007 | 1.016 | 0.9155 | unknown |
| 125 | vng125 | −0.024 | 0.946 | 0.362433 | 0.008 | 1.019 | 0.865 | unknown |
| 127 | vng127 | 0.070 | 1.174 | 0.251614 | −0.042 | 0.908 | 0.5225 | unknown |
| 128 | rfr | 0.042 | 1.101 | 0.515686 | −0.037 | 0.919 | 0.501667 | general function prediction only |
| 129 | hsp4 | 0.012 | 1.028 | 0.392245 | 0.014 | 1.032 | 0.71925 | cellular process |
| 130 | vng130 | −0.100 | 0.794 | 0.388444 | −0.021 | 0.953 | 0.72775 | unknown |
| 132 | vng132 | 0.047 | 1.115 | 0.40274 | 0.025 | 1.060 | 0.612 | unknown |
| 133 | rfa1 | −0.039 | 0.914 | 0.38935 | 0.029 | 1.070 | 0.382125 | DNA metabolism |
| 134 | hpyA | −0.099 | 0.797 | 0.28588 | −0.047 | 0.896 | 0.445583 | DNA metabolism |
| 136 | hdf | −0.004 | 0.990 | 0.383891 | −0.010 | 0.977 | 0.759 | cellular process |
| 137 | cca | −0.038 | 0.917 | 0.513241 | 0.062 | 1.154 | 0.251783 | translation |
| 138 | vng138 | 0.054 | 1.132 | 0.510784 | −0.008 | 0.981 | 0.79025 | unknown |
| 139 | vng139 | −0.016 | 0.965 | 0.811185 | 0.050 | 1.121 | 0.4435 | unknown |
| 140 | vng140 | 0.026 | 1.061 | 0.391067 | 0.059 | 1.146 | 0.334167 | unknown |
| 141 | vng141 | −0.001 | 0.997 | 0.351493 | 0.005 | 1.012 | 0.415067 | unknown |
| 142 | arlR2 | 0.054 | 1.131 | 0.028005 | 0.053 | 1.131 | 0.437253 | transcription and regulation |
| 143 | vng143 | −0.051 | 0.888 | 0.564655 | 0.001 | 1.003 | 0.45165 | unknown |
| 144 | vng144 | 0.191 | 1.551 | 0.166972 | −0.037 | 0.919 | 0.58775 | unknown |
| 146 | vng146 | 0.199 | 1.580 | 0.083652 | −0.207 | 0.622 | 0.002927 | unknown |
| 147 | hmr | −0.068 | 0.854 | 0.362329 | 0.010 | 1.023 | 0.682083 | transcription and regulation |
| 148 | vng148 | 0.136 | 1.368 | 0.180353 | −0.062 | 0.868 | 0.3375 | unknown |
| 149 | zntA | −0.075 | 0.841 | 0.056399 | −0.070 | 0.850 | 0.275583 | transport |
| 150 | vng150 | −0.027 | 0.940 | 0.577426 | 0.004 | 1.010 | 0.675542 | unknown |
| 151 | vng151 | −0.021 | 0.953 | 0.674698 | 0.062 | 1.152 | 0.237 | unknown |
| 152 | prrC | −0.024 | 0.947 | 0.593101 | 0.004 | 1.010 | 0.79725 | transcription and regulation |
| 153 | vng153 | 0.167 | 1.470 | 0.157761 | −0.012 | 0.972 | 0.78 | unknown |
| 154 | merA | −0.012 | 0.973 | 0.563188 | 0.007 | 1.015 | 0.692417 | cellular process |
| 156 | bolR2 | −0.138 | 0.728 | 0.648671 | 0.010 | 1.022 | 0.85075 | transcription and regulation |
| 157 | oxlT | 0.002 | 1.004 | 0.372859 | 0.030 | 1.072 | 0.745333 | transport |
| 158 | kdgK | 0.078 | 1.197 | 0.217223 | 0.025 | 1.060 | 0.6405 | carbohydrate and lipid metabolism |
| 159 | mutL | −0.036 | 0.920 | 0.514836 | 0.000 | 0.999 | 0.538667 | DNA metabolism |
| 160 | boa1 | −0.022 | 0.950 | 0.44896 | −0.003 | 0.993 | 0.849333 | transcription and regulation |
| 161 | gdhB | −0.009 | 0.980 | 0.343149 | −0.199 | 0.632 | 0.012428 | amino acid metabolism |
| 162 | alkK | −0.286 | 0.518 | 0.000248 | −0.133 | 0.736 | 0.028662 | carbohydrate and lipid metabolism |
| 163 | mutS1a | −0.121 | 0.757 | 0.246969 | 0.023 | 1.055 | 0.6125 | DNA metabolism |
| 164 | hef | −0.017 | 0.963 | 0.695047 | 0.037 | 1.089 | 0.593567 | cellular process |
| 166 | psmB | 0.227 | 1.686 | 0.00103 | −0.011 | 0.976 | 0.825167 | cellular process |
| 167 | vng167 | 0.051 | 1.124 | 0.207542 | −0.136 | 0.731 | 0.008588 | unknown |
| 168 | vng168 | −0.165 | 0.684 | 0.204453 | −0.068 | 0.855 | 0.32125 | unknown |
| 170 | vng170 | 0.097 | 1.251 | 0.364998 | 0.040 | 1.097 | 0.7435 | cofactor and secondary |
| | | | | | | | | metabolites |
| 171 | vng171 | 0.075 | 1.188 | 0.851924 | −0.015 | 0.966 | 0.89 | DNA metabolism |
| 172 | mutS1b | −0.001 | 0.999 | 0.65583 | 0.006 | 1.013 | 0.781167 | DNA metabolism |
| 174 | cat1 | −0.084 | 0.825 | 0.391066 | 0.006 | 1.013 | 0.717167 | transport |
| 175 | trkA1 | 0.086 | 1.218 | 0.151163 | −0.019 | 0.958 | 0.7175 | transport |
| 176 | act1 | −0.030 | 0.934 | 0.530723 | 0.028 | 1.066 | 0.474833 | transcription and regulation |
| 177 | rpl15e | −0.017 | 0.961 | 0.571833 | 0.071 | 1.179 | 0.317433 | translation |
| 178 | vng178 | −0.039 | 0.914 | 0.517479 | 0.019 | 1.046 | 0.47475 | unknown |
| 179 | stk2 | −0.067 | 0.857 | 0.285492 | −0.050 | 0.891 | 0.513523 | cellular process |
| 180 | hop | −0.002 | 0.996 | 0.54996 | 0.037 | 1.090 | 0.5898 | cellular process |
| 181 | lpl | −0.012 | 0.972 | 0.443423 | −0.058 | 0.874 | 0.527417 | cofactor and secondary |
| | | | | | | | | metabolites |
| 183 | xthA | 0.037 | 1.089 | 0.193463 | 0.018 | 1.041 | 0.569625 | DNA metabolism |
| 184 | vng184 | 0.058 | 1.142 | 0.414216 | 0.017 | 1.041 | 0.465417 | cofactor and secondary |
| | | | | | | | | metabolites |
| 186 | pepB1 | 0.044 | 1.107 | 0.342953 | −0.019 | 0.957 | 0.719667 | amino acid metabolism |
| 187 | vng187 | −0.021 | 0.953 | 0.74123 | 0.020 | 1.046 | 0.9125 | unknown |
| 188 | vng188 | 0.045 | 1.109 | 0.436291 | −0.030 | 0.933 | 0.807333 | carbohydrate and lipid metabolism |
| 189 | vng189 | 0.077 | 1.195 | 0.432247 | 0.013 | 1.030 | 0.6215 | unknown |
| 190 | vng190 | −0.087 | 0.818 | 0.11388 | −0.033 | 0.927 | 0.546583 | cellular process |
| 192 | ftsZ2 | 0.272 | 1.870 | 0.004604 | 0.024 | 1.056 | 0.515667 | cellular process |
| 194 | camR1 | 0.162 | 1.451 | 0.007993 | −0.060 | 0.870 | 0.278225 | transcription and regulation |
| 195 | vng195 | −0.017 | 0.961 | 0.484248 | −0.024 | 0.947 | 0.50675 | unknown |
| 196 | vng196 | −0.093 | 0.807 | 0.25834 | 0.052 | 1.127 | 0.497125 | unknown |
| 197 | vng197 | −0.096 | 0.802 | 0.466287 | −0.004 | 0.991 | 0.612 | unknown |
| 198 | vng198 | −0.019 | 0.957 | 0.701149 | 0.030 | 1.072 | 0.6755 | unknown |
| 199 | vng199 | −0.045 | 0.901 | 0.297535 | 0.027 | 1.063 | 0.64675 | unknown |
| 200 | vng200 | −0.058 | 0.875 | 0.692867 | −0.029 | 0.935 | 0.879667 | unknown |
| 203 | vng204 | −0.014 | 0.967 | 0.585967 | 0.008 | 1.019 | 0.8135 | unknown |
| 204 | vng203 | 0.003 | 1.006 | 0.40445 | 0.007 | 1.016 | 0.75075 | unknown |
| 205 | vng205 | −0.088 | 0.817 | 0.081727 | −0.405 | 0.394 | 9.87E−12 | unknown |
| 207 | vng207 | −0.024 | 0.947 | 0.631356 | 0.042 | 1.101 | 0.397 | unknown |
| 208 | vng208 | −0.152 | 0.704 | 0.003963 | 0.014 | 1.033 | 0.615917 | unknown |
| 209 | vng209 | −0.052 | 0.888 | 0.197705 | 0.090 | 1.229 | 0.259094 | DNA metabolism |
| 212 | vng212 | 0.019 | 1.044 | 0.745517 | −0.102 | 0.791 | 0.0601 | unknown |
| 213 | vng213 | 0.111 | 1.290 | 0.155914 | 0.098 | 1.252 | 0.26785 | unknown |
| 214 | vng214 | −0.073 | 0.846 | 0.236461 | 0.036 | 1.085 | 0.60475 | unknown |
| 215 | vng215 | −0.176 | 0.667 | 0.227618 | 0.008 | 1.019 | 0.6735 | unknown |
| 216 | vng216 | 0.016 | 1.038 | 0.789819 | 0.059 | 1.146 | 0.449 | unknown |
| 217 | vng217 | −0.127 | 0.746 | 0.050703 | −0.128 | 0.745 | 0.065025 | unknown |
| 218 | gspE1 | −0.069 | 0.853 | 0.370364 | −0.045 | 0.902 | 0.697225 | cellular process |
| 219 | vng219 | 0.045 | 1.110 | 0.425138 | −0.038 | 0.916 | 0.498 | unknown |
| 220 | vng220 | −0.076 | 0.839 | 0.266212 | −0.023 | 0.948 | 0.5318 | unknown |
| 222 | vng222 | 0.022 | 1.052 | 0.594932 | 0.006 | 1.014 | 0.71465 | unknown |
| 223 | moxR | 0.030 | 1.072 | 0.592606 | −0.021 | 0.954 | 0.562767 | cofactor and secondary |
| | | | | | | | | metabolites |
| 226 | htrA | −0.005 | 0.990 | 0.602046 | −0.010 | 0.977 | 0.822333 | cellular process |
| 227 | vng227 | −0.055 | 0.881 | 0.317551 | 0.051 | 1.125 | 0.4037 | unknown |
| 228 | guaAa | 0.013 | 1.029 | 0.345802 | −0.068 | 0.856 | 0.296875 | nucleotide metabolism |
| 230 | vng230 | 0.048 | 1.116 | 0.407908 | −0.008 | 0.982 | 0.651167 | unknown |
| 231 | vng231 | 0.061 | 1.151 | 0.617818 | −0.110 | 0.776 | 0.537 | unknown |
| 233 | vng233 | −0.145 | 0.716 | 0.17625 | −0.003 | 0.992 | 0.854625 | unknown |
| 234 | vng234 | 0.001 | 1.002 | 0.695085 | −0.029 | 0.936 | 0.21875 | cellular process |
| 237 | rpoP | −0.004 | 0.991 | 0.597038 | 0.013 | 1.031 | 0.7265 | transcription and regulation |
| 238 | vng238 | 0.170 | 1.480 | 0.001395 | 0.032 | 1.077 | 0.541 | unknown |
| 239 | vng239 | −0.014 | 0.969 | 0.426686 | −0.050 | 0.892 | 0.424942 | translation |
| 240 | vng240 | 0.001 | 1.003 | 0.567339 | 0.004 | 1.008 | 0.809167 | unknown |
| 243 | vng243 | 0.005 | 1.011 | 0.641892 | −0.044 | 0.904 | 0.494517 | unknown |
| 244 | vng244 | −0.007 | 0.985 | 0.869048 | 0.017 | 1.039 | 0.66725 | unknown |
| 245 | dtd | −0.075 | 0.841 | 0.302198 | 0.008 | 1.019 | 0.5225 | nucleotide metabolism |
| 247 | xrlR1 | 0.008 | 1.018 | 0.691131 | 0.011 | 1.025 | 0.853 | transcription and regulation |
| 248 | vng248 | 0.005 | 1.012 | 0.507417 | 0.006 | 1.014 | 0.7575 | unknown |
| 249 | hlp1 | −0.189 | 0.647 | 0.223379 | −0.032 | 0.929 | 0.623667 | energy metabolism |
| 250 | vng250 | −0.006 | 0.986 | 0.749851 | 0.019 | 1.046 | 0.50645 | unknown |
| 251 | vng251 | −0.046 | 0.899 | 0.535244 | −0.017 | 0.962 | 0.54375 | unknown |
| 252 | vng252 | −0.021 | 0.953 | 0.722025 | 0.018 | 1.043 | 0.478917 | unknown |
| 254 | tfbG | 0.155 | 1.428 | 0.256884 | 0.010 | 1.023 | 0.748917 | transcription and regulation |
| 255 | vng255 | 0.079 | 1.200 | 0.273682 | −0.023 | 0.948 | 0.59125 | DNA metabolism |
| 256 | vng256 | 0.054 | 1.131 | 0.422752 | −0.031 | 0.930 | 0.5475 | unknown |
| 258 | prlR1 | −0.079 | 0.833 | 0.12329 | 0.026 | 1.061 | 0.6035 | transcription and regulation |
| 259 | ipp | 0.085 | 1.216 | 0.322213 | −0.022 | 0.951 | 0.41025 | energy metabolism |
| 261 | vng261 | −0.129 | 0.742 | 0.139952 | 0.022 | 1.053 | 0.67275 | unknown |
| 262 | vng262 | −0.013 | 0.970 | 0.652189 | −0.037 | 0.917 | 0.488825 | unknown |
| 264 | vng264 | 0.121 | 1.320 | 0.073369 | −0.060 | 0.871 | 0.400537 | unknown |
| 265 | ftsZ4 | −0.096 | 0.802 | 0.10058 | 0.065 | 1.160 | 0.2794 | cellular process |
| 266 | vng266 | −0.039 | 0.914 | 0.577998 | −0.008 | 0.981 | 0.879375 | unknown |
| 267 | vng267 | 0.016 | 1.039 | 0.307238 | 0.004 | 1.010 | 0.81975 | unknown |
| 268 | vng268 | −0.036 | 0.921 | 0.461072 | −0.023 | 0.949 | 0.499667 | energy metabolism |
| 270 | vng270 | −0.005 | 0.988 | 0.406569 | −0.018 | 0.959 | 0.493833 | carbohydrate and lipid metabolism |
| 271 | vng271 | 0.137 | 1.371 | 0.024743 | −0.078 | 0.835 | 0.13 | unknown |
| 272 | vng272 | −0.018 | 0.960 | 0.585445 | −0.006 | 0.987 | 0.605917 | unknown |
| 274 | vng274 | 0.019 | 1.044 | 0.562292 | −0.013 | 0.971 | 0.561333 | DNA metabolism |
| 277 | crtl3 | 0.065 | 1.160 | 0.543831 | 0.023 | 1.054 | 0.643333 | cofactor and secondary |
| | | | | | | | | metabolites |
| 280 | vng280 | 0.004 | 1.009 | 0.47997 | 0.034 | 1.080 | 0.491567 | unknown |
| 281 | soxB | 0.099 | 1.256 | 0.109359 | −0.001 | 0.999 | 0.482583 | amino acid metabolism |
| 282 | vng282 | 0.062 | 1.154 | 0.220826 | 0.007 | 1.016 | 0.63625 | unknown |
| 283 | vng283 | −0.048 | 0.896 | 0.247582 | −0.071 | 0.850 | 0.22315 | cellular process |
| 284 | vng284 | −0.033 | 0.927 | 0.443279 | −0.049 | 0.894 | 0.478451 | energy metabolism |
| 285 | vng285 | −0.070 | 0.851 | 0.571998 | 0.041 | 1.098 | 0.41725 | DNA metabolism |
| 286 | vng286 | 0.046 | 1.112 | 0.271233 | −0.212 | 0.614 | 0.080287 | DNA metabolism |
| 287 | vng287 | 0.165 | 1.463 | 0.083116 | −0.046 | 0.899 | 0.618 | unknown |
| 289 | vng289 | −0.088 | 0.817 | 0.281945 | 0.025 | 1.059 | 0.621125 | unknown |
| 291 | vng291 | −0.003 | 0.992 | 0.52887 | 0.011 | 1.027 | 0.436842 | unknown |
| 293 | arlR3 | −0.094 | 0.806 | 0.121295 | 0.026 | 1.062 | 0.6005 | transcription and regulation |
| 294 | pnm | −0.084 | 0.823 | 0.206742 | −0.076 | 0.840 | 0.144433 | cofactor and secondary |
| | | | | | | | | metabolites |
| 296 | vng296 | 0.008 | 1.019 | 0.401346 | 0.006 | 1.015 | 0.65175 | unknown |
| 297 | vng297 | −0.024 | 0.946 | 0.366822 | −0.003 | 0.992 | 0.285183 | unknown |
| 298 | vng298 | 0.025 | 1.058 | 0.627964 | 0.039 | 1.095 | 0.53425 | unknown |
| 300 | vng300 | −0.065 | 0.861 | 0.324095 | −0.009 | 0.979 | 0.2879 | unknown |
| 301 | vng301 | 0.023 | 1.053 | 0.785162 | −0.017 | 0.961 | 0.5745 | cofactor and secondary |
| | | | | | | | | metabolites |
| 303 | lon | 0.199 | 1.580 | 0.204784 | −0.087 | 0.819 | 0.231828 | cellular process |
| 305 | ogt | 0.061 | 1.150 | 0.341725 | −0.042 | 0.907 | 0.447017 | DNA metabolism |
| 306 | trpC | −0.024 | 0.946 | 0.40266 | −0.062 | 0.867 | 0.410583 | amino acid metabolism |
| 307 | trpB | −0.012 | 0.974 | 0.405143 | −0.117 | 0.764 | 0.577417 | amino acid metabolism |
| 308 | trpA | −0.052 | 0.888 | 0.352507 | −0.013 | 0.971 | 0.675 | amino acid metabolism |
| 309 | vng309 | 0.141 | 1.383 | 0.057599 | −0.073 | 0.846 | 0.2447 | carbohydrate and lipid metabolism |
| 310 | vng311 | 0.031 | 1.075 | 0.197754 | −0.060 | 0.870 | 0.229 | amino acid metabolism |
| 311 | vng310 | 0.006 | 1.014 | 0.688905 | −0.008 | 0.982 | 0.227375 | unknown |
| 312 | vng312 | 0.081 | 1.204 | 0.554831 | 0.005 | 1.011 | 0.75 | unknown |
| 314 | aroD | 0.016 | 1.038 | 0.488896 | −0.038 | 0.915 | 0.623133 | amino acid metabolism |
| 315 | tfbF | −0.007 | 0.983 | 0.411002 | 0.016 | 1.039 | 0.7415 | transcription and regulation |
| 316 | vng316 | −0.014 | 0.968 | 0.745772 | −0.019 | 0.957 | 0.727 | unknown |
| 318 | dpg | −0.053 | 0.885 | 0.427936 | −0.058 | 0.876 | 0.586671 | cellular process |
| 319 | vng319 | 0.035 | 1.083 | 0.542096 | −0.118 | 0.762 | 0.066235 | transcription and regulation |
| 320 | arlR4 | −0.111 | 0.775 | 0.275232 | 0.054 | 1.131 | 0.407133 | transcription and regulation |
| 321 | ids | 0.048 | 1.117 | 0.459917 | 0.015 | 1.035 | 0.537083 | cellular process |
| 322 | vng322 | 0.003 | 1.006 | 0.631188 | 0.006 | 1.014 | 0.592875 | cellular process |
| 323 | vng323 | 0.036 | 1.086 | 0.53138 | −0.016 | 0.965 | 0.699 | unknown |
| 324 | pykA | −0.212 | 0.613 | 0.198102 | 0.033 | 1.080 | 0.46605 | energy metabolism |
| 326 | metS | −0.006 | 0.986 | 0.593786 | 0.022 | 1.052 | 0.5259 | translation |
| 327 | gadD | −0.047 | 0.897 | 0.508424 | −0.016 | 0.965 | 0.685083 | amino acid metabolism |
| 329 | caaX | 0.020 | 1.047 | 0.350884 | −0.001 | 0.998 | 0.641167 | cellular process |
| 330 | ppsA | 0.299 | 1.990 | 0.000269 | 0.084 | 1.213 | 0.274869 | carbohydrate and lipid metabolism |
| 331 | vng331 | −0.068 | 0.855 | 0.124214 | −0.105 | 0.785 | 0.0378 | unknown |
| 332 | vng332 | −0.010 | 0.978 | 0.666212 | −0.100 | 0.794 | 0.188 | unknown |
| 335 | gptA1 | 0.127 | 1.341 | 0.21277 | −0.012 | 0.973 | 0.715833 | nucleotide metabolism |
| 337 | vng337 | −0.283 | 0.522 | 5.08E−06 | 0.030 | 1.072 | 0.551 | DNA metabolism |
| 339 | vng339 | 0.048 | 1.118 | 0.571744 | −0.078 | 0.835 | 0.267 | unknown |
| 340 | vng340 | 0.017 | 1.041 | 0.665899 | −0.016 | 0.963 | 0.447858 | unknown |
| 342 | smc1 | −0.001 | 0.999 | 0.509779 | −0.019 | 0.957 | 0.664 | cellular process |
| 343 | vng343 | 0.127 | 1.339 | 0.130597 | −0.081 | 0.829 | 0.21575 | unknown |
| 345 | gatB2 | −0.154 | 0.702 | 0.284616 | 0.015 | 1.035 | 0.724167 | translation |
| 346 | vng346 | −0.014 | 0.967 | 0.779659 | 0.026 | 1.061 | 0.888 | unknown |
| 347 | vng347 | 0.003 | 1.008 | 0.362037 | 0.040 | 1.096 | 0.475167 | unknown |
| 349 | topA | −0.022 | 0.951 | 0.243338 | −0.003 | 0.993 | 0.642167 | DNA metabolism |
| 351 | vng351 | 0.022 | 1.052 | 0.212565 | 0.013 | 1.029 | 0.760583 | unknown |
| 352 | vng352 | 0.111 | 1.291 | 0.243077 | −0.006 | 0.987 | 0.6485 | unknown |
| 354 | vng354 | 0.115 | 1.305 | 0.241255 | −0.024 | 0.945 | 0.628583 | unknown |
| 355 | htr14 | −0.067 | 0.856 | 0.576293 | 0.060 | 1.149 | 0.36665 | cellular process |
| 357 | vng357 | 0.057 | 1.141 | 0.26771 | −0.011 | 0.974 | 0.643 | unknown |
| 359 | vng359 | 0.075 | 1.188 | 0.378703 | −0.060 | 0.871 | 0.644917 | unknown |
| 360 | vng360 | 0.026 | 1.062 | 0.47728 | −0.017 | 0.961 | 0.710833 | unknown |
| 361 | vng361 | 0.014 | 1.032 | 0.138702 | 0.007 | 1.017 | 0.729167 | unknown |
| 363 | nfi | 0.019 | 1.044 | 0.631898 | −0.010 | 0.978 | 0.779417 | DNA metabolism |
| 364 | yusZ3 | 0.067 | 1.168 | 0.318905 | −0.005 | 0.988 | 0.84575 | general function prediction only |
| 365 | arsA1 | 0.025 | 1.058 | 0.6947 | −0.045 | 0.902 | 0.567333 | transport |
| 367 | vng367 | 0.021 | 1.049 | 0.285685 | −0.027 | 0.941 | 0.609917 | unknown |
| 368 | vng368 | 0.053 | 1.130 | 0.422475 | −0.040 | 0.911 | 0.429 | unknown |
| 370 | vng370 | −0.088 | 0.816 | 0.137316 | 0.012 | 1.028 | 0.763 | general function prediction only |
| 371 | acd1 | 0.066 | 1.164 | 0.493743 | −0.081 | 0.830 | 0.213658 | carbohydrate and lipid metabolism |
| 373 | vng373 | −0.111 | 0.775 | 0.235297 | −0.027 | 0.939 | 0.479667 | unknown |
| 374 | nusG | −0.005 | 0.988 | 0.494148 | −0.023 | 0.948 | 0.600333 | transcription and regulation |
| 375 | secE | 0.061 | 1.150 | 0.341978 | −0.057 | 0.877 | 0.32475 | cellular process |
| 376 | ftsZ1 | 0.065 | 1.160 | 0.22801 | 0.011 | 1.026 | 0.686333 | cellular process |
| 378 | vng378 | −0.058 | 0.875 | 0.412749 | 0.049 | 1.119 | 0.518317 | unknown |
| 380 | caa | 0.016 | 1.037 | 0.603882 | −0.017 | 0.963 | 0.625083 | transport |
| 382 | aroE | −0.040 | 0.912 | 0.393967 | 0.005 | 1.012 | 0.769125 | amino acid metabolism |
| 383 | vng383 | −0.020 | 0.955 | 0.698128 | −0.023 | 0.948 | 0.63675 | unknown |
| 384 | trpE2 | −0.151 | 0.706 | 0.240605 | −0.054 | 0.882 | 0.414717 | amino acid metabolism |
| 386 | trpG2 | −0.014 | 0.969 | 0.20572 | −0.034 | 0.924 | 0.518 | amino acid metabolism |
| 387 | ilvE1 | −0.056 | 0.879 | 0.510405 | −0.067 | 0.857 | 0.3142 | amino acid metabolism |
| 388 | vng388 | 0.109 | 1.285 | 0.202928 | −0.023 | 0.948 | 0.72075 | unknown |
| 389 | tzhR1 | −0.021 | 0.952 | 0.46123 | −0.018 | 0.960 | 0.786 | transcription and regulation |
| 390 | graD1 | −0.050 | 0.891 | 0.052766 | −0.060 | 0.870 | 0.292875 | nucleotide metabolism |
| 391 | vng391 | 0.003 | 1.006 | 0.546894 | −0.042 | 0.909 | 0.52975 | unknown |
| 393 | vng393 | 0.013 | 1.031 | 0.511304 | 0.028 | 1.067 | 0.625167 | unknown |
| 394 | vng394 | 0.087 | 1.221 | 0.095675 | 0.038 | 1.090 | 0.465 | unknown |
| 396 | vng396 | 0.068 | 1.170 | 0.429092 | −0.053 | 0.885 | 0.3636 | unknown |
| 398 | nthA1 | 0.063 | 1.157 | 0.447504 | 0.009 | 1.022 | 0.758833 | DNA metabolism |
| 399 | vng399 | −0.085 | 0.822 | 0.424255 | 0.012 | 1.029 | 0.640667 | unknown |
| 401 | epf2 | −0.067 | 0.856 | 0.331069 | −0.008 | 0.983 | 0.59125 | transcription and regulation |
| 402 | vng402 | 0.138 | 1.374 | 0.39376 | −0.132 | 0.738 | 0.036406 | unknown |
| 403 | proS | −0.174 | 0.670 | 0.175104 | −0.007 | 0.983 | 0.158065 | translation |
| 404 | vng404 | −0.052 | 0.887 | 0.566009 | −0.021 | 0.954 | 0.785833 | unknown |
| 405 | vng405 | 0.015 | 1.035 | 0.524954 | −0.085 | 0.823 | 0.422391 | unknown |
| 406 | vng406 | −0.038 | 0.916 | 0.584345 | 0.017 | 1.041 | 0.607417 | energy metabolism |
| 407 | vng407 | −0.027 | 0.940 | 0.511424 | 0.036 | 1.086 | 0.5335 | unknown |
| 408 | vng408 | 0.032 | 1.078 | 0.54097 | −0.025 | 0.943 | 0.757333 | unknown |
| 409 | vng409 | −0.019 | 0.958 | 0.30561 | 0.021 | 1.050 | 0.5296 | amino acid metabolism |
| 410 | rfbU2 | −0.004 | 0.990 | 0.589505 | −0.025 | 0.944 | 0.605083 | translation |
| 412 | folP | −0.101 | 0.793 | 0.1038 | 0.057 | 1.140 | 0.301867 | cofactor and secondary |
| | | | | | | | | metabolites |
| 414 | purH | 0.079 | 1.199 | 0.14638 | 0.105 | 1.275 | 0.131875 | nucleotide metabolism |
| 415 | purB | 0.131 | 1.352 | 0.253717 | 0.054 | 1.131 | 0.436167 | nucleotide metabolism |
| 417 | vng417 | −0.097 | 0.801 | 0.219127 | −0.071 | 0.848 | 0.257633 | unknown |
| 419 | vng419 | 0.037 | 1.089 | 0.344767 | −0.038 | 0.917 | 0.52075 | unknown |
| 420 | vng420 | 0.026 | 1.062 | 0.259805 | −0.068 | 0.854 | 0.193625 | unknown |
| 421 | vng421 | −0.044 | 0.904 | 0.375783 | −0.032 | 0.929 | 0.5675 | cellular process |
| 422 | cyc | 0.108 | 1.283 | 0.440029 | 0.008 | 1.018 | 0.749 | cofactor and secondary |
| | | | | | | | | metabolites |
| 424 | nacR | 0.055 | 1.136 | 0.266418 | −0.015 | 0.966 | 0.7615 | transcription and regulation |
| 425 | pimT2 | −0.008 | 0.981 | 0.138417 | −0.021 | 0.953 | 0.50425 | translation |
| 426 | rpoM | −0.147 | 0.712 | 0.021842 | −0.028 | 0.938 | 0.2857 | transcription and regulation |
| 427 | yfmO2 | −0.042 | 0.908 | 0.35065 | 0.022 | 1.053 | 0.792333 | transport |
| 428 | fad2 | 0.017 | 1.040 | 0.422599 | −0.038 | 0.916 | 0.4867 | carbohydrate and lipid metabolism |
| 429 | vng429 | 0.067 | 1.167 | 0.249856 | 0.002 | 1.005 | 0.783 | unknown |
| 430 | vng430 | −0.139 | 0.727 | 0.007715 | 0.093 | 1.239 | 0.071975 | unknown |
| 431 | apa | −0.066 | 0.858 | 0.277148 | −0.001 | 0.998 | 0.461917 | DNA metabolism |
| 433 | vng433 | −0.021 | 0.953 | 0.593453 | −0.070 | 0.851 | 0.17425 | translation |
| 435 | vng435 | −0.078 | 0.836 | 0.31763 | −0.148 | 0.711 | 0.111905 | unknown |
| 436 | nhaC1 | 0.098 | 1.254 | 0.202716 | −0.123 | 0.753 | 0.07535 | transport |
| 437 | vng437 | 0.034 | 1.083 | 0.519363 | 0.027 | 1.065 | 0.306667 | cellular process |
| 438 | vng439 | 0.089 | 1.228 | 0.116572 | −0.035 | 0.923 | 0.618625 | unknown |
| 439 | ferA3 | 0.016 | 1.038 | 0.375113 | 0.062 | 1.154 | 0.452857 | energy metabolism |
| 440 | vng440 | −0.036 | 0.920 | 0.532323 | 0.067 | 1.167 | 0.2289 | unknown |
| 441 | vng441 | 0.020 | 1.046 | 0.666214 | −0.066 | 0.859 | 0.3263 | unknown |
| 442 | rspA | 0.001 | 1.003 | 0.578023 | 0.025 | 1.058 | 0.490917 | cellular process |
| 444 | dapA | −0.122 | 0.755 | 0.575413 | 0.006 | 1.014 | 0.45225 | amino acid metabolism |
| 446 | tdh1 | −0.003 | 0.993 | 0.27478 | −0.250 | 0.562 | 0.021493 | amino acid metabolism |
| 447 | gcd | 0.018 | 1.042 | 0.708412 | 0.036 | 1.086 | 0.548333 | unknown |
| 448 | pyrE1 | −0.005 | 0.990 | 0.424552 | −0.023 | 0.949 | 0.299667 | nucleotide metabolism |
| 450 | vng450 | 0.014 | 1.033 | 0.656325 | −0.066 | 0.859 | 0.183 | cellular process |
| 451 | phoU | −0.135 | 0.734 | 0.203795 | −0.027 | 0.940 | 0.698958 | transcription and regulation |
| 452 | pstB2 | 0.022 | 1.052 | 0.652816 | −0.070 | 0.851 | 0.16995 | transport |
| 453 | pstA2 | −0.531 | 0.294 | 3.92E−13 | −0.030 | 0.933 | 0.5765 | transport |
| 455 | pstC2 | −0.306 | 0.494 | 0.141415 | −0.054 | 0.883 | 0.333 | transport |
| 457 | phoX | −0.170 | 0.675 | 0.372558 | −0.031 | 0.931 | 0.55975 | transport |
| 458 | prp1 | −0.032 | 0.928 | 0.509126 | −0.093 | 0.808 | 0.205993 | transcription and regulation |
| 459 | cysH | 0.177 | 1.505 | 0.00417 | −0.095 | 0.804 | 0.124958 | amino acid metabolism |
| 461 | aspS | −0.004 | 0.990 | 0.612547 | −0.048 | 0.895 | 0.3383 | translation |
| 462 | mrlR | −0.061 | 0.870 | 0.903282 | 0.033 | 1.078 | 0.8875 | transcription and regulation |
| 463 | vng463 | 0.063 | 1.156 | 0.342466 | −0.036 | 0.920 | 0.52201 | unknown |
| 465 | nosF2 | 0.097 | 1.251 | 0.217381 | 0.013 | 1.031 | 0.684083 | transport |
| 466 | vng466 | 0.090 | 1.232 | 0.24497 | 0.011 | 1.025 | 0.7905 | unknown |
| 467 | yafB | 0.203 | 1.598 | 0.061325 | 0.026 | 1.063 | 0.6095 | general function prediction only |
| 468 | vng468 | −0.043 | 0.907 | 0.383695 | 0.005 | 1.011 | 0.735 | energy metabolism |
| 469 | vng469 | 0.091 | 1.233 | 0.278493 | 0.017 | 1.041 | 0.657083 | unknown |
| 470 | trp3 | 0.027 | 1.063 | 0.729602 | −0.023 | 0.948 | 0.700375 | transport |
| 471 | xrlR2 | 0.053 | 1.130 | 0.295843 | −0.022 | 0.951 | 0.662 | transcription and regulation |
| 472 | vng472 | 0.069 | 1.173 | 0.166856 | 0.005 | 1.012 | 0.716167 | unknown |
| 473 | korB | 0.058 | 1.144 | 0.212098 | 0.005 | 1.012 | 0.688083 | energy metabolism |
| 474 | korA | 0.058 | 1.143 | 0.291271 | 0.010 | 1.024 | 0.37375 | energy metabolism |
| 475 | fpr | 0.012 | 1.029 | 0.459809 | 0.002 | 1.004 | 0.553917 | cellular process |
| 478 | vng478 | 0.123 | 1.326 | 0.236624 | 0.014 | 1.032 | 0.83125 | amino acid metabolism |
| 479 | yajO1 | 0.041 | 1.098 | 0.65205 | 0.005 | 1.012 | 0.504 | cofactor and secondary |
| | | | | | | | | metabolites |
| 481 | mcmA1 | −0.009 | 0.979 | 0.677365 | −0.029 | 0.936 | 0.522833 | carbohydrate and lipid metabolism |
| 482 | vng482 | −0.132 | 0.738 | 0.098029 | 0.039 | 1.094 | 0.473125 | transcription and regulation |
| 483 | vng483 | −0.058 | 0.875 | 0.444473 | 0.015 | 1.036 | 0.560417 | unknown |
| 484 | acs1 | 0.004 | 1.008 | 0.497161 | 0.022 | 1.052 | 0.7685 | carbohydrate and lipid metabolism |
| 485 | vng485 | 0.013 | 1.030 | 0.727553 | −0.018 | 0.959 | 0.6525 | unknown |
| 486 | hat1 | 0.048 | 1.116 | 0.5362 | −0.010 | 0.977 | 0.737875 | translation |
| 487 | vng487 | −0.046 | 0.899 | 0.552503 | 0.016 | 1.037 | 0.724583 | translation |
| 488 | vng488 | −0.003 | 0.993 | 0.360988 | 0.034 | 1.082 | 0.545917 | unknown |
| 489 | dnaJ | −0.116 | 0.766 | 0.051251 | 0.041 | 1.099 | 0.414 | cellular process |
| 491 | dnaK | −0.059 | 0.872 | 0.41602 | −0.004 | 0.991 | 0.894667 | cellular process |
| 492 | vng492 | 0.022 | 1.053 | 0.70206 | 0.024 | 1.057 | 0.69575 | unknown |
| 494 | grpE | −0.005 | 0.989 | 0.616965 | −0.033 | 0.926 | 0.499292 | cellular process |
| 496 | vng496 | −0.016 | 0.964 | 0.401727 | 0.016 | 1.038 | 0.4609 | unknown |
| 498 | vng498 | −0.050 | 0.891 | 0.566657 | 0.031 | 1.074 | 0.72025 | unknown |
| 499 | cna | −0.050 | 0.892 | 0.369277 | 0.047 | 1.114 | 0.45725 | translation |
| 500 | ppd | 0.079 | 1.199 | 0.256448 | −0.062 | 0.866 | 0.364503 | carbohydrate and lipid metabolism |
| 501 | yfmO1 | −0.004 | 0.992 | 0.47004 | 0.060 | 1.148 | 0.3335 | transport |
| 502 | aspB1 | 0.144 | 1.393 | 0.016395 | −0.015 | 0.965 | 0.7455 | amino acid metabolism |
| 503 | vng503 | 0.013 | 1.030 | 0.500639 | −0.076 | 0.840 | 0.851875 | cofactor and secondary |
| | | | | | | | | metabolites |
| 504 | gbp5 | −0.088 | 0.817 | 0.295371 | 0.025 | 1.058 | 0.787167 | translation |
| 505 | vng505 | 0.009 | 1.021 | 0.370059 | 0.075 | 1.189 | 0.446467 | unknown |
| 506 | vng506 | 0.030 | 1.073 | 0.844418 | −0.002 | 0.996 | 0.887 | unknown |
| 507 | vng507 | 0.055 | 1.134 | 0.526433 | −0.007 | 0.983 | 0.779 | unknown |
| 508 | vng508 | −0.037 | 0.918 | 0.700464 | −0.038 | 0.916 | 0.5355 | unknown |
| 509 | vng509 | −0.034 | 0.925 | 0.485897 | −0.047 | 0.897 | 0.4305 | unknown |
| 510 | prrIV2 | 0.160 | 1.447 | 0.121503 | 0.056 | 1.137 | 0.4331 | cellular process |
| 511 | arlR5 | −0.006 | 0.987 | 0.409182 | −0.043 | 0.905 | 0.488125 | transcription and regulation |
| 512 | ppe | 0.017 | 1.039 | 0.626528 | −0.010 | 0.977 | 0.61525 | DNA metabolism |
| 514 | vng514 | −0.149 | 0.710 | 0.252428 | 0.013 | 1.031 | 0.6855 | DNA metabolism |
| 516 | vng516 | 0.015 | 1.036 | 0.126104 | −0.051 | 0.889 | 0.481 | unknown |
| 518 | vng518 | −0.073 | 0.845 | 0.439726 | −0.036 | 0.920 | 0.614 | unknown |
| 520 | vng520 | −0.015 | 0.966 | 0.409923 | −0.059 | 0.873 | 0.514 | unknown |
| 521 | polB1 | 0.018 | 1.042 | 0.670085 | −0.019 | 0.957 | 0.541583 | DNA metabolism |
| 523 | inb | 0.028 | 1.066 | 0.471942 | 0.000 | 1.000 | 0.679917 | general function prediction only |
| 524 | yurY | −0.193 | 0.641 | 0.006591 | 0.078 | 1.198 | 0.113 | transport |
| 525 | vng525 | −0.230 | 0.589 | 0.109609 | 0.035 | 1.084 | 0.51575 | cellular process |
| 527 | vng527 | −0.044 | 0.904 | 0.45367 | −0.007 | 0.984 | 0.53775 | cellular process |
| 529 | vng529 | −0.049 | 0.893 | 0.602143 | −0.045 | 0.902 | 0.6605 | unknown |
| 530 | troR | −0.110 | 0.777 | 0.32615 | −0.061 | 0.869 | 0.2797 | transcription and regulation |
| 532 | vng532 | −0.023 | 0.949 | 0.455806 | −0.049 | 0.894 | 0.688875 | unknown |
| 533 | vng533 | 0.081 | 1.205 | 0.471712 | 0.011 | 1.026 | 0.734167 | unknown |
| 534 | vng534 | −0.022 | 0.950 | 0.208194 | −0.022 | 0.950 | 0.65475 | unknown |
| 535 | vng535 | −0.111 | 0.775 | 0.368574 | 0.010 | 1.024 | 0.7815 | unknown |
| 536 | sirR | −0.087 | 0.819 | 0.299135 | 0.021 | 1.049 | 0.688 | transcription and regulation |
| 537 | vng537 | 0.020 | 1.048 | 0.601641 | 0.006 | 1.015 | 0.6285 | unknown |
| 539 | vng539 | 0.075 | 1.189 | 0.36017 | −0.048 | 0.895 | 0.439217 | unknown |
| 540 | imp | 0.177 | 1.502 | 0.157427 | −0.033 | 0.926 | 0.502917 | general function prediction only |
| 541 | thrC2 | −0.001 | 0.997 | 0.606394 | −0.055 | 0.881 | 0.394245 | amino acid metabolism |
| 542 | vng542 | −0.041 | 0.911 | 0.457424 | −0.014 | 0.969 | 0.279867 | cofactor and secondary |
| | | | | | | | | metabolites |
| 543 | vng543 | −0.076 | 0.840 | 0.454057 | −0.085 | 0.822 | 0.518833 | unknown |
| 544 | vng544 | −0.024 | 0.947 | 0.691544 | 0.003 | 1.007 | 0.484583 | unknown |
| 546 | vng546 | −0.107 | 0.782 | 0.127827 | 0.080 | 1.202 | 0.175183 | unknown |
| 548 | vng548 | −0.017 | 0.961 | 0.549501 | 0.013 | 1.031 | 0.7905 | translation |
| 549 | eif2a | −0.045 | 0.902 | 0.489441 | 0.088 | 1.223 | 0.092025 | translation |
| 550 | rps27e | −0.063 | 0.866 | 0.537067 | 0.081 | 1.204 | 0.10535 | translation |
| 551 | rpl44e | −0.075 | 0.841 | 0.137356 | 0.026 | 1.061 | 0.63125 | translation |
| 553 | vng553 | −0.090 | 0.813 | 0.206597 | −0.045 | 0.901 | 0.3945 | unknown |
| 555 | vng555 | −0.071 | 0.848 | 0.340857 | 0.039 | 1.094 | 0.590667 | unknown |
| 556 | sgb | 0.033 | 1.078 | 0.366605 | 0.015 | 1.034 | 0.676667 | general function prediction only |
| 557 | vng557 | −0.009 | 0.980 | 0.356378 | 0.029 | 1.070 | 0.59225 | unknown |
| 559 | apt | 0.024 | 1.057 | 0.443812 | −0.038 | 0.917 | 0.4541 | nucleotide metabolism |
| 560 | vng560 | −0.026 | 0.941 | 0.744985 | 0.002 | 1.004 | 0.941833 | energy metabolism |
| 562 | vng562 | 0.010 | 1.023 | 0.555804 | 0.026 | 1.062 | 0.660333 | energy metabolism |
| 563 | ndhG2 | −0.026 | 0.943 | 0.470904 | 0.020 | 1.047 | 0.667833 | energy metabolism |
| 564 | vng565 | 0.028 | 1.066 | 0.537833 | −0.010 | 0.977 | 0.6295 | unknown |
| 565 | vng564 | 0.008 | 1.018 | 0.210054 | −0.003 | 0.994 | 0.516 | unknown |
| 566 | vng566 | −0.049 | 0.894 | 0.452059 | −0.001 | 0.998 | 0.64125 | unknown |
| 568 | vng570 | 0.043 | 1.104 | 0.39948 | 0.014 | 1.032 | 0.8 | unknown |
| 570 | vng568 | 0.117 | 1.310 | 0.0931 | −0.052 | 0.887 | 0.289 | unknown |
| 571 | vng571 | 0.030 | 1.072 | 0.341458 | −0.014 | 0.967 | 0.618917 | unknown |
| 572 | dfp | −0.067 | 0.857 | 0.440488 | 0.006 | 1.015 | 0.667 | cofactor and secondary |
| | | | | | | | | metabolites |
| 573 | hcpE | −0.008 | 0.981 | 0.340217 | −0.003 | 0.992 | 0.7475 | energy metabolism |
| 574 | vng574 | 0.032 | 1.077 | 0.442131 | −0.025 | 0.945 | 0.63875 | unknown |
| 575 | ywaD | −0.044 | 0.903 | 0.554021 | −0.033 | 0.926 | 0.608883 | amino acid metabolism |
| 576 | vng576 | 0.065 | 1.161 | 0.456273 | −0.054 | 0.884 | 0.382333 | carbohydrate and lipid metabolism |
| 578 | vng578 | 0.111 | 1.290 | 0.44419 | −0.082 | 0.827 | 0.15046 | unknown |
| 579 | vng579 | −0.040 | 0.912 | 0.552485 | 0.012 | 1.028 | 0.581333 | unknown |
| 581 | vng581 | 0.021 | 1.049 | 0.591083 | −0.007 | 0.984 | 0.7195 | unknown |
| 582 | cybB1 | 0.059 | 1.146 | 0.225487 | −0.014 | 0.969 | 0.675167 | energy metabolism |
| 583 | cybB2 | 0.029 | 1.068 | 0.486285 | −0.019 | 0.958 | 0.537067 | energy metabolism |
| 584 | cybA | −0.089 | 0.814 | 0.129117 | 0.026 | 1.063 | 0.599625 | energy metabolism |
| 585 | cybD | −0.058 | 0.875 | 0.379463 | 0.056 | 1.138 | 0.264667 | energy metabolism |
| 586 | cybE | −0.077 | 0.838 | 0.212524 | 0.036 | 1.086 | 0.495417 | energy metabolism |
| 587 | vng587 | −0.052 | 0.887 | 0.496892 | −0.007 | 0.983 | 0.35965 | unknown |
| 590 | vng590 | 0.099 | 1.255 | 0.277559 | −0.046 | 0.899 | 0.576167 | unknown |
| 591 | arlR6 | 0.037 | 1.088 | 0.548129 | −0.004 | 0.991 | 0.458 | transcription and regulation |
| 592 | nthA2 | −0.010 | 0.977 | 0.664499 | 0.013 | 1.030 | 0.6342 | DNA metabolism |
| 593 | dmd | −0.005 | 0.989 | 0.512368 | −0.003 | 0.992 | 0.674667 | carbohydrate and lipid metabolism |
| 594 | vng594 | −0.058 | 0.875 | 0.266383 | −0.004 | 0.992 | 0.804 | unknown |
| 595 | vng595 | 0.096 | 1.247 | 0.354933 | −0.038 | 0.916 | 0.652333 | amino acid metabolism |
| 597 | vng597 | 0.035 | 1.085 | 0.617992 | −0.047 | 0.897 | 0.386 | unknown |
| 598 | vng598 | 0.013 | 1.032 | 0.519409 | 0.027 | 1.064 | 0.707417 | unknown |
| 599 | vng600 | 0.076 | 1.193 | 0.21008 | −0.070 | 0.851 | 0.2622 | translation |
| 600 | str | 0.026 | 1.062 | 0.472171 | 0.068 | 1.170 | 0.33665 | general function prediction only |
| 601 | vng601 | −0.207 | 0.621 | 0.024266 | 0.036 | 1.087 | 0.482 | translation |
| 602 | vng602 | 0.073 | 1.182 | 0.548005 | 0.043 | 1.105 | 0.599625 | cellular process |
| 604 | vng604 | 0.102 | 1.265 | 0.136631 | −0.149 | 0.709 | 0.011199 | unknown |
| 606 | yrhA | 0.113 | 1.297 | 0.286319 | −0.045 | 0.901 | 0.379667 | amino acid metabolism |
| 608 | vng608 | −0.022 | 0.951 | 0.608573 | 0.051 | 1.124 | 0.462625 | unknown |
| 609 | vng609 | −0.131 | 0.739 | 0.129568 | 0.096 | 1.246 | 0.113307 | unknown |
| 610 | hhoA | −0.005 | 0.988 | 0.560297 | 0.029 | 1.070 | 0.503667 | cofactor and secondary |
| | | | | | | | | metabolites |
| 611 | vng611 | −0.133 | 0.736 | 0.063719 | 0.041 | 1.100 | 0.532467 | unknown |
| 612 | vng612 | −0.095 | 0.804 | 0.299925 | 0.042 | 1.100 | 0.602875 | unknown |
| 613 | vng613 | −0.033 | 0.927 | 0.464975 | 0.004 | 1.009 | 0.36475 | unknown |
| 614 | htr16 | −0.022 | 0.951 | 0.641788 | −0.029 | 0.935 | 0.691 | cellular process |
| 615 | trkA7 | −0.022 | 0.950 | 0.660868 | −0.003 | 0.994 | 0.87725 | transport |
| 617 | vng617 | −0.048 | 0.895 | 0.561773 | −0.046 | 0.899 | 0.4296 | unknown |
| 620 | edp | 0.096 | 1.248 | 0.218498 | −0.032 | 0.929 | 0.574 | cellular process |
| 622 | vng622 | 0.012 | 1.028 | 0.582856 | 0.001 | 1.003 | 0.647333 | unknown |
| 623 | yngE | 0.051 | 1.124 | 0.478868 | 0.015 | 1.036 | 0.674833 | carbohydrate and lipid metabolism |
| 624 | vng624 | −0.024 | 0.946 | 0.377876 | 0.031 | 1.074 | 0.61875 | unknown |
| 626 | maoC2 | 0.108 | 1.284 | 0.303125 | −0.029 | 0.935 | 0.693417 | carbohydrate and lipid metabolism |
| 627 | citE | −0.077 | 0.837 | 0.657542 | 0.033 | 1.078 | 0.6715 | energy metabolism |
| 628 | gdhA1 | 0.222 | 1.668 | 0.212167 | −0.023 | 0.948 | 0.449 | amino acid metabolism |
| 629 | aspB2 | −0.042 | 0.909 | 0.499587 | −0.034 | 0.924 | 0.5592 | amino acid metabolism |
| 630 | ribE | −0.039 | 0.913 | 0.44244 | 0.036 | 1.086 | 0.537 | cofactor and secondary |
| | | | | | | | | metabolites |
| 631 | vng631 | −0.070 | 0.852 | 0.272573 | −0.031 | 0.931 | 0.591167 | cellular process |
| 632 | purK | −0.029 | 0.935 | 0.283154 | 0.022 | 1.052 | 0.604133 | nucleotide metabolism |
| 633 | purE | 0.078 | 1.197 | 0.2524 | 0.031 | 1.075 | 0.5928 | nucleotide metabolism |
| 635 | nolB | −0.079 | 0.834 | 0.278209 | −0.005 | 0.988 | 0.757333 | energy metabolism |
| 636 | ndhG1 | −0.119 | 0.761 | 0.099946 | 0.009 | 1.021 | 0.846833 | energy metabolism |
| 637 | ndhG5 | −0.100 | 0.793 | 0.237864 | 0.010 | 1.023 | 0.430433 | energy metabolism |
| 639 | ndhG4 | 0.005 | 1.011 | 0.389529 | 0.014 | 1.032 | 0.621167 | energy metabolism |
| 640 | nolD | −0.105 | 0.786 | 0.185639 | 0.021 | 1.048 | 0.661667 | energy metabolism |
| 641 | vng641 | 0.072 | 1.180 | 0.299618 | −0.002 | 0.995 | 0.891667 | energy metabolism |
| 642 | vng642 | 0.036 | 1.086 | 0.505511 | −0.017 | 0.961 | 0.7265 | energy metabolism |
| 643 | nolC | 0.003 | 1.007 | 0.575539 | 0.018 | 1.042 | 0.67 | energy metabolism |
| 646 | nuoL | −0.014 | 0.967 | 0.253379 | 0.052 | 1.127 | 0.3155 | energy metabolism |
| 647 | nuoM | 0.019 | 1.046 | 0.200753 | 0.052 | 1.127 | 0.323883 | energy metabolism |
| 648 | ndhG3 | 0.040 | 1.096 | 0.417393 | −0.026 | 0.943 | 0.480333 | energy metabolism |
| 650 | vng650 | 0.004 | 1.010 | 0.602308 | −0.003 | 0.992 | 0.828333 | unknown |
| 651 | imd1 | 0.029 | 1.069 | 0.43492 | 0.000 | 1.000 | 0.650667 | transcription and regulation |
| 652 | vng652 | 0.038 | 1.092 | 0.175695 | 0.022 | 1.053 | 0.43275 | unknown |
| 653 | mcmA1 | 0.133 | 1.359 | 0.089582 | −0.009 | 0.980 | 0.570583 | carbohydrate and lipid metabolism |
| 654 | act3 | −0.050 | 0.892 | 0.350046 | 0.042 | 1.102 | 0.514867 | transcription and regulation |
| 656 | vng656 | −0.047 | 0.898 | 0.350533 | −0.014 | 0.968 | 0.5825 | unknown |
| 657 | coxA | −0.053 | 0.885 | 0.178845 | 0.002 | 1.004 | 0.371017 | energy metabolism |
| 659 | vng659 | −0.043 | 0.905 | 0.681927 | −0.071 | 0.850 | 0.1555 | unknown |
| 660 | vng660 | 0.013 | 1.030 | 0.504271 | −0.038 | 0.917 | 0.49 | unknown |
| 662 | coxC | 0.125 | 1.332 | 0.330643 | −0.053 | 0.885 | 0.375333 | energy metabolism |
| 664 | birA | −0.058 | 0.875 | 0.578022 | 0.009 | 1.021 | 0.749917 | cofactor and secondary |
| | | | | | | | | metabolites |
| 665 | coxB | 0.053 | 1.129 | 0.306202 | −0.074 | 0.844 | 0.1797 | energy metabolism |
| 666 | ctaB | −0.005 | 0.989 | 0.644785 | 0.018 | 1.043 | 0.664833 | cofactor and secondary |
| | | | | | | | | metabolites |
| 667 | trp4 | −0.014 | 0.969 | 0.543478 | 0.026 | 1.060 | 0.671333 | transport |
| 668 | vng668 | 0.054 | 1.131 | 0.381902 | −0.025 | 0.944 | 0.738333 | cellular process |
| 669 | vng669 | 0.139 | 1.376 | 0.268165 | −0.028 | 0.937 | 0.730667 | cellular process |
| 670 | vng670 | −0.006 | 0.987 | 0.446081 | −0.014 | 0.967 | 0.759292 | unknown |
| 671 | vng671 | 0.092 | 1.236 | 0.259417 | −0.030 | 0.934 | 0.558 | unknown |
| 673 | mcmA3 | 0.021 | 1.049 | 0.444664 | −0.031 | 0.932 | 0.641625 | carbohydrate and lipid metabolism |
| 674 | tsk | 0.117 | 1.309 | 0.264866 | −0.006 | 0.987 | 0.821375 | amino acid metabolism |
| 675 | vng675 | 0.119 | 1.317 | 0.134657 | −0.032 | 0.930 | 0.55725 | unknown |
| 676 | nbp2 | 0.084 | 1.213 | 0.408295 | −0.006 | 0.987 | 0.3125 | transcription and regulation |
| 677 | Vng0677h | −0.003 | 0.992 | 0.44238 | −0.002 | 0.995 | 0.728 | unknown |
| 678 | acaB1 | 0.030 | 1.072 | 0.427171 | 0.036 | 1.088 | 0.52725 | carbohydrate and lipid metabolism |
| 679 | acd4 | 0.296 | 1.979 | 0.008147 | −0.025 | 0.944 | 0.53925 | carbohydrate and lipid metabolism |
| 680 | fdfT | 0.051 | 1.125 | 0.219759 | −0.074 | 0.843 | 0.224033 | carbohydrate and lipid metabolism |
| 681 | hbd1 | 0.040 | 1.096 | 0.401768 | −0.063 | 0.866 | 0.327925 | carbohydrate and lipid metabolism |
| 682 | vng682 | −0.037 | 0.919 | 0.380406 | 0.041 | 1.100 | 0.518167 | unknown |
| 683 | vng683 | 0.155 | 1.427 | 0.174676 | −0.057 | 0.876 | 0.252667 | carbohydrate and lipid metabolism |
| 684 | fbp | 0.150 | 1.413 | 0.002952 | 0.006 | 1.015 | 0.877 | carbohydrate and lipid metabolism |
| 686 | vng686 | 0.021 | 1.050 | 0.490526 | −0.014 | 0.969 | 0.6965 | unknown |
| 688 | vng688 | −0.007 | 0.983 | 0.733566 | −0.030 | 0.932 | 0.838125 | unknown |
| 689 | trp5 | 0.041 | 1.099 | 0.550906 | −0.136 | 0.732 | 0.108028 | transport |
| 690 | vng690 | 0.070 | 1.174 | 0.334592 | 0.033 | 1.079 | 0.53625 | transport |
| 692 | vng692 | −0.145 | 0.716 | 0.26897 | 0.007 | 1.017 | 0.69175 | transport |
| 694 | nthB | 0.006 | 1.013 | 0.17786 | 0.026 | 1.062 | 0.59775 | DNA metabolism |
| 697 | vng697 | 0.023 | 1.055 | 0.649551 | −0.016 | 0.963 | 0.75475 | unknown |
| 698 | vng698 | 0.031 | 1.073 | 0.618764 | −0.017 | 0.962 | 0.66675 | unknown |
| 700 | yvgX | −0.067 | 0.858 | 0.280395 | 0.028 | 1.067 | 0.817667 | transport |
| 702 | vng702 | 0.240 | 1.737 | 4.45E−06 | −0.054 | 0.883 | 0.2775 | unknown |
| 703 | arlR7 | −0.060 | 0.870 | 0.395812 | −0.028 | 0.937 | 0.46225 | transcription and regulation |
| 704 | marR | 0.063 | 1.157 | 0.543245 | −0.047 | 0.897 | 0.615417 | transcription and regulation |
| 705 | vng705 | 0.071 | 1.178 | 0.251751 | 0.026 | 1.061 | 0.681667 | unknown |
| 707 | vng707 | 0.015 | 1.035 | 0.726168 | −0.034 | 0.926 | 0.550917 | DNA metabolism |
| 708 | vng708 | 0.045 | 1.109 | 0.372804 | 0.063 | 1.155 | 0.31115 | unknown |
| 709 | vng709 | 0.156 | 1.432 | 0.0835 | −0.026 | 0.941 | 0.552917 | unknown |
| 711 | vng711 | 0.242 | 1.746 | 8.53E−05 | −0.018 | 0.958 | 0.728 | cellular process |
| 713 | vng713 | 0.023 | 1.054 | 0.645286 | 0.017 | 1.040 | 0.4975 | unknown |
| 715 | thiC | −0.020 | 0.955 | 0.201141 | 0.139 | 1.377 | 0.21353 | cofactor and secondary |
| | | | | | | | | metabolites |
| 716 | afsQ2 | 0.000 | 1.001 | 0.387037 | 0.025 | 1.059 | 0.571525 | cellular process |
| 717 | vng717 | 0.162 | 1.451 | 0.00477 | 0.002 | 1.004 | 0.73125 | unknown |
| 718 | vng718 | 0.012 | 1.028 | 0.583029 | −0.002 | 0.995 | 0.62875 | unknown |
| 719 | araL | 0.007 | 1.015 | 0.398167 | −0.024 | 0.946 | 0.366567 | carbohydrate and lipid metabolism |
| 720 | dip2 | 0.046 | 1.111 | 0.454673 | −0.021 | 0.952 | 0.810417 | DNA metabolism |
| 723 | pepQ1 | 0.034 | 1.082 | 0.423297 | 0.008 | 1.018 | 0.816167 | amino acid metabolism |
| 724 | vng724 | −0.201 | 0.629 | 0.123871 | 0.043 | 1.105 | 0.45775 | unknown |
| 725 | vng725 | 0.022 | 1.052 | 0.178341 | −0.009 | 0.978 | 0.576583 | unknown |
| 726 | acrR2 | −0.015 | 0.967 | 0.304513 | −0.037 | 0.917 | 0.52075 | transcription and regulation |
| 727 | vng727 | 0.139 | 1.376 | 0.122478 | −0.082 | 0.828 | 0.132933 | cellular process |
| 729 | vng729 | 0.077 | 1.195 | 0.402676 | −0.035 | 0.922 | 0.167683 | unknown |
| 730 | vng730 | −0.079 | 0.833 | 0.469092 | −0.012 | 0.972 | 0.654458 | cofactor and secondary |
| | | | | | | | | metabolites |
| 731 | vng731 | −0.013 | 0.971 | 0.178394 | −0.014 | 0.968 | 0.7005 | unknown |
| 732 | tpc | 0.009 | 1.021 | 0.394393 | 0.022 | 1.052 | 0.695667 | translation |
| 733 | vng733 | −0.032 | 0.928 | 0.507604 | −0.018 | 0.958 | 0.680667 | unknown |
| 734 | tfbB | 0.011 | 1.026 | 0.22611 | 0.072 | 1.179 | 0.389997 | transcription and regulation |
| 735 | hlx2 | 0.086 | 1.220 | 0.253248 | −0.084 | 0.825 | 0.122588 | transcription and regulation |
| 736 | kinA2 | −0.061 | 0.868 | 0.569208 | −0.136 | 0.731 | 0.023839 | cellular process |
| 737 | vng738 | −0.033 | 0.927 | 0.393548 | 0.007 | 1.016 | 0.873 | unknown |
| 738 | vng737 | −0.070 | 0.852 | 0.516854 | 0.012 | 1.028 | 0.726 | unknown |
| 740 | vng740 | 0.034 | 1.081 | 0.554923 | −0.014 | 0.967 | 0.706917 | unknown |
| 741 | vng741 | 0.080 | 1.203 | 0.155794 | 0.077 | 1.195 | 0.1394 | unknown |
| 742 | vng742 | −0.049 | 0.893 | 0.208323 | −0.033 | 0.927 | 0.598083 | cellular process |
| 743 | vng743 | 0.132 | 1.355 | 0.155256 | −0.133 | 0.736 | 0.0343 | cellular process |
| 745 | spoVR | −0.020 | 0.954 | 0.496586 | 0.008 | 1.018 | 0.448617 | cellular process |
| 746 | vng746 | −0.040 | 0.912 | 0.613304 | 0.037 | 1.089 | 0.597833 | unknown |
| 748 | prkA | −0.065 | 0.862 | 0.462524 | 0.058 | 1.142 | 0.551083 | cellular process |
| 749 | prk | 0.016 | 1.038 | 0.33866 | 0.041 | 1.099 | 0.556167 | cellular process |
| 750 | gdp | 0.164 | 1.458 | 0.292417 | −0.073 | 0.844 | 0.361975 | cellular process |
| 751 | prlR2 | −0.021 | 0.953 | 0.239206 | −0.050 | 0.892 | 0.34665 | transcription and regulation |
| 752 | galE1 | −0.008 | 0.981 | 0.274297 | 0.025 | 1.059 | 0.373183 | nucleotide metabolism |
| 753 | ylnA | −0.053 | 0.886 | 0.652972 | 0.011 | 1.026 | 0.4657 | transport |
| 754 | vng754 | −0.057 | 0.877 | 0.194771 | −0.004 | 0.990 | 0.854 | unknown |
| 755 | vng755 | 0.055 | 1.134 | 0.288634 | 0.016 | 1.037 | 0.6225 | unknown |
| 757 | tfeA | 0.063 | 1.157 | 0.338689 | 0.007 | 1.017 | 0.763 | transcription and regulation |
| 758 | vng758 | −0.072 | 0.847 | 0.370816 | 0.057 | 1.140 | 0.3005 | unknown |
| 759 | vng759 | 0.078 | 1.196 | 0.302758 | −0.024 | 0.947 | 0.545417 | unknown |
| 761 | dip3 | −0.006 | 0.987 | 0.357181 | −0.045 | 0.902 | 0.508293 | general function prediction only |
| 762 | vng762 | 0.092 | 1.236 | 0.181836 | −0.104 | 0.787 | 0.0648 | unknown |
| 763 | ybjG | −0.009 | 0.980 | 0.670123 | 0.024 | 1.057 | 0.426617 | general function prediction only |
| 764 | vng764 | −0.003 | 0.994 | 0.489762 | −0.010 | 0.976 | 0.81925 | unknown |
| 765 | vng765 | −0.122 | 0.755 | 0.214194 | −0.129 | 0.742 | 0.015958 | unknown |
| 766 | prp2 | 0.039 | 1.094 | 0.637986 | −0.043 | 0.905 | 0.568833 | transcription and regulation |
| 767 | vng767 | 0.054 | 1.134 | 0.530118 | −0.052 | 0.888 | 0.3165 | unknown |
| 768 | vng768 | 0.008 | 1.018 | 0.672453 | −0.038 | 0.916 | 0.532125 | transcription and regulation |
| 769 | vng769 | −0.198 | 0.634 | 0.231079 | 0.018 | 1.043 | 0.202038 | unknown |
| 771 | aldY2 | 0.224 | 1.676 | 0.010862 | 0.021 | 1.049 | 0.605583 | cofactor and secondary |
| | | | | | | | | metabolites |
| 772 | vng772 | −0.060 | 0.872 | 0.697918 | 0.025 | 1.059 | 0.77175 | unknown |
| 775 | acd2 | 0.030 | 1.073 | 0.370766 | −0.001 | 0.997 | 0.823583 | carbohydrate and lipid metabolism |
| 777 | taqD | 0.022 | 1.051 | 0.646261 | −0.042 | 0.907 | 0.4035 | nucleotide metabolism |
| 778 | vng778 | −0.001 | 0.998 | 0.829305 | −0.074 | 0.844 | 0.221463 | unknown |
| 779 | vng779 | −0.100 | 0.794 | 0.120347 | −0.039 | 0.914 | 0.51375 | DNA metabolism |
| 780 | vng780 | −0.020 | 0.956 | 0.696668 | −0.022 | 0.951 | 0.68425 | unknown |
| 782 | vng782 | −0.020 | 0.956 | 0.677642 | −0.005 | 0.989 | 0.709667 | unknown |
| 784 | pssA | −0.006 | 0.986 | 0.323705 | 0.012 | 1.027 | 0.423 | carbohydrate and lipid metabolism |
| 786 | hcpF | −0.044 | 0.903 | 0.432997 | −0.006 | 0.986 | 0.745417 | energy metabolism |
| 787 | rps3e | −0.075 | 0.841 | 0.164931 | 0.006 | 1.013 | 0.572892 | translation |
| 788 | vng788 | −0.086 | 0.821 | 0.479316 | 0.070 | 1.174 | 0.1739 | unknown |
| 789 | vng789 | −0.225 | 0.595 | 0.036856 | 0.032 | 1.076 | 0.557083 | unknown |
| 790 | rps15p | −0.070 | 0.851 | 0.450988 | 0.039 | 1.094 | 0.443 | translation |
| 793 | htr6 | −0.031 | 0.931 | 0.635321 | −0.025 | 0.945 | 0.684083 | cellular process |
| 794 | yufN | 0.030 | 1.072 | 0.512484 | −0.036 | 0.921 | 0.53 | transport |
| 795 | hcpC | −0.021 | 0.952 | 0.192274 | −0.050 | 0.891 | 0.283334 | energy metabolism |
| 798 | vng798 | −0.032 | 0.929 | 0.385428 | 0.000 | 1.000 | 0.711083 | unknown |
| 799 | vng799 | 0.014 | 1.032 | 0.230035 | −0.048 | 0.895 | 0.509588 | cellular process |
| 800 | vng800 | 0.001 | 1.002 | 0.386756 | −0.005 | 0.989 | 0.481917 | cellular process |
| 801 | vng801 | 0.062 | 1.153 | 0.328484 | −0.098 | 0.798 | 0.0547 | cellular process |
| 804 | vng804 | 0.007 | 1.016 | 0.410894 | −0.047 | 0.898 | 0.546167 | unknown |
| 806 | htr4 | 0.013 | 1.030 | 0.348368 | 0.010 | 1.024 | 0.62375 | cellular process |
| 808 | gabD | 0.014 | 1.034 | 0.343736 | 0.003 | 1.006 | 0.825 | amino acid metabolism |
| 810 | vng810 | −0.007 | 0.985 | 0.532548 | 0.000 | 0.999 | 0.684833 | unknown |
| 811 | vng811 | 0.095 | 1.244 | 0.320443 | 0.026 | 1.062 | 0.638375 | unknown |
| 812 | htr18 | −0.053 | 0.884 | 0.367674 | −0.005 | 0.989 | 0.55975 | cellular process |
| 813 | potD | −0.071 | 0.849 | 0.519118 | 0.022 | 1.052 | 0.63275 | transport |
| 814 | vng814 | −0.010 | 0.978 | 0.349053 | −0.072 | 0.847 | 0.494674 | unknown |
| 815 | yfmJ | 0.059 | 1.147 | 0.362008 | −0.013 | 0.970 | 0.655 | general function prediction only |
| 816 | chi | 0.018 | 1.043 | 0.123618 | −0.050 | 0.892 | 0.432597 | carbohydrate and lipid metabolism |
| 818 | vng818 | −0.064 | 0.862 | 0.510174 | 0.009 | 1.021 | 0.510417 | unknown |
| 819 | vng819 | 0.048 | 1.117 | 0.663665 | 0.001 | 1.001 | 0.6985 | unknown |
| 821 | vng821 | −0.017 | 0.961 | 0.618133 | 0.043 | 1.105 | 0.491325 | cofactor and secondary |
| | | | | | | | | metabolites |
| 822 | vng822 | −0.163 | 0.688 | 0.221229 | 0.037 | 1.089 | 0.463833 | cellular process |
| 823 | gspE2 | −0.105 | 0.785 | 0.103595 | 0.017 | 1.041 | 0.793917 | cellular process |
| 824 | gdb | −0.083 | 0.825 | 0.509461 | −0.043 | 0.906 | 0.6615 | cofactor and secondary |
| | | | | | | | | metabolites |
| 825 | mobB | 0.032 | 1.076 | 0.353873 | 0.000 | 0.999 | 0.649375 | cofactor and secondary |
| | | | | | | | | metabolites |
| 826 | dmsR | 0.025 | 1.060 | 0.473831 | −0.067 | 0.857 | 0.5225 | transcription and regulation |
| 828 | dmsE | −0.047 | 0.898 | 0.32189 | −0.137 | 0.729 | 0.4065 | energy metabolism |
| 829 | dmsA | −0.009 | 0.978 | 0.324467 | −0.027 | 0.939 | 0.512212 | energy metabolism |
| 830 | dmsB | 0.041 | 1.099 | 0.481828 | 0.008 | 1.018 | 0.795 | energy metabolism |
| 831 | dmsC | 0.045 | 1.108 | 0.657679 | 0.026 | 1.063 | 0.658042 | energy metabolism |
| 832 | dmsD | 0.025 | 1.060 | 0.53112 | −0.043 | 0.906 | 0.4727 | energy metabolism |
| 834 | vng834 | −0.010 | 0.977 | 0.442555 | 0.004 | 1.010 | 0.781333 | unknown |
| 835 | idr2 | −0.118 | 0.761 | 0.162074 | −0.003 | 0.993 | 0.77175 | transcription and regulation |
| 836 | vng836 | 0.024 | 1.057 | 0.406367 | 0.021 | 1.050 | 0.579667 | unknown |
| 837 | vng837 | 0.046 | 1.111 | 0.416966 | 0.075 | 1.188 | 0.172567 | transcription and regulation |
| 838 | ssrA | −0.071 | 0.849 | 0.548462 | 0.028 | 1.067 | 0.695833 | DNA metabolism |
| 840 | vng840 | −0.058 | 0.874 | 0.29178 | 0.007 | 1.017 | 0.835 | unknown |
| 841 | icfA | 0.001 | 1.002 | 0.730065 | −0.052 | 0.887 | 0.583167 | cellular process |
| 845 | vng845 | 0.028 | 1.067 | 0.348646 | −0.080 | 0.833 | 0.360733 | cofactor and secondary |
| | | | | | | | | metabolites |
| 846 | vng846 | −0.056 | 0.878 | 0.48813 | 0.029 | 1.069 | 0.633083 | unknown |
| 847 | vng847 | −0.103 | 0.789 | 0.212003 | −0.051 | 0.889 | 0.396475 | unknown |
| 849 | vng849 | 0.105 | 1.273 | 0.33967 | −0.033 | 0.927 | 0.5375 | unknown |
| 851 | vng851 | −0.040 | 0.912 | 0.566698 | −0.124 | 0.752 | 0.045673 | unknown |
| 852 | bolR3 | −0.002 | 0.996 | 0.679231 | −0.009 | 0.980 | 0.797667 | transcription and regulation |
| 853 | vng853 | 0.112 | 1.295 | 0.086699 | −0.079 | 0.835 | 0.1304 | unknown |
| 854 | vng854 | 0.051 | 1.125 | 0.518284 | −0.075 | 0.841 | 0.2015 | translation |
| 857 | vng857 | 0.064 | 1.158 | 0.191784 | 0.037 | 1.089 | 0.52575 | translation |
| 858 | vng858 | −0.031 | 0.930 | 0.620378 | −0.001 | 0.997 | 0.22051 | unknown |
| 860 | rpoL | 0.022 | 1.052 | 0.573552 | −0.027 | 0.940 | 0.5825 | transcription and regulation |
| 861 | vng861 | −0.033 | 0.926 | 0.392668 | 0.063 | 1.157 | 0.256 | unknown |
| 862 | hisF | −0.042 | 0.908 | 0.708508 | −0.016 | 0.963 | 0.809625 | amino acid metabolism |
| 863 | vng863 | −0.042 | 0.908 | 0.494235 | −0.023 | 0.949 | 0.66325 | unknown |
| 864 | purL1 | −0.032 | 0.930 | 0.660088 | 0.052 | 1.127 | 0.413117 | nucleotide metabolism |
| 865 | vng865 | 0.051 | 1.125 | 0.268493 | −0.009 | 0.981 | 0.565833 | unknown |
| 867 | asnA | −0.071 | 0.848 | 0.313126 | 0.007 | 1.015 | 0.800167 | amino acid metabolism |
| 868 | vng868 | −0.039 | 0.915 | 0.336631 | −0.022 | 0.951 | 0.683333 | DNA metabolism |
| 869 | tfbD | 0.004 | 1.008 | 0.496811 | −0.005 | 0.988 | 0.7085 | transcription and regulation |
| 870 | gatC | 0.015 | 1.036 | 0.62291 | −0.031 | 0.931 | 0.4895 | translation |
| 872 | gatA | 0.011 | 1.026 | 0.374214 | −0.020 | 0.956 | 0.695 | translation |
| 873 | ykhA | 0.106 | 1.275 | 0.386193 | −0.050 | 0.891 | 0.58875 | carbohydrate and lipid metabolism |
| 874 | traB | 0.019 | 1.046 | 0.363798 | −0.003 | 0.993 | 0.67675 | cellular process |
| 875 | vng875 | −0.023 | 0.947 | 0.575988 | −0.009 | 0.979 | 0.569417 | cellular process |
| 876 | purM | 0.016 | 1.037 | 0.859986 | 0.121 | 1.321 | 0.053425 | nucleotide metabolism |
| 878 | gul1 | 0.206 | 1.606 | 0.013778 | −0.017 | 0.962 | 0.6365 | transcription and regulation |
| 879 | vng879 | −0.033 | 0.927 | 0.532298 | −0.033 | 0.927 | 0.5225 | unknown |
| 880 | psmA | 0.042 | 1.101 | 0.337975 | −0.004 | 0.990 | 0.690917 | cellular process |
| 881 | lig | −0.088 | 0.816 | 0.31356 | −0.028 | 0.937 | 0.553275 | DNA metabolism |
| 882 | pho2 | −0.043 | 0.906 | 0.198569 | 0.007 | 1.015 | 0.75425 | carbohydrate and lipid metabolism |
| 883 | vng883 | 0.002 | 1.004 | 0.52167 | −0.010 | 0.977 | 0.626 | unknown |
| 884 | top6A | −0.036 | 0.921 | 0.510293 | −0.054 | 0.882 | 0.395325 | DNA metabolism |
| 885 | top6B | −0.052 | 0.888 | 0.423964 | −0.029 | 0.936 | 0.594 | DNA metabolism |
| 887 | gyrB | −0.114 | 0.770 | 0.146203 | 0.017 | 1.039 | 0.540417 | DNA metabolism |
| 889 | gyrA | −0.118 | 0.763 | 0.405683 | 0.023 | 1.054 | 0.59425 | DNA metabolism |
| 890 | imd2 | 0.012 | 1.028 | 0.500132 | −0.009 | 0.980 | 0.807917 | transcription and regulation |
| 891 | yjlD | 0.038 | 1.092 | 0.455105 | 0.030 | 1.072 | 0.659067 | general function prediction only |
| 892 | vng892 | 0.090 | 1.231 | 0.258028 | −0.006 | 0.987 | 0.703875 | unknown |
| 893 | udp2 | 0.102 | 1.264 | 0.284005 | −0.051 | 0.890 | 0.438242 | nucleotide metabolism |
| 896 | cda | −0.015 | 0.966 | 0.764715 | −0.020 | 0.955 | 0.704 | nucleotide metabolism |
| 897 | rbsC1 | −0.016 | 0.963 | 0.316193 | 0.018 | 1.043 | 0.597417 | transport |
| 898 | rbsC2 | 0.014 | 1.032 | 0.462766 | 0.050 | 1.121 | 0.376333 | transport |
| 901 | rbsA | −0.085 | 0.823 | 0.221997 | −0.002 | 0.996 | 0.6425 | transport |
| 903 | vng903 | −0.053 | 0.885 | 0.40185 | 0.031 | 1.075 | 0.57025 | unknown |
| 905 | pmu2 | −0.044 | 0.904 | 0.579744 | 0.014 | 1.033 | 0.588167 | carbohydrate and lipid metabolism |
| 906 | vng906 | −0.044 | 0.905 | 0.401614 | 0.035 | 1.084 | 0.508933 | unknown |
| 907 | vng907 | −0.003 | 0.994 | 0.637359 | 0.040 | 1.098 | 0.444 | unknown |
| 908 | putP | −0.060 | 0.871 | 0.364541 | 0.041 | 1.100 | 0.514833 | transport |
| 909 | vng909 | −0.027 | 0.940 | 0.391095 | −0.031 | 0.932 | 0.63 | unknown |
| 911 | vng911 | −0.040 | 0.911 | 0.479268 | −0.022 | 0.951 | 0.640317 | unknown |
| 913 | vng913 | −0.006 | 0.987 | 0.81696 | −0.023 | 0.948 | 0.616833 | unknown |
| 914 | vng914 | −0.005 | 0.989 | 0.702931 | −0.012 | 0.973 | 0.77125 | unknown |
| 915 | hakA | −0.003 | 0.994 | 0.84594 | −0.005 | 0.990 | 0.4985 | amino acid metabolism |
| 916 | ark | −0.028 | 0.937 | 0.17242 | 0.005 | 1.011 | 0.644583 | cellular process |
| 917 | hrg | −0.051 | 0.889 | 0.440022 | −0.056 | 0.878 | 0.502378 | transcription and regulation |
| 919 | gst | 0.008 | 1.019 | 0.41126 | −0.131 | 0.740 | 0.020481 | DNA metabolism |
| 920 | vng920 | −0.064 | 0.862 | 0.407646 | 0.025 | 1.060 | 0.625367 | carbohydrate and lipid metabolism |
| 921 | potA1 | −0.154 | 0.702 | 0.306684 | 0.027 | 1.063 | 0.554417 | unknown |
| 923 | sfuB | −0.242 | 0.573 | 0.13551 | −0.020 | 0.955 | 0.728333 | transport |
| 924 | ibp | −0.076 | 0.840 | 0.210472 | −0.106 | 0.783 | 0.04045 | transport |
| 925 | vng925 | 0.017 | 1.041 | 0.778909 | −0.075 | 0.841 | 0.3025 | transport |
| 926 | vng926 | −0.061 | 0.870 | 0.306288 | 0.020 | 1.047 | 0.67725 | unknown |
| 927 | vng927 | −0.032 | 0.930 | 0.562769 | 0.017 | 1.040 | 0.496917 | unknown |
| 928 | mak | −0.042 | 0.908 | 0.683934 | 0.032 | 1.077 | 0.67325 | unknown |
| 930 | yvbT | 0.125 | 1.332 | 0.122455 | −0.010 | 0.977 | 0.771833 | general function prediction only |
| 931 | acaB2 | 0.097 | 1.250 | 0.226041 | −0.038 | 0.916 | 0.501 | energy metabolism |
| 932 | nbp3 | 0.046 | 1.112 | 0.5009 | −0.030 | 0.932 | 0.548333 | carbohydrate and lipid metabolism |
| 933 | yqjM | 0.090 | 1.231 | 0.383245 | −0.034 | 0.926 | 0.588 | transcription and regulation |
| 934 | vng934 | 0.050 | 1.121 | 0.473159 | −0.076 | 0.840 | 0.269975 | energy metabolism |
| 935 | noxC | 0.107 | 1.278 | 0.223584 | −0.066 | 0.859 | 0.28 | unknown |
| 936 | vng936 | 0.108 | 1.281 | 0.375812 | −0.063 | 0.865 | 0.292175 | energy metabolism |
| 937 | gap | −0.321 | 0.477 | 0.085932 | −0.019 | 0.957 | 0.786333 | unknown |
| 938 | gufA | 0.066 | 1.165 | 0.401043 | 0.007 | 1.017 | 0.292375 | energy metabolism |
| 940 | acs3 | −0.181 | 0.659 | 0.136972 | −0.061 | 0.869 | 0.255367 | general function prediction only |
| 941 | vng941 | −0.164 | 0.685 | 0.016846 | −0.045 | 0.901 | 0.389083 | energy metabolism |
| 942 | cheW2 | −0.015 | 0.967 | 0.535698 | −0.065 | 0.861 | 0.2615 | unknown |
| 943 | arlR8 | −0.004 | 0.990 | 0.25724 | −0.065 | 0.861 | 0.2505 | cellular process |
| 945 | vng945 | 0.083 | 1.209 | 0.514541 | −0.095 | 0.803 | 0.222625 | transcription and regulation |
| 946 | minD1 | 0.034 | 1.080 | 0.405147 | −0.025 | 0.944 | 0.636667 | unknown |
| 947 | fapJ | −0.021 | 0.953 | 0.551658 | −0.015 | 0.967 | 0.544667 | cellular process |
| 949 | gspE3 | −0.028 | 0.938 | 0.552958 | 0.044 | 1.106 | 0.516167 | cellular process |
| 950 | flaH | −0.056 | 0.880 | 0.328039 | 0.034 | 1.081 | 0.516 | cellular process |
| 953 | vng953 | 0.004 | 1.010 | 0.264 | −0.003 | 0.993 | 0.651417 | cellular process |
| 954 | vng954 | −0.041 | 0.909 | 0.364709 | −0.004 | 0.991 | 0.678583 | cellular process |
| 955 | fapE | 0.000 | 1.000 | 0.810065 | 0.015 | 1.034 | 0.467583 | cellular process |
| 958 | htr15 | −0.007 | 0.984 | 0.704299 | 0.007 | 1.016 | 0.629333 | cellular process |
| 959 | vng959 | 0.044 | 1.106 | 0.429496 | −0.020 | 0.955 | 0.8135 | cellular process |
| 963 | flaB2 | −0.106 | 0.784 | 0.08514 | 0.027 | 1.063 | 0.570833 | cellular process |
| 964 | flaB3 | −0.234 | 0.584 | 0.000194 | −0.006 | 0.986 | 0.576958 | cellular process |
| 965 | cpcE | 0.033 | 1.080 | 0.662817 | −0.028 | 0.938 | 0.620975 | cellular process |
| 966 | cheR | 0.002 | 1.005 | 0.659296 | −0.024 | 0.947 | 0.658583 | cellular process |
| 967 | cheD | −0.069 | 0.854 | 0.178879 | 0.027 | 1.065 | 0.588 | cellular process |
| 969 | vng969 | −0.030 | 0.934 | 0.356562 | 0.004 | 1.010 | 0.715833 | cellular process |
| 970 | cheC1 | −0.181 | 0.659 | 0.176017 | 0.021 | 1.049 | 0.537 | cellular process |
| 971 | cheA | −0.153 | 0.703 | 0.100628 | 0.007 | 1.016 | 0.532667 | cellular process |
| 973 | cheB | −0.120 | 0.759 | 0.290401 | 0.009 | 1.021 | 0.656833 | cellular process |
| 974 | cheY | −0.129 | 0.743 | 0.024369 | 0.009 | 1.020 | 0.677083 | cellular process |
| 976 | cheW1 | −0.013 | 0.971 | 0.494337 | 0.026 | 1.062 | 0.55875 | cellular process |
| 978 | vng978 | −0.020 | 0.956 | 0.166385 | −0.016 | 0.964 | 0.5411 | unknown |
| 979 | vng979 | 0.059 | 1.147 | 0.309989 | 0.025 | 1.060 | 0.722167 | unknown |
| 981 | vng981 | 0.070 | 1.175 | 0.344463 | −0.264 | 0.545 | 0.491833 | unknown |
| 982 | vng982 | 0.064 | 1.159 | 0.333538 | −0.045 | 0.902 | 0.367417 | amino acid metabolism |
| 983 | vng983 | −0.022 | 0.951 | 0.666291 | −0.027 | 0.939 | 0.506333 | unknown |
| 985 | vng985 | −0.079 | 0.833 | 0.157392 | 0.115 | 1.304 | 0.2784 | unknown |
| 986 | vng986 | 0.070 | 1.174 | 0.203805 | 0.047 | 1.116 | 0.459067 | unknown |
| 989 | vng989 | −0.051 | 0.889 | 0.328333 | 0.004 | 1.008 | 0.547033 | unknown |
| 990 | vng990 | 0.093 | 1.239 | 0.214042 | 0.025 | 1.059 | 0.74865 | DNA metabolism |
| 991 | vng991 | −0.023 | 0.947 | 0.227832 | 0.118 | 1.313 | 0.187375 | unknown |
| 992 | vng992 | −0.065 | 0.860 | 0.337294 | 0.066 | 1.163 | 0.37645 | unknown |
| 993 | vng993 | −0.161 | 0.691 | 0.105334 | 0.001 | 1.003 | 0.4808 | unknown |
| 995 | vng995 | −0.243 | 0.572 | 0.003639 | −0.029 | 0.935 | 0.412 | unknown |
| 996 | boa4 | −0.045 | 0.901 | 0.641746 | 0.028 | 1.068 | 0.654667 | unknown |
| 997 | acs2 | 0.103 | 1.267 | 0.424028 | 0.012 | 1.028 | 0.76975 | transcription and regulation |
| 998 | yajO2 | 0.183 | 1.525 | 0.181311 | −0.094 | 0.806 | 0.19575 | energy metabolism |
| 999 | vng999 | −0.048 | 0.895 | 0.51294 | 0.095 | 1.245 | 0.140275 | energy metabolism |
| 1000 | vng1000 | 0.036 | 1.087 | 0.405356 | −0.001 | 0.998 | 0.819 | unknown |
| 1001 | guaB | −0.014 | 0.968 | 0.461407 | −0.004 | 0.992 | 0.745 | unknown |
| 1002 | vng1002 | 0.028 | 1.066 | 0.563044 | −0.034 | 0.925 | 0.58675 | nucleotide metabolism |
| 1003 | vng1003 | 0.081 | 1.206 | 0.315701 | −0.055 | 0.880 | 0.357075 | unknown |
| 1005 | vng1005 | −0.101 | 0.792 | 0.110106 | −0.065 | 0.862 | 0.6155 | unknown |
| 1006 | vng1006 | −0.075 | 0.841 | 0.423475 | −0.066 | 0.859 | 0.1965 | unknown |
| 1007 | vng1007 | −0.090 | 0.814 | 0.128251 | −0.058 | 0.875 | 0.3584 | unknown |
| 1009 | flaA2 | 0.192 | 1.556 | 0.002995 | 0.011 | 1.026 | 0.817 | cellular process |
| 1011 | vng1011 | −0.185 | 0.653 | 0.195647 | 0.017 | 1.041 | 0.625833 | unknown |
| 1012 | vng1012 | −0.191 | 0.644 | 0.013253 | 0.012 | 1.029 | 0.764 | cellular process |
| 1013 | htr13 | 0.015 | 1.035 | 0.475379 | −0.024 | 0.946 | 0.185567 | cellular process |
| 1014 | yqjH | −0.001 | 0.998 | 0.810109 | 0.016 | 1.038 | 0.600933 | DNA metabolism |
| 1015 | vng1015 | 0.033 | 1.078 | 0.871611 | −0.023 | 0.948 | 0.6135 | unknown |
| 1017 | vng1017 | 0.052 | 1.127 | 0.476939 | −0.055 | 0.882 | 0.434 | unknown |
| 1018 | adh3 | 0.053 | 1.131 | 0.705646 | 0.043 | 1.103 | 0.773167 | general function prediction only |
| 1020 | vng1020 | −0.004 | 0.991 | 0.327583 | 0.023 | 1.054 | 0.540417 | carbohydrate and lipid metabolism |
| 1021 | vng1021 | 0.001 | 1.003 | 0.638616 | −0.053 | 0.884 | 0.452 | unknown |
| 1023 | vng1023 | 0.096 | 1.248 | 0.422631 | −0.120 | 0.758 | 0.216378 | amino acid metabolism |
| 1024 | vng1024 | −0.027 | 0.939 | 0.677495 | −0.045 | 0.902 | 0.395 | cofactor and secondary |
| | | | | | | | | metabolites |
| 1025 | vng1025 | 0.015 | 1.035 | 0.750514 | −0.117 | 0.763 | 0.18225 | unknown |
| 1026 | vng1026 | 0.027 | 1.065 | 0.546529 | 0.003 | 1.006 | 0.804 | unknown |
| 1027 | tpiA | 0.002 | 1.004 | 0.856761 | −0.002 | 0.995 | 0.272625 | carbohydrate and lipid metabolism |
| 1029 | xrlR3 | 0.120 | 1.318 | 0.284335 | −0.017 | 0.961 | 0.677833 | transcription and regulation |
| 1030 | pgsA | 0.029 | 1.070 | 0.743894 | 0.031 | 1.075 | 0.669333 | carbohydrate and lipid metabolism |
| 1031 | vng1031 | 0.035 | 1.083 | 0.643884 | 0.006 | 1.014 | 0.802 | nucleotide metabolism |
| 1033 | hisC1 | −0.012 | 0.972 | 0.64651 | −0.016 | 0.964 | 0.696167 | amino acid metabolism |
| 1034 | vng1034 | −0.049 | 0.894 | 0.356317 | −0.078 | 0.835 | 0.216583 | unknown |
| 1035 | vng1035 | 0.005 | 1.012 | 0.654434 | −0.024 | 0.947 | 0.512092 | amino acid metabolism |
| 1036 | vng1036 | 0.124 | 1.329 | 0.406429 | −0.049 | 0.892 | 0.688 | unknown |
| 1037 | hpcE | 0.120 | 1.318 | 0.393561 | −0.032 | 0.930 | 0.571333 | cofactor and secondary |
| | | | | | | | | metabolites |
| 1038 | vng1038 | 0.176 | 1.501 | 0.05085 | −0.077 | 0.838 | 0.16645 | unknown |
| 1039 | vng1039 | −0.058 | 0.875 | 0.241484 | 0.067 | 1.167 | 0.2035 | unknown |
| 1041 | vng1041 | −0.034 | 0.924 | 0.468855 | −0.057 | 0.877 | 0.417661 | unknown |
| 1042 | vng1042 | −0.093 | 0.807 | 0.072402 | −0.045 | 0.902 | 0.399 | unknown |
| 1046 | vng1047 | 0.271 | 1.866 | 0.003291 | −0.019 | 0.956 | 0.745 | unknown |
| 1047 | vng1046 | 0.078 | 1.198 | 0.173519 | 0.013 | 1.031 | 0.83425 | unknown |
| 1048 | udg1 | 0.149 | 1.410 | 0.182709 | 0.010 | 1.023 | 0.6855 | nucleotide metabolism |
| 1050 | vng1050 | −0.021 | 0.952 | 0.577754 | −0.042 | 0.909 | 0.2709 | unknown |
| 1052 | vng1052 | 0.063 | 1.155 | 0.475718 | −0.072 | 0.847 | 0.197 | unknown |
| 1053 | gtl | −0.011 | 0.976 | 0.572412 | 0.000 | 1.001 | 0.642667 | general function prediction only |
| 1054 | ptp | −0.018 | 0.960 | 0.525221 | −0.001 | 0.998 | 0.755333 | transport |
| 1055 | graD4 | 0.075 | 1.190 | 0.245496 | −0.046 | 0.900 | 0.4478 | nucleotide metabolism |
| 1056 | vng1056 | −0.114 | 0.769 | 0.096511 | −0.013 | 0.971 | 0.737917 | unknown |
| 1057 | vng1057 | 0.014 | 1.032 | 0.470302 | −0.030 | 0.933 | 0.592 | unknown |
| 1058 | vng1058 | −0.137 | 0.729 | 0.139628 | 0.009 | 1.020 | 0.610917 | unknown |
| 1059 | gyf | −0.066 | 0.859 | 0.380489 | −0.033 | 0.926 | 0.6945 | general function prediction only |
| 1060 | vng1060 | −0.017 | 0.962 | 0.684987 | −0.029 | 0.935 | 0.67325 | unknown |
| 1062 | rfbQ | −0.014 | 0.969 | 0.713298 | −0.001 | 0.997 | 0.681667 | carbohydrate and lipid metabolism |
| 1063 | vng1063 | −0.009 | 0.979 | 0.539069 | 0.000 | 0.999 | 0.535167 | unknown |
| 1064 | vng1064 | −0.118 | 0.762 | 0.095203 | 0.024 | 1.058 | 0.7415 | unknown |
| 1065 | vng1065 | 0.027 | 1.064 | 0.350991 | 0.019 | 1.044 | 0.658833 | cofactor and secondary |
| | | | | | | | | metabolites |
| 1066 | vng1066 | 0.056 | 1.137 | 0.389052 | −0.012 | 0.972 | 0.712417 | unknown |
| 1067 | exoM | 0.017 | 1.041 | 0.678835 | −0.029 | 0.935 | 0.657333 | cellular process |
| 1068 | tot | −0.017 | 0.962 | 0.567372 | −0.012 | 0.972 | 0.5415 | general function prediction only |
| 1069 | vng1069 | −0.051 | 0.890 | 0.326703 | 0.047 | 1.115 | 0.41 | unknown |
| 1070 | gpdA1 | −0.003 | 0.994 | 0.539187 | −0.034 | 0.925 | 0.531625 | carbohydrate and lipid metabolism |
| 1071 | cinR | −0.040 | 0.912 | 0.317238 | −0.048 | 0.896 | 0.360333 | transcription and regulation |
| 1073 | menE | −0.019 | 0.956 | 0.509357 | −0.025 | 0.945 | 0.604333 | cofactor and secondary |
| | | | | | | | | metabolites |
| 1074 | menC | 0.000 | 0.999 | 0.747879 | −0.001 | 0.998 | 0.840875 | cofactor and secondary |
| | | | | | | | | metabolites |
| 1075 | menA | −0.132 | 0.738 | 0.163209 | 0.036 | 1.086 | 0.536367 | cofactor and secondary |
| | | | | | | | | metabolites |
| 1077 | opuD | −0.013 | 0.971 | 0.481046 | −0.006 | 0.986 | 0.682917 | transport |
| 1079 | menB | 0.013 | 1.029 | 0.459929 | 0.010 | 1.024 | 0.653083 | cofactor and secondary |
| | | | | | | | | metabolites |
| 1081 | menD | −0.048 | 0.896 | 0.463531 | 0.021 | 1.049 | 0.723083 | cofactor and secondary |
| | | | | | | | | metabolites |
| 1083 | menF | −0.001 | 0.997 | 0.180573 | −0.010 | 0.977 | 0.321383 | cofactor and secondary |
| | | | | | | | | metabolites |
| 1084 | yuiH | −0.055 | 0.882 | 0.482705 | 0.026 | 1.061 | 0.65875 | general function prediction only |
| 1085 | vng1085 | 0.135 | 1.364 | 0.204113 | −0.059 | 0.873 | 0.289063 | unknown |
| 1086 | vng1086 | 0.102 | 1.263 | 0.141586 | −0.036 | 0.921 | 0.543588 | unknown |
| 1087 | vng1087 | 0.019 | 1.044 | 0.55465 | −0.004 | 0.992 | 0.543133 | unknown |
| 1088 | vng1088 | −0.017 | 0.962 | 0.285517 | −0.083 | 0.825 | 0.0966 | unknown |
| 1089 | purA | 0.017 | 1.040 | 0.403098 | −0.024 | 0.947 | 0.644167 | nucleotide metabolism |
| 1090 | vng1090 | 0.050 | 1.121 | 0.47325 | −0.059 | 0.872 | 0.24225 | unknown |
| 1092 | vng1092 | 0.032 | 1.077 | 0.554096 | −0.030 | 0.934 | 0.5605 | unknown |
| 1093 | vng1093 | 0.054 | 1.132 | 0.291097 | −0.004 | 0.992 | 0.69175 | unknown |
| 1094 | vng1094 | −0.172 | 0.674 | 0.172013 | 0.010 | 1.023 | 0.580917 | unknown |
| 1095 | vng1095 | 0.133 | 1.359 | 0.142545 | −0.009 | 0.979 | 0.80975 | unknown |
| 1096 | arlR9 | −0.074 | 0.844 | 0.27607 | −0.008 | 0.981 | 0.77175 | transcription and regulation |
| 1097 | cysS | 0.012 | 1.028 | 0.635173 | 0.035 | 1.085 | 0.521 | translation |
| 1099 | vng1099 | −0.148 | 0.711 | 0.15267 | −0.020 | 0.956 | 0.68625 | unknown |
| 1100 | vng1100 | −0.147 | 0.713 | 0.079953 | −0.018 | 0.960 | 0.7535 | cofactor and secondary |
| | | | | | | | | metabolites |
| 1101 | vng1101 | −0.018 | 0.958 | 0.544671 | 0.041 | 1.100 | 0.559625 | unknown |
| 1103 | rpl12p | 0.165 | 1.462 | 0.01298 | 0.086 | 1.220 | 0.0843 | translation |
| 1104 | rpl10p | −0.003 | 0.993 | 0.267947 | 0.047 | 1.114 | 0.36975 | translation |
| 1105 | rpl1p | 0.095 | 1.245 | 0.150856 | 0.074 | 1.186 | 0.188983 | translation |
| 1108 | rpl11p | −0.019 | 0.957 | 0.54545 | −0.033 | 0.927 | 0.5385 | translation |
| 1110 | vng1110 | 0.087 | 1.221 | 0.455198 | −0.054 | 0.882 | 0.274 | unknown |
| 1111 | drg | −0.049 | 0.894 | 0.305074 | 0.027 | 1.064 | 0.501917 | translation |
| 1112 | vng1112 | −0.039 | 0.915 | 0.543402 | −0.003 | 0.993 | 0.705 | unknown |
| 1114 | glo1 | −0.008 | 0.983 | 0.470328 | −0.012 | 0.972 | 0.556667 | amino acid metabolism |
| 1115 | vng1115 | −0.014 | 0.968 | 0.822756 | 0.065 | 1.161 | 0.2885 | unknown |
| 1117 | vng1117 | 0.054 | 1.132 | 0.556431 | −0.034 | 0.925 | 0.749 | unknown |
| 1118 | vng1118 | −0.094 | 0.805 | 0.3112 | −0.049 | 0.893 | 0.420333 | translation |
| 1119 | vng1119 | −0.179 | 0.662 | 0.189 | 0.014 | 1.033 | 0.7985 | unknown |
| 1120 | vng1120 | 0.048 | 1.116 | 0.520379 | 0.006 | 1.013 | 0.63425 | unknown |
| 1121 | aspC2 | −0.136 | 0.731 | 0.499114 | −0.009 | 0.979 | 0.740833 | amino acid metabolism |
| 1123 | asxR1 | −0.079 | 0.834 | 0.26063 | 0.011 | 1.026 | 0.490375 | transcription and regulation |
| 1125 | porB | −0.040 | 0.913 | 0.546015 | 0.074 | 1.186 | 0.197785 | energy metabolism |
| 1128 | porA | −0.045 | 0.901 | 0.413681 | 0.101 | 1.262 | 0.0697 | energy metabolism |
| 1130 | vng1130 | −0.050 | 0.891 | 0.367382 | −0.010 | 0.978 | 0.8345 | unknown |
| 1131 | mrp | −0.022 | 0.951 | 0.412424 | −0.023 | 0.948 | 0.662417 | cellular process |
| 1132 | rps13p | −0.199 | 0.632 | 0.001548 | 0.048 | 1.116 | 0.34225 | translation |
| 1133 | rps4p | −0.164 | 0.685 | 0.008361 | 0.040 | 1.097 | 0.446417 | translation |
| 1134 | rps11p | −0.166 | 0.682 | 0.046558 | 0.038 | 1.092 | 0.44525 | translation |
| 1136 | rpb3 | −0.153 | 0.703 | 0.007778 | 0.057 | 1.140 | 0.329213 | transcription and regulation |
| 1137 | rpl18e | −0.218 | 0.606 | 0.003761 | 0.053 | 1.130 | 0.339117 | translation |
| 1138 | rpl13p | −0.226 | 0.594 | 0.003456 | 0.077 | 1.195 | 0.169975 | translation |
| 1139 | rps9p | −0.170 | 0.676 | 0.000959 | 0.079 | 1.199 | 0.1171 | translation |
| 1140 | rpoN | −0.269 | 0.538 | 4.4E−07 | 0.042 | 1.102 | 0.392 | transcription and regulation |
| 1141 | rpoK | −0.209 | 0.618 | 4.81E−05 | 0.074 | 1.187 | 0.13825 | transcription and regulation |
| 1142 | eno | −0.268 | 0.539 | 6.82E−05 | 0.058 | 1.144 | 0.313917 | carbohydrate and lipid metabolism |
| 1143 | rps2p | −0.133 | 0.736 | 0.279954 | 0.062 | 1.152 | 0.344133 | translation |
| 1144 | vng1144 | 0.020 | 1.047 | 0.372186 | −0.107 | 0.782 | 0.090829 | unknown |
| 1145 | mvk | −0.010 | 0.977 | 0.564774 | 0.034 | 1.081 | 0.609067 | carbohydrate and lipid metabolism |
| 1148 | argB | −0.060 | 0.870 | 0.222978 | 0.056 | 1.139 | 0.28575 | amino acid metabolism |
| 1149 | vng1149 | −0.061 | 0.870 | 0.296968 | 0.034 | 1.082 | 0.485833 | general function prediction only |
| 1150 | idsA | −0.079 | 0.834 | 0.245832 | 0.045 | 1.108 | 0.425483 | cofactor and secondary |
| | | | | | | | | metabolites |
| 1151 | vng1151 | −0.108 | 0.781 | 0.205974 | −0.013 | 0.971 | 0.7745 | unknown |
| 1153 | gltS | 0.001 | 1.002 | 0.498423 | 0.004 | 1.008 | 0.706 | translation |
| 1154 | vng1154 | 0.011 | 1.025 | 0.538912 | 0.031 | 1.073 | 0.601375 | unknown |
| 1156 | hsp3 | 0.045 | 1.110 | 0.404091 | −0.047 | 0.897 | 0.447833 | cellular process |
| 1157 | rphs6 | 0.076 | 1.192 | 0.310131 | 0.053 | 1.129 | 0.31875 | translation |
| 1158 | rps28e | 0.081 | 1.204 | 0.105495 | 0.069 | 1.172 | 0.2467 | translation |
| 1159 | rpl24e | 0.034 | 1.082 | 0.512475 | 0.056 | 1.138 | 0.32285 | translation |
| 1160 | ndk | 0.036 | 1.087 | 0.565343 | 0.058 | 1.142 | 0.3531 | nucleotide metabolism |
| 1162 | vng1162 | 0.027 | 1.065 | 0.334177 | −0.016 | 0.963 | 0.75875 | unknown |
| 1163 | hemK | −0.050 | 0.891 | 0.384562 | −0.035 | 0.923 | 0.498 | cofactor and secondary |
| | | | | | | | | metabolites |
| 1164 | vng1164 | 0.008 | 1.018 | 0.668004 | −0.002 | 0.995 | 0.789333 | unknown |
| 1165 | ksgA | 0.037 | 1.089 | 0.577529 | 0.015 | 1.035 | 0.778417 | translation |
| 1168 | vng1168 | 0.115 | 1.303 | 0.227096 | 0.022 | 1.051 | 0.62175 | translation |
| 1169 | vng1169 | 0.035 | 1.085 | 0.500083 | −0.008 | 0.983 | 0.794 | unknown |
| 1170 | rpl21e | 0.070 | 1.176 | 0.41396 | −0.019 | 0.957 | 0.705 | translation |
| 1172 | metB | 0.139 | 1.379 | 0.156709 | −0.017 | 0.961 | 0.685833 | amino acid metabolism |
| 1173 | eef1b | −0.068 | 0.856 | 0.270058 | −0.042 | 0.908 | 0.52645 | translation |
| 1174 | nop58 | 0.032 | 1.076 | 0.358708 | −0.060 | 0.870 | 0.390267 | translation |
| 1175 | phoR | −0.245 | 0.569 | 0.268902 | −0.034 | 0.925 | 0.483092 | cellular process |
| 1176 | fib | −0.022 | 0.951 | 0.202874 | −0.018 | 0.959 | 0.507083 | translation |
| 1178 | vng1178 | −0.050 | 0.892 | 0.669247 | −0.008 | 0.982 | 0.9285 | unknown |
| 1179 | acxR | 0.038 | 1.091 | 0.646511 | −0.053 | 0.885 | 0.55725 | transcription and regulation |
| 1180 | msrA | 0.003 | 1.008 | 0.34428 | −0.004 | 0.990 | 0.66 | cellular process |
| 1181 | flaA1b | 0.031 | 1.074 | 0.492426 | −0.049 | 0.894 | 0.573483 | cellular process |
| 1182 | vng1182 | 0.000 | 1.000 | 0.545927 | 0.012 | 1.027 | 0.806583 | unknown |
| 1183 | nirJ | 0.023 | 1.055 | 0.640616 | −0.107 | 0.782 | 0.082355 | unknown |
| 1184 | vng1183 | 0.064 | 1.159 | 0.567241 | 0.029 | 1.068 | 0.602583 | cofactor and secondary |
| | | | | | | | | metabolites |
| 1185 | pqqE | 0.040 | 1.096 | 0.410609 | 0.064 | 1.158 | 0.23535 | cofactor and secondary |
| | | | | | | | | metabolites |
| 1187 | pan1 | 0.037 | 1.089 | 0.509893 | −0.054 | 0.883 | 0.555258 | energy metabolism |
| 1188 | hcpD | −0.012 | 0.974 | 0.814744 | 0.049 | 1.119 | 0.3755 | energy metabolism |
| 1189 | vng1189 | 0.038 | 1.090 | 0.516765 | −0.106 | 0.784 | 0.04545 | unknown |
| 1190 | sod1 | 0.394 | 2.477 | 9.03E−09 | 0.022 | 1.052 | 0.661 | cellular process |
| 1191 | acd3 | 0.025 | 1.060 | 0.692548 | 0.001 | 1.003 | 0.859083 | carbohydrate and lipid metabolism |
| 1193 | hik1 | −0.043 | 0.907 | 0.39923 | −0.112 | 0.772 | 0.264817 | cellular process |
| 1194 | vng1194 | −0.028 | 0.938 | 0.559434 | −0.043 | 0.905 | 0.4285 | unknown |
| 1196 | vng1196 | −0.042 | 0.908 | 0.546216 | −0.176 | 0.666 | 0.000753 | unknown |
| 1197 | bcp | 0.156 | 1.434 | 0.043264 | −0.068 | 0.855 | 0.217583 | cellular process |
| 1198 | vng1198 | 0.021 | 1.049 | 0.554699 | 0.005 | 1.013 | 0.711917 | energy metabolism |
| 1200 | vng1200 | 0.048 | 1.118 | 0.615038 | 0.078 | 1.196 | 0.324125 | unknown |
| 1201 | fucA | −0.034 | 0.925 | 0.339155 | −0.061 | 0.870 | 0.39725 | carbohydrate and lipid metabolism |
| 1202 | vng1202 | −0.122 | 0.754 | 0.334159 | 0.004 | 1.009 | 0.842083 | unknown |
| 1204 | gdhA2 | −0.067 | 0.858 | 0.257105 | −0.004 | 0.990 | 0.8025 | amino acid metabolism |
| 1205 | vng1205 | 0.023 | 1.054 | 0.271139 | 0.082 | 1.208 | 0.287982 | nucleotide metabolism |
| 1207 | bolR4 | −0.038 | 0.917 | 0.533929 | −0.044 | 0.903 | 0.598625 | transcription and regulation |
| 1208 | hutU | −0.032 | 0.930 | 0.541442 | 0.027 | 1.065 | 0.555417 | amino acid metabolism |
| 1209 | hutG | 0.007 | 1.016 | 0.366907 | −0.007 | 0.983 | 0.758083 | amino acid metabolism |
| 1211 | hutI | 0.004 | 1.009 | 0.449435 | 0.000 | 1.000 | 0.3881 | amino acid metabolism |
| 1212 | hutH | 0.022 | 1.051 | 0.700657 | 0.015 | 1.034 | 0.6907 | amino acid metabolism |
| 1213 | vng1213 | 0.091 | 1.233 | 0.26307 | −0.005 | 0.989 | 0.853583 | DNA metabolism |
| 1214 | vng1214 | −0.043 | 0.906 | 0.476107 | 0.005 | 1.013 | 0.83575 | unknown |
| 1215 | pai1 | 0.020 | 1.046 | 0.668489 | −0.010 | 0.977 | 0.3715 | transcription and regulation |
| 1216 | pgk | 0.010 | 1.023 | 0.069506 | 0.007 | 1.015 | 0.8645 | carbohydrate and lipid metabolism |
| 1218 | vng1218 | 0.046 | 1.113 | 0.760099 | −0.036 | 0.920 | 0.684833 | unknown |
| 1219 | urk | −0.092 | 0.808 | 0.411449 | −0.016 | 0.963 | 0.558667 | nucleotide metabolism |
| 1220 | vng1220 | 0.011 | 1.025 | 0.373897 | 0.000 | 0.999 | 0.7155 | unknown |
| 1224 | orc8 | −0.168 | 0.680 | 0.198518 | 0.050 | 1.123 | 0.477283 | DNA metabolism |
| 1226 | vng1226 | −0.040 | 0.913 | 0.425506 | 0.035 | 1.083 | 0.6105 | unknown |
| 1227 | vng1227 | 0.026 | 1.062 | 0.582669 | −0.001 | 0.998 | 0.678583 | unknown |
| 1228 | vng1228 | 0.065 | 1.161 | 0.460962 | −0.042 | 0.908 | 0.489667 | DNA metabolism |
| 1229 | vng1229 | 0.054 | 1.133 | 0.448475 | 0.021 | 1.050 | 0.732583 | transcription and regulation |
| 1230 | aroC | −0.021 | 0.952 | 0.692906 | 0.039 | 1.093 | 0.540417 | amino acid metabolism |
| 1232 | psc | −0.006 | 0.987 | 0.539962 | 0.045 | 1.110 | 0.526667 | amino acid metabolism |
| 1233 | pepQ2 | 0.172 | 1.485 | 0.15379 | −0.045 | 0.902 | 0.47475 | amino acid metabolism |
| 1234 | hik2 | −0.030 | 0.934 | 0.685947 | −0.020 | 0.956 | 0.699667 | cellular process |
| 1235 | vng1235 | −0.056 | 0.879 | 0.398733 | −0.040 | 0.911 | 0.474125 | amino acid metabolism |
| 1236 | vng1236 | −0.031 | 0.931 | 0.631792 | −0.044 | 0.904 | 0.390893 | unknown |
| 1237 | asxR2 | −0.038 | 0.917 | 0.159942 | −0.010 | 0.976 | 0.576167 | transcription and regulation |
| 1238 | vng1239 | 0.016 | 1.037 | 0.461979 | −0.360 | 0.437 | 0.000286 | unknown |
| 1239 | vng1238 | −0.024 | 0.947 | 0.640347 | 0.056 | 1.139 | 0.34305 | unknown |
| 1240 | yhdG | −0.096 | 0.801 | 0.338979 | −0.679 | 0.210 | 9.5E−14 | transport |
| 1241 | surE | 0.040 | 1.097 | 0.248556 | −0.091 | 0.811 | 0.19539 | general function prediction only |
| 1244 | vng1244 | −0.025 | 0.943 | 0.752072 | 0.103 | 1.267 | 0.126 | amino acid metabolism |
| 1245 | vng1245 | 0.062 | 1.154 | 0.34147 | 0.028 | 1.067 | 0.581625 | amino acid metabolism |
| 1246 | vng1246 | 0.015 | 1.036 | 0.464561 | −0.060 | 0.872 | 0.30905 | unknown |
| 1247 | ybhF | −0.045 | 0.902 | 0.416675 | 0.012 | 1.028 | 0.7115 | transport |
| 1249 | vng1249 | −0.141 | 0.723 | 0.433738 | 0.060 | 1.149 | 0.3765 | unknown |
| 1250 | vng1250 | −0.089 | 0.814 | 0.286173 | 0.069 | 1.172 | 0.2051 | unknown |
| 1251 | helA | −0.051 | 0.889 | 0.445395 | 0.026 | 1.063 | 0.691833 | DNA metabolism |
| 1252 | yhcR | 0.056 | 1.138 | 0.371538 | −0.058 | 0.875 | 0.317533 | general function prediction only |
| 1253 | vng1253 | −0.220 | 0.602 | 0.167789 | −0.039 | 0.914 | 0.69625 | unknown |
| 1255 | vng1255 | −0.064 | 0.863 | 0.270715 | −0.154 | 0.701 | 0.239056 | DNA metabolism |
| 1256 | ribG | −0.041 | 0.910 | 0.327279 | −0.024 | 0.947 | 0.65722 | cofactor and secondary |
| | | | | | | | | metabolites |
| 1257 | vng1257 | 0.008 | 1.019 | 0.684337 | −0.047 | 0.897 | 0.3815 | unknown |
| 1259 | trx82 | 0.014 | 1.033 | 0.371511 | 0.031 | 1.073 | 0.480083 | nucleotide metabolism |
| 1260 | vng1260 | 0.131 | 1.352 | 0.277642 | −0.048 | 0.896 | 0.547 | unknown |
| 1261 | vng1261 | 0.245 | 1.759 | 0.000239 | −0.177 | 0.666 | 0.002834 | unknown |
| 1262 | eif2b | 0.030 | 1.071 | 0.587663 | −0.046 | 0.899 | 0.38825 | translation |
| 1263 | vng1263 | 0.056 | 1.137 | 0.385844 | −0.003 | 0.993 | 0.9475 | unknown |
| 1264 | vng1264 | 0.018 | 1.043 | 0.747845 | 0.003 | 1.006 | 0.642875 | unknown |
| 1266 | ogg | 0.022 | 1.051 | 0.079735 | −0.009 | 0.980 | 0.787083 | DNA metabolism |
| 1268 | vng1268 | −0.216 | 0.608 | 0.182681 | 0.064 | 1.159 | 0.298083 | unknown |
| 1270 | vng1270 | −0.076 | 0.840 | 0.544644 | −0.027 | 0.939 | 0.89775 | unknown |
| 1271 | vng1271 | 0.101 | 1.262 | 0.296749 | −0.041 | 0.911 | 0.593625 | unknown |
| 1272 | vng1272 | 0.036 | 1.087 | 0.545394 | −0.019 | 0.957 | 0.693583 | unknown |
| 1273 | moaC | −0.001 | 0.997 | 0.277206 | −0.005 | 0.988 | 0.320875 | cofactor and secondary |
| | | | | | | | | metabolites |
| 1275 | hflX2 | 0.020 | 1.047 | 0.590433 | −0.022 | 0.950 | 0.682417 | cellular process |
| 1276 | vng1276 | −0.003 | 0.994 | 0.406927 | −0.018 | 0.959 | 0.748833 | translation |
| 1279 | vng1279 | −0.045 | 0.902 | 0.564437 | 0.031 | 1.074 | 0.72125 | translation |
| 1280 | vng1280 | −0.013 | 0.970 | 0.498362 | 0.015 | 1.035 | 0.768 | cofactor and secondary |
| | | | | | | | | metabolites |
| 1281 | vng1281 | −0.181 | 0.658 | 0.192621 | 0.077 | 1.195 | 0.178325 | translation |
| 1282 | trkA5 | −0.032 | 0.929 | 0.275835 | 0.000 | 1.000 | 0.74125 | transport |
| 1283 | vng1283 | −0.128 | 0.745 | 0.456531 | −0.019 | 0.958 | 0.86325 | unknown |
| 1284 | trkH1 | −0.075 | 0.842 | 0.345179 | 0.020 | 1.047 | 0.458317 | transport |
| 1285 | asxR3 | 0.020 | 1.046 | 0.527123 | 0.026 | 1.061 | 0.617017 | transcription and regulation |
| 1287 | vng1287 | −0.019 | 0.958 | 0.398989 | −0.019 | 0.958 | 0.7325 | translation |
| 1289 | vng1289 | −0.068 | 0.855 | 0.306832 | −0.165 | 0.684 | 0.00139 | unknown |
| 1291 | vng1291 | 0.007 | 1.017 | 0.74382 | 0.013 | 1.031 | 0.7685 | unknown |
| 1292 | vng1292 | 0.001 | 1.002 | 0.742682 | −0.003 | 0.993 | 0.839 | unknown |
| 1294 | slyD | 0.020 | 1.048 | 0.569987 | −0.004 | 0.990 | 0.809917 | cellular process |
| 1295 | vng1295 | 0.023 | 1.054 | 0.252637 | −0.011 | 0.975 | 0.73075 | unknown |
| 1296 | vng1296 | −0.099 | 0.796 | 0.386504 | −0.021 | 0.952 | 0.72375 | nucleotide metabolism |
| 1297 | vng1297 | 0.040 | 1.098 | 0.514493 | −0.065 | 0.860 | 0.27165 | amino acid metabolism |
| 1299 | vng1299 | 0.064 | 1.160 | 0.347439 | −0.008 | 0.982 | 0.741417 | cofactor and secondary |
| | | | | | | | | metabolites |
| 1300 | vng1300 | −0.027 | 0.941 | 0.359237 | −0.025 | 0.943 | 0.417875 | unknown |
| 1301 | cysK | −0.007 | 0.983 | 0.689341 | −0.047 | 0.898 | 0.562525 | amino acid metabolism |
| 1302 | vng1302 | 0.147 | 1.403 | 0.014266 | −0.081 | 0.829 | 0.13045 | cellular process |
| 1303 | vng1303 | −0.022 | 0.951 | 0.566574 | 0.009 | 1.022 | 0.528167 | cellular process |
| 1305 | purD | 0.002 | 1.004 | 0.523444 | 0.033 | 1.080 | 0.546833 | nucleotide metabolism |
| 1306 | sdhA | −0.088 | 0.817 | 0.212496 | 0.054 | 1.132 | 0.32575 | energy metabolism |
| 1308 | sdhB | −0.040 | 0.911 | 0.538863 | 0.049 | 1.119 | 0.37225 | energy metabolism |
| 1309 | sdhD | 0.048 | 1.117 | 0.262107 | 0.073 | 1.182 | 0.3271 | energy metabolism |
| 1310 | sdhC | 0.004 | 1.010 | 0.531501 | 0.065 | 1.162 | 0.249233 | energy metabolism |
| 1311 | alkA | 0.011 | 1.025 | 0.833652 | 0.047 | 1.114 | 0.7885 | DNA metabolism |
| 1313 | hbd2 | 0.176 | 1.499 | 0.083115 | −0.015 | 0.967 | 0.78575 | carbohydrate and lipid metabolism |
| 1314 | vng1314 | 0.007 | 1.016 | 0.6968 | 0.084 | 1.213 | 0.13205 | unknown |
| 1315 | vng1315 | 0.021 | 1.050 | 0.690545 | 0.096 | 1.247 | 0.08465 | unknown |
| 1317 | vng1317 | 0.018 | 1.042 | 0.512463 | 0.024 | 1.057 | 0.531583 | unknown |
| 1318 | vng1318 | −0.016 | 0.963 | 0.569509 | −0.085 | 0.823 | 0.20665 | unknown |
| 1319 | vng1319 | 0.047 | 1.113 | 0.157177 | 0.025 | 1.059 | 0.646125 | unknown |
| 1320 | cbp | −0.247 | 0.567 | 0.237123 | 0.052 | 1.126 | 0.500833 | cellular process |
| 1323 | vng1323 | −0.132 | 0.737 | 0.316899 | 0.017 | 1.040 | 0.349317 | unknown |
| 1324 | vng1324 | −0.035 | 0.923 | 0.642943 | −0.035 | 0.923 | 0.60275 | unknown |
| 1325 | vng1325 | 0.120 | 1.318 | 0.191145 | 0.015 | 1.036 | 0.754417 | nucleotide metabolism |
| 1326 | vng1326 | 0.027 | 1.064 | 0.234208 | −0.038 | 0.916 | 0.474875 | unknown |
| 1327 | fprA | 0.126 | 1.336 | 0.035299 | −0.016 | 0.964 | 0.727 | general function prediction only |
| 1329 | vng1329 | 0.110 | 1.289 | 0.047948 | 0.015 | 1.035 | 0.7685 | unknown |
| 1330 | vng1330 | −0.007 | 0.984 | 0.311984 | −0.048 | 0.896 | 0.428367 | unknown |
| 1332 | sod2 | 0.404 | 2.536 | 1.21E−08 | 0.034 | 1.080 | 0.503 | cellular process |
| 1335 | phr2 | 0.113 | 1.298 | 0.247921 | −0.018 | 0.958 | 0.739833 | DNA metabolism |
| 1336 | vng1336 | −0.001 | 0.997 | 0.296433 | −0.004 | 0.991 | 0.512583 | cofactor and secondary |
| | | | | | | | | metabolites |
| 1337 | vng1337 | −0.060 | 0.871 | 0.491862 | −0.030 | 0.934 | 0.72725 | unknown |
| 1339 | vng1339 | 0.373 | 2.361 | 0.0049 | 0.007 | 1.017 | 0.640458 | carbohydrate and lipid metabolism |
| 1340 | vng1340 | 0.064 | 1.159 | 0.454011 | −0.013 | 0.970 | 0.640442 | unknown |
| 1341 | fabG | 0.143 | 1.389 | 0.007682 | −0.006 | 0.987 | 0.78825 | cofactor and secondary |
| | | | | | | | | metabolites |
| 1342 | mer | 0.052 | 1.128 | 0.50106 | −0.006 | 0.987 | 0.77525 | energy metabolism |
| 1343 | vng1343 | −0.016 | 0.963 | 0.520386 | −0.011 | 0.975 | 0.783333 | unknown |
| 1344 | dhpS | −0.022 | 0.951 | 0.553255 | 0.010 | 1.023 | 0.430733 | cofactor and secondary |
| | | | | | | | | metabolites |
| 1345 | vng1345 | 0.019 | 1.045 | 0.670066 | −0.017 | 0.961 | 0.73175 | unknown |
| 1347 | vng1347 | 0.008 | 1.019 | 0.692549 | −0.232 | 0.586 | 0.12975 | translation |
| 1349 | vng1349 | −0.141 | 0.723 | 0.012778 | 0.024 | 1.058 | 0.652 | carbohydrate and lipid metabolism |
| 1350 | vng1350 | −0.016 | 0.965 | 0.761595 | 0.040 | 1.095 | 0.509667 | carbohydrate and lipid metabolism |
| 1351 | asxR4 | 0.062 | 1.154 | 0.210371 | −0.061 | 0.870 | 0.361925 | transcription and regulation |
| 1352 | gatB1 | −0.117 | 0.763 | 0.479398 | 0.020 | 1.047 | 0.729667 | translation |
| 1353 | vng1353 | 0.023 | 1.055 | 0.527519 | 0.031 | 1.073 | 0.66575 | unknown |
| 1355 | vng1355 | −0.024 | 0.947 | 0.632185 | −0.013 | 0.970 | 0.69675 | unknown |
| 1356 | fumC | 0.216 | 1.643 | 0.073567 | −0.010 | 0.976 | 0.738583 | energy metabolism |
| 1357 | balR | −0.074 | 0.844 | 0.390655 | 0.004 | 1.010 | 0.8395 | transcription and regulation |
| 1359 | rad2 | 0.011 | 1.025 | 0.569664 | −0.081 | 0.831 | 0.417464 | DNA metabolism |
| 1360 | vng1360 | 0.055 | 1.134 | 0.254793 | 0.001 | 1.002 | 0.9375 | unknown |
| 1362 | vng1362 | 0.073 | 1.184 | 0.389231 | 0.011 | 1.025 | 0.776 | unknown |
| 1364 | ocd2 | 0.121 | 1.323 | 0.087478 | −0.031 | 0.930 | 0.5065 | amino acid metabolism |
| 1365 | vng1365 | 0.005 | 1.012 | 0.680168 | −0.012 | 0.973 | 0.775167 | unknown |
| 1366 | vng1366 | 0.041 | 1.098 | 0.696743 | −0.017 | 0.961 | 0.840875 | translation |
| 1367 | srp19 | 0.059 | 1.145 | 0.277273 | 0.004 | 1.010 | 0.89225 | cellular process |
| 1369 | hemV1 | 0.078 | 1.197 | 0.307635 | −0.035 | 0.923 | 0.562 | transport |
| 1370 | hemU | 0.002 | 1.005 | 0.468647 | 0.035 | 1.083 | 0.6445 | transport |
| 1371 | hemV2 | −0.086 | 0.820 | 0.433523 | −0.016 | 0.965 | 0.669667 | transport |
| 1372 | vng1372 | 0.030 | 1.071 | 0.215736 | −0.087 | 0.819 | 0.349601 | unknown |
| 1374 | kinA1 | −0.020 | 0.956 | 0.249695 | −0.040 | 0.911 | 0.253334 | cellular process |
| 1375 | vng1375 | −0.078 | 0.836 | 0.153444 | −0.098 | 0.797 | 0.251465 | cellular process |
| 1376 | vng1376 | −0.058 | 0.875 | 0.350247 | 0.006 | 1.014 | 0.784625 | unknown |
| 1377 | asnC | 0.002 | 1.005 | 0.58864 | −0.032 | 0.929 | 0.247525 | transcription and regulation |
| 1379 | suhB | −0.013 | 0.971 | 0.799067 | 0.044 | 1.106 | 0.40375 | carbohydrate and lipid metabolism |
| 1380 | vng1380 | 0.109 | 1.284 | 0.353529 | −0.006 | 0.987 | 0.58925 | unknown |
| 1381 | vng1381 | 0.117 | 1.310 | 0.068696 | −0.015 | 0.966 | 0.6455 | unknown |
| 1382 | vng1382 | −0.023 | 0.949 | 0.600692 | −0.140 | 0.724 | 0.533833 | unknown |
| 1383 | rad3a | 0.010 | 1.023 | 0.485189 | −0.020 | 0.954 | 0.536633 | DNA metabolism |
| 1384 | vng1384 | −0.070 | 0.851 | 0.485715 | −0.055 | 0.881 | 0.43075 | unknown |
| 1385 | yvoF | 0.012 | 1.027 | 0.45117 | −0.028 | 0.937 | 0.62825 | general function prediction only |
| 1387 | vng1387 | 0.055 | 1.134 | 0.452404 | 0.031 | 1.075 | 0.515767 | unknown |
| 1388 | vng1388 | −0.026 | 0.942 | 0.522302 | −0.004 | 0.990 | 0.313067 | unknown |
| 1389 | vng1389 | 0.070 | 1.175 | 0.443469 | −0.046 | 0.900 | 0.452417 | unknown |
| 1390 | tfl | 0.140 | 1.379 | 0.140177 | −0.046 | 0.899 | 0.6925 | transcription and regulation |
| 1394 | arlR10 | 0.110 | 1.288 | 0.091138 | −0.068 | 0.856 | 0.237825 | transcription and regulation |
| 1395 | htr9 | 0.022 | 1.053 | 0.517603 | −0.008 | 0.982 | 0.424208 | cellular process |
| 1397 | vng1397 | 0.024 | 1.057 | 0.43971 | −0.013 | 0.971 | 0.697 | unknown |
| 1398 | act4 | −0.019 | 0.958 | 0.714138 | 0.025 | 1.060 | 0.543 | transcription and regulation |
| 1401 | vng1401 | 0.037 | 1.089 | 0.29498 | 0.012 | 1.027 | 0.8045 | unknown |
| 1402 | vng1402 | 0.034 | 1.081 | 0.563308 | −0.018 | 0.960 | 0.85125 | unknown |
| 1403 | vng1403 | −0.001 | 0.997 | 0.276913 | −0.221 | 0.601 | 0.39375 | unknown |
| 1404 | trh1 | 0.068 | 1.170 | 0.054831 | −0.081 | 0.831 | 0.26785 | cellular process |
| 1405 | mrlR | −0.091 | 0.811 | 0.268966 | 0.036 | 1.086 | 0.565 | transcription and regulation |
| 1406 | rad25b | −0.017 | 0.963 | 0.633603 | −0.046 | 0.900 | 0.42035 | DNA metabolism |
| 1407 | vng1407 | 0.021 | 1.050 | 0.661305 | −0.044 | 0.903 | 0.409375 | unknown |
| 1408 | ush | 0.230 | 1.696 | 0.000239 | 0.029 | 1.070 | 0.601167 | nucleotide metabolism |
| 1409 | vng1409 | 0.078 | 1.196 | 0.047544 | 0.002 | 1.004 | 0.764833 | unknown |
| 1410 | vng1410 | −0.036 | 0.920 | 0.55512 | −0.006 | 0.985 | 0.736 | unknown |
| 1412 | vng1412 | 0.081 | 1.205 | 0.37828 | −0.018 | 0.959 | 0.730375 | unknown |
| 1413 | vng1413 | −0.019 | 0.958 | 0.548887 | 0.027 | 1.063 | 0.547483 | unknown |
| 1414 | glyA | 0.128 | 1.343 | 0.096834 | 0.068 | 1.169 | 0.3196 | amino acid metabolism |
| 1416 | folD | 0.076 | 1.190 | 0.365463 | 0.063 | 1.157 | 0.284375 | cofactor and secondary |
| | | | | | | | | metabolites |
| 1417 | vng1417 | 0.034 | 1.081 | 0.246601 | −0.004 | 0.991 | 0.729917 | unknown |
| 1418 | vng1418 | 0.030 | 1.073 | 0.621395 | −0.069 | 0.853 | 0.381507 | unknown |
| 1420 | vng1420 | −0.149 | 0.709 | 0.073131 | −0.003 | 0.993 | 0.858 | unknown |
| 1422 | vng1422 | 0.033 | 1.079 | 0.507841 | −0.025 | 0.944 | 0.615875 | unknown |
| 1423 | vng1423 | −0.084 | 0.824 | 0.255802 | −0.060 | 0.871 | 0.456313 | unknown |
| 1425 | vng1425 | 0.024 | 1.056 | 0.748285 | −0.001 | 0.998 | 0.69525 | unknown |
| 1426 | prlR2 | 0.105 | 1.274 | 0.175212 | −0.024 | 0.946 | 0.64925 | transcription and regulation |
| 1427 | vng1427 | 0.040 | 1.096 | 0.612385 | −0.069 | 0.854 | 0.29157 | unknown |
| 1428 | htlA | −0.017 | 0.961 | 0.721196 | 0.035 | 1.084 | 0.47925 | cellular process |
| 1429 | vng1429 | 0.035 | 1.084 | 0.473861 | 0.048 | 1.116 | 0.359 | unknown |
| 1431 | vng1431 | 0.038 | 1.090 | 0.275885 | 0.003 | 1.008 | 0.76825 | translation |
| 1432 | dys | −0.149 | 0.709 | 0.365183 | 0.055 | 1.134 | 0.364458 | translation |
| 1433 | rps17e | 0.085 | 1.216 | 0.274871 | −0.098 | 0.798 | 0.05635 | translation |
| 1435 | asd | 0.005 | 1.011 | 0.577351 | 0.034 | 1.081 | 0.51875 | amino acid metabolism |
| 1437 | serA2 | 0.122 | 1.324 | 0.027276 | −0.056 | 0.880 | 0.29825 | amino acid metabolism |
| 1438 | arlR10 | 0.154 | 1.424 | 0.028858 | −0.015 | 0.966 | 0.6165 | transcription and regulation |
| 1440 | vng1440 | −0.003 | 0.993 | 0.415811 | 0.021 | 1.049 | 0.689 | unknown |
| 1442 | htr12 | −0.106 | 0.783 | 0.102934 | 0.026 | 1.061 | 0.636 | cellular process |
| 1444 | hisD | 0.062 | 1.153 | 0.390048 | 0.012 | 1.027 | 0.519067 | amino acid metabolism |
| 1446 | vng1446 | −0.103 | 0.789 | 0.144529 | 0.087 | 1.222 | 0.1153 | unknown |
| 1447 | vng1447 | 0.015 | 1.036 | 0.340384 | −0.036 | 0.921 | 0.534 | unknown |
| 1448 | vng1448 | −0.054 | 0.882 | 0.298868 | 0.017 | 1.040 | 0.439358 | unknown |
| 1450 | yfkN | 0.006 | 1.014 | 0.661665 | −0.027 | 0.939 | 0.59175 | cofactor and secondary |
| | | | | | | | | metabolites |
| 1451 | sstR1 | −0.009 | 0.980 | 0.509143 | −0.025 | 0.944 | 0.663917 | transcription and regulation |
| 1452 | eif2bd | −0.165 | 0.683 | 0.401074 | 0.032 | 1.078 | 0.475571 | translation |
| 1453 | vng1453 | 0.125 | 1.333 | 0.150685 | 0.030 | 1.072 | 0.566167 | unknown |
| 1454 | vng1454 | 0.035 | 1.083 | 0.659511 | 0.016 | 1.037 | 0.633158 | unknown |
| 1455 | vng1455 | 0.092 | 1.236 | 0.354031 | 0.000 | 1.000 | 0.519083 | unknown |
| 1456 | vng1456 | 0.051 | 1.125 | 0.520253 | −0.026 | 0.943 | 0.81425 | unknown |
| 1457 | vng1457 | −0.119 | 0.760 | 0.079643 | −0.046 | 0.900 | 0.3895 | unknown |
| 1458 | crtB1 | −0.064 | 0.864 | 0.490885 | 0.060 | 1.147 | 0.605 | carbohydrate and lipid metabolism |
| 1459 | vng1459 | −0.051 | 0.889 | 0.469871 | 0.087 | 1.221 | 0.1245 | unknown |
| 1461 | vng1461 | −0.090 | 0.813 | 0.544937 | 0.062 | 1.152 | 0.42225 | unknown |
| 1462 | cdc48a | 0.023 | 1.054 | 0.64727 | 0.016 | 1.037 | 0.532083 | cellular process |
| 1463 | blp | −0.003 | 0.994 | 0.380257 | 0.031 | 1.074 | 0.635917 | energy metabolism |
| 1464 | bat | −0.049 | 0.893 | 0.651315 | 0.024 | 1.056 | 0.846417 | transcription and regulation |
| 1465 | brp | −0.020 | 0.956 | 0.678546 | 0.038 | 1.091 | 0.700333 | energy metabolism |
| 1466 | vng1466 | 0.073 | 1.183 | 0.426187 | −0.094 | 0.806 | 0.242463 | energy metabolism |
| 1467 | bop | −0.219 | 0.604 | 0.280747 | 0.206 | 1.606 | 0.206848 | energy metabolism |
| 1468 | vng1468 | 0.084 | 1.213 | 0.437013 | 0.113 | 1.298 | 0.0652 | unknown |
| 1470 | pri | −0.067 | 0.857 | 0.395277 | 0.020 | 1.048 | 0.463583 | DNA metabolism |
| 1471 | vng1471 | −0.046 | 0.899 | 0.378799 | 0.027 | 1.065 | 0.587208 | unknown |
| 1472 | cdc48b | 0.052 | 1.128 | 0.119714 | 0.039 | 1.094 | 0.464667 | cellular process |
| 1473 | vng1473 | 0.172 | 1.485 | 0.363164 | 0.083 | 1.210 | 0.12265 | unknown |
| 1474 | est | 0.095 | 1.244 | 0.270725 | −0.043 | 0.905 | 0.483367 | general function prediction only |
| 1475 | vng1475 | 0.006 | 1.014 | 0.585737 | −0.005 | 0.989 | 0.755833 | unknown |
| 1476 | vng1476 | 0.055 | 1.135 | 0.410343 | −0.036 | 0.921 | 0.573292 | unknown |
| 1478 | panB | 0.224 | 1.675 | 0.113975 | −0.022 | 0.951 | 0.695875 | cofactor and secondary |
| | | | | | | | | metabolites |
| 1479 | vng1479 | −0.108 | 0.779 | 0.059563 | −0.071 | 0.849 | 0.25125 | unknown |
| 1480 | vng1480 | −0.057 | 0.877 | 0.379361 | 0.010 | 1.022 | 0.843 | unknown |
| 1481 | sat | 0.105 | 1.274 | 0.140907 | 0.014 | 1.033 | 0.7775 | amino acid metabolism |
| 1482 | acd5 | 0.082 | 1.208 | 0.299358 | −0.020 | 0.954 | 0.65075 | carbohydrate and lipid metabolism |
| 1483 | mbf | 0.045 | 1.109 | 0.459602 | −0.030 | 0.934 | 0.555367 | transcription and regulation |
| 1484 | vng1484 | 0.011 | 1.027 | 0.722924 | −0.067 | 0.858 | 0.500915 | unknown |
| 1486 | vng1486 | 0.019 | 1.046 | 0.275346 | −0.030 | 0.934 | 0.6365 | unknown |
| 1487 | vng1487 | −0.051 | 0.890 | 0.20714 | −0.011 | 0.976 | 0.473 | unknown |
| 1488 | boa2 | 0.102 | 1.265 | 0.147692 | −0.016 | 0.964 | 0.7585 | transcription and regulation |
| 1490 | arlR11 | 0.105 | 1.275 | 0.376588 | −0.044 | 0.904 | 0.325375 | transcription and regulation |
| 1492 | vng1492 | −0.011 | 0.976 | 0.722637 | −0.017 | 0.961 | 0.757167 | unknown |
| 1493 | purF | 0.052 | 1.126 | 0.439948 | 0.125 | 1.335 | 0.024154 | nucleotide metabolism |
| 1494 | rpl37e | 0.135 | 1.366 | 0.047862 | −0.062 | 0.866 | 0.27025 | translation |
| 1496 | snp | 0.027 | 1.063 | 0.011742 | −0.100 | 0.793 | 0.0578 | transcription and regulation |
| 1497 | Vng1497 | −0.080 | 0.832 | 0.287317 | 0.013 | 1.030 | 0.702917 | unknown |
| 1498 | celM | 0.155 | 1.429 | 0.037008 | 0.056 | 1.138 | 0.358 | carbohydrate and lipid metabolism |
| 1500 | vng1500 | 0.058 | 1.144 | 0.366672 | −0.005 | 0.989 | 0.8685 | unknown |
| 1501 | hel | 0.067 | 1.167 | 0.430406 | −0.008 | 0.982 | 0.638 | DNA metabolism |
| 1503 | vng1503 | 0.011 | 1.026 | 0.585421 | −0.137 | 0.730 | 0.2719 | unknown |
| 1505 | htr10 | −0.090 | 0.812 | 0.606968 | 0.011 | 1.026 | 0.8435 | cellular process |
| 1506 | pelA | −0.020 | 0.956 | 0.49381 | 0.020 | 1.048 | 0.700667 | cellular process |
| 1508 | snp2 | 0.021 | 1.049 | 0.275967 | 0.013 | 1.030 | 0.753333 | transcription and regulation |
| 1510 | act5 | 0.029 | 1.068 | 0.630277 | 0.029 | 1.069 | 0.620167 | transcription and regulation |
| 1511 | vng1511 | −0.015 | 0.965 | 0.371943 | −0.011 | 0.975 | 0.723167 | DNA metabolism |
| 1513 | vng1513 | 0.038 | 1.090 | 0.547906 | −0.216 | 0.608 | 0.00029 | unknown |
| 1514 | vng1514 | 0.063 | 1.155 | 0.506648 | 0.040 | 1.097 | 0.493375 | unknown |
| 1515 | tdk | −0.048 | 0.895 | 0.41244 | 0.059 | 1.145 | 0.29975 | nucleotide metabolism |
| 1518 | vng1518 | 0.054 | 1.131 | 0.254564 | 0.003 | 1.007 | 0.515875 | cellular process |
| 1519 | vng1519 | −0.047 | 0.898 | 0.483436 | −0.012 | 0.973 | 0.8675 | unknown |
| 1520 | mutY | 0.010 | 1.022 | 0.369763 | 0.024 | 1.057 | 0.62275 | DNA metabolism |
| 1523 | htr8 | −0.033 | 0.927 | 0.288662 | 0.004 | 1.010 | 0.855667 | cellular process |
| 1524 | vng1524 | 0.233 | 1.708 | 0.048145 | 0.011 | 1.025 | 0.6665 | amino acid metabolism |
| 1525 | vng1525 | −0.039 | 0.914 | 0.554599 | 0.030 | 1.071 | 0.633217 | unknown |
| 1526 | trp7 | 0.075 | 1.188 | 0.429332 | −0.014 | 0.968 | 0.519083 | transport |
| 1528 | nac | 0.086 | 1.219 | 0.504901 | −0.038 | 0.915 | 0.79425 | transport |
| 1529 | mmdA | 0.066 | 1.164 | 0.302065 | 0.034 | 1.082 | 0.502417 | carbohydrate and lipid metabolism |
| 1530 | vng1530 | −0.137 | 0.730 | 0.639408 | 0.012 | 1.029 | 0.83025 | unknown |
| 1532 | acc | −0.007 | 0.983 | 0.459025 | 0.073 | 1.183 | 0.254142 | carbohydrate and lipid metabolism |
| 1533 | vng1533 | −0.026 | 0.942 | 0.686406 | 0.035 | 1.084 | 0.605333 | unknown |
| 1534 | birA | −0.130 | 0.742 | 0.036442 | −0.006 | 0.987 | 0.5605 | transcription and regulation |
| 1536 | birL | 0.148 | 1.405 | 0.247226 | 0.008 | 1.018 | 0.8965 | cofactor and secondary |
| | | | | | | | | metabolites |
| 1537 | vng1536 | −0.018 | 0.959 | 0.000354 | 0.044 | 1.107 | 0.43775 | cellular process |
| 1538 | vng1537 | 0.097 | 1.252 | 0.203805 | 0.022 | 1.052 | 0.697417 | unknown |
| 1540 | vng1538 | 0.105 | 1.274 | 0.302284 | −0.030 | 0.934 | 0.714875 | unknown |
| 1541 | ywfD | 0.182 | 1.521 | 0.017798 | 0.036 | 1.085 | 0.496583 | general function prediction only |
| 1542 | sucC | 0.250 | 1.778 | 0.000202 | 0.066 | 1.165 | 0.256275 | energy metabolism |
| 1543 | sucD | −0.033 | 0.926 | 0.447098 | 0.063 | 1.157 | 0.306333 | energy metabolism |
| 1544 | zim | −0.010 | 0.976 | 0.261691 | −0.058 | 0.875 | 0.46067 | DNA metabolism |
| 1546 | clc | 0.066 | 1.163 | 0.186064 | −0.032 | 0.929 | 0.55825 | transport |
| 1547 | vng1547 | 0.033 | 1.080 | 0.534491 | −0.037 | 0.918 | 0.5115 | unknown |
| 1548 | calR | −0.102 | 0.791 | 0.366083 | −0.009 | 0.980 | 0.46455 | transcription and regulation |
| 1550 | cbiT | −0.080 | 0.832 | 0.351607 | 0.004 | 1.010 | 0.899 | cofactor and secondary |
| | | | | | | | | metabolites |
| 1551 | cbiL | −0.061 | 0.869 | 0.218684 | −0.032 | 0.929 | 0.5235 | cofactor and secondary |
| | | | | | | | | metabolites |
| 1553 | cbiF | −0.246 | 0.567 | 0.006521 | 0.017 | 1.040 | 0.7315 | cofactor and secondary |
| | | | | | | | | metabolites |
| 1554 | cbiG | −0.271 | 0.536 | 0.271921 | 0.047 | 1.114 | 0.325083 | cofactor and secondary |
| | | | | | | | | metabolites |
| 1555 | cobH | −0.324 | 0.475 | 0.002074 | 0.028 | 1.067 | 0.585417 | cofactor and secondary |
| | | | | | | | | metabolites |
| 1557 | cbiH | −0.245 | 0.569 | 0.075976 | 0.067 | 1.167 | 0.224325 | cofactor and secondary |
| | | | | | | | | metabolites |
| 1558 | vng1558 | −0.532 | 0.294 | 7.58E−13 | 0.060 | 1.148 | 0.278825 | unknown |
| 1559 | vng1559 | −0.472 | 0.338 | 1.83E−06 | 0.023 | 1.055 | 0.610667 | unknown |
| 1561 | vng1561 | −0.349 | 0.448 | 0.131437 | 0.002 | 1.004 | 0.6205 | cofactor and secondary |
| | | | | | | | | metabolites |
| 1562 | vng1562 | −0.059 | 0.874 | 0.430925 | 0.009 | 1.021 | 0.584167 | unknown |
| 1564 | vng1564 | −0.030 | 0.933 | 0.679055 | −0.063 | 0.864 | 0.729 | unknown |
| 1565 | hmcA | 0.073 | 1.183 | 0.490704 | −0.030 | 0.932 | 0.610167 | cofactor and secondary |
| | | | | | | | | metabolites |
| 1566 | cobN | −0.243 | 0.571 | 0.014044 | 0.067 | 1.166 | 0.226383 | cofactor and secondary |
| | | | | | | | | metabolites |
| 1567 | cbiC | −0.355 | 0.442 | 0.000111 | 0.086 | 1.220 | 0.105275 | cofactor and secondary |
| | | | | | | | | metabolites |
| 1568 | cbiJ | −0.098 | 0.798 | 0.162053 | 0.019 | 1.044 | 0.597917 | cofactor and secondary |
| | | | | | | | | metabolites |
| 1570 | vng1570 | 0.032 | 1.076 | 0.531556 | −0.098 | 0.799 | 0.0762 | unknown |
| 1572 | vng1572 | −0.103 | 0.789 | 0.098356 | 0.037 | 1.090 | 0.473 | cofactor and secondary |
| | | | | | | | | metabolites |
| 1573 | cbiA | −0.151 | 0.707 | 0.28479 | 0.126 | 1.337 | 0.094253 | cofactor and secondary |
| | | | | | | | | metabolites |
| 1574 | cobl | −0.290 | 0.513 | 0.131664 | 0.091 | 1.232 | 0.225985 | cofactor and secondary |
| | | | | | | | | metabolites |
| 1576 | cbiP | −0.251 | 0.561 | 0.077747 | 0.092 | 1.236 | 0.13905 | cofactor and secondary |
| | | | | | | | | metabolites |
| 1577 | vng1577 | −0.037 | 0.919 | 0.510268 | 0.006 | 1.013 | 0.523983 | unknown |
| 1578 | vng1578 | −0.157 | 0.696 | 0.128275 | 0.036 | 1.087 | 0.585375 | cofactor and secondary |
| | | | | | | | | metabolites |
| 1580 | vng1580 | −0.112 | 0.774 | 0.7545 | 0.123 | 1.328 | 0.745 | cofactor and secondary |
| | | | | | | | | metabolites |
| 1581 | cobY | −0.190 | 0.646 | 0.163124 | −0.021 | 0.953 | 0.6615 | cofactor and secondary |
| | | | | | | | | metabolites |
| 1582 | hisC2 | −0.052 | 0.886 | 0.028586 | −0.008 | 0.981 | 0.522333 | cofactor and secondary |
| | | | | | | | | metabolites |
| 1583 | cbiZ | −0.014 | 0.969 | 0.382893 | −0.016 | 0.965 | 0.75075 | cofactor and secondary |
| | | | | | | | | metabolites |
| 1585 | vng1585 | −0.118 | 0.762 | 0.30521 | −0.087 | 0.818 | 0.13545 | unknown |
| 1587 | vng1587 | 0.139 | 1.379 | 0.334132 | 0.084 | 1.214 | 0.306 | unknown |
| 1589 | vng1589 | −0.111 | 0.775 | 0.089612 | 0.009 | 1.021 | 0.76225 | unknown |
| 1590 | vng1590 | 0.034 | 1.082 | 0.595058 | 0.088 | 1.226 | 0.271267 | unknown |
| 1591 | vng1591 | 0.074 | 1.185 | 0.135883 | 0.000 | 1.000 | 0.942 | unknown |
| 1592 | cysU | −0.085 | 0.823 | 0.124076 | −0.018 | 0.959 | 0.727 | transport |
| 1595 | modA | 0.010 | 1.022 | 0.495597 | −0.052 | 0.888 | 0.359333 | transport |
| 1598 | vng1598 | −0.018 | 0.959 | 0.542147 | 0.060 | 1.148 | 0.441 | unknown |
| 1601 | gcvP2 | 0.101 | 1.262 | 0.185857 | 0.050 | 1.123 | 0.403583 | amino acid metabolism |
| 1603 | gcvP1 | 0.152 | 1.419 | 0.12656 | 0.078 | 1.198 | 0.161967 | amino acid metabolism |
| 1605 | gdcH | 0.197 | 1.574 | 0.141592 | 0.013 | 1.029 | 0.7695 | amino acid metabolism |
| 1606 | gcvT1 | 0.060 | 1.149 | 0.171592 | 0.033 | 1.079 | 0.5085 | amino acid metabolism |
| 1607 | cheC2 | 0.185 | 1.530 | 0.050691 | −0.057 | 0.878 | 0.325217 | cellular process |
| 1608 | vng1608 | −0.034 | 0.924 | 0.480229 | 0.054 | 1.131 | 0.539667 | unknown |
| 1609 | vng1609 | −0.048 | 0.895 | 0.450133 | −0.012 | 0.974 | 0.813 | unknown |
| 1610 | vng1610 | −0.033 | 0.928 | 0.563145 | 0.035 | 1.085 | 0.639333 | unknown |
| 1611 | vng1611 | 0.114 | 1.300 | 0.083029 | 0.029 | 1.069 | 0.558625 | unknown |
| 1613 | vng1613 | −0.121 | 0.758 | 0.134102 | 0.096 | 1.247 | 0.231617 | unknown |
| 1615 | mvaB | 0.333 | 2.154 | 0.026326 | −0.030 | 0.933 | 0.548833 | carbohydrate and lipid metabolism |
| 1616 | xrlR4 | 0.076 | 1.190 | 0.369572 | −0.036 | 0.921 | 0.711083 | transcription and regulation |
| 1617 | act6 | 0.088 | 1.224 | 0.406789 | 0.022 | 1.053 | 0.7285 | transcription and regulation |
| 1618 | vng1617 | 0.254 | 1.796 | 0.390496 | 0.016 | 1.038 | 0.78475 | unknown |
| 1619 | vng1619 | 0.026 | 1.061 | 0.529299 | −0.067 | 0.856 | 0.336753 | unknown |
| 1621 | vng1621 | −0.015 | 0.966 | 0.074647 | −0.033 | 0.926 | 0.51225 | unknown |
| 1622 | rfcB | 0.028 | 1.066 | 0.484108 | 0.006 | 1.014 | 0.886917 | DNA metabolism |
| 1623 | ccp | 0.160 | 1.447 | 0.013171 | −0.014 | 0.968 | 0.732583 | cellular process |
| 1624 | mdh | 0.028 | 1.066 | 0.495941 | 0.017 | 1.040 | 0.62175 | energy metabolism |
| 1625 | vng1625 | 0.021 | 1.050 | 0.559069 | −0.170 | 0.677 | 0.31625 | unknown |
| 1626 | vng1626 | 0.196 | 1.570 | 0.026241 | −0.017 | 0.962 | 0.595 | carbohydrate and lipid metabolism |
| 1628 | aad | −0.040 | 0.912 | 0.565468 | −0.015 | 0.967 | 0.719 | energy metabolism |
| 1630 | vng1630 | 0.055 | 1.135 | 0.177612 | −0.065 | 0.862 | 0.244325 | unknown |
| 1631 | cbiO2 | 0.008 | 1.019 | 0.415672 | −0.055 | 0.880 | 0.3624 | transport |
| 1632 | cbiQ | 0.145 | 1.396 | 0.167905 | −0.012 | 0.974 | 0.7 | transport |
| 1634 | cbiN | 0.458 | 2.871 | 6.25E−13 | −0.005 | 0.989 | 0.82125 | transport |
| 1635 | cbiM | 0.453 | 2.836 | 2.42E−09 | −0.016 | 0.965 | 0.705417 | cofactor and secondary |
| | | | | | | | | metabolites |
| 1637 | hcpA | −0.021 | 0.952 | 0.464282 | −0.015 | 0.966 | 0.659917 | energy metabolism |
| 1638 | vng1638 | 0.191 | 1.552 | 0.002236 | 0.021 | 1.050 | 0.703875 | unknown |
| 1640 | vng1640 | 0.012 | 1.028 | 0.878842 | 0.008 | 1.020 | 0.928 | transcription and regulation |
| 1641 | vng1641 | 0.050 | 1.121 | 0.416988 | 0.011 | 1.027 | 0.695667 | unknown |
| 1642 | vng1642 | −0.394 | 0.403 | 1.9E−08 | 0.060 | 1.147 | 0.2805 | unknown |
| 1644 | nrdJ | −0.164 | 0.686 | 0.080157 | 0.149 | 1.410 | 0.008456 | nucleotide metabolism |
| 1645 | vng1645 | 0.043 | 1.104 | 0.396648 | 0.031 | 1.075 | 0.54875 | unknown |
| 1646 | trpG1 | 0.020 | 1.048 | 0.300146 | −0.012 | 0.974 | 0.477133 | amino acid metabolism |
| 1647 | trpE1 | 0.004 | 1.008 | 0.597741 | 0.025 | 1.060 | 0.78625 | amino acid metabolism |
| 1648 | trpF | 0.024 | 1.057 | 0.471844 | −0.011 | 0.976 | 0.782 | amino acid metabolism |
| 1649 | trpD1 | 0.017 | 1.039 | 0.423988 | −0.144 | 0.717 | 0.260965 | amino acid metabolism |
| 1650 | vng1650 | −0.030 | 0.933 | 0.258877 | 0.061 | 1.152 | 0.65675 | unknown |
| 1654 | cdc48E | −0.146 | 0.714 | 0.223 | 0.035 | 1.084 | 0.427075 | cellular process |
| 1656 | vng1656 | −0.019 | 0.958 | 0.506309 | 0.034 | 1.081 | 0.607083 | unknown |
| 1657 | vng1657 | −0.004 | 0.991 | 0.161869 | 0.017 | 1.039 | 0.3669 | unknown |
| 1658 | vng1658 | 0.089 | 1.228 | 0.077122 | −0.021 | 0.953 | 0.6865 | cellular process |
| 1659 | htr1 | 0.056 | 1.139 | 0.450364 | 0.007 | 1.016 | 0.81975 | cellular process |
| 1660 | sop1 | 0.059 | 1.144 | 0.38024 | −0.006 | 0.986 | 0.686833 | cellular process |
| 1663 | vng1663 | 0.194 | 1.563 | 0.13974 | 0.010 | 1.023 | 0.575167 | unknown |
| 1664 | vng1664 | 0.009 | 1.022 | 0.836139 | −0.040 | 0.911 | 0.52 | unknown |
| 1665 | radA2 | 0.009 | 1.021 | 0.728358 | −0.091 | 0.811 | 0.517667 | DNA metabolism |
| 1666 | vng1666 | 0.085 | 1.216 | 0.460238 | −0.078 | 0.836 | 0.186 | unknown |
| 1667 | cdc48c | 0.111 | 1.293 | 0.260082 | 0.020 | 1.047 | 0.577167 | cellular process |
| 1668 | rps8e | 0.204 | 1.601 | 0.001341 | −0.061 | 0.870 | 0.2195 | translation |
| 1670 | vng1670 | −0.004 | 0.991 | 0.795174 | 0.034 | 1.082 | 0.519 | unknown |
| 1672 | vng1672 | −0.034 | 0.925 | 0.289108 | 0.043 | 1.105 | 0.5143 | unknown |
| 1673 | pyrF | 0.083 | 1.211 | 0.309934 | 0.021 | 1.050 | 0.667625 | nucleotide metabolism |
| 1674 | vng1674 | −0.001 | 0.998 | 0.86 | 0.006 | 1.014 | 0.902 | unknown |
| 1675 | vng1675 | 0.061 | 1.150 | 0.210749 | 0.057 | 1.140 | 0.2765 | general function prediction only |
| 1676 | gbp2 | −0.001 | 0.999 | 0.384977 | 0.023 | 1.054 | 0.230117 | cellular process |
| 1678 | vng1678 | −0.046 | 0.900 | 0.420869 | −0.005 | 0.988 | 0.5405 | unknown |
| 1679 | vng1679 | 0.156 | 1.431 | 0.081182 | −0.057 | 0.878 | 0.282 | unknown |
| 1680 | crtB2 | 0.070 | 1.176 | 0.380366 | 0.030 | 1.071 | 0.586 | cofactor and secondary |
| | | | | | | | | metabolites |
| 1681 | vng1681 | 0.024 | 1.057 | 0.223927 | 0.002 | 1.005 | 0.662083 | unknown |
| 1682 | vng1682 | −0.048 | 0.896 | 0.458255 | 0.018 | 1.043 | 0.6485 | cofactor and secondary |
| | | | | | | | | metabolites |
| 1684 | crtl1 | 0.004 | 1.010 | 0.511177 | −0.024 | 0.946 | 0.48 | cofactor and secondary |
| | | | | | | | | metabolites |
| 1686 | mch | 0.055 | 1.136 | 0.647264 | −0.037 | 0.919 | 0.615 | cofactor and secondary |
| | | | | | | | | metabolites |
| 1687 | vng1687 | −0.074 | 0.843 | 0.23937 | 0.019 | 1.044 | 0.537583 | carbohydrate and lipid metabolism |
| 1688 | vng1688 | −0.338 | 0.459 | 9.7E−07 | 0.086 | 1.220 | 0.105383 | unknown |
| 1689 | rpl3p | −0.364 | 0.433 | 4.72E−08 | 0.118 | 1.312 | 0.038568 | translation |
| 1690 | rpl4e | −0.401 | 0.397 | 0.034 | 0.090 | 1.229 | 0.125008 | translation |
| 1691 | rpl23p | −0.535 | 0.292 | 4.23E−13 | 0.127 | 1.339 | 0.011298 | translation |
| 1692 | rpl2p | −0.531 | 0.294 | 0.006 | 0.099 | 1.257 | 0.066385 | translation |
| 1693 | rps19p | −0.551 | 0.281 | 1.32E−11 | 0.088 | 1.226 | 0.110233 | translation |
| 1695 | rpl22p | −0.636 | 0.231 | 6.54E−14 | 0.106 | 1.277 | 0.047112 | translation |
| 1697 | rps3p | −0.689 | 0.205 | 1.17E−14 | 0.131 | 1.352 | 0.011325 | translation |
| 1698 | rpl29p | −0.677 | 0.210 | 6.33E−15 | 0.101 | 1.261 | 0.04265 | translation |
| 1699 | vng1699 | −0.725 | 0.188 | 1.07E−14 | 0.075 | 1.188 | 0.134767 | translation |
| 1700 | rps17p | −0.474 | 0.336 | 0.066 | 0.088 | 1.224 | 0.080933 | translation |
| 1701 | rpl14p | −0.747 | 0.179 | 1.29E−15 | 0.071 | 1.178 | 0.161317 | translation |
| 1702 | rpl24p | −0.597 | 0.253 | 2.28E−09 | 0.073 | 1.182 | 0.15535 | translation |
| 1703 | rps4e | −0.510 | 0.309 | 0.181664 | 0.054 | 1.133 | 0.299833 | translation |
| 1705 | rpl5p | −0.828 | 0.149 | 1.87E−18 | 0.071 | 1.177 | 0.157983 | translation |
| 1706 | rps14p | −0.770 | 0.170 | 3.43E−17 | 0.068 | 1.169 | 0.293125 | translation |
| 1707 | rps8p | −0.452 | 0.353 | 0.13175 | 0.101 | 1.261 | 0.04925 | translation |
| 1709 | rpl6p | −0.668 | 0.215 | 1.47E−13 | 0.091 | 1.234 | 0.072842 | translation |
| 1711 | rpl32e | −0.482 | 0.330 | 0.01657 | 0.074 | 1.187 | 0.139492 | translation |
| 1713 | rpl19e | −0.654 | 0.222 | 0.000792 | 0.088 | 1.226 | 0.082633 | translation |
| 1714 | rpl18p | −0.674 | 0.212 | 6.87E−13 | 0.081 | 1.204 | 0.128179 | translation |
| 1715 | rps5p | −0.573 | 0.268 | 6.28E−11 | 0.112 | 1.294 | 0.03128 | translation |
| 1716 | rpl30p | −0.627 | 0.236 | 2.51E−16 | 0.106 | 1.275 | 0.04819 | translation |
| 1718 | rpl15p | −0.283 | 0.521 | 0.000697 | 0.073 | 1.183 | 0.224075 | translation |
| 1719 | secY | −0.402 | 0.397 | 7.49E−09 | 0.053 | 1.131 | 0.293083 | cellular process |
| 1720 | vng1720 | −0.095 | 0.804 | 0.311375 | −0.011 | 0.975 | 0.803583 | unknown |
| 1721 | trkH2 | 0.049 | 1.119 | 0.252163 | 0.012 | 1.028 | 0.552667 | transport |
| 1723 | vng1723 | −0.019 | 0.956 | 0.726015 | −0.102 | 0.790 | 0.144963 | unknown |
| 1724 | adk | 0.040 | 1.096 | 0.132656 | 0.035 | 1.084 | 0.5005 | nucleotide metabolism |
| 1726 | htlB | 0.074 | 1.187 | 0.28616 | 0.008 | 1.020 | 0.69425 | cellular process |
| 1727 | cmk | 0.029 | 1.068 | 0.602322 | 0.025 | 1.060 | 0.609 | nucleotide metabolism |
| 1729 | sus | 0.022 | 1.053 | 0.610823 | 0.013 | 1.031 | 0.632417 | translation |
| 1732 | vng1732 | 0.049 | 1.119 | 0.395043 | 0.029 | 1.070 | 0.67225 | unknown |
| 1733 | htr17 | −0.098 | 0.798 | 0.368807 | 0.035 | 1.084 | 0.618333 | cellular process |
| 1734 | vng1734 | 0.112 | 1.296 | 0.065118 | 0.029 | 1.069 | 0.6355 | unknown |
| 1801 | hsp1 | 0.052 | 1.128 | 0.276433 | −0.049 | 0.892 | 0.34395 | cellular process |
| 1802 | vng1802 | 0.067 | 1.166 | 0.214429 | −0.201 | 0.629 | 0.219283 | unknown |
| 1804 | noxA | 0.019 | 1.045 | 0.342404 | 0.072 | 1.179 | 0.262133 | general function prediction only |
| 1806 | vng1806 | 0.045 | 1.108 | 0.296062 | −0.128 | 0.744 | 0.01771 | unknown |
| 1807 | vng1807 | 0.094 | 1.243 | 0.143642 | −0.057 | 0.877 | 0.372113 | unknown |
| 1809 | vng1809 | −0.016 | 0.964 | 0.808534 | 0.007 | 1.016 | 0.532625 | unknown |
| 1811 | eye | 0.035 | 1.084 | 0.260153 | −0.035 | 0.922 | 0.455308 | general function prediction only |
| 1814 | carB | −0.086 | 0.820 | 0.232255 | 0.076 | 1.192 | 0.417533 | amino acid metabolism |
| 1815 | carA | −0.087 | 0.818 | 0.228573 | −0.073 | 0.845 | 0.238967 | amino acid metabolism |
| 1816 | trh3 | −0.130 | 0.740 | 0.382062 | 0.023 | 1.055 | 0.7225 | transcription and regulation |
| 1818 | idi | 0.064 | 1.160 | 0.239956 | −0.026 | 0.942 | 0.60825 | carbohydrate and lipid metabolism |
| 1820 | vng1820 | 0.086 | 1.219 | 0.254923 | 0.017 | 1.040 | 0.645917 | unknown |
| 1821 | adh4 | 0.065 | 1.161 | 0.151721 | 0.005 | 1.012 | 0.613917 | energy metabolism |
| 1822 | moaB | 0.022 | 1.051 | 0.627611 | 0.007 | 1.016 | 0.602375 | cofactor and secondary |
| | | | | | | | | metabolites |
| 1823 | vng1823 | 0.092 | 1.235 | 0.092464 | −0.181 | 0.659 | 0.000677 | cofactor and secondary |
| | | | | | | | | metabolites |
| 1826 | vng1826 | 0.083 | 1.209 | 0.156379 | 0.008 | 1.018 | 0.90875 | unknown |
| 1827 | vng1827 | 0.060 | 1.149 | 0.49016 | 0.024 | 1.057 | 0.6815 | unknown |
| 1829 | guaAb | −0.105 | 0.784 | 0.16964 | −0.006 | 0.987 | 0.715583 | nucleotide metabolism |
| 1830 | pyrG | −0.083 | 0.826 | 0.257739 | 0.026 | 1.061 | 0.4845 | nucleotide metabolism |
| 1831 | vng1831 | −0.032 | 0.928 | 0.406937 | 0.052 | 1.126 | 0.48975 | unknown |
| 1832 | vng1832 | −0.118 | 0.762 | 0.220399 | 0.083 | 1.209 | 0.5455 | unknown |
| 1833 | vng1833 | 0.078 | 1.196 | 0.553673 | −0.059 | 0.873 | 0.37015 | unknown |
| 1834 | hyrA | 0.048 | 1.117 | 0.29789 | −0.014 | 0.968 | 0.682917 | cellular process |
| 1835 | thrS | 0.166 | 1.466 | 0.006851 | −0.048 | 0.895 | 0.356417 | translation |
| 1836 | cspD2 | 0.123 | 1.329 | 0.229 | 0.055 | 1.134 | 0.267 | cellular process |
| 1837 | ykfB1 | 0.052 | 1.127 | 0.402999 | −0.016 | 0.964 | 0.669583 | amino acid metabolism |
| 1839 | vng1838 | 0.024 | 1.057 | 0.440999 | −0.046 | 0.899 | 0.41575 | unknown |
| 1842 | vng1842 | 0.076 | 1.191 | 0.432405 | −0.003 | 0.994 | 0.659833 | translation |
| 1843 | arlR12 | 0.010 | 1.022 | 0.40252 | −0.036 | 0.920 | 0.535583 | transcription and regulation |
| 1844 | ansA | −0.030 | 0.933 | 0.493237 | 0.029 | 1.070 | 0.61625 | amino acid metabolism |
| 1845 | act7 | −0.007 | 0.984 | 0.722771 | 0.046 | 1.111 | 0.447317 | transcription and regulation |
| 1846 | ugpQ | 0.070 | 1.175 | 0.322716 | 0.005 | 1.011 | 0.704333 | energy metabolism |
| 1847 | pchA | 0.025 | 1.058 | 0.577165 | 0.020 | 1.047 | 0.664333 | transport |
| 1848 | Vng1848 | 0.036 | 1.085 | 0.495706 | −0.049 | 0.893 | 0.33975 | cofactor and secondary |
| | | | | | | | | metabolites |
| 1849 | vng1849 | 0.082 | 1.208 | 0.466898 | 0.000 | 1.000 | 0.85375 | unknown |
| 1850 | udp1 | 0.146 | 1.398 | 0.106064 | 0.019 | 1.045 | 0.66375 | nucleotide metabolism |
| 1851 | suk | 0.120 | 1.317 | 0.07097 | 0.040 | 1.096 | 0.42925 | carbohydrate and lipid metabolism |
| 1852 | vng1852 | 0.129 | 1.346 | 0.03062 | −0.007 | 0.984 | 0.472833 | unknown |
| 1853 | eif2ba | 0.006 | 1.015 | 0.481623 | 0.002 | 1.004 | 0.819833 | translation |
| 1856 | htr3 | 0.059 | 1.145 | 0.592714 | 0.001 | 1.001 | 0.753833 | cellular process |
| 1857 | vng1857 | 0.044 | 1.106 | 0.372054 | −0.024 | 0.946 | 0.652667 | amino acid metabolism |
| 1859 | deoC | 0.020 | 1.047 | 0.361866 | −0.036 | 0.919 | 0.610975 | nucleotide metabolism |
| 1861 | vng1861 | −0.019 | 0.957 | 0.535718 | 0.037 | 1.089 | 0.495667 | translation |
| 1862 | cef | −0.048 | 0.896 | 0.445559 | −0.032 | 0.929 | 0.5235 | transport |
| 1864 | hit2 | 0.178 | 1.505 | 0.014859 | −0.092 | 0.809 | 0.127758 | nucleotide metabolism |
| 1865 | vng1865 | 0.186 | 1.535 | 0.016289 | −0.127 | 0.747 | 0.092175 | unknown |
| 1866 | map | −0.035 | 0.923 | 0.508438 | 0.016 | 1.037 | 0.66925 | translation |
| 1867 | potC | −0.127 | 0.746 | 0.708211 | 0.013 | 1.031 | 0.9265 | transport |
| 1868 | potB | 0.038 | 1.091 | 0.597431 | 0.029 | 1.069 | 0.634083 | transport |
| 1869 | vng1869 | −0.006 | 0.987 | 0.624373 | −0.243 | 0.571 | 0.276034 | amino acid metabolism |
| 1871 | potA2 | −0.013 | 0.970 | 0.783287 | 0.084 | 1.213 | 0.128175 | transport |
| 1872 | vng1872 | −0.059 | 0.873 | 0.38892 | −0.015 | 0.966 | 0.76325 | amino acid metabolism |
| 1873 | icd | 0.172 | 1.487 | 0.06846 | 0.079 | 1.200 | 0.22754 | energy metabolism |
| 1874 | vng1874 | 0.089 | 1.229 | 0.528493 | 0.017 | 1.040 | 0.70075 | unknown |
| 1875 | mvaA | 0.088 | 1.225 | 0.164048 | 0.006 | 1.015 | 0.633125 | carbohydrate and lipid metabolism |
| 1877 | vng1877 | 0.227 | 1.686 | 0.000478 | 0.024 | 1.058 | 0.57225 | unknown |
| 1879 | thb | 0.117 | 1.309 | 0.325983 | 0.019 | 1.045 | 0.572 | cofactor and secondary |
| | | | | | | | | metabolites |
| 1880 | vng1880 | −0.011 | 0.974 | 0.652405 | 0.027 | 1.065 | 0.565267 | unknown |
| 1882 | nadA | 0.025 | 1.060 | 0.669766 | 0.061 | 1.152 | 0.408417 | cofactor and secondary |
| | | | | | | | | metabolites |
| 1883 | nadB | 0.001 | 1.002 | 0.714427 | 0.027 | 1.064 | 0.660625 | cofactor and secondary |
| | | | | | | | | metabolites |
| 1884 | nadC | −0.019 | 0.956 | 0.410053 | −0.027 | 0.939 | 0.6465 | cofactor and secondary |
| | | | | | | | | metabolites |
| 1886 | sstR2 | 0.124 | 1.330 | 0.42394 | −0.070 | 0.851 | 0.650167 | transcription and regulation |
| 1887 | gpm | 0.320 | 2.091 | 0.211412 | 0.003 | 1.008 | 0.57875 | carbohydrate and lipid metabolism |
| 1889 | vng1889 | 0.703 | 5.048 | 8.75E−14 | 0.044 | 1.107 | 0.4005 | unknown |
| 1890 | vng1890 | 0.034 | 1.082 | 0.211881 | 0.067 | 1.166 | 0.407158 | unknown |
| 1891 | vng1891 | 0.002 | 1.006 | 0.554434 | −0.004 | 0.990 | 0.874167 | unknown |
| 1893 | trp2 | 0.011 | 1.026 | 0.324019 | −0.012 | 0.972 | 0.669667 | transport |
| 1894 | vng1894 | 0.017 | 1.040 | 0.470177 | 0.014 | 1.032 | 0.710833 | unknown |
| 1895 | vng1895 | −0.019 | 0.957 | 0.859417 | 0.032 | 1.077 | 0.7835 | unknown |
| 1896 | vng1896 | 0.009 | 1.021 | 0.47383 | 0.017 | 1.041 | 0.657667 | unknown |
| 1898 | vng1898 | 0.164 | 1.460 | 0.267335 | −0.036 | 0.921 | 0.511417 | cellular process |
| 1899 | kaiC | 0.131 | 1.354 | 0.203073 | 0.002 | 1.004 | 0.731583 | cellular process |
| 1900 | vng1900 | 0.037 | 1.088 | 0.598583 | 0.011 | 1.025 | 0.770458 | carbohydrate and lipid metabolism |
| 1901 | vng1901 | 0.121 | 1.320 | 0.148302 | −0.041 | 0.910 | 0.549583 | unknown |
| 1902 | vng1902 | 0.178 | 1.506 | 0.106379 | 0.008 | 1.019 | 0.922 | unknown |
| 1903 | sstR3 | 0.125 | 1.333 | 0.135295 | −0.006 | 0.987 | 0.65875 | transcription and regulation |
| 1904 | vng1904 | 0.023 | 1.056 | 0.221579 | −0.083 | 0.825 | 0.151775 | unknown |
| 1905 | vng1905 | −0.046 | 0.899 | 0.518161 | 0.031 | 1.074 | 0.523 | cellular process |
| 1906 | vng1906 | 0.077 | 1.194 | 0.655368 | −0.059 | 0.873 | 0.5505 | unknown |
| 1907 | vng1907 | −0.133 | 0.737 | 0.261155 | 0.021 | 1.049 | 0.201075 | unknown |
| 1910 | vng1910 | 0.075 | 1.190 | 0.569997 | −0.066 | 0.858 | 0.607838 | unknown |
| 1911 | nirD | 0.049 | 1.119 | 0.571057 | −0.027 | 0.940 | 0.711 | transcription and regulation |
| 1912 | trpD2 | 0.077 | 1.194 | 0.496589 | 0.001 | 1.002 | 0.7455 | amino acid metabolism |
| 1914 | ppiA | 0.107 | 1.280 | 0.133449 | −0.026 | 0.941 | 0.603 | cellular process |
| 1916 | vng1916 | 0.068 | 1.169 | 0.190182 | 0.005 | 1.012 | 0.9175 | unknown |
| 1917 | vng1917 | −0.130 | 0.742 | 0.080056 | 0.050 | 1.122 | 0.3245 | unknown |
| 1918 | vng1918 | 0.018 | 1.042 | 0.459584 | −0.023 | 0.949 | 0.5375 | unknown |
| 1919 | vng1919 | −0.179 | 0.663 | 0.001495 | 0.024 | 1.057 | 0.643 | cellular process |
| 1920 | vng1920 | 0.016 | 1.037 | 0.667851 | −0.005 | 0.990 | 0.9135 | unknown |
| 1921 | vng1921 | −0.027 | 0.940 | 0.531242 | −0.018 | 0.959 | 0.810125 | cellular process |
| 1922 | asxR5 | −0.040 | 0.912 | 0.541076 | −0.012 | 0.972 | 0.4215 | transcription and regulation |
| 1924 | trkA6 | 0.103 | 1.267 | 0.118077 | −0.059 | 0.873 | 0.368008 | transport |
| 1925 | vng1925 | 0.009 | 1.021 | 0.788098 | 0.020 | 1.048 | 0.805 | unknown |
| 1926 | pdhA1 | 0.111 | 1.292 | 0.365774 | −0.012 | 0.973 | 0.749417 | energy metabolism |
| 1927 | vng1927 | 0.017 | 1.040 | 0.732062 | 0.022 | 1.053 | 0.67175 | unknown |
| 1929 | tmk | 0.007 | 1.016 | 0.712735 | −0.038 | 0.917 | 0.53525 | nucleotide metabolism |
| 1932 | nolA | −0.144 | 0.717 | 0.49722 | 0.071 | 1.177 | 0.243225 | general function prediction only |
| 1933 | ftsZ3 | 0.098 | 1.253 | 0.210732 | −0.014 | 0.969 | 0.714583 | cellular process |
| 1934 | vng1934 | 0.072 | 1.181 | 0.394378 | −0.020 | 0.954 | 0.566917 | unknown |
| 1935 | vng1935 | 0.083 | 1.210 | 0.362747 | 0.043 | 1.105 | 0.470625 | unknown |
| 1937 | vng1937 | 0.033 | 1.078 | 0.598473 | 0.038 | 1.092 | 0.579167 | cofactor and secondary |
| | | | | | | | | metabolites |
| 1938 | vng1938 | 0.082 | 1.208 | 0.166409 | −0.074 | 0.843 | 0.19735 | cofactor and secondary |
| | | | | | | | | metabolites |
| 1939 | purC | 0.012 | 1.029 | 0.552259 | 0.116 | 1.305 | 0.064518 | nucleotide metabolism |
| 1940 | vng1940 | 0.252 | 1.787 | 4.96E−05 | 0.023 | 1.055 | 0.6455 | unknown |
| 1941 | vng1941 | 0.052 | 1.127 | 0.504984 | 0.023 | 1.055 | 0.718917 | unknown |
| 1942 | vng1942 | 0.055 | 1.134 | 0.376123 | 0.089 | 1.226 | 0.1007 | unknown |
| 1943 | vng1943 | 0.000 | 1.001 | 0.651319 | 0.023 | 1.054 | 0.46325 | unknown |
| 1944 | purS | 0.046 | 1.112 | 0.417102 | 0.123 | 1.329 | 0.018715 | nucleotide metabolism |
| 1945 | purL2 | 0.008 | 1.018 | 0.742295 | 0.111 | 1.292 | 0.0385 | nucleotide metabolism |
| 1946 | purU | 0.049 | 1.118 | 0.487562 | −0.002 | 0.994 | 0.76225 | nucleotide metabolism |
| 1948 | vng1948 | 0.082 | 1.206 | 0.364974 | −0.037 | 0.919 | 0.250243 | unknown |
| 1949 | gul2 | 0.007 | 1.017 | 0.707418 | −0.043 | 0.905 | 0.4 | general function prediction only |
| 1951 | sub | 0.046 | 1.111 | 0.38595 | −0.054 | 0.883 | 0.357 | cellular process |
| 1952 | vng1952 | 0.032 | 1.076 | 0.644795 | −0.030 | 0.933 | 0.567222 | unknown |
| 1953 | vng1953 | 0.006 | 1.014 | 0.803718 | −0.063 | 0.865 | 0.324417 | unknown |
| 1954 | vng1954 | −0.056 | 0.879 | 0.171752 | −0.007 | 0.983 | 0.47925 | unknown |
| 1956 | vng1956 | 0.080 | 1.202 | 0.333025 | 0.027 | 1.065 | 0.7075 | unknown |
| 1957 | tgtA2 | 0.023 | 1.054 | 0.635061 | −0.004 | 0.992 | 0.5585 | translation |
| 1959 | tgtA1 | 0.005 | 1.011 | 0.455188 | −0.090 | 0.813 | 0.256993 | translation |
| 1960 | vng1960 | 0.046 | 1.111 | 0.408219 | −0.066 | 0.859 | 0.269 | unknown |
| 1962 | vng1962 | 0.003 | 1.007 | 0.656069 | −0.003 | 0.993 | 0.67775 | DNA metabolism |
| 1963 | vng1963 | 0.042 | 1.101 | 0.621103 | 0.030 | 1.071 | 0.573417 | unknown |
| 1964 | csl | −0.109 | 0.778 | 0.344833 | −0.131 | 0.740 | 0.438252 | cellular process |
| 1965 | vng1965 | −0.342 | 0.455 | 0.001274 | 0.015 | 1.036 | 0.470483 | unknown |
| 1967 | glpK | −0.162 | 0.689 | 0.080945 | 0.023 | 1.054 | 0.6465 | carbohydrate and lipid metabolism |
| 1969 | gpdA2 | −0.465 | 0.343 | 4.07E−06 | 0.031 | 1.074 | 0.665917 | carbohydrate and lipid metabolism |
| 1971 | gpdB | 0.028 | 1.067 | 0.412384 | 0.021 | 1.050 | 0.555917 | carbohydrate and lipid metabolism |
| 1972 | gpdC | −0.223 | 0.598 | 0.169934 | −0.010 | 0.976 | 0.564217 | carbohydrate and lipid metabolism |
| 1973 | vng1973 | 0.003 | 1.006 | 0.473629 | 0.064 | 1.159 | 0.2984 | unknown |
| 1974 | vng1974 | −0.052 | 0.887 | 0.55905 | 0.011 | 1.027 | 0.692167 | unknown |
| 1976 | vng1976 | −0.031 | 0.931 | 0.633216 | 0.028 | 1.066 | 0.6575 | unknown |
| 1977 | vng1977 | 0.045 | 1.110 | 0.456487 | −0.076 | 0.840 | 0.21345 | unknown |
| 1978 | vng1978 | −0.109 | 0.779 | 0.143267 | −0.066 | 0.859 | 0.30425 | unknown |
| 1980 | vng1980 | 0.045 | 1.108 | 0.376687 | 0.024 | 1.058 | 0.6205 | unknown |
| 1982 | vng1983 | −0.012 | 0.973 | 0.376605 | 0.002 | 1.006 | 0.6945 | translation |
| 1983 | vng1982 | −0.050 | 0.892 | 0.456695 | −0.035 | 0.923 | 0.536667 | unknown |
| 1984 | rnh | −0.050 | 0.892 | 0.7743 | 0.063 | 1.156 | 0.479783 | DNA metabolism |
| 1985 | nhaC4 | 0.148 | 1.405 | 0.2808 | −0.019 | 0.958 | 0.64925 | energy metabolism |
| 1986 | vng1986 | 0.029 | 1.069 | 0.548295 | −0.016 | 0.963 | 0.715917 | DNA metabolism |
| 1987 | secD | 0.144 | 1.392 | 0.188086 | 0.004 | 1.009 | 0.9025 | cellular process |
| 1988 | secF | 0.139 | 1.378 | 0.090823 | −0.002 | 0.994 | 0.841 | cellular process |
| 1989 | vng1989 | 0.016 | 1.038 | 0.345498 | −0.216 | 0.608 | 0.028625 | unknown |
| 1991 | vng1991 | −0.017 | 0.961 | 0.534395 | 0.007 | 1.015 | 0.724625 | unknown |
| 1992 | pgi | −0.009 | 0.980 | 0.513707 | −0.002 | 0.996 | 0.575167 | carbohydrate and lipid metabolism |
| 1993 | vng1993 | −0.011 | 0.975 | 0.655962 | 0.035 | 1.083 | 0.44625 | unknown |
| 1994 | pzb | 0.126 | 1.338 | 0.174265 | −0.013 | 0.970 | 0.795 | transcription and regulation |
| 1995 | vng1995 | 0.195 | 1.567 | 0.198254 | −0.062 | 0.867 | 0.237875 | unknown |
| 1997 | infB | −0.069 | 0.853 | 0.508947 | 0.035 | 1.084 | 0.554667 | translation |
| 1998 | vng1998 | −0.003 | 0.993 | 0.231139 | −0.027 | 0.939 | 0.604667 | unknown |
| 1999 | vng1999 | 0.059 | 1.145 | 0.15957 | −0.002 | 0.994 | 0.86775 | unknown |
| 2000 | prrIV1 | 0.100 | 1.258 | 0.196791 | −0.038 | 0.915 | 0.469167 | cellular process |
| 2001 | yjbG | 0.360 | 2.289 | 6.63E−09 | −0.087 | 0.818 | 0.0999 | amino acid metabolism |
| 2002 | vng2002 | 0.025 | 1.060 | 0.554226 | −0.003 | 0.992 | 0.577267 | unknown |
| 2003 | truA | −0.038 | 0.916 | 0.334242 | −0.042 | 0.907 | 0.39 | translation |
| 2005 | hisS | −0.029 | 0.935 | 0.487202 | 0.051 | 1.123 | 0.399783 | translation |
| 2006 | vng2006 | −0.004 | 0.990 | 0.374034 | −0.012 | 0.973 | 0.429725 | translation |
| 2008 | pdb | 0.001 | 1.001 | 0.195291 | 0.043 | 1.104 | 0.41125 | transcription and regulation |
| 2010 | rps19e | 0.067 | 1.167 | 0.308057 | 0.021 | 1.049 | 0.6745 | translation |
| 2011 | thiL | −0.001 | 0.997 | 0.371261 | 0.028 | 1.066 | 0.64375 | cofactor and secondary |
| | | | | | | | | metabolites |
| 2012 | vng2012 | 0.077 | 1.193 | 0.258664 | −0.030 | 0.934 | 0.386133 | unknown |
| 2014 | vng2014 | −0.092 | 0.808 | 0.297924 | −0.199 | 0.632 | 0.000486 | transcription and regulation |
| 2015 | vng2015 | −0.054 | 0.884 | 0.45461 | 0.008 | 1.018 | 0.877 | nucleotide metabolism |
| 2017 | lysS | 0.027 | 1.063 | 0.540913 | 0.045 | 1.110 | 0.430333 | translation |
| 2019 | vng2019 | −0.010 | 0.976 | 0.660823 | −0.047 | 0.898 | 0.483742 | unknown |
| 2020 | prlR4 | 0.026 | 1.063 | 0.561503 | 0.023 | 1.054 | 0.702 | transcription and regulation |
| 2021 | vng2021 | 0.070 | 1.175 | 0.265696 | 0.001 | 1.003 | 0.64025 | unknown |
| 2023 | gsp | 0.084 | 1.214 | 0.143243 | 0.037 | 1.089 | 0.472333 | cellular process |
| 2024 | vng2024 | 0.056 | 1.138 | 0.563073 | 0.036 | 1.088 | 0.689 | unknown |
| 2025 | yyal | −0.040 | 0.912 | 0.455392 | 0.013 | 1.029 | 0.342917 | general function prediction only |
| 2027 | vng2027 | 0.050 | 1.123 | 0.699132 | 0.003 | 1.007 | 0.8855 | unknown |
| 2028 | vng2028 | 0.021 | 1.049 | 0.584069 | 0.023 | 1.055 | 0.340125 | unknown |
| 2029 | vng2029 | 0.018 | 1.043 | 0.669797 | 0.007 | 1.016 | 0.8835 | unknown |
| 2031 | nadE | 0.174 | 1.492 | 0.007158 | −0.088 | 0.817 | 0.09405 | cofactor and secondary |
| | | | | | | | | metabolites |
| 2032 | fad1 | −0.171 | 0.674 | 0.53999 | −0.007 | 0.984 | 0.973 | carbohydrate and lipid metabolism |
| 2034 | vng2034 | 0.040 | 1.096 | 0.541442 | 0.049 | 1.119 | 0.642 | unknown |
| 2035 | vng2035 | −0.063 | 0.865 | 0.220585 | −0.029 | 0.935 | 0.5885 | unknown |
| 2036 | hlx1 | 0.087 | 1.223 | 0.22502 | −0.054 | 0.883 | 0.379925 | transcription and regulation |
| 2037 | hik4 | −0.020 | 0.956 | 0.352579 | −0.067 | 0.856 | 0.221883 | cellular process |
| 2039 | vng2039 | 0.006 | 1.015 | 0.585631 | 0.025 | 1.058 | 0.658083 | unknown |
| 2041 | vng2041 | −0.039 | 0.913 | 0.473542 | 0.062 | 1.154 | 0.53445 | unknown |
| 2042 | vng2042 | 0.042 | 1.102 | 0.483261 | 0.000 | 1.000 | 0.839 | unknown |
| 2043 | ham1 | 0.009 | 1.022 | 0.476445 | 0.025 | 1.058 | 0.662792 | nucleotide metabolism |
| 2044 | vng2044 | 0.154 | 1.427 | 0.170271 | −0.007 | 0.985 | 0.418 | unknown |
| 2045 | gcp | −0.131 | 0.740 | 0.215579 | 0.049 | 1.119 | 0.500417 | cellular process |
| 2047 | rps27ae | 0.173 | 1.490 | 0.00755 | 0.047 | 1.116 | 0.39325 | translation |
| 2048 | rps24e | −0.064 | 0.862 | 0.438755 | 0.007 | 1.017 | 0.554833 | translation |
| 2051 | rpoE″ | −0.092 | 0.810 | 0.117016 | 0.046 | 1.113 | 0.4135 | transcription and regulation |
| 2053 | rpoE′ | −0.056 | 0.880 | 0.419683 | 0.066 | 1.165 | 0.2176 | transcription and regulation |
| 2054 | vng2054 | −0.078 | 0.837 | 0.32123 | 0.044 | 1.107 | 0.47195 | unknown |
| 2056 | eif2g | −0.037 | 0.919 | 0.592128 | 0.052 | 1.128 | 0.400433 | translation |
| 2059 | vng2059 | −0.122 | 0.756 | 0.460634 | 0.027 | 1.063 | 0.811083 | unknown |
| 2060 | phnP | −0.032 | 0.930 | 0.187925 | −0.077 | 0.837 | 0.152888 | general function prediction only |
| 2062 | atp | −0.110 | 0.777 | 0.147101 | −0.054 | 0.884 | 0.381933 | cellular process |
| 2063 | aca | 0.108 | 1.282 | 0.362032 | −0.005 | 0.988 | 0.695 | general function prediction only |
| 2064 | Vng2064 | −0.103 | 0.788 | 0.1172 | 0.006 | 1.014 | 0.690167 | cellular process |
| 2065 | dgs | −0.054 | 0.884 | 0.373969 | −0.005 | 0.989 | 0.646667 | cofactor and secondary |
| | | | | | | | | metabolites |
| 2067 | vng2067 | 0.005 | 1.012 | 0.721965 | 0.016 | 1.038 | 0.823 | unknown |
| 2068 | trp11 | −0.051 | 0.889 | 0.673947 | −0.021 | 0.952 | 0.666167 | transport |
| 2071 | lfl2 | 0.065 | 1.162 | 0.328226 | −0.028 | 0.937 | 0.4815 | carbohydrate and lipid metabolism |
| 2072 | serS | −0.001 | 0.998 | 0.512463 | 0.060 | 1.148 | 0.29865 | translation |
| 2073 | vng2073 | 0.118 | 1.313 | 0.452332 | −0.044 | 0.903 | 0.4275 | unknown |
| 2074 | vng2074 | 0.000 | 1.000 | 0.801362 | −0.044 | 0.903 | 0.417 | transcription and regulation |
| 2075 | vng2075 | −0.010 | 0.978 | 0.755666 | 0.037 | 1.088 | 0.72925 | unknown |
| 2076 | rpl40e | 0.105 | 1.274 | 0.370388 | −0.048 | 0.896 | 0.602 | translation |
| 2078 | hat2 | 0.049 | 1.120 | 0.528524 | −0.123 | 0.752 | 0.039923 | translation |
| 2079 | muT | 0.162 | 1.453 | 0.005086 | −0.085 | 0.821 | 0.18025 | DNA metabolism |
| 2080 | gloB | 0.020 | 1.048 | 0.723991 | −0.055 | 0.882 | 0.3645 | general function prediction only |
| 2081 | vng2081 | −0.017 | 0.962 | 0.561715 | −0.043 | 0.907 | 0.396325 | unknown |
| 2082 | urg3 | 0.032 | 1.077 | 0.561231 | −0.028 | 0.937 | 0.798 | DNA metabolism |
| 2084 | phnE | −0.148 | 0.711 | 0.166231 | 0.017 | 1.041 | 0.680667 | transport |
| 2085 | phnC | −0.088 | 0.817 | 0.212973 | −0.019 | 0.957 | 0.7055 | transport |
| 2086 | hpb | 0.090 | 1.230 | 0.372385 | −0.097 | 0.799 | 0.346826 | general function prediction only |
| 2087 | hisH2 | 0.043 | 1.103 | 0.321835 | −0.106 | 0.783 | 0.476667 | amino acid metabolism |
| 2088 | trm1 | 0.019 | 1.046 | 0.702846 | 0.042 | 1.102 | 0.520083 | translation |
| 2089 | vng2089 | −0.053 | 0.884 | 0.528667 | 0.015 | 1.034 | 0.462583 | unknown |
| 2091 | vng2091 | −0.060 | 0.871 | 0.526031 | −0.046 | 0.900 | 0.456 | carbohydrate and lipid metabolism |
| 2093 | glnA | 0.053 | 1.131 | 0.282434 | −0.021 | 0.953 | 0.546417 | amino acid metabolism |
| 2094 | asxR6 | 0.097 | 1.249 | 0.191693 | −0.014 | 0.969 | 0.6685 | transcription and regulation |
| 2096 | cctB | −0.043 | 0.906 | 0.489706 | 0.064 | 1.160 | 0.302925 | cellular process |
| 2097 | vng2097 | 0.064 | 1.159 | 0.397044 | 0.053 | 1.129 | 0.411917 | unknown |
| 2098 | vng2098 | 0.192 | 1.557 | 0.012559 | −0.022 | 0.951 | 0.687 | unknown |
| 2099 | vng2099 | 0.201 | 1.589 | 0.0081 | −0.056 | 0.880 | 0.31085 | translation |
| 2100 | iluA | 0.056 | 1.137 | 0.31572 | −0.027 | 0.940 | 0.628083 | amino acid metabolism |
| 2101 | vng2101 | 0.014 | 1.033 | 0.422934 | −0.063 | 0.864 | 0.521849 | unknown |
| 2102 | citZ | 0.251 | 1.783 | 0.196011 | 0.016 | 1.038 | 0.720125 | energy metabolism |
| 2104 | pchB | 0.060 | 1.147 | 0.467067 | 0.009 | 1.020 | 0.463583 | transport |
| 2105 | vng2105 | −0.105 | 0.785 | 0.278884 | −0.049 | 0.893 | 0.35225 | unknown |
| 2106 | sdh | −0.004 | 0.990 | 0.315272 | 0.012 | 1.029 | 0.575283 | general function prediction only |
| 2108 | thrC3 | 0.031 | 1.073 | 0.551031 | 0.041 | 1.100 | 0.51175 | amino acid metabolism |
| 2109 | vng2109 | 0.022 | 1.051 | 0.618623 | −0.137 | 0.729 | 0.238 | unknown |
| 2110 | dzr | −0.048 | 0.896 | 0.532162 | −0.052 | 0.887 | 0.359083 | transcription and regulation |
| 2112 | xrlR5 | −0.188 | 0.649 | 0.312602 | 0.059 | 1.146 | 0.41724 | transcription and regulation |
| 2113 | imp3 | −0.007 | 0.985 | 0.693679 | −0.054 | 0.882 | 0.440117 | general function prediction only |
| 2115 | vng2115 | 0.101 | 1.261 | 0.441006 | −0.113 | 0.770 | 0.08344 | cellular process |
| 2116 | vng2116 | 0.037 | 1.089 | 0.522369 | 0.096 | 1.247 | 0.075912 | unknown |
| 2117 | vng2117 | 0.013 | 1.031 | 0.19535 | −0.045 | 0.902 | 0.515077 | nucleotide metabolism |
| 2118 | pyrE2 | 0.053 | 1.130 | 0.50254 | −0.002 | 0.995 | 0.802 | nucleotide metabolism |
| 2119 | vng2119 | 0.049 | 1.119 | 0.375934 | −0.044 | 0.903 | 0.538833 | carbohydrate and lipid metabolism |
| 2120 | yusM | 0.144 | 1.394 | 0.154824 | 0.020 | 1.048 | 0.47075 | amino acid metabolism |
| 2121 | vng2121 | −0.007 | 0.984 | 0.42486 | −0.003 | 0.993 | 0.77125 | unknown |
| 2122 | ilvE2 | 0.221 | 1.664 | 0.001272 | −0.028 | 0.937 | 0.528167 | amino acid metabolism |
| 2123 | nhaC2 | 0.010 | 1.024 | 0.673686 | 0.019 | 1.044 | 0.607 | transport |
| 2124 | nhaC5 | 0.012 | 1.027 | 0.581206 | 0.016 | 1.038 | 0.748167 | transport |
| 2125 | ribA | 0.007 | 1.017 | 0.375225 | −0.065 | 0.862 | 0.2518 | cofactor and secondary |
| | | | | | | | | metabolites |
| 2126 | rflrR | −0.063 | 0.864 | 0.369653 | 0.053 | 1.131 | 0.447058 | transcription and regulation |
| 2128 | vng2128 | −0.141 | 0.723 | 0.466301 | 0.024 | 1.056 | 0.692833 | energy metabolism |
| 2129 | vng2129 | 0.060 | 1.147 | 0.506008 | −0.042 | 0.908 | 0.394 | unknown |
| 2130 | minD2 | −0.029 | 0.935 | 0.727395 | 0.023 | 1.054 | 0.63475 | cellular process |
| 2131 | erf1 | 0.028 | 1.066 | 0.50281 | 0.016 | 1.037 | 0.771417 | translation |
| 2132 | ferA1 | −0.001 | 0.998 | 0.962146 | 0.043 | 1.104 | 0.5985 | energy metabolism |
| 2133 | vng2133 | −0.033 | 0.927 | 0.594622 | −0.004 | 0.990 | 0.644917 | transcription and regulation |
| 2135 | atpD | −0.115 | 0.767 | 0.154208 | 0.026 | 1.061 | 0.2832 | energy metabolism |
| 2136 | blh | 0.076 | 1.192 | 0.374093 | −0.012 | 0.972 | 0.6715 | energy metabolism |
| 2137 | crtY | −0.014 | 0.968 | 0.698247 | 0.041 | 1.100 | 0.496875 | cofactor and secondary |
| | | | | | | | | metabolites |
| 2138 | atpB | −0.004 | 0.992 | 0.147408 | 0.017 | 1.039 | 0.631833 | energy metabolism |
| 2139 | atpA | −0.204 | 0.625 | 0.010679 | 0.080 | 1.203 | 0.147792 | energy metabolism |
| 2140 | atpF | −0.064 | 0.863 | 0.292572 | 0.099 | 1.257 | 0.0506 | energy metabolism |
| 2141 | atpC | −0.254 | 0.557 | 0.000548 | 0.078 | 1.198 | 0.12855 | energy metabolism |
| 2142 | atpE | −0.142 | 0.721 | 0.010262 | 0.055 | 1.134 | 0.27 | energy metabolism |
| 2143 | atpK | −0.206 | 0.622 | 0.014382 | −0.014 | 0.968 | 0.53375 | energy metabolism |
| 2144 | atpI | −0.165 | 0.684 | 0.009025 | 0.007 | 1.016 | 0.857833 | energy metabolism |
| 2146 | atpH | 0.078 | 1.198 | 0.238585 | 0.066 | 1.164 | 0.1975 | energy metabolism |
| 2147 | hmp | 0.020 | 1.047 | 0.637103 | −0.002 | 0.995 | 0.636833 | cofactor and secondary |
| | | | | | | | | metabolites |
| 2148 | vng2148 | −0.012 | 0.972 | 0.577312 | −0.080 | 0.832 | 0.259933 | unknown |
| 2149 | vng2149 | −0.021 | 0.952 | 0.620455 | 0.054 | 1.133 | 0.31845 | unknown |
| 2150 | etfB | 0.129 | 1.347 | 0.21052 | 0.011 | 1.027 | 0.663 | energy metabolism |
| 2151 | etfA | 0.019 | 1.045 | 0.529026 | 0.016 | 1.038 | 0.72275 | energy metabolism |
| 2152 | vng2152 | 0.107 | 1.279 | 0.174937 | −0.021 | 0.954 | 0.643167 | unknown |
| 2153 | fps | −0.065 | 0.861 | 0.429542 | 0.094 | 1.243 | 0.2227 | cofactor and secondary |
| | | | | | | | | metabolites |
| 2154 | vng2154 | 0.116 | 1.307 | 0.16839 | −0.228 | 0.592 | 0.36025 | unknown |
| 2155 | vng2155 | −0.070 | 0.851 | 0.252917 | 0.008 | 1.018 | 0.55805 | unknown |
| 2156 | vng2156 | 0.028 | 1.067 | 0.020845 | −0.080 | 0.831 | 0.159417 | cellular process |
| 2157 | vng2157 | 0.030 | 1.071 | 0.381953 | −0.001 | 0.997 | 0.602 | unknown |
| 2158 | oxrA | −0.114 | 0.770 | 0.125582 | 0.069 | 1.171 | 0.414922 | cofactor and secondary |
| | | | | | | | | metabolites |
| 2159 | manC | 0.100 | 1.260 | 0.124616 | −0.066 | 0.859 | 0.216 | energy metabolism |
| 2160 | rfa3 | 0.508 | 3.220 | 7.66E−13 | 0.737 | 5.458 | 2.86E−23 | DNA metabolism |
| 2162 | rfa8 | 0.284 | 1.922 | 0.002387 | 0.558 | 3.617 | 2.88E−12 | DNA metabolism |
| 2163 | ral | 0.355 | 2.264 | 6.5E−08 | 0.529 | 3.382 | 4.09E−15 | DNA metabolism |
| 2164 | vng2164 | −0.034 | 0.924 | 0.503439 | −0.128 | 0.744 | 0.014185 | unknown |
| 2165 | vng2165 | −0.072 | 0.847 | 0.422535 | 0.034 | 1.082 | 0.559925 | unknown |
| 2166 | vng2166 | −0.002 | 0.996 | 0.70604 | −0.240 | 0.575 | 8.8E−06 | unknown |
| 2167 | dbp | 0.027 | 1.063 | 0.478625 | −0.005 | 0.989 | 0.250325 | DNA metabolism |
| 2168 | vng2168 | 0.013 | 1.029 | 0.117274 | 0.007 | 1.017 | 0.42475 | unknown |
| 2170 | vng2170 | 0.184 | 1.528 | 0.334009 | 0.032 | 1.076 | 0.668 | unknown |
| 2171 | adh1 | 0.004 | 1.009 | 0.423139 | −0.053 | 0.885 | 0.3896 | energy metabolism |
| 2172 | vng2172 | 0.042 | 1.102 | 0.447229 | −0.035 | 0.922 | 0.593055 | unknown |
| 2173 | rad24a | −0.079 | 0.833 | 0.299522 | −0.024 | 0.946 | 0.28158 | DNA metabolism |
| 2176 | vng2176 | −0.108 | 0.781 | 0.451537 | −0.029 | 0.936 | 0.56975 | unknown |
| 2178 | phlR | −0.056 | 0.878 | 0.315725 | 0.023 | 1.055 | 0.607333 | transcription and regulation |
| 2179 | vng2179 | 0.008 | 1.019 | 0.612451 | −0.010 | 0.976 | 0.787417 | unknown |
| 2180 | hik5 | −0.237 | 0.580 | 0.000494 | 0.002 | 1.004 | 0.687125 | cellular process |
| 2181 | mcm | −0.046 | 0.899 | 0.434572 | 0.019 | 1.045 | 0.459083 | DNA metabolism |
| 2182 | vng2182 | −0.057 | 0.878 | 0.374596 | 0.059 | 1.146 | 0.365817 | unknown |
| 2183 | vng2183 | −0.057 | 0.877 | 0.605615 | 0.055 | 1.134 | 0.400925 | unknown |
| 2184 | tfbA | 0.132 | 1.354 | 0.323264 | −0.027 | 0.941 | 0.522833 | transcription and regulation |
| 2185 | vng2185 | −0.007 | 0.984 | 0.667239 | 0.015 | 1.036 | 0.559083 | unknown |
| 2186 | hit1 | 0.057 | 1.140 | 0.383497 | −0.199 | 0.633 | 0.000278 | nucleotide metabolism |
| 2187 | vng2187 | −0.017 | 0.962 | 0.432278 | 0.057 | 1.140 | 0.4687 | unknown |
| 2189 | vng2189 | −0.038 | 0.915 | 0.490245 | −0.041 | 0.909 | 0.55775 | unknown |
| 2190 | ileS | 0.025 | 1.059 | 0.448124 | 0.043 | 1.105 | 0.446575 | translation |
| 2191 | vng2191 | 0.050 | 1.123 | 0.379248 | −0.024 | 0.947 | 0.65575 | unknown |
| 2193 | cbaA | 0.087 | 1.221 | 0.274076 | −0.040 | 0.912 | 0.489583 | energy metabolism |
| 2195 | cbaB | 0.072 | 1.181 | 0.46526 | −0.150 | 0.709 | 0.5075 | energy metabolism |
| 2196 | cbaD | 0.171 | 1.483 | 0.046399 | −0.022 | 0.951 | 0.669583 | energy metabolism |
| 2197 | vng2197 | −0.006 | 0.985 | 0.643322 | −0.020 | 0.955 | 0.76375 | unknown |
| 2199 | vng2199 | −0.003 | 0.993 | 0.646935 | −0.008 | 0.983 | 0.7475 | unknown |
| 2201 | cpx | 0.032 | 1.077 | 0.613862 | 0.027 | 1.065 | 0.65875 | transport |
| 2202 | vng2202 | −0.007 | 0.983 | 0.774283 | 0.008 | 1.018 | 0.80225 | unknown |
| 2203 | prsA | 0.018 | 1.043 | 0.572861 | −0.030 | 0.933 | 0.5655 | nucleotide metabolism |
| 2204 | vng2204 | 0.021 | 1.050 | 0.718788 | −0.019 | 0.956 | 0.7145 | unknown |
| 2205 | vng2205 | −0.095 | 0.804 | 0.249033 | 0.030 | 1.072 | 0.412833 | unknown |
| 2206 | pmu1 | −0.060 | 0.872 | 0.463959 | −0.051 | 0.889 | 0.433907 | carbohydrate and lipid metabolism |
| 2207 | vng2207 | −0.019 | 0.957 | 0.528572 | −0.040 | 0.912 | 0.513667 | unknown |
| 2208 | trpS1 | −0.032 | 0.930 | 0.515054 | −0.027 | 0.941 | 0.6864 | translation |
| 2210 | endA | −0.173 | 0.672 | 0.178954 | 0.006 | 1.014 | 0.817 | translation |
| 2211 | vng2211 | 0.010 | 1.024 | 0.65054 | 0.019 | 1.045 | 0.648558 | unknown |
| 2213 | brr2 | −0.084 | 0.825 | 0.231261 | −0.009 | 0.979 | 0.524 | transcription and regulation |
| 2214 | dinF | 0.050 | 1.121 | 0.449526 | 0.029 | 1.069 | 0.526833 | DNA metabolism |
| 2216 | lip | 0.091 | 1.234 | 0.296179 | 0.007 | 1.016 | 0.76625 | cofactor and secondary |
| | | | | | | | | metabolites |
| 2217 | pdhA2 | −0.046 | 0.900 | 0.25058 | 0.036 | 1.087 | 0.477083 | energy metabolism |
| 2218 | pdhB | −0.053 | 0.885 | 0.427174 | 0.001 | 1.002 | 0.291867 | energy metabolism |
| 2219 | dsa | −0.068 | 0.854 | 0.327566 | −0.029 | 0.936 | 0.5615 | energy metabolism |
| 2220 | lpdA | 0.002 | 1.004 | 0.504137 | −0.004 | 0.991 | 0.675875 | energy metabolism |
| 2222 | pheA | −0.016 | 0.965 | 0.290437 | 0.006 | 1.014 | 0.504783 | amino acid metabolism |
| 2223 | leuS | 0.058 | 1.142 | 0.371222 | 0.038 | 1.092 | 0.468 | translation |
| 2224 | ocd1 | 0.188 | 1.541 | 0.001031 | −0.053 | 0.885 | 0.293 | amino acid metabolism |
| 2226 | cctA | 0.373 | 2.361 | 0.019116 | −0.036 | 0.920 | 0.4125 | cellular process |
| 2227 | vng2227 | 0.045 | 1.108 | 0.448412 | 0.005 | 1.011 | 0.747375 | unknown |
| 2230 | vng2230 | 0.008 | 1.017 | 0.43886 | −0.008 | 0.982 | 0.634917 | unknown |
| 2231 | vng2231 | 0.040 | 1.096 | 0.468644 | 0.010 | 1.024 | 0.719167 | carbohydrate and lipid metabolism |
| 2232 | trpS2 | −0.078 | 0.836 | 0.504575 | 0.005 | 1.011 | 0.492683 | translation |
| 2233 | stk | −0.097 | 0.800 | 0.302935 | −0.015 | 0.967 | 0.726833 | cellular process |
| 2234 | eif1a1 | −0.009 | 0.979 | 0.490118 | −0.061 | 0.869 | 0.2845 | translation |
| 2236 | vng2236 | −0.035 | 0.922 | 0.510146 | −0.008 | 0.982 | 0.799 | unknown |
| 2237 | tyrS | 0.011 | 1.026 | 0.504099 | 0.055 | 1.134 | 0.331467 | translation |
| 2238 | vng2238 | −0.098 | 0.798 | 0.387372 | 0.052 | 1.127 | 0.348167 | unknown |
| 2239 | vng2239 | 0.011 | 1.025 | 0.531665 | 0.015 | 1.034 | 0.78325 | unknown |
| 2240 | rfcC | 0.074 | 1.185 | 0.274386 | 0.001 | 1.003 | 0.892833 | DNA metabolism |
| 2241 | vng2241 | −0.037 | 0.919 | 0.524524 | 0.017 | 1.040 | 0.7725 | unknown |
| 2243 | tbpE | 0.221 | 1.665 | 0.000393 | −0.046 | 0.899 | 0.381 | transcription and regulation |
| 2244 | vng2244 | 0.036 | 1.087 | 0.630468 | 0.025 | 1.060 | 0.629 | unknown |
| 2246 | vng2246 | −0.030 | 0.933 | 0.652328 | 0.029 | 1.069 | 0.792833 | unknown |
| 2247 | hisG | 0.010 | 1.023 | 0.531374 | 0.001 | 1.003 | 0.558375 | amino acid metabolism |
| 2248 | vng2248 | 0.001 | 1.002 | 0.669256 | −0.014 | 0.969 | 0.519375 | unknown |
| 2249 | trzA | 0.030 | 1.072 | 0.557669 | −0.007 | 0.984 | 0.4005 | nucleotide metabolism |
| 2251 | achY | 0.115 | 1.304 | 0.153944 | −0.002 | 0.996 | 0.742917 | cofactor and secondary |
| | | | | | | | | metabolites |
| 2252 | hjr | 0.067 | 1.166 | 0.247087 | −0.022 | 0.951 | 0.6675 | DNA metabolism |
| 2253 | vng2253 | −0.006 | 0.987 | 0.623704 | −0.064 | 0.864 | 0.293563 | unknown |
| 2254 | vng2254 | −0.070 | 0.851 | 0.296257 | −0.059 | 0.874 | 0.401118 | DNA metabolism |
| 2255 | vng2255 | −0.030 | 0.934 | 0.393631 | 0.030 | 1.071 | 0.561675 | carbohydrate and lipid metabolism |
| 2256 | pcn | 0.130 | 1.348 | 0.144808 | −0.033 | 0.926 | 0.4115 | DNA metabolism |
| 2257 | nce | 0.061 | 1.151 | 0.466715 | 0.035 | 1.084 | 0.692833 | transport |
| 2259 | vng2259 | 0.017 | 1.040 | 0.674269 | −0.003 | 0.992 | 0.64225 | unknown |
| 2260 | vng2260 | −0.040 | 0.912 | 0.429781 | 0.005 | 1.012 | 0.656583 | unknown |
| 2262 | vng2262 | 0.020 | 1.047 | 0.534759 | 0.006 | 1.015 | 0.542083 | unknown |
| 2263 | ftsJ | 0.001 | 1.002 | 0.338866 | 0.076 | 1.192 | 0.223233 | cellular process |
| 2264 | vng2264 | −0.077 | 0.838 | 0.380579 | −0.011 | 0.975 | 0.7745 | unknown |
| 2267 | tatC2 | −0.034 | 0.925 | 0.362707 | −0.014 | 0.967 | 0.687 | cellular process |
| 2268 | Vng2268 | −0.034 | 0.925 | 0.309178 | −0.060 | 0.871 | 0.4213 | unknown |
| 2269 | tatC1 | −0.007 | 0.985 | 0.550073 | 0.033 | 1.079 | 0.43625 | cellular process |
| 2270 | mutS3 | −0.091 | 0.811 | 0.259735 | 0.040 | 1.097 | 0.586258 | DNA metabolism |
| 2271 | orc6 | −0.017 | 0.962 | 0.523595 | 0.023 | 1.054 | 0.512525 | DNA metabolism |
| 2272 | rpi | −0.048 | 0.895 | 0.356592 | 0.030 | 1.071 | 0.614033 | carbohydrate and lipid metabolism |
| 2273 | vng2273 | 0.020 | 1.047 | 0.008926 | | 1.000 | | unknown |
| 2274 | vng2274 | −0.036 | 0.919 | 0.099675 | 0.022 | 1.053 | 0.8625 | DNA metabolism |
| 2276 | pmm | 0.090 | 1.231 | 0.194974 | 0.017 | 1.041 | 0.558 | carbohydrate and lipid metabolism |
| 2277 | act8 | 0.044 | 1.107 | 0.430875 | −0.004 | 0.990 | 0.7395 | transcription and regulation |
| 2279 | vng2279 | 0.047 | 1.115 | 0.479517 | 0.014 | 1.034 | 0.775583 | cofactor and secondary |
| | | | | | | | | metabolites |
| 2280 | rfcA | 0.015 | 1.036 | 0.757344 | 0.028 | 1.067 | 0.593 | DNA metabolism |
| 2281 | vng2281 | 0.098 | 1.253 | 0.230547 | −0.065 | 0.861 | 0.328725 | cellular process |
| 2282 | vng2282 | 0.107 | 1.278 | 0.258354 | −0.005 | 0.988 | 0.826 | cellular process |
| 2283 | alaS | −0.051 | 0.890 | 0.414808 | 0.005 | 1.012 | 0.732583 | translation |
| 2284 | hisJ | 0.083 | 1.211 | 0.109422 | −0.223 | 0.599 | 5.03E−05 | amino acid metabolism |
| 2285 | vng2285 | −0.090 | 0.813 | 0.20644 | −0.032 | 0.928 | 0.515167 | cellular process |
| 2286 | mamA | 0.052 | 1.127 | 0.435249 | −0.080 | 0.832 | 0.375067 | amino acid metabolism |
| 2288 | mamB | 0.010 | 1.023 | 0.395241 | 0.005 | 1.012 | 0.488825 | amino acid metabolism |
| 2289 | mal | 0.006 | 1.014 | 0.295012 | 0.058 | 1.142 | 0.362483 | amino acid metabolism |
| 2290 | maoC1 | −0.042 | 0.908 | 0.397832 | 0.013 | 1.031 | 0.786 | carbohydrate and lipid metabolism |
| 2291 | cat | 0.018 | 1.042 | 0.542339 | 0.030 | 1.071 | 0.568725 | carbohydrate and lipid metabolism |
| 2292 | vng2292 | 0.065 | 1.162 | 0.405372 | 0.050 | 1.121 | 0.384708 | unknown |
| 2293 | ferA2 | 0.251 | 1.782 | 0.000102 | 0.026 | 1.063 | 0.606833 | energy metabolism |
| 2294 | hisA | 0.000 | 1.001 | 0.541644 | 0.005 | 1.012 | 0.653583 | amino acid metabolism |
| 2295 | hisB | 0.045 | 1.108 | 0.371411 | −0.069 | 0.853 | 0.763 | amino acid metabolism |
| 2296 | aamR | 0.035 | 1.083 | 0.713386 | −0.024 | 0.946 | 0.8085 | transcription and regulation |
| 2297 | vng2296 | 0.101 | 1.262 | 0.257707 | −0.036 | 0.919 | 0.47425 | unknown |
| 2298 | vng2298 | 0.090 | 1.230 | 0.135419 | −0.041 | 0.909 | 0.436083 | unknown |
| 2299 | vng2299 | 0.018 | 1.042 | 0.538846 | −0.032 | 0.929 | 0.55425 | unknown |
| 2301 | txrB3 | 0.058 | 1.143 | 0.521288 | 0.057 | 1.141 | 0.3215 | cellular process |
| 2302 | yuxL | 0.041 | 1.099 | 0.367651 | 0.030 | 1.073 | 0.596792 | amino acid metabolism |
| 2304 | vng2304 | 0.017 | 1.040 | 0.652362 | 0.050 | 1.122 | 0.39325 | unknown |
| 2305 | vng2305 | 0.073 | 1.182 | 0.251823 | −0.026 | 0.941 | 0.6545 | nucleotide metabolism |
| 2306 | vng2306 | −0.010 | 0.976 | 0.6808 | 0.021 | 1.050 | 0.6185 | unknown |
| 2307 | vng2307 | −0.043 | 0.906 | 0.39772 | 0.024 | 1.057 | 0.603417 | unknown |
| 2308 | hlp | 0.088 | 1.225 | 0.254834 | 0.025 | 1.059 | 0.629167 | general function prediction only |
| 2310 | vng2310 | 0.009 | 1.022 | 0.030703 | 0.028 | 1.068 | 0.63175 | cellular process |
| 2311 | vng2311 | 0.110 | 1.289 | 0.499353 | 0.026 | 1.063 | 0.800125 | cellular process |
| 2312 | vng2312 | −0.040 | 0.912 | 0.548566 | 0.020 | 1.046 | 0.78675 | translation |
| 2313 | vng2313 | 0.030 | 1.070 | 0.575668 | −0.013 | 0.971 | 0.807417 | unknown |
| 2314 | vng2314 | −0.021 | 0.952 | 0.424493 | 0.004 | 1.009 | 0.658833 | unknown |
| 2315 | vng2315 | −0.021 | 0.952 | 0.406354 | −0.121 | 0.757 | 0.050818 | unknown |
| 2316 | vng2316 | 0.005 | 1.012 | 0.433808 | 0.008 | 1.019 | 0.8775 | unknown |
| 2317 | cbiO1 | 0.080 | 1.204 | 0.698951 | 0.033 | 1.080 | 0.624 | transport |
| 2320 | hdrD | −0.048 | 0.895 | 0.133947 | 0.029 | 1.069 | 0.547417 | energy metabolism |
| 2321 | ydaJ | 0.139 | 1.376 | 0.223619 | −0.108 | 0.780 | 0.087663 | translation |
| 2322 | hem2 | −0.003 | 0.992 | 0.62497 | 0.002 | 1.006 | 0.785125 | cofactor and secondary |
| | | | | | | | | metabolites |
| 2323 | vng2323 | 0.009 | 1.021 | 0.726968 | 0.010 | 1.022 | 0.596558 | unknown |
| 2324 | vng2324 | −0.179 | 0.662 | 0.260391 | −0.030 | 0.932 | 0.696125 | unknown |
| 2326 | hemL | 0.015 | 1.035 | 0.465293 | 0.034 | 1.081 | 0.771833 | cofactor and secondary |
| | | | | | | | | metabolites |
| 2328 | vng2328 | −0.140 | 0.724 | 0.387544 | −0.015 | 0.967 | 0.464634 | unknown |
| 2329 | vng2329 | 0.036 | 1.086 | 0.423829 | 0.016 | 1.037 | 0.6995 | unknown |
| 2330 | hem3 | −0.066 | 0.860 | 0.270148 | 0.039 | 1.094 | 0.5955 | cofactor and secondary |
| | | | | | | | | metabolites |
| 2331 | uroM | −0.072 | 0.848 | 0.411412 | 0.054 | 1.131 | 0.411225 | cofactor and secondary |
| | | | | | | | | metabolites |
| 2332 | cysG | −0.011 | 0.975 | 0.826147 | 0.018 | 1.042 | 0.7175 | cofactor and secondary |
| | | | | | | | | metabolites |
| 2333 | vng2333 | −0.003 | 0.993 | 0.696406 | 0.031 | 1.074 | 0.608667 | DNA metabolism |
| 2334 | prh | 0.002 | 1.004 | 0.661316 | 0.015 | 1.036 | 0.727333 | cellular process |
| 2335 | vng2335 | −0.011 | 0.975 | 0.606829 | −0.008 | 0.982 | 0.829333 | unknown |
| 2337 | vng2337 | 0.069 | 1.172 | 0.266377 | −0.010 | 0.977 | 0.618333 | cellular process |
| 2338 | polA2 | −0.178 | 0.664 | 0.063699 | −0.038 | 0.915 | 0.484167 | DNA metabolism |
| 2339 | vng2339 | 0.053 | 1.131 | 0.350946 | −0.032 | 0.928 | 0.5855 | unknown |
| 2340 | vng2340 | 0.114 | 1.299 | 0.127392 | −0.057 | 0.877 | 0.4304 | unknown |
| 2341 | vng2341 | 0.179 | 1.509 | 0.047625 | −0.093 | 0.807 | 0.13775 | unknown |
| 2342 | vng2342 | 0.045 | 1.109 | 0.28339 | −0.030 | 0.933 | 0.567167 | unknown |
| 2343 | ykfD | 0.067 | 1.166 | 0.336737 | −0.041 | 0.910 | 0.485042 | transport |
| 2344 | oppD2 | −0.064 | 0.863 | 0.217566 | 0.006 | 1.013 | 0.85325 | transport |
| 2346 | dppC2 | −0.094 | 0.806 | 0.103825 | 0.034 | 1.081 | 0.536033 | transport |
| 2347 | dpp81 | 0.224 | 1.674 | 0.000157 | −0.015 | 0.967 | 0.77225 | transport |
| 2349 | dppA | 0.387 | 2.441 | 0.149 | 0.003 | 1.007 | 0.583417 | transport |
| 2351 | vng2351 | −0.056 | 0.879 | 0.443427 | 0.016 | 1.038 | 0.610333 | unknown |
| 2352 | glyS | −0.030 | 0.934 | 0.508785 | 0.006 | 1.014 | 0.658917 | translation |
| 2353 | vng2353 | 0.047 | 1.114 | 0.445628 | 0.042 | 1.102 | 0.421225 | unknown |
| 2356 | eif4a | 0.000 | 1.000 | 0.57953 | 0.035 | 1.084 | 0.578275 | translation |
| 2358 | appA | 0.033 | 1.079 | 0.617939 | 0.042 | 1.101 | 0.662917 | transport |
| 2359 | appB | 0.011 | 1.026 | 0.516325 | 0.059 | 1.145 | 0.435667 | transport |
| 2361 | appC | 0.058 | 1.142 | 0.516136 | 0.052 | 1.128 | 0.41 | transport |
| 2363 | oppD1 | −0.057 | 0.876 | 0.390609 | 0.015 | 1.036 | 0.52945 | transport |
| 2365 | appF | 0.025 | 1.059 | 0.431455 | 0.003 | 1.007 | 0.503688 | transport |
| 2366 | vng2366 | −0.005 | 0.988 | 0.916638 | −0.065 | 0.861 | 0.1895 | unknown |
| 2367 | mdhA | 0.032 | 1.076 | 0.459433 | 0.049 | 1.118 | 0.341 | energy metabolism |
| 2368 | rad24b | −0.076 | 0.840 | 0.271346 | 0.033 | 1.080 | 0.578583 | DNA metabolism |
| 2369 | vng2369 | −0.028 | 0.937 | 0.574845 | 0.053 | 1.130 | 0.41035 | unknown |
| 2370 | vng2370 | −0.041 | 0.909 | 0.570157 | −0.041 | 0.911 | 0.487 | energy metabolism |
| 2371 | vng2371 | 0.020 | 1.048 | 0.523469 | 0.123 | 1.327 | 0.116591 | nucleotide metabolism |
| 2372 | rad24c | 0.025 | 1.059 | 0.520318 | −0.014 | 0.969 | 0.354983 | DNA metabolism |
| 2373 | tnaA | 0.046 | 1.112 | 0.223564 | 0.013 | 1.031 | 0.595667 | amino acid metabolism |
| 2374 | lysC | −0.056 | 0.880 | 0.205806 | −0.045 | 0.902 | 0.386875 | amino acid metabolism |
| 2376 | vng2376 | −0.065 | 0.860 | 0.433963 | −0.007 | 0.983 | 0.851167 | unknown |
| 2377 | nosY | 0.019 | 1.046 | 0.549836 | 0.013 | 1.030 | 0.804 | transport |
| 2378 | nosF1 | 0.030 | 1.072 | 0.340644 | −0.021 | 0.952 | 0.488267 | transport |
| 2379 | vng2379 | −0.048 | 0.896 | 0.711 | −0.088 | 0.817 | 0.0848 | unknown |
| 2380 | vng2380 | 0.211 | 1.626 | 0.000836 | 0.002 | 1.004 | 0.92225 | unknown |
| 2381 | uvrC | 0.021 | 1.049 | 0.533392 | 0.014 | 1.032 | 0.695 | DNA metabolism |
| 2383 | nrdA | 0.219 | 1.657 | 0.03815 | 0.010 | 1.024 | 0.859 | nucleotide metabolism |
| 2384 | nrdB | 0.166 | 1.464 | 0.125019 | 0.001 | 1.002 | 0.579667 | nucleotide metabolism |
| 2385 | vng2385 | 0.156 | 1.433 | 0.141497 | −0.016 | 0.965 | 0.823 | unknown |
| 2386 | vng2386 | −0.017 | 0.962 | 0.616474 | 0.006 | 1.013 | 0.676833 | cellular process |
| 2387 | vng2387 | −0.066 | 0.860 | 0.288233 | −0.041 | 0.910 | 0.645538 | unknown |
| 2390 | uvrB | −0.025 | 0.944 | 0.313061 | −0.046 | 0.899 | 0.293917 | DNA metabolism |
| 2392 | vng2392 | 0.016 | 1.038 | 0.492938 | −0.018 | 0.959 | 0.737125 | unknown |
| 2393 | tssA | 0.086 | 1.220 | 0.309275 | −0.013 | 0.970 | 0.615083 | cellular process |
| 2394 | tssB | 0.004 | 1.010 | 0.825886 | 0.010 | 1.024 | 0.668125 | cellular process |
| 2395 | vng2395 | 0.032 | 1.077 | 0.611506 | 0.045 | 1.108 | 0.388625 | cofactor and secondary |
| | | | | | | | | metabolites |
| 2396 | cysT1 | 0.033 | 1.079 | 0.373368 | −0.008 | 0.982 | 0.602525 | transport |
| 2397 | cysA | −0.105 | 0.785 | 0.310065 | 0.040 | 1.097 | 0.396667 | transport |
| 2398 | scm | −0.001 | 0.997 | 0.646497 | 0.023 | 1.054 | 0.706 | cofactor and secondary |
| | | | | | | | | metabolites |
| 2399 | vng2399 | −0.069 | 0.853 | 0.470261 | 0.006 | 1.014 | 0.599 | unknown |
| 2400 | vng2400 | 0.044 | 1.108 | 0.421413 | −0.045 | 0.902 | 0.487196 | unknown |
| 2402 | vng2403 | 0.059 | 1.145 | 0.265632 | −0.023 | 0.947 | 0.64 | unknown |
| 2403 | vng2402 | 0.039 | 1.095 | 0.380656 | −0.001 | 0.998 | 0.7875 | unknown |
| 2404 | dip1 | 0.011 | 1.025 | 0.546046 | −0.005 | 0.988 | 0.708833 | DNA metabolism |
| 2406 | vng2406 | 0.117 | 1.311 | 0.080672 | −0.035 | 0.922 | 0.50075 | unknown |
| 2408 | vng2408 | 0.110 | 1.288 | 0.056412 | 0.001 | 1.002 | 0.8705 | unknown |
| 2410 | gbp3 | 0.131 | 1.351 | 0.082133 | −0.037 | 0.919 | 0.528917 | cellular process |
| 2411 | orc7 | −0.049 | 0.893 | 0.091293 | 0.024 | 1.057 | 0.262258 | DNA metabolism |
| 2412 | vng2412 | −0.113 | 0.771 | 0.140508 | 0.096 | 1.246 | 0.387 | unknown |
| 2413 | vng2413 | 0.003 | 1.007 | 0.46014 | 0.047 | 1.114 | 0.395833 | unknown |
| 2414 | arlR13 | −0.024 | 0.946 | 0.518617 | −0.073 | 0.845 | 0.205583 | transcription and regulation |
| 2415 | vng2415 | 0.000 | 1.000 | 0.477017 | −0.022 | 0.951 | 0.70625 | unknown |
| 2416 | sec11 | −0.078 | 0.836 | 0.37419 | 0.019 | 1.046 | 0.716833 | cellular process |
| 2417 | polA1 | −0.288 | 0.516 | 0.211251 | −0.037 | 0.919 | 0.525833 | DNA metabolism |
| 2418 | aspC1 | −0.035 | 0.923 | 0.494732 | 0.048 | 1.117 | 0.5065 | amino acid metabolism |
| 2419 | vng2419 | 0.003 | 1.007 | 0.387445 | −0.054 | 0.883 | 0.079063 | carbohydrate and lipid metabolism |
| 2420 | metA | 0.032 | 1.077 | 0.555679 | −0.091 | 0.811 | 0.149604 | amino acid metabolism |
| 2421 | hal | 0.033 | 1.079 | 0.503206 | −0.037 | 0.918 | 0.523973 | amino acid metabolism |
| 2422 | glcD | −0.103 | 0.788 | 0.481729 | −0.061 | 0.869 | 0.509942 | carbohydrate and lipid metabolism |
| 2423 | serB | −0.009 | 0.979 | 0.528675 | 0.025 | 1.059 | 0.685 | amino acid metabolism |
| 2424 | serA1 | −0.010 | 0.977 | 0.289087 | −0.089 | 0.816 | 0.208512 | amino acid metabolism |
| 2426 | act | −0.073 | 0.846 | 0.361922 | −0.077 | 0.838 | 0.300042 | carbohydrate and lipid metabolism |
| 2429 | dld | 0.106 | 1.277 | 0.244355 | 0.025 | 1.060 | 0.6476 | carbohydrate and lipid metabolism |
| 2430 | thrC1 | 0.000 | 1.000 | 0.508736 | 0.004 | 1.010 | 0.69975 | amino acid metabolism |
| 2431 | vng2431 | 0.060 | 1.148 | 0.377641 | −0.092 | 0.809 | 0.0724 | unknown |
| 2432 | vng2432 | 0.015 | 1.035 | 0.447959 | −0.021 | 0.954 | 0.69 | unknown |
| 2433 | vng2433 | −0.083 | 0.827 | 0.096604 | 0.085 | 1.216 | 0.11275 | unknown |
| 2436 | argH | −0.054 | 0.883 | 0.327982 | −0.045 | 0.901 | 0.29735 | amino acid metabolism |
| 2437 | argG | −0.103 | 0.789 | 0.462693 | 0.045 | 1.109 | 0.4535 | amino acid metabolism |
| 2439 | vng2439 | −0.005 | 0.989 | 0.282746 | 0.045 | 1.109 | 0.452083 | unknown |
| 2440 | vng2440 | 0.131 | 1.353 | 0.010451 | −0.043 | 0.905 | 0.4135 | unknown |
| 2441 | rad3b | −0.052 | 0.886 | 0.433729 | 0.037 | 1.090 | 0.460417 | DNA metabolism |
| 2442 | vng2442 | −0.051 | 0.890 | 0.211057 | −0.056 | 0.878 | 0.3105 | unknown |
| 2443 | dpsA | 0.179 | 1.510 | 0.081429 | 0.058 | 1.144 | 0.409665 | cellular process |
| 2444 | vng2444 | −0.016 | 0.964 | 0.349418 | −0.238 | 0.579 | 0.009485 | unknown |
| 2445 | arlR14 | −0.041 | 0.910 | 0.391935 | 0.056 | 1.138 | 0.34045 | transcription and regulation |
| 2446 | vng2446 | 0.012 | 1.029 | 0.189149 | −0.095 | 0.803 | 0.0979 | unknown |
| 2447 | lta | −0.071 | 0.849 | 0.25951 | 0.035 | 1.085 | 0.60245 | amino acid metabolism |
| 2449 | pepB2 | 0.039 | 1.095 | 0.499342 | −0.121 | 0.758 | 0.020733 | amino acid metabolism |
| 2451 | vng2451 | 0.114 | 1.301 | 0.046012 | −0.106 | 0.784 | 0.038 | unknown |
| 2452 | vng2452 | −0.059 | 0.872 | 0.414113 | −0.066 | 0.859 | 0.2668 | cofactor and secondary |
| | | | | | | | | metabolites |
| 2454 | vng2454 | −0.029 | 0.935 | 0.643082 | −0.088 | 0.817 | 0.14685 | unknown |
| 2455 | vng2455 | 0.011 | 1.026 | 0.595945 | 0.026 | 1.062 | 0.589333 | unknown |
| 2456 | vng2456 | −0.085 | 0.822 | 0.418346 | 0.081 | 1.206 | 0.1927 | unknown |
| 2458 | vng2458 | −0.003 | 0.993 | 0.778293 | 0.021 | 1.050 | 0.68325 | unknown |
| 2459 | srp54 | −0.042 | 0.908 | 0.428198 | 0.064 | 1.159 | 0.310783 | cellular process |
| 2461 | vng2461 | 0.015 | 1.034 | 0.205803 | −0.057 | 0.878 | 0.303 | unknown |
| 2462 | dpa | −0.010 | 0.978 | 0.730025 | 0.026 | 1.062 | 0.541 | cellular process |
| 2465 | vng2465 | −0.001 | 0.999 | 0.646022 | 0.052 | 1.126 | 0.332 | cellular process |
| 2466 | eif6 | −0.058 | 0.875 | 0.34924 | −0.015 | 0.967 | 0.747583 | translation |
| 2467 | rpl31e | 0.048 | 1.117 | 0.224373 | −0.021 | 0.954 | 0.545167 | translation |
| 2468 | vng2468 | −0.067 | 0.857 | 0.449315 | 0.015 | 1.035 | 0.801 | unknown |
| 2469 | rpl39e | −0.033 | 0.927 | 0.305033 | −0.080 | 0.832 | 0.21605 | translation |
| 2470 | vng2470 | −0.230 | 0.589 | 0.001759 | 0.026 | 1.063 | 0.69 | unknown |
| 2471 | nifS | 0.032 | 1.076 | 0.260587 | −0.022 | 0.950 | 0.677833 | amino acid metabolism |
| 2472 | nifU | 0.150 | 1.411 | 0.123653 | −0.030 | 0.934 | 0.568333 | transport |
| 2473 | radA1 | 0.211 | 1.624 | 0.06592 | 0.012 | 1.027 | 0.821917 | DNA metabolism |
| 2474 | vng2474 | −0.083 | 0.826 | 0.334646 | 0.036 | 1.085 | 0.495767 | translation |
| 2475 | vng2475 | −0.042 | 0.907 | 0.219993 | −0.008 | 0.982 | 0.64825 | unknown |
| 2476 | vng2476 | −0.038 | 0.917 | 0.457262 | 0.021 | 1.050 | 0.822417 | DNA metabolism |
| 2477 | vng2477 | −0.093 | 0.807 | 0.219659 | 0.009 | 1.021 | 0.8845 | unknown |
| 2480 | phoU | −0.003 | 0.992 | 0.384744 | 0.067 | 1.167 | 0.269017 | transcription and regulation |
| 2482 | pstB1 | −0.045 | 0.902 | 0.029694 | 0.013 | 1.030 | 0.663958 | transport |
| 2483 | pstA1 | 0.010 | 1.023 | 0.626526 | 0.026 | 1.063 | 0.37375 | transport |
| 2484 | pstC1 | −0.062 | 0.867 | 0.267088 | 0.014 | 1.033 | 0.82275 | transport |
| 2486 | yqgG | −0.052 | 0.887 | 0.343091 | 0.050 | 1.121 | 0.533583 | transport |
| 2488 | epi | 0.115 | 1.304 | 0.163143 | −0.013 | 0.971 | 0.718875 | general function prediction only |
| 2490 | vng2490 | 0.050 | 1.122 | 0.282426 | 0.011 | 1.026 | 0.82575 | unknown |
| 2493 | vng2493 | 0.071 | 1.177 | 0.333295 | −0.083 | 0.826 | 0.157813 | unknown |
| 2495 | vng2495 | 0.045 | 1.108 | 0.488344 | 0.006 | 1.014 | 0.665917 | unknown |
| 2496 | ferB | −0.108 | 0.779 | 0.421787 | −0.028 | 0.938 | 0.604833 | energy metabolism |
| 2497 | ucp | 0.018 | 1.042 | 0.225635 | −0.041 | 0.910 | 0.4585 | transcription and regulation |
| 2498 | vng2498 | 0.075 | 1.190 | 0.522521 | −0.034 | 0.925 | 0.631 | unknown |
| 2499 | gcdH | 0.150 | 1.413 | 0.03066 | −0.029 | 0.936 | 0.613 | carbohydrate and lipid metabolism |
| 2501 | vng2501 | −0.013 | 0.972 | 0.455631 | 0.077 | 1.193 | 0.18125 | nucleotide metabolism |
| 2502 | vng2502 | −0.005 | 0.989 | 0.612496 | −0.076 | 0.839 | 0.163125 | unknown |
| 2504 | pheS | −0.151 | 0.706 | 0.110857 | −0.020 | 0.956 | 0.52725 | translation |
| 2505 | pheY | 0.014 | 1.032 | 0.219198 | −0.066 | 0.860 | 0.263 | translation |
| 2507 | pyrD | 0.070 | 1.176 | 0.318644 | 0.020 | 1.048 | 0.30925 | nucleotide metabolism |
| 2508 | vng2508 | 0.107 | 1.279 | 0.187142 | −0.008 | 0.982 | 0.833083 | unknown |
| 2509 | vng2509 | 0.041 | 1.100 | 0.400897 | −0.074 | 0.843 | 0.199108 | unknown |
| 2510 | vng2510 | 0.048 | 1.116 | 0.417863 | −0.026 | 0.941 | 0.613 | unknown |
| 2511 | vng2511 | −0.096 | 0.801 | 0.058973 | 0.072 | 1.180 | 0.155 | unknown |
| 2512 | epf1 | 0.103 | 1.269 | 0.109343 | −0.021 | 0.954 | 0.6625 | translation |
| 2513 | aldY1 | 0.238 | 1.730 | 0.150774 | −0.008 | 0.982 | 0.618 | cofactor and secondary |
| | | | | | | | | metabolites |
| 2514 | rps6e | 0.111 | 1.291 | 0.1886 | −0.030 | 0.934 | 0.600888 | translation |
| 2515 | vng2515 | −0.025 | 0.945 | 0.616 | 0.038 | 1.091 | 0.5265 | unknown |
| 2516 | vng2516 | −0.013 | 0.969 | 0.784358 | 0.009 | 1.021 | 0.9075 | carbohydrate and lipid metabolism |
| 2518 | vng2518 | 0.076 | 1.191 | 0.541824 | 0.001 | 1.002 | 0.40775 | unknown |
| 2519 | vng2519 | 0.111 | 1.290 | 0.092951 | −0.004 | 0.992 | 0.546125 | unknown |
| 2520 | vng2520 | −0.042 | 0.908 | 0.507985 | 0.050 | 1.122 | 0.3265 | cellular process |
| 2521 | vng2521 | 0.099 | 1.257 | 0.303538 | −0.039 | 0.915 | 0.4385 | cellular process |
| 2522 | act9 | 0.058 | 1.144 | 0.326159 | 0.050 | 1.122 | 0.3895 | transcription and regulation |
| 2523 | vng2523 | −0.001 | 0.997 | 0.34141 | 0.076 | 1.191 | 0.245133 | cellular process |
| 2524 | vng2524 | 0.060 | 1.148 | 0.466121 | 0.000 | 1.000 | 0.578 | unknown |
| 2525 | vng2525 | 0.033 | 1.080 | 0.562385 | 0.014 | 1.033 | 0.4385 | unknown |
| 2526 | dppF | 0.095 | 1.245 | 0.188662 | 0.001 | 1.002 | 0.682 | transport |
| 2527 | dppD | 0.450 | 2.820 | 1.67E−11 | −0.064 | 0.863 | 0.249367 | transport |
| 2529 | dppB2 | 0.104 | 1.270 | 0.324618 | −0.025 | 0.943 | 0.552167 | transport |
| 2531 | dppC1 | 0.054 | 1.132 | 0.33708 | −0.006 | 0.987 | 0.765083 | transport |
| 2532 | vng2532 | 0.025 | 1.060 | 0.679691 | 0.040 | 1.096 | 0.4865 | unknown |
| 2533 | pyrC | 0.050 | 1.122 | 0.475958 | −0.016 | 0.964 | 0.693583 | nucleotide metabolism |
| 2534 | vng2536 | 0.005 | 1.011 | 0.485484 | −0.008 | 0.981 | 0.713875 | unknown |
| 2535 | vng2534 | −0.010 | 0.977 | 0.532838 | 0.015 | 1.036 | 0.75825 | unknown |
| 2536 | vng2535 | −0.081 | 0.830 | 0.107381 | −0.009 | 0.979 | 0.865 | cofactor and secondary |
| | | | | | | | | metabolites |
| 2537 | entB | 0.114 | 1.300 | 0.388164 | −0.026 | 0.943 | 0.484167 | cofactor and secondary |
| | | | | | | | | metabolites |
| 2539 | vng2539 | 0.313 | 2.055 | 0.115334 | −0.088 | 0.817 | 0.373574 | unknown |
| 2541 | cobB | 0.131 | 1.352 | 0.340151 | −0.012 | 0.972 | 0.791333 | cofactor and secondary |
| | | | | | | | | metabolites |
| 2543 | vng2543 | 0.134 | 1.362 | 0.150902 | 0.005 | 1.013 | 0.637333 | unknown |
| 2546 | pepB3 | 0.124 | 1.331 | 0.225931 | −0.005 | 0.989 | 0.7645 | amino acid metabolism |
| 2547 | valS | −0.084 | 0.824 | 0.314893 | −0.135 | 0.733 | 0.263483 | translation |
| 2549 | vng2549 | 0.066 | 1.164 | 0.231819 | −0.078 | 0.835 | 0.150792 | unknown |
| 2551 | fhuG | 0.126 | 1.336 | 0.110575 | −0.062 | 0.866 | 0.24175 | transport |
| 2552 | yfmF | 0.012 | 1.028 | 0.03777 | −0.018 | 0.960 | 0.680017 | transport |
| 2553 | yqeC | 0.086 | 1.218 | 0.31068 | −0.017 | 0.962 | 0.735667 | carbohydrate and lipid metabolism |
| 2554 | vng2554 | 0.020 | 1.046 | 0.608693 | −0.028 | 0.938 | 0.572 | unknown |
| 2555 | vng2555 | 0.123 | 1.327 | 0.037084 | 0.007 | 1.017 | 0.854167 | energy metabolism |
| 2556 | vng2556 | 0.147 | 1.404 | 0.155321 | 0.005 | 1.013 | 0.666333 | unknown |
| 2558 | vng2558 | −0.086 | 0.819 | 0.476545 | −0.020 | 0.956 | 0.3936 | unknown |
| 2560 | vng2560 | 0.019 | 1.046 | 0.566883 | −0.059 | 0.873 | 0.483333 | unknown |
| 2562 | vng2562 | 0.074 | 1.185 | 0.421214 | −0.129 | 0.744 | 0.04515 | unknown |
| 2563 | vng2563 | −0.049 | 0.894 | 0.576981 | 0.022 | 1.052 | 0.826333 | unknown |
| 2565 | trkH3 | 0.038 | 1.091 | 0.328615 | −0.031 | 0.931 | 0.66 | transport |
| 2566 | vng2566 | 0.087 | 1.221 | 0.401213 | 0.009 | 1.022 | 0.804083 | unknown |
| 2567 | vng2567 | 0.035 | 1.083 | 0.430535 | −0.028 | 0.937 | 0.55075 | cellular process |
| 2569 | vng2569 | 0.033 | 1.080 | 0.614734 | 0.034 | 1.082 | 0.59375 | unknown |
| 2570 | dcd | −0.005 | 0.987 | 0.568604 | 0.009 | 1.021 | 0.734667 | nucleotide metabolism |
| 2573 | hly | 0.210 | 1.621 | 0.04487 | −0.020 | 0.955 | 0.742 | cellular process |
| 2574 | can | 0.079 | 1.199 | 0.408713 | −0.114 | 0.769 | 0.089211 | energy metabolism |
| 2575 | riml | −0.176 | 0.667 | 0.07569 | 0.019 | 1.045 | 0.66175 | translation |
| 2576 | vng2576 | −0.116 | 0.766 | 0.39341 | −0.003 | 0.994 | 0.911875 | unknown |
| 2577 | vng2577 | −0.108 | 0.779 | 0.097568 | 0.000 | 0.999 | 0.85125 | unknown |
| 2579 | idr1 | 0.103 | 1.268 | 0.385097 | 0.017 | 1.040 | 0.754625 | transcription and regulation |
| 2580 | vng2580 | 0.024 | 1.056 | 0.627005 | −0.045 | 0.901 | 0.460383 | DNA metabolism |
| 2581 | vng2581 | 0.251 | 1.780 | 0.000651 | −0.048 | 0.895 | 0.329 | unknown |
| 2582 | vng2582 | 0.168 | 1.472 | 0.001368 | −0.023 | 0.948 | 0.639 | unknown |
| 2584 | vng2584 | −0.004 | 0.990 | 0.419066 | −0.077 | 0.838 | 0.159725 | translation |
| 2585 | vng2585 | 0.106 | 1.277 | 0.108486 | −0.070 | 0.851 | 0.1858 | unknown |
| 2586 | vng2586 | 0.013 | 1.031 | 0.650875 | −0.021 | 0.954 | 0.63725 | unknown |
| 2587 | vng2587 | 0.006 | 1.013 | 0.585327 | −0.017 | 0.961 | 0.6125 | unknown |
| 2589 | vng2589 | 0.105 | 1.275 | 0.055001 | −0.010 | 0.978 | 0.674 | unknown |
| 2591 | vng2591 | −0.021 | 0.952 | 0.233491 | −0.021 | 0.953 | 0.699667 | unknown |
| 2593 | vng2593 | 0.062 | 1.152 | 0.595942 | −0.086 | 0.820 | 0.308 | unknown |
| 2594 | Vng2594 | −0.034 | 0.925 | 0.546655 | 0.032 | 1.077 | 0.50675 | unknown |
| 2595 | gbp4 | 0.046 | 1.112 | 0.301914 | −0.004 | 0.992 | 0.61375 | cellular process |
| 2596 | hisl | −0.015 | 0.967 | 0.705647 | −0.007 | 0.984 | 0.68875 | amino acid metabolism |
| 2597 | vng2597 | −0.031 | 0.931 | 0.806684 | −0.030 | 0.933 | 0.686 | unknown |
| 2598 | hisH1 | 0.070 | 1.174 | 0.360664 | −0.004 | 0.990 | 0.843417 | amino acid metabolism |
| 2599 | vng2599 | 0.005 | 1.012 | 0.530047 | 0.002 | 1.004 | 0.604625 | cellular process |
| 2600 | trxA2 | 0.034 | 1.082 | 0.4858 | −0.085 | 0.821 | 0.189075 | nucleotide metabolism |
| 2602 | arsB | 0.012 | 1.029 | 0.438893 | −0.013 | 0.970 | 0.76275 | transport |
| 2603 | vng2603 | 0.099 | 1.257 | 0.065337 | 0.011 | 1.027 | 0.733375 | unknown |
| 2604 | thi1 | −0.025 | 0.944 | 0.42192 | −0.027 | 0.939 | 0.486733 | cofactor and secondary |
| | | | | | | | | metabolites |
| 2606 | thiD | −0.238 | 0.578 | 0.079359 | 0.012 | 1.027 | 0.615417 | cofactor and secondary |
| | | | | | | | | metabolites |
| 2607 | vng2607 | 0.014 | 1.034 | 0.550069 | −0.042 | 0.907 | 0.47125 | unknown |
| 2608 | vng2608 | −0.018 | 0.960 | 0.688723 | 0.018 | 1.042 | 0.506583 | unknown |
| 2610 | vng2610 | 0.067 | 1.167 | 0.416026 | −0.042 | 0.909 | 0.446583 | unknown |
| 2611 | hflX1 | −0.200 | 0.632 | 0.039538 | 0.048 | 1.118 | 0.513667 | cellular process |
| 2612 | rli | −0.100 | 0.793 | 0.314128 | −0.009 | 0.979 | 0.68275 | general function prediction only |
| 2613 | vng2613 | −0.011 | 0.976 | 0.563307 | −0.044 | 0.903 | 0.509917 | unknown |
| 2614 | arl15 | 0.139 | 1.377 | 0.208005 | −0.099 | 0.797 | 0.063767 | transcription and regulation |
| 2615 | vng2615 | −0.075 | 0.842 | 0.388695 | 0.032 | 1.077 | 0.459667 | unknown |
| 2616 | cxp | 0.609 | 4.065 | 6.66E−07 | −0.061 | 0.870 | 0.261242 | amino acid metabolism |
| 2617 | tdh2 | 0.026 | 1.062 | 0.686868 | 0.029 | 1.069 | 0.613 | amino acid metabolism |
| 2619 | vng2619 | 0.003 | 1.008 | 0.615648 | −0.065 | 0.861 | 0.20125 | unknown |
| 2620 | uvrD | −0.173 | 0.671 | 0.111419 | 0.061 | 1.150 | 0.242 | DNA metabolism |
| 2622 | vng2622 | −0.159 | 0.694 | 0.023899 | 0.011 | 1.025 | 0.76025 | unknown |
| 2624 | ribC | −0.094 | 0.806 | 0.371276 | 0.028 | 1.068 | 0.69575 | cofactor and secondary |
| | | | | | | | | metabolites |
| 2625 | vng2625 | 0.046 | 1.113 | 0.354867 | 0.049 | 1.119 | 0.444108 | unknown |
| 2626 | vng2626 | −0.016 | 0.964 | 0.558173 | 0.061 | 1.152 | 0.2275 | unknown |
| 2627 | vng2627 | −0.029 | 0.936 | 0.591399 | 0.004 | 1.009 | 0.75375 | translation |
| 2628 | vng2628 | −0.015 | 0.966 | 0.616968 | 0.024 | 1.057 | 0.606833 | cellular process |
| 2629 | glcK | −0.011 | 0.975 | 0.429521 | 0.027 | 1.064 | 0.690833 | carbohydrate and lipid metabolism |
| 2631 | vng2631 | −0.018 | 0.959 | 0.453936 | 0.037 | 1.090 | 0.56425 | unknown |
| 2632 | yusZ2 | 0.071 | 1.178 | 0.337041 | −0.016 | 0.963 | 0.77725 | cofactor and secondary |
| | | | | | | | | metabolites |
| 2633 | vng2633 | −0.046 | 0.899 | 0.362468 | 0.008 | 1.018 | 0.6205 | unknown |
| 2634 | vng2634 | −0.041 | 0.909 | 0.4176 | 0.001 | 1.003 | 0.70425 | unknown |
| 2636 | uvrA | −0.112 | 0.773 | 0.221422 | −0.017 | 0.961 | 0.727417 | DNA metabolism |
| 2637 | vng2637 | −0.138 | 0.727 | 0.383158 | −0.013 | 0.970 | 0.765917 | unknown |
| 2638 | bchP | −0.037 | 0.919 | 0.484228 | −0.046 | 0.899 | 0.388333 | cofactor and secondary |
| | | | | | | | | metabolites |
| 2639 | uae | −0.017 | 0.962 | 0.466289 | −0.021 | 0.952 | 0.454583 | nucleotide metabolism |
| 2640 | gcvT2 | −0.033 | 0.927 | 0.54365 | 0.043 | 1.105 | 0.508883 | amino acid metabolism |
| 2641 | vng2641 | −0.047 | 0.897 | 0.595283 | 0.005 | 1.011 | 0.824875 | transcription and regulation |
| 2642 | vng2642 | 0.027 | 1.064 | 0.397711 | −0.041 | 0.910 | 0.437083 | unknown |
| 2643 | vng2643 | −0.015 | 0.966 | 0.476732 | −0.035 | 0.922 | 0.492833 | unknown |
| 2644 | vng2644 | 0.165 | 1.463 | 0.036533 | −0.019 | 0.958 | 0.728 | unknown |
| 2645 | vng2646 | −0.002 | 0.996 | 0.427516 | −0.049 | 0.892 | 0.318 | unknown |
| 2646 | vng2645 | −0.078 | 0.836 | 0.458903 | −0.001 | 0.998 | 0.552 | translation |
| 2647 | vacB | 0.029 | 1.069 | 0.622027 | −0.057 | 0.877 | 0.294217 | transcription and regulation |
| 2648 | rps10p | 0.119 | 1.316 | 0.212525 | 0.004 | 1.009 | 0.8065 | translation |
| 2649 | eef1a | 0.126 | 1.335 | 0.194422 | 0.068 | 1.168 | 0.257875 | translation |
| 2650 | hom | −0.015 | 0.966 | 0.386164 | 0.016 | 1.039 | 0.473583 | amino acid metabolism |
| 2652 | vng2652 | 0.269 | 1.860 | 0.000529 | −0.095 | 0.804 | 0.365338 | DNA metabolism |
| 2653 | vng2653 | −0.017 | 0.963 | 0.570863 | 0.063 | 1.157 | 0.3324 | DNA metabolism |
| 2654 | eef2 | −0.032 | 0.929 | 0.345396 | 0.058 | 1.143 | 0.246667 | translation |
| 2656 | vng2656 | 0.073 | 1.183 | 0.438809 | −0.067 | 0.857 | 0.22065 | unknown |
| 2657 | rps7p | 0.041 | 1.098 | 0.361187 | 0.027 | 1.065 | 0.57525 | translation |
| 2658 | rps12p | −0.049 | 0.894 | 0.340266 | 0.030 | 1.072 | 0.54075 | translation |
| 2661 | nusA | −0.090 | 0.813 | 0.077868 | −0.048 | 0.895 | 0.33225 | transcription and regulation |
| 2662 | rpoC | −0.274 | 0.533 | 1.22E−05 | 0.043 | 1.103 | 0.430333 | transcription and regulation |
| 2664 | rpoA | −0.223 | 0.599 | 0.106787 | −0.005 | 0.988 | 0.22885 | transcription and regulation |
| 2665 | rpoB′ | −0.575 | 0.266 | 4.12E−13 | 0.091 | 1.234 | 0.080829 | transcription and regulation |
| 2666 | rpoB″ | −0.069 | 0.853 | 0.390009 | 0.089 | 1.226 | 0.188313 | transcription and regulation |
| 2668 | rpoH | −0.106 | 0.784 | 0.305791 | 0.102 | 1.265 | 0.046275 | transcription and regulation |
| 2669 | imp7 | 0.045 | 1.110 | 0.535939 | 0.024 | 1.056 | 0.686417 | energy metabolism |
| 2673 | vng2673 | 0.023 | 1.055 | 0.682917 | 0.077 | 1.194 | 0.2487 | unknown |
| 2674 | vng2674 | 0.019 | 1.046 | 0.595962 | 0.082 | 1.207 | 0.196333 | unknown |
| 2675 | acrR | −0.033 | 0.928 | 0.402689 | −0.016 | 0.963 | 0.664483 | transcription and regulation |
| 2677 | vng2677 | −0.068 | 0.854 | 0.261076 | −0.005 | 0.988 | 0.545417 | unknown |
| 2678 | vng2678 | 0.089 | 1.228 | 0.248809 | 0.038 | 1.092 | 0.4755 | unknown |
| 2679 | csg | 0.153 | 1.421 | 0.010772 | −0.007 | 0.985 | 0.751 | cellular process |
| 3000 | tRNA | 0.119 | 1.314 | 0.035567 | 0.016 | 1.038 | 0.40152 | translation |
| 3001 | tRNA | 0.314 | 2.063 | 2.51E−08 | 0.106 | 1.277 | 0.055528 | translation |
| 3002 | tRNA | 0.000 | 0.999 | 0.131238 | −0.078 | 0.835 | 0.258925 | translation |
| 3003 | tRNA | 0.393 | 2.473 | 3.4E−08 | 0.219 | 1.658 | 7.73E−05 | translation |
| 3004 | tRNA | 0.025 | 1.060 | 0.470113 | 0.041 | 1.100 | 0.173504 | translation |
| 3005 | tRNA | −0.028 | 0.938 | 0.555293 | −0.026 | 0.941 | 0.8155 | translation |
| 3006 | tRNA | −0.177 | 0.666 | 0.46896 | −0.010 | 0.978 | 0.9275 | translation |
| 3007 | tRNA | −0.062 | 0.868 | 0.621429 | 0.064 | 1.159 | 0.56075 | translation |
| 3008 | tRNA | −0.050 | 0.892 | 0.565564 | −0.046 | 0.900 | 0.564 | translation |
| 3009 | tRNA | −0.178 | 0.664 | 0.171007 | −0.198 | 0.633 | 0.004584 | translation |
| 3010 | tRNA | −0.106 | 0.783 | 0.410509 | −0.173 | 0.671 | 0.1248 | translation |
| 3011 | tRNA | −0.019 | 0.957 | 0.541344 | −0.089 | 0.815 | 0.169308 | translation |
| 3012 | tRNA | 0.010 | 1.023 | 0.605356 | −0.007 | 0.983 | 0.88825 | translation |
| 3013 | tRNA | −0.014 | 0.967 | 0.003602 | 0.059 | 1.146 | 0.256 | translation |
| 3014 | tRNA | −0.158 | 0.695 | 0.01848 | 0.060 | 1.148 | 0.2295 | translation |
| 3015 | tRNA | 0.163 | 1.455 | 0.474648 | −0.003 | 0.993 | 0.7565 | translation |
| 3016 | tRNA | −0.087 | 0.818 | 0.387377 | 0.027 | 1.063 | 0.7085 | translation |
| 3017 | tRNA | −0.149 | 0.709 | 0.363234 | −0.059 | 0.873 | 0.619 | translation |
| 3018 | tRNA | 0.200 | 1.584 | 0.015527 | −0.019 | 0.957 | 0.72175 | translation |
| 3019 | tRNA | 0.200 | 1.585 | 0.003996 | 0.036 | 1.087 | 0.489 | translation |
| 3021 | tRNA | −0.092 | 0.809 | 0.271202 | −0.045 | 0.901 | 0.4365 | translation |
| 3022 | tRNA | −0.134 | 0.735 | 0.402319 | 0.015 | 1.036 | 0.7585 | translation |
| 3023 | tRNA | 0.027 | 1.065 | 0.142714 | −0.024 | 0.947 | 0.68075 | translation |
| 3024 | tRNA | −0.127 | 0.747 | 0.198157 | −0.039 | 0.914 | 0.5015 | translation |
| 3025 | tRNA | −0.045 | 0.902 | 0.3946 | −0.118 | 0.762 | 0.03243 | translation |
| 3026 | tRNA | −0.093 | 0.807 | 0.340357 | −0.074 | 0.842 | 0.66175 | translation |
| 3027 | tRNA | −0.169 | 0.678 | 0.046252 | −0.110 | 0.777 | 0.10105 | translation |
| 3028 | tRNA | 0.014 | 1.033 | 0.701634 | 0.024 | 1.056 | 0.699 | translation |
| 3029 | tRNA | −0.125 | 0.751 | 0.126396 | −0.001 | 0.998 | 0.2345 | translation |
| 3030 | tRNA | −0.100 | 0.794 | 0.161178 | 0.045 | 1.109 | 0.377 | translation |
| 3031 | tRNA | −0.133 | 0.737 | 0.239301 | −0.023 | 0.948 | 0.6365 | translation |
| 3032 | tRNA | −0.088 | 0.817 | 0.430897 | −0.160 | 0.692 | 0.00622 | translation |
| 3033 | tRNA | 0.015 | 1.034 | 0.579953 | 0.039 | 1.094 | 0.58 | translation |
| 3034 | tRNA | 0.062 | 1.155 | 0.673442 | 0.010 | 1.023 | 0.866 | translation |
| 3035 | tRNA | −0.108 | 0.781 | 0.211551 | −0.007 | 0.985 | 0.77425 | translation |
| 3036 | tRNA | −0.103 | 0.790 | 0.131444 | −0.031 | 0.931 | 0.53425 | translation |
| 3037 | tRNA | 0.044 | 1.106 | 0.607989 | −0.097 | 0.800 | 0.0649 | translation |
| 3038 | tRNA | 0.078 | 1.197 | 0.430678 | −0.155 | 0.700 | 0.0355 | translation |
| 3039 | tRNA | −0.117 | 0.764 | 0.316588 | −0.027 | 0.940 | 0.6755 | translation |
| 3040 | tRNA | −0.358 | 0.438 | 2.51E−07 | −0.150 | 0.709 | 0.037272 | translation |
| 3041 | tRNA | −0.029 | 0.935 | 0.8084 | −0.067 | 0.857 | 0.3365 | translation |
| 3042 | tRNA | −0.069 | 0.854 | 0.448467 | −0.208 | 0.620 | 0.012852 | translation |
| 3043 | tRNA | −0.176 | 0.666 | 0.075531 | −0.033 | 0.927 | 0.7455 | translation |
| 3044 | tRNA | 0.120 | 1.318 | 0.069364 | 0.036 | 1.087 | 0.722 | translation |
| 3045 | tRNA | −0.132 | 0.738 | 0.118692 | −0.062 | 0.868 | 0.325175 | translation |
| 3046 | tRNA | −0.256 | 0.554 | 0.250283 | −0.130 | 0.741 | 0.028435 | translation |
| 3047 | tRNA | −0.001 | 0.997 | 0.880274 | 0.088 | 1.225 | 0.1907 | translation |
| 3048 | tRNA | 0.085 | 1.215 | 0.166421 | −0.115 | 0.768 | 0.191775 | translation |
| 3049 | tRNA | 0.067 | 1.167 | 0.355877 | −0.151 | 0.706 | 0.029033 | translation |
| 3050 | tRNA | −0.169 | 0.678 | 0.199877 | −0.116 | 0.766 | 0.2047 | translation |
| 3051 | tRNA | 0.025 | 1.058 | 0.787865 | −0.013 | 0.970 | 0.7975 | translation |
| 5001 | vng5001 | −0.179 | 0.662 | 0.02356 | −0.050 | 0.891 | 0.4828 | unknown |
| 5003 | vng5003 | −0.256 | 0.555 | 1.24E−06 | −0.067 | 0.857 | 0.3401 | unknown |
| 5005 | vng5005 | −0.088 | 0.816 | 0.291859 | −0.009 | 0.979 | 0.8685 | unknown |
| 5007 | vng5007 | −0.023 | 0.949 | 0.07113 | 0.039 | 1.094 | 0.4415 | unknown |
| 5008 | vng5008 | 0.046 | 1.111 | 0.357729 | −0.028 | 0.937 | 0.5835 | unknown |
| 5010 | sojA | 0.345 | 2.213 | 6.45E−10 | −0.063 | 0.864 | 0.28005 | transcription and regulation |
| 5011 | vng5011 | −0.193 | 0.641 | 0.051648 | −0.071 | 0.849 | 0.2335 | unknown |
| 5013 | repH | −0.069 | 0.853 | 0.510309 | −0.041 | 0.910 | 0.4905 | DNA metabolism |
| 5015 | vng5015 | −0.058 | 0.874 | 0.300351 | −0.091 | 0.811 | 0.30695 | unknown |
| 5016 | vng5016 | 0.061 | 1.152 | 0.293605 | −0.043 | 0.906 | 0.4365 | unknown |
| 5017 | vng5017 | 0.005 | 1.012 | 0.62617 | 0.043 | 1.105 | 0.416 | unknown |
| 5019 | gvpM1 | −0.308 | 0.492 | 0.4065 | 0.079 | 1.198 | 0.20785 | cellular process |
| 5020 | gvpL1 | −0.538 | 0.290 | 3.08E−14 | 0.080 | 1.201 | 0.1126 | cellular process |
| 5021 | gvpK1 | −1.084 | 0.082 | 5.93E−25 | 0.056 | 1.137 | 0.28725 | cellular process |
| 5022 | gvpJ1 | −0.989 | 0.102 | 6.5E−28 | 0.070 | 1.176 | 0.306175 | cellular process |
| 5023 | gvpI1 | −0.701 | 0.199 | 2.06E−18 | 0.077 | 1.194 | 0.12825 | cellular process |
| 5025 | gvpH1 | −1.176 | 0.067 | 3.99E−28 | 0.048 | 1.116 | 0.3635 | cellular process |
| 5026 | gvpG1 | −1.021 | 0.095 | 6E−29 | 0.043 | 1.105 | 0.3935 | cellular process |
| 5027 | gvpF1 | −0.910 | 0.123 | 2.47E−27 | 0.051 | 1.125 | 0.313 | cellular process |
| 5028 | gvpE1 | −0.530 | 0.295 | 4.43E−15 | 0.055 | 1.134 | 0.3095 | cellular process |
| 5029 | gvpD1 | −0.273 | 0.534 | 5.2E−05 | 0.080 | 1.202 | 0.202 | cellular process |
| 5030 | gvpA1 | −0.271 | 0.536 | 5.46E−07 | 0.059 | 1.145 | 0.233 | cellular process |
| 5032 | gvpC1 | −0.198 | 0.634 | 0.275 | 0.064 | 1.159 | 0.42585 | cellular process |
| 5033 | gvpN1 | −0.644 | 0.227 | 1.65E−18 | 0.036 | 1.086 | 0.49125 | cellular process |
| 5034 | gvpO1 | −0.478 | 0.333 | 5.2E−12 | 0.020 | 1.046 | 0.708 | cellular process |
| 5035 | sojB | −0.350 | 0.446 | 1.52E−09 | −0.006 | 0.986 | 0.81525 | transcription and regulation |
| 5037 | htlD | −0.118 | 0.763 | 0.033357 | 0.047 | 1.113 | 0.4718 | cellular process |
| 5038 | vng5038 | −0.017 | 0.961 | 0.450768 | 0.067 | 1.167 | 0.20775 | unknown |
| 5039 | tbpA | 0.096 | 1.247 | 0.120559 | −0.034 | 0.926 | 0.612 | transcription and regulation |
| 5040 | vng5040 | 0.022 | 1.053 | 0.501255 | −0.070 | 0.851 | 0.20955 | unknown |
| 5041 | vng5041 | −0.081 | 0.830 | 0.15135 | 0.093 | 1.240 | 0.1877 | unknown |
| 5042 | vng5042 | 0.008 | 1.019 | 0.739453 | 0.047 | 1.115 | 0.68475 | unknown |
| 5044 | vng5044 | −0.040 | 0.911 | 0.660686 | 0.059 | 1.147 | 0.4705 | unknown |
| 5045 | vng5045 | 0.209 | 1.619 | 0.015669 | 0.091 | 1.233 | 0.2815 | unknown |
| 5047 | vng5047 | 0.046 | 1.113 | 0.353352 | −0.035 | 0.922 | 0.579 | unknown |
| 5048 | vng5048 | 0.077 | 1.193 | 0.479092 | −0.043 | 0.905 | 0.3815 | unknown |
| 5052 | tbpB | −0.034 | 0.924 | 0.558184 | −0.107 | 0.781 | 0.03685 | transcription and regulation |
| 5053 | vng5053 | −0.136 | 0.731 | 0.169416 | −0.858 | 0.139 | 1.04E−08 | unknown |
| 5055 | cydA | −0.021 | 0.952 | 0.43635 | 0.026 | 1.063 | 0.75 | energy metabolism |
| 5057 | cydB | 0.093 | 1.240 | 0.430982 | −0.009 | 0.981 | 0.822 | energy metabolism |
| 5059 | vng5059 | 0.136 | 1.368 | 0.067808 | −0.572 | 0.268 | 3.45E−14 | unknown |
| 5061 | vng5061 | 0.011 | 1.026 | 0.757023 | −0.624 | 0.237 | 1.72E−19 | unknown |
| 5062 | vng5062 | −0.021 | 0.953 | 0.563709 | −0.183 | 0.657 | 0.034763 | unknown |
| 5063 | vng5063 | 0.066 | 1.165 | 0.363293 | −0.103 | 0.789 | 0.18 | unknown |
| 5064 | vng5064 | −0.033 | 0.927 | 0.776754 | −0.384 | 0.413 | 0.1605 | unknown |
| 5066 | phoT1 | −0.016 | 0.964 | 0.39697 | 0.054 | 1.133 | 0.4525 | transport |
| 5068 | boa3 | −0.297 | 0.505 | 0.233 | −0.592 | 0.256 | 1.35E−09 | transcription and regulation |
| 5069 | vng5069 | −0.053 | 0.884 | 0.4974 | −0.048 | 0.896 | 0.8725 | unknown |
| 5071 | yfmO2 | −0.014 | 0.968 | 0.646364 | −0.100 | 0.795 | 0.366 | general function prediction only |
| 5073 | vng5073 | −0.027 | 0.941 | 0.689483 | −0.006 | 0.987 | 0.70875 | unknown |
| 5074 | vng5074 | −0.032 | 0.929 | 0.8177 | −0.032 | 0.929 | 0.838 | unknown |
| 5075 | vng5075 | 0.086 | 1.218 | 0.132667 | −0.100 | 0.795 | 0.0534 | unknown |
| 5076 | trxA1 | −0.310 | 0.490 | 8.54E−07 | −0.140 | 0.724 | 0.025503 | nucleotide metabolism |
| 5077 | trxB1 | 0.014 | 1.033 | 0.450191 | −0.137 | 0.730 | 0.069675 | nucleotide metabolism |
| 5078 | trh | 0.004 | 1.010 | 0.644885 | −0.006 | 0.986 | 0.889 | general function prediction only |
| 5079 | vng5079 | 0.006 | 1.013 | 0.711857 | −0.266 | 0.542 | 0.17 | unknown |
| 5080 | vng5080 | −0.037 | 0.918 | 0.468685 | −0.001 | 0.999 | 0.642 | unknown |
| 5081 | vng5081 | −0.109 | 0.778 | 0.099572 | −0.324 | 0.474 | 1.73E−07 | unknown |
| 5082 | vng5082 | −0.291 | 0.512 | 1.71E−07 | 0.027 | 1.063 | 0.633 | unknown |
| 5084 | crt | −0.066 | 0.860 | 0.329583 | −0.147 | 0.712 | 0.006885 | cofactor and secondary |
| | | | | | | | | metabolites |
| 5085 | vng5085 | −0.130 | 0.742 | 0.604864 | −0.054 | 0.883 | 0.4145 | unknown |
| 5086 | vng5086 | −0.006 | 0.986 | 0.568228 | 0.044 | 1.106 | 0.48925 | unknown |
| 5087 | vng5087 | −0.493 | 0.321 | 0.131372 | −0.019 | 0.956 | 0.6845 | unknown |
| 5088 | vng5088 | −0.017 | 0.961 | 0.863342 | −0.072 | 0.847 | 0.1985 | unknown |
| 5089 | sojC1 | 0.039 | 1.094 | 0.457159 | −0.076 | 0.840 | 0.1509 | transcription and regulation |
| 5090 | vng5090 | 0.005 | 1.012 | 0.754081 | 0.013 | 1.031 | 0.8105 | unknown |
| 5091 | vng5091 | 0.033 | 1.078 | 0.414643 | −0.041 | 0.909 | 0.4725 | unknown |
| 5093 | vng5093 | 0.062 | 1.153 | 0.300155 | −0.080 | 0.832 | 0.12795 | unknown |
| 5094 | orc9 | 0.037 | 1.088 | 0.626685 | −0.112 | 0.772 | 0.0692 | cellular process |
| 5100 | vng5100 | 0.131 | 1.352 | 0.253496 | −0.191 | 0.645 | 0.012245 | unknown |
| 5102 | vng5102 | −0.030 | 0.934 | 0.594186 | −0.268 | 0.539 | 0.36625 | unknown |
| 5104 | vng5104 | 0.003 | 1.006 | 0.729016 | −0.042 | 0.907 | 0.6585 | unknown |
| 5105 | vng5105 | 0.012 | 1.028 | 0.591297 | −0.431 | 0.370 | 8.28E−07 | unknown |
| 5106 | vng5106 | 0.033 | 1.079 | 0.511855 | −0.181 | 0.659 | 0.000491 | unknown |
| 5108 | vng5108 | −0.218 | 0.606 | 0.081388 | −0.198 | 0.633 | 0.000216 | transcription and regulation |
| 5109 | vng5109 | −0.092 | 0.809 | 0.178308 | −0.122 | 0.755 | 0.048294 | unknown |
| 5114 | vng5114 | −0.084 | 0.823 | 0.233614 | −0.095 | 0.804 | 0.335175 | unknown |
| 5115 | vng5115 | 0.001 | 1.003 | 0.978503 | 0.025 | 1.060 | 0.6145 | unknown |
| 5116 | vng5116 | −0.032 | 0.928 | 0.582065 | 0.032 | 1.077 | 0.528 | unknown |
| 5118 | vng5118 | −0.114 | 0.769 | 0.284 | 0.010 | 1.023 | 0.844 | unknown |
| 5119 | vng5119 | −0.015 | 0.966 | 0.78151 | −0.027 | 0.939 | 0.623 | unknown |
| 5122 | vng5122 | −0.061 | 0.869 | 0.157769 | 0.022 | 1.051 | 0.69125 | unknown |
| 5123 | vng5123 | −0.004 | 0.991 | 0.195699 | −0.014 | 0.968 | 0.6925 | unknown |
| 5124 | vng5124 | 0.107 | 1.280 | 0.045045 | −0.029 | 0.936 | 0.53975 | unknown |
| 5126 | sojD | −0.017 | 0.962 | 0.566517 | −0.039 | 0.915 | 0.509 | transcription and regulation |
| 5129 | vng5129 | −0.071 | 0.850 | 0.350411 | 0.081 | 1.205 | 0.12435 | unknown |
| 5130 | vng5130 | 0.016 | 1.038 | 0.759651 | −0.111 | 0.775 | 0.0949 | unknown |
| 5131 | vng5131 | 0.051 | 1.125 | 0.456072 | −0.065 | 0.861 | 0.268 | unknown |
| 5132 | vng5132 | −0.007 | 0.984 | 0.652234 | −0.012 | 0.972 | 0.79075 | unknown |
| 5133 | vng5133 | −0.126 | 0.747 | 0.103952 | −0.008 | 0.982 | 0.928 | unknown |
| 5134 | rad25 | −0.018 | 0.959 | 0.730852 | −0.043 | 0.906 | 0.438 | DNA metabolism |
| 5135 | vng5135 | −0.063 | 0.865 | 0.363958 | −0.031 | 0.931 | 0.515 | unknown |
| 5136 | repl | 0.004 | 1.009 | 0.134443 | 0.023 | 1.053 | 0.709167 | DNA metabolism |
| 5138 | rme | −0.002 | 0.996 | 0.169013 | 0.012 | 1.029 | 0.8585 | DNA metabolism |
| 5139 | vng5139 | −0.073 | 0.846 | 0.375888 | −0.147 | 0.713 | 0.0494 | unknown |
| 5141 | hepA | −0.162 | 0.689 | 0.073816 | −0.094 | 0.806 | 0.2565 | translation |
| 5142 | tbpC | 0.053 | 1.131 | 0.520235 | −0.019 | 0.957 | 0.768 | transcription and regulation |
| 5143 | vng5143 | 0.126 | 1.338 | 0.190458 | −0.066 | 0.860 | 0.42575 | unknown |
| 5144 | prlR5 | 0.066 | 1.165 | 0.200995 | −0.030 | 0.933 | 0.5925 | transcription and regulation |
| 5145 | vng5145 | 0.442 | 2.769 | 7.37E−05 | −0.037 | 0.919 | 0.56155 | unknown |
| 5146 | vng5146 | −0.031 | 0.931 | 0.393972 | −0.006 | 0.986 | 0.732083 | unknown |
| 5147 | repJ | 0.004 | 1.009 | 0.610011 | 0.056 | 1.138 | 0.558667 | DNA metabolism |
| 5148 | vng5148 | −0.039 | 0.915 | 0.277127 | −0.048 | 0.896 | 0.5615 | unknown |
| 5149 | vng5149 | −0.071 | 0.849 | 0.246492 | 0.035 | 1.084 | 0.5495 | unknown |
| 5150 | vng5150 | −0.250 | 0.562 | 0.45277 | 0.029 | 1.069 | 0.714542 | unknown |
| 5152 | vng5152 | −0.095 | 0.804 | 0.298356 | 0.078 | 1.196 | 0.549625 | unknown |
| 5153 | vng5153 | −0.062 | 0.868 | 0.409672 | 0.072 | 1.181 | 0.621833 | unknown |
| 5154 | vng5154 | 0.025 | 1.059 | 0.186227 | 0.095 | 1.244 | 0.4285 | unknown |
| 5156 | arlR16 | −0.074 | 0.843 | 0.239924 | 0.057 | 1.141 | 0.460458 | transcription and regulation |
| 5157 | vng5157 | −0.068 | 0.855 | 0.228948 | 0.083 | 1.211 | 0.262117 | unknown |
| 5160 | vng5160 | −0.099 | 0.796 | 0.266025 | 0.081 | 1.204 | 0.440517 | unknown |
| 5163 | tbpD | −0.025 | 0.944 | 0.566874 | 0.071 | 1.177 | 0.390233 | transcription and regulation |
| 5164 | vng5164 | −0.067 | 0.857 | 0.35442 | 0.121 | 1.322 | 0.058075 | unknown |
| 5165 | vng5165 | 0.019 | 1.045 | 0.63621 | 0.077 | 1.194 | 0.22925 | unknown |
| 5166 | vng5166 | −0.061 | 0.870 | 0.291869 | 0.089 | 1.228 | 0.26615 | unknown |
| 5167 | vng5167 | −0.049 | 0.893 | 0.552739 | 0.027 | 1.064 | 0.796125 | unknown |
| 5168 | vng5168 | −0.102 | 0.791 | 0.230491 | −0.160 | 0.692 | 0.075083 | unknown |
| 5169 | vng5169 | −0.012 | 0.973 | 0.754996 | 0.092 | 1.236 | 0.266583 | unknown |
| 5170 | vng5170 | −0.054 | 0.882 | 0.287528 | −0.076 | 0.840 | 0.1938 | unknown |
| 5173 | vng5173 | −0.189 | 0.648 | 0.119037 | 0.008 | 1.019 | 0.68875 | unknown |
| 5174 | vng5174 | −0.048 | 0.895 | 0.275433 | 0.079 | 1.199 | 0.33475 | unknown |
| 5175 | vng5175 | 0.104 | 1.269 | 0.365722 | −0.129 | 0.743 | 0.01164 | unknown |
| 5176 | arsR2 | 0.092 | 1.235 | 0.25243 | −0.009 | 0.980 | 0.747667 | transcription and regulation |
| 5177 | arsM | 0.043 | 1.104 | 0.274777 | 0.032 | 1.078 | 0.585417 | cofactor and secondary |
| | | | | | | | | metabolites |
| 5178 | vng5178 | −0.149 | 0.709 | 0.131267 | 0.049 | 1.119 | 0.6585 | unknown |
| 5180 | arsA | −0.052 | 0.888 | 0.391327 | 0.098 | 1.252 | 0.263942 | transport |
| 5181 | arsD | −0.082 | 0.827 | 0.164743 | 0.041 | 1.100 | 0.45125 | transcription and regulation |
| 5182 | arsR | −0.106 | 0.783 | 0.456566 | 0.099 | 1.255 | 0.103367 | transcription and regulation |
| 5183 | arsC | −0.579 | 0.264 | 7.59E−15 | 0.174 | 1.491 | 0.004506 | cellular process |
| 5185 | vng5185 | 0.081 | 1.205 | 0.380166 | 0.013 | 1.030 | 0.644833 | unknown |
| 5186 | vng5186 | −0.030 | 0.933 | 0.915752 | 0.083 | 1.211 | 0.563 | unknown |
| 5189 | vng5189 | 0.379 | 2.394 | 0.255115 | −0.022 | 0.952 | 0.620333 | unknown |
| 5191 | vng5191 | 0.228 | 1.689 | 0.266131 | −0.017 | 0.963 | 0.706 | unknown |
| 5192 | vng5192 | 0.268 | 1.851 | 0.042732 | −0.018 | 0.960 | 0.561113 | unknown |
| 5194 | vng5194 | 0.102 | 1.265 | 0.473793 | 0.025 | 1.059 | 0.616167 | unknown |
| 5195 | vng5195 | 0.112 | 1.293 | 0.430313 | 0.034 | 1.081 | 0.67975 | unknown |
| 5197 | vng5197 | 0.158 | 1.440 | 0.305654 | 0.084 | 1.213 | 0.418483 | unknown |
| 5199 | vng5199 | −0.071 | 0.850 | 0.393978 | −0.056 | 0.879 | 0.40045 | unknown |
| 5200 | vng5200 | −0.313 | 0.487 | 3.04E−06 | −0.215 | 0.610 | 0.000118 | unknown |
| 5203 | vng5203 | 0.116 | 1.306 | 0.024495 | −0.003 | 0.992 | 0.60725 | unknown |
| 5204 | vng5204 | 0.093 | 1.238 | 0.068704 | −0.071 | 0.849 | 0.179775 | unknown |
| 5206 | vng5206 | 0.002 | 1.004 | 0.592389 | −0.041 | 0.911 | 0.453 | unknown |
| 5207 | vng5207 | −0.028 | 0.937 | 0.731538 | 0.024 | 1.057 | 0.677 | unknown |
| 5208 | sojC1 | 0.017 | 1.040 | 0.745484 | −0.063 | 0.866 | 0.225 | transcription and regulation |
| 5209 | vng5209 | −0.011 | 0.974 | 0.564611 | −0.071 | 0.850 | 0.20745 | unknown |
| 5210 | vng5210 | −0.098 | 0.798 | 0.104479 | −0.016 | 0.964 | 0.847 | unknown |
| 5211 | vng5211 | −0.041 | 0.910 | 0.405311 | 0.045 | 1.110 | 0.46 | unknown |
| 5212 | vng5212 | −0.043 | 0.905 | 0.489506 | −0.051 | 0.889 | 0.51375 | unknown |
| 5213 | crt | −0.047 | 0.897 | 0.54385 | −0.140 | 0.724 | 0.008105 | cofactor and secondary |
| | | | | | | | | metabolites |
| 5215 | vng5215 | −0.294 | 0.508 | 1.98E−07 | 0.015 | 1.035 | 0.631 | unknown |
| 5216 | vng5216 | −0.101 | 0.793 | 0.277906 | −0.335 | 0.463 | 7.43E−05 | unknown |
| 5217 | vng5217 | −0.038 | 0.916 | 0.454505 | −0.001 | 0.998 | 0.573 | unknown |
| 5218 | vng5218 | −0.022 | 0.950 | 0.66829 | −0.202 | 0.628 | 0.254 | unknown |
| 5219 | trh | 0.026 | 1.061 | 0.628 | −0.004 | 0.991 | 0.8765 | general function prediction only |
| 5220 | trxB1 | 0.038 | 1.091 | 0.749105 | −0.137 | 0.729 | 0.04305 | nucleotide metabolism |
| 5221 | trxA1 | −0.290 | 0.513 | 1.03E−05 | −0.132 | 0.737 | 0.0709 | nucleotide metabolism |
| 5222 | vng5222 | 0.016 | 1.038 | 0.084072 | −0.099 | 0.795 | 0.0468 | unknown |
| 5223 | vng5223 | −0.089 | 0.815 | 0.614116 | 0.005 | 1.011 | 0.948 | unknown |
| 5224 | vng5224 | −0.005 | 0.988 | 0.852418 | −0.031 | 0.932 | 0.8355 | unknown |
| 5225 | yfmO2 | 0.010 | 1.024 | 0.630995 | −0.054 | 0.883 | 0.485 | general function prediction only |
| 5228 | vng5228 | −0.070 | 0.851 | 0.353413 | −0.036 | 0.920 | 0.9 | unknown |
| 5229 | boa3 | −0.082 | 0.828 | 0.466238 | −0.575 | 0.266 | 6.81E−09 | transcription and regulation |
| 5231 | phoT1 | −0.029 | 0.935 | 0.616443 | 0.079 | 1.200 | 0.28 | transport |
| 5233 | vng5233 | −0.009 | 0.979 | 0.447077 | −0.283 | 0.521 | 0.1645 | unknown |
| 5234 | vng5234 | 0.037 | 1.089 | 0.666635 | −0.109 | 0.778 | 0.21625 | unknown |
| 5235 | vng5235 | −0.006 | 0.987 | 0.652245 | −0.207 | 0.621 | 0.016703 | unknown |
| 5236 | vng5236 | −0.014 | 0.968 | 0.748799 | −0.643 | 0.227 | 1.87E−19 | unknown |
| 5238 | vng5238 | 0.084 | 1.215 | 0.399293 | −0.485 | 0.327 | 6.94E−14 | unknown |
| 5240 | cydB | 0.095 | 1.244 | 0.119387 | −0.024 | 0.946 | 0.71075 | energy metabolism |
| 5242 | cydA | 0.016 | 1.037 | 0.640042 | 0.021 | 1.049 | 0.525 | energy metabolism |
| 5244 | vng5244 | −0.125 | 0.749 | 0.323011 | −0.916 | 0.121 | 3.83E−06 | unknown |
| 5245 | tbpB | −0.007 | 0.984 | 0.231474 | −0.098 | 0.798 | 0.0823 | transcription and regulation |
| 5249 | vng5249 | 0.046 | 1.111 | 0.420097 | −0.042 | 0.908 | 0.3935 | unknown |
| 5250 | vng5250 | 0.051 | 1.124 | 0.331549 | −0.040 | 0.912 | 0.4675 | unknown |
| 5252 | vng5252 | 0.084 | 1.213 | 0.503962 | 0.088 | 1.225 | 0.2995 | unknown |
| 5253 | vng5253 | −0.058 | 0.875 | 0.546774 | 0.046 | 1.111 | 0.5905 | unknown |
| 5255 | vng5255 | −0.033 | 0.928 | 0.619803 | 0.078 | 1.198 | 0.5035 | unknown |
| 5256 | vng5256 | −0.087 | 0.819 | 0.08774 | 0.124 | 1.331 | 0.06344 | unknown |
| 6131 | vng6131 | −0.069 | 0.854 | 0.230798 | 0.070 | 1.175 | 0.241 | unknown |
| 6133 | vng6133 | −0.061 | 0.869 | 0.3165 | 0.049 | 1.118 | 0.598538 | unknown |
| 6139 | hepA | −0.033 | 0.928 | 0.555602 | −0.056 | 0.878 | 0.66225 | translation |
| 6142 | vng6142 | 0.033 | 1.079 | 0.545301 | 0.056 | 1.138 | 0.610583 | unknown |
| 6143 | vng6143 | 0.014 | 1.032 | 0.831241 | 0.050 | 1.123 | 0.707 | unknown |
| 6144 | trsE | 0.002 | 1.004 | 0.426854 | 0.047 | 1.114 | 0.451167 | transport |
| 6145 | vng6145 | 0.165 | 1.461 | 0.089378 | 0.061 | 1.150 | 0.408667 | unknown |
| 6148 | vng6148 | 0.038 | 1.090 | 0.347085 | −0.006 | 0.987 | 0.296 | unknown |
| 6149 | vng6149 | 0.058 | 1.144 | 0.579402 | 0.067 | 1.168 | 0.286342 | unknown |
| 6150 | orc1 | 0.093 | 1.238 | 0.279266 | 0.021 | 1.049 | 0.697417 | cellular process |
| 6152 | vng6152 | −0.038 | 0.917 | 0.290921 | 0.054 | 1.132 | 0.5337 | unknown |
| 6153 | sojC2 | −0.088 | 0.817 | 0.460563 | 0.030 | 1.072 | 0.749 | transcription and regulation |
| 6155 | vng6155 | −0.086 | 0.821 | 0.663422 | 0.020 | 1.048 | 0.821583 | unknown |
| 6156 | vng6156 | 0.028 | 1.068 | 0.537293 | 0.024 | 1.057 | 0.6195 | unknown |
| 6157 | vng6157 | 0.004 | 1.009 | 0.420349 | 0.109 | 1.286 | 0.204175 | unknown |
| 6158 | vng6158 | 0.044 | 1.107 | 0.853 | 0.086 | 1.220 | 0.4505 | unknown |
| 6159 | vng6159 | 0.805 | 6.388 | 3.09E−18 | −0.018 | 0.960 | 0.89925 | unknown |
| 6160 | vng6160 | 0.114 | 1.301 | 0.561316 | 0.063 | 1.157 | 0.490575 | unknown |
| 6162 | vng6162 | 0.013 | 1.031 | 0.539605 | 0.010 | 1.024 | 0.906333 | unknown |
| 6163 | vng6163 | 0.056 | 1.138 | 0.696495 | 0.066 | 1.164 | 0.77225 | unknown |
| 6164 | orc2 | −0.006 | 0.987 | 0.890419 | 0.095 | 1.243 | 0.086 | cellular process |
| 6165 | vng6165 | −0.097 | 0.800 | 0.398768 | 0.053 | 1.130 | 0.4205 | unknown |
| 6166 | nbp4 | 0.037 | 1.090 | 0.326717 | −0.032 | 0.930 | 0.582 | transcription and regulation |
| 6168 | vng6168 | 0.046 | 1.111 | 0.301336 | 0.044 | 1.108 | 0.492 | unknown |
| 6170 | vng6170 | 0.084 | 1.214 | 0.517926 | −0.009 | 0.979 | 0.66725 | unknown |
| 6171 | vng6171 | −0.135 | 0.733 | 0.187153 | 0.063 | 1.157 | 0.40725 | unknown |
| 6173 | vng6173 | −0.011 | 0.974 | 0.597229 | −0.057 | 0.877 | 0.502333 | unknown |
| 6175 | trkA2 | 0.017 | 1.039 | 0.348707 | −0.023 | 0.948 | 0.616083 | transport |
| 6176 | kdpA | −0.010 | 0.977 | 0.68634 | 0.022 | 1.052 | 0.843917 | transport |
| 6177 | kdpB | −0.024 | 0.947 | 0.583726 | 0.049 | 1.120 | 0.450167 | transport |
| 6178 | kdpC | −0.064 | 0.864 | 0.632841 | −0.025 | 0.943 | 0.534117 | transport |
| 6179 | cat3 | −0.180 | 0.660 | 0.510806 | 0.014 | 1.033 | 0.821833 | transport |
| 6180 | vng6180 | 0.027 | 1.063 | 0.624142 | 0.012 | 1.029 | 0.528417 | unknown |
| 6181 | vng6181 | −0.070 | 0.851 | 0.27556 | −0.096 | 0.802 | 0.246654 | unknown |
| 6182 | vng6182 | −0.009 | 0.979 | 0.538828 | −0.022 | 0.950 | 0.402667 | unknown |
| 6183 | vng6183 | −0.130 | 0.742 | 0.248762 | 0.011 | 1.026 | 0.704417 | unknown |
| 6184 | cat4 | 0.007 | 1.016 | 0.726784 | −0.014 | 0.969 | 0.893583 | transport |
| 6185 | vng6185 | 0.062 | 1.154 | 0.520823 | −0.022 | 0.950 | 0.694 | unknown |
| 6186 | vng6186 | 0.085 | 1.217 | 0.513169 | −0.054 | 0.884 | 0.39 | unknown |
| 6187 | orc3 | −0.010 | 0.977 | 0.361439 | 0.049 | 1.120 | 0.547 | cellular process |
| 6188 | vng6188 | −0.013 | 0.971 | 0.685961 | −0.122 | 0.755 | 0.091539 | unknown |
| 6189 | vng6189 | −0.021 | 0.953 | 0.422301 | 0.060 | 1.149 | 0.505333 | unknown |
| 6191 | vng6191 | 0.102 | 1.265 | 0.208376 | 0.023 | 1.055 | 0.615 | unknown |
| 6193 | camR3 | −0.030 | 0.932 | 0.432379 | −0.004 | 0.990 | 0.728083 | transcription and regulation |
| 6195 | vng6195 | −0.048 | 0.895 | 0.385064 | −0.112 | 0.773 | 0.16657 | unknown |
| 6196 | phoT2 | −0.021 | 0.954 | 0.608671 | 0.044 | 1.107 | 0.624083 | transport |
| 6197 | vng6197 | −0.018 | 0.960 | 0.726929 | 0.061 | 1.150 | 0.3441 | unknown |
| 6198 | vng6198 | −0.040 | 0.911 | 0.523206 | 0.017 | 1.041 | 0.9265 | unknown |
| 6199 | cdc48d | −0.166 | 0.683 | 0.393715 | −0.008 | 0.982 | 0.877167 | cellular process |
| 6201 | hsp5 | 0.072 | 1.179 | 0.47186 | −0.100 | 0.794 | 0.173186 | cellular process |
| 6203 | vng6203 | −0.077 | 0.838 | 0.602084 | 0.018 | 1.043 | 0.822917 | unknown |
| 6204 | vng6204 | −0.039 | 0.913 | 0.410954 | 0.002 | 1.005 | 0.7275 | unknown |
| 6205 | vng6205 | −0.113 | 0.771 | 0.045563 | −0.008 | 0.982 | 0.6645 | unknown |
| 6206 | vng6206 | 0.043 | 1.104 | 0.554267 | −0.025 | 0.943 | 0.760333 | unknown |
| 6208 | imp5 | −0.199 | 0.632 | 0.035321 | −0.053 | 0.886 | 0.3895 | general function prediction only |
| 6209 | vng6209 | −0.045 | 0.901 | 0.400675 | −0.012 | 0.974 | 0.682 | unknown |
| 6210 | gabT1 | 0.002 | 1.005 | 0.307169 | −0.010 | 0.976 | 0.378333 | amino acid metabolism |
| 6211 | bdb | −0.200 | 0.630 | 0.413012 | −0.049 | 0.893 | 0.54896 | amino acid metabolism |
| 6212 | iucA | −0.045 | 0.901 | 0.657355 | −0.035 | 0.923 | 0.845208 | transport |
| 6213 | iucB | −0.127 | 0.746 | 0.32608 | −0.047 | 0.898 | 0.505333 | transport |
| 6214 | hxyA | −0.072 | 0.847 | 0.685793 | −0.075 | 0.842 | 0.3885 | cofactor and secondary |
| | | | | | | | | metabolites |
| 6216 | iucC | −0.015 | 0.967 | 0.617274 | −0.021 | 0.954 | 0.52055 | transport |
| 6218 | trkA3 | −0.078 | 0.836 | 0.353607 | −0.020 | 0.954 | 0.644875 | transport |
| 6220 | cat2 | 0.018 | 1.042 | 0.182528 | −0.072 | 0.847 | 0.189067 | transport |
| 6221 | vng6221 | 0.012 | 1.028 | 0.612577 | 0.021 | 1.049 | 0.587833 | unknown |
| 6222 | vng6222 | −0.139 | 0.726 | 0.126748 | −0.019 | 0.958 | 0.673083 | unknown |
| 6223 | vng6223 | 0.049 | 1.119 | 0.315965 | −0.051 | 0.889 | 0.4085 | unknown |
| 6224 | vng6224 | 0.116 | 1.307 | 0.301554 | 0.024 | 1.057 | 0.666667 | unknown |
| 6225 | vng6225 | 0.009 | 1.020 | 0.628128 | −0.032 | 0.930 | 0.578667 | unknown |
| 6226 | vng6226 | 0.011 | 1.027 | 0.651675 | −0.023 | 0.949 | 0.714417 | unknown |
| 6229 | gvpL2 | −0.186 | 0.652 | 0.434416 | 0.057 | 1.141 | 0.6565 | cellular process |
| 6230 | gvpK2 | 0.044 | 1.107 | 0.729793 | 0.016 | 1.038 | 0.919 | cellular process |
| 6232 | gvpJ2 | −0.140 | 0.725 | 0.162329 | 0.021 | 1.048 | 0.818 | cellular process |
| 6233 | gvpI2 | 0.072 | 1.179 | 0.342415 | −0.024 | 0.947 | 0.55575 | cellular process |
| 6235 | gvpH2 | 0.029 | 1.070 | 0.352745 | 0.004 | 1.008 | 0.387 | cellular process |
| 6236 | gvpG2 | −0.040 | 0.913 | 0.247989 | −0.021 | 0.953 | 0.5049 | cellular process |
| 6237 | gvpF2 | −0.004 | 0.991 | 0.281062 | −0.060 | 0.872 | 0.386 | cellular process |
| 6239 | gvpE2 | −0.111 | 0.775 | 0.281722 | 0.003 | 1.008 | 0.622083 | cellular process |
| 6240 | gvpD2 | −0.086 | 0.820 | 0.37545 | −0.080 | 0.832 | 0.356947 | cellular process |
| 6241 | gvpA2 | −0.549 | 0.283 | 6.09E−17 | 0.025 | 1.060 | 0.6355 | cellular process |
| 6242 | gvpC2 | −0.109 | 0.778 | 0.558507 | 0.017 | 1.041 | 0.661317 | cellular process |
| 6244 | gvpN2 | −0.062 | 0.867 | 0.51432 | 0.012 | 1.028 | 0.604 | cellular process |
| 6246 | gvpO2 | 0.101 | 1.260 | 0.368747 | −0.025 | 0.944 | 0.7375 | cellular process |
| 6247 | trkA4 | 0.061 | 1.150 | 0.422389 | −0.023 | 0.948 | 0.675083 | transport |
| 6250 | phoT3 | −0.036 | 0.921 | 0.404966 | −0.018 | 0.959 | 0.62375 | transport |
| 6251 | vng6251 | 0.016 | 1.039 | 0.556447 | −0.061 | 0.868 | 0.427193 | transcription and regulation |
| 6254 | vng6254 | 0.059 | 1.146 | 0.427799 | −0.107 | 0.782 | 0.157663 | unknown |
| 6255 | vng6255 | 0.044 | 1.106 | 0.584681 | −0.094 | 0.805 | 0.2076 | unknown |
| 6256 | lipB | −0.020 | 0.955 | 0.597847 | 0.022 | 1.052 | 0.667417 | cofactor and secondary |
| | | | | | | | | metabolites |
| 6258 | hacA | 0.067 | 1.167 | 0.372381 | −0.061 | 0.869 | 0.390917 | amino acid metabolism |
| 6260 | ftsZ5 | −0.022 | 0.952 | 0.654784 | −0.007 | 0.984 | 0.767417 | cellular process |
| 6261 | yocR | −0.084 | 0.824 | 0.23238 | −0.138 | 0.728 | 0.074431 | transport |
| 6262 | zurM | −0.163 | 0.687 | 0.311826 | 0.008 | 1.019 | 0.815833 | transport |
| 6264 | zurA | −0.096 | 0.801 | 0.250059 | 0.004 | 1.008 | 0.3245 | transport |
| 6265 | adcH | −0.112 | 0.773 | 0.416416 | −0.019 | 0.957 | 0.707917 | transport |
| 6266 | vng6266 | −0.040 | 0.913 | 0.233503 | 0.049 | 1.120 | 0.480083 | unknown |
| 6268 | imp6 | 0.042 | 1.103 | 0.290067 | −0.004 | 0.990 | 0.543333 | general function prediction only |
| 6269 | vng6269 | −0.061 | 0.870 | 0.555645 | −0.040 | 0.911 | 0.705167 | unknown |
| 6270 | gldA | −0.187 | 0.650 | 0.003691 | −0.024 | 0.946 | 0.5205 | energy metabolism |
| 6272 | orc5 | −0.111 | 0.775 | 0.41789 | 0.039 | 1.095 | 0.492833 | cellular process |
| 6275 | vng6275 | −0.040 | 0.912 | 0.447014 | 0.003 | 1.006 | 0.733167 | unknown |
| 6276 | vng6276 | −0.295 | 0.507 | 6.09E−06 | −0.120 | 0.758 | 0.0691 | unknown |
| 6277 | ugpB | −0.294 | 0.509 | 0.132493 | −0.030 | 0.933 | 0.610333 | transport |
| 6279 | ugpA | −0.216 | 0.608 | 0.221475 | −0.001 | 0.998 | 0.702833 | transport |
| 6280 | ugpE | −0.023 | 0.948 | 0.456176 | −0.033 | 0.927 | 0.677583 | transport |
| 6281 | ugpC | −0.138 | 0.727 | 0.172784 | −0.015 | 0.966 | 0.671667 | transport |
| 6283 | vng6283 | −0.101 | 0.793 | 0.2549 | 0.032 | 1.076 | 0.734 | unknown |
| 6284 | vng6284 | −0.029 | 0.936 | 0.287351 | 0.018 | 1.043 | 0.717167 | unknown |
| 6286 | vng6286 | −0.113 | 0.770 | 0.285807 | 0.019 | 1.045 | 0.746833 | unknown |
| 6287 | arlR17 | −0.135 | 0.732 | 0.029158 | 0.050 | 1.122 | 0.39875 | transcription and regulation |
| 6288 | vng6288 | −0.173 | 0.671 | 0.074871 | −0.022 | 0.951 | 0.616917 | unknown |
| 6290 | vng6290 | −0.106 | 0.783 | 0.312608 | 0.074 | 1.186 | 0.305625 | unknown |
| 6291 | vng6291 | −0.006 | 0.985 | 0.450263 | 0.042 | 1.100 | 0.768125 | unknown |
| 6292 | vng6292 | −0.078 | 0.836 | 0.213617 | −0.016 | 0.964 | 0.82125 | unknown |
| 6293 | vng6293 | −0.042 | 0.907 | 0.580362 | −0.090 | 0.813 | 0.2444 | unknown |
| 6294 | perA | −0.266 | 0.542 | 0.008448 | 0.026 | 1.061 | 0.6105 | cellular process |
| 6296 | vng6296 | 0.051 | 1.124 | 0.345447 | −0.107 | 0.782 | 0.088108 | unknown |
| 6297 | vng6297 | −0.110 | 0.777 | 0.274909 | −0.003 | 0.993 | 0.6975 | unknown |
| 6298 | pct | 0.013 | 1.030 | 0.439028 | 0.001 | 1.002 | 0.813 | general function prediction only |
| 6299 | hypE | 0.006 | 1.014 | 0.573681 | −0.021 | 0.953 | 0.629333 | translation |
| 6301 | aph | −0.090 | 0.812 | 0.526895 | −0.049 | 0.894 | 0.759833 | cellular process |
| 6302 | tlyC | −0.045 | 0.903 | 0.536962 | −0.006 | 0.986 | 0.855667 | general function prediction only |
| 6303 | exsB | 0.005 | 1.012 | 0.265803 | 0.051 | 1.125 | 0.4995 | cellular process |
| 6305 | vng6305 | −0.177 | 0.665 | 0.154728 | 0.009 | 1.021 | 0.162645 | unknown |
| 6306 | vng6306 | −0.172 | 0.673 | 0.127188 | 0.111 | 1.291 | 0.197065 | unknown |
| 6308 | gltP | 0.054 | 1.134 | 0.418731 | −0.111 | 0.774 | 0.118808 | transport |
| 6309 | pyrB | 0.293 | 1.965 | 2.77E−06 | −0.125 | 0.749 | 0.02214 | nucleotide metabolism |
| 6311 | pyrl | 0.298 | 1.985 | 3.8E−06 | −0.133 | 0.736 | 0.009068 | nucleotide metabolism |
| 6312 | argS | 0.117 | 1.308 | 0.182389 | −0.142 | 0.721 | 0.00933 | translation |
| 6313 | nhaC3 | −0.055 | 0.881 | 0.544415 | −0.779 | 0.166 | 1.95E−14 | transport |
| 6315 | arcB | 0.183 | 1.524 | 0.090477 | −0.643 | 0.227 | 1.02E−20 | amino acid metabolism |
| 6316 | arcC | 0.130 | 1.348 | 0.154476 | −0.773 | 0.169 | 2.28E−24 | amino acid metabolism |
| 6317 | arcA | 0.193 | 1.559 | 0.163895 | −0.351 | 0.446 | 5.39E−07 | amino acid metabolism |
| 6318 | arcR | −0.042 | 0.908 | 0.603536 | −0.078 | 0.835 | 0.198883 | transcription and regulation |
| 6319 | vng6319 | 0.165 | 1.462 | 0.013309 | −0.060 | 0.871 | 0.407508 | unknown |
| 6320 | vng6320 | −0.030 | 0.933 | 0.559741 | 0.060 | 1.147 | 0.275667 | unknown |
| 6321 | vng6321 | −0.066 | 0.859 | 0.554369 | −0.015 | 0.967 | 0.500583 | unknown |
| 6322 | vng6322 | −0.038 | 0.916 | 0.523955 | 0.035 | 1.085 | 0.640875 | unknown |
| 6323 | vng6323 | 0.078 | 1.196 | 0.40583 | 0.003 | 1.006 | 0.715 | unknown |
| 6325 | vng6325 | 0.018 | 1.041 | 0.585006 | −0.041 | 0.909 | 0.492933 | unknown |
| 6326 | trp6 | −0.036 | 0.920 | 0.585213 | −0.036 | 0.920 | 0.564933 | transport |
| 6327 | vng6327 | −0.056 | 0.880 | 0.341407 | 0.078 | 1.197 | 0.324183 | unknown |
| 6329 | vng6329 | −0.070 | 0.851 | 0.280217 | −0.480 | 0.331 | 0.1805 | unknown |
| 6332 | vng6332 | −0.042 | 0.907 | 0.377391 | −0.072 | 0.846 | 0.163725 | unknown |
| 6334 | vng6334 | 0.008 | 1.018 | 0.275542 | −0.466 | 0.342 | 3.86E−11 | unknown |
| 6335 | vng6335 | 0.063 | 1.155 | 0.565122 | −0.980 | 0.105 | 1.8E−20 | unknown |
| 6337 | sojE | 0.113 | 1.297 | 0.295145 | −0.143 | 0.720 | 0.272031 | transcription and regulation |
| 6339 | vng6339 | −0.044 | 0.903 | 0.640285 | −0.074 | 0.843 | 0.324875 | unknown |
| 6340 | vng6340 | −0.009 | 0.979 | 0.581887 | −0.204 | 0.625 | 0.007696 | unknown |
| 6343 | vng6343 | −0.037 | 0.919 | 0.509537 | 0.052 | 1.127 | 0.40525 | unknown |
| 6346 | vng6346 | −0.014 | 0.969 | 0.749952 | 0.041 | 1.100 | 0.543667 | unknown |
| 6347 | vng6347 | −0.084 | 0.825 | 0.268967 | −0.126 | 0.749 | 0.020798 | unknown |
| 6348 | vng6348 | 0.010 | 1.023 | 0.474254 | −0.016 | 0.963 | 0.479167 | unknown |
| 6349 | vng6349 | 0.128 | 1.344 | 0.293615 | −0.024 | 0.947 | 0.602083 | unknown |
| 6351 | tfbC | −0.058 | 0.876 | 0.248649 | 0.004 | 1.009 | 0.5965 | transcription and regulation |
| 6353 | vng6353 | −0.023 | 0.949 | 0.715471 | −0.008 | 0.982 | 0.72075 | unknown |
| 6354 | comA | −0.044 | 0.903 | 0.470743 | −0.017 | 0.962 | 0.65125 | cellular process |
| 6355 | vng6355 | 0.078 | 1.196 | 0.3832 | −0.083 | 0.827 | 0.268267 | unknown |
| 6357 | vng6357 | 0.024 | 1.058 | 0.454862 | −0.049 | 0.894 | 0.471333 | unknown |
| 6359 | vng6359 | −0.019 | 0.958 | 0.710433 | 0.041 | 1.098 | 0.675 | unknown |
| 6361 | tnp2 | −0.040 | 0.911 | 0.401015 | 0.176 | 1.499 | 0.026733 | cellular process |
| 6362 | polB2 | 0.008 | 1.018 | 0.523754 | 0.023 | 1.054 | 0.411233 | DNA metabolism |
| 6363 | orc4 | 0.096 | 1.248 | 0.209379 | 0.034 | 1.081 | 0.283942 | cellular process |
| 6364 | vng6364 | 0.019 | 1.045 | 0.768319 | 0.056 | 1.139 | 0.502 | unknown |
| 6365 | vng6365 | −0.035 | 0.923 | 0.513882 | 0.084 | 1.213 | 0.09615 | unknown |
| 6366 | vng6366 | −0.024 | 0.946 | 0.628875 | 0.052 | 1.126 | 0.555 | unknown |
| 6367 | vng6367 | 0.010 | 1.024 | 0.54956 | 0.049 | 1.118 | 0.729 | unknown |
| 6368 | vng6368 | 0.087 | 1.223 | 0.282801 | 0.069 | 1.171 | 0.2066 | unknown |
| 6370 | vng6370 | 0.077 | 1.194 | 0.497675 | 0.028 | 1.068 | 0.633 | unknown |
| 6371 | mrr | −0.172 | 0.674 | 0.294422 | 0.025 | 1.060 | 0.039408 | DNA metabolism |
| 6372 | vng6372 | 0.026 | 1.062 | 0.311504 | 0.124 | 1.330 | 0.03015 | unknown |
| 6373 | phrH | 0.180 | 1.514 | 0.021568 | 0.016 | 1.038 | 0.754 | transcription and regulation |
| 6375 | vng6375 | 0.100 | 1.260 | 0.427789 | 0.069 | 1.173 | 0.56325 | unknown |
| 6377 | vng6377 | 0.075 | 1.187 | 0.499467 | 0.044 | 1.107 | 0.6515 | unknown |
| 6378 | vng6378 | 0.092 | 1.236 | 0.283053 | 0.078 | 1.196 | 0.404 | unknown |
| 6379 | fes | −0.047 | 0.897 | 0.390906 | 0.137 | 1.371 | 0.039003 | general function prediction only |
| 6381 | vng6381 | −0.017 | 0.962 | 0.477945 | 0.120 | 1.318 | 0.048958 | unknown |
| 6383 | lctP | 0.097 | 1.249 | 0.235913 | 0.015 | 1.034 | 0.494583 | transport |
| 6384 | vng6384 | 0.021 | 1.049 | 0.761104 | 0.048 | 1.118 | 0.8285 | unknown |
| 6385 | vng6385 | 0.022 | 1.052 | 0.774203 | 0.076 | 1.193 | 0.375317 | unknown |
| 6387 | vng6387 | 0.039 | 1.095 | 0.510805 | 0.037 | 1.088 | 0.553917 | unknown |
| 6389 | tfbE | 0.015 | 1.035 | 0.662484 | 0.050 | 1.122 | 0.651658 | transcription and regulation |
| 6390 | vng6390 | 0.032 | 1.078 | 0.561096 | 0.054 | 1.133 | 0.35525 | unknown |
| 6393 | vng6393 | 0.114 | 1.300 | 0.357035 | 0.200 | 1.586 | 0.315517 | unknown |
| 6396 | nbp5 | −0.038 | 0.916 | 0.40082 | 0.392 | 2.468 | 0.023164 | transcription and regulation |
| 6397 | vng6397 | −0.036 | 0.921 | 0.791768 | 0.215 | 1.640 | 0.328083 | unknown |
| 6400 | vng6400 | −0.067 | 0.856 | 0.664055 | 0.278 | 1.898 | 0.475833 | unknown |
| 6401 | vng6401 | −0.033 | 0.927 | 0.441862 | 0.185 | 1.530 | 0.043093 | unknown |
| 6402 | arlR18 | −0.043 | 0.906 | 0.411958 | 0.515 | 3.274 | 0.00531 | transcription and regulation |
| 6403 | vng6403 | −0.040 | 0.913 | 0.458883 | 0.298 | 1.985 | 0.000405 | unknown |
| 6404 | vng6404 | −0.023 | 0.949 | 0.645973 | 0.054 | 1.133 | 0.584917 | unknown |
| 6406 | vng6406 | −0.036 | 0.920 | 0.404944 | 0.005 | 1.012 | 0.628167 | unknown |
| 6407 | vng6407 | 0.182 | 1.521 | 0.002876 | 0.000 | 1.000 | 0.286 | unknown |
| 6408 | phzF | 0.125 | 1.334 | 0.267353 | 0.439 | 2.751 | 0.001187 | cofactor and secondary |
| | | | | | | | | metabolites |
| 6409 | vng6409 | 0.080 | 1.202 | 0.502999 | 0.441 | 2.758 | 5.33E−05 | unknown |
| 6411 | vng6411 | 0.081 | 1.205 | 0.288498 | 0.311 | 2.045 | 0.087643 | unknown |
| 6412 | vng6412 | −0.056 | 0.880 | 0.532677 | 0.290 | 1.949 | 0.008309 | unknown |
| 6413 | vng6413 | −0.041 | 0.909 | 0.520033 | 0.893 | 7.823 | 6.7E−12 | unknown |
| 6416 | vng6416 | 0.079 | 1.200 | 0.405132 | 0.104 | 1.271 | 0.335217 | unknown |
| 6418 | vng6418 | 0.001 | 1.001 | 0.501978 | −0.014 | 0.969 | 0.505708 | unknown |
| 6419 | vng6419 | 0.137 | 1.371 | 0.259116 | 0.050 | 1.121 | 0.7625 | unknown |
| 6420 | vng6420 | 0.063 | 1.156 | 0.094898 | 0.498 | 3.150 | 6.12E−08 | unknown |
| 6424 | vng6424 | 0.026 | 1.061 | 0.486663 | 0.018 | 1.043 | 0.789917 | unknown |
| 6427 | vng6427 | −0.015 | 0.965 | 0.46848 | 0.047 | 1.114 | 0.619833 | unknown |
| 6429 | vng6429 | −0.030 | 0.934 | 0.51214 | 0.040 | 1.097 | 0.5159 | unknown |
| 6430 | vng6430 | 0.070 | 1.175 | 0.448742 | 0.043 | 1.104 | 0.5355 | unknown |
| 6431 | vng6431 | −0.132 | 0.738 | 0.513332 | 0.063 | 1.155 | 0.6725 | unknown |
| 6432 | vng6432 | 0.058 | 1.144 | 0.456059 | 0.065 | 1.160 | 0.373717 | unknown |
| 6434 | vng6434 | 0.019 | 1.045 | 0.182211 | 0.017 | 1.040 | 0.628875 | unknown |
| 6437 | vng6437 | −0.012 | 0.973 | 0.430677 | 0.052 | 1.127 | 0.513833 | unknown |
| 6438 | tbpF | −0.051 | 0.888 | 0.553077 | 0.028 | 1.066 | 0.90725 | transcription and regulation |
| 6439 | vng6439 | 0.022 | 1.053 | 0.37037 | 0.040 | 1.095 | 0.497167 | unknown |
| 6441 | vng6441 | −0.378 | 0.419 | 6.8E−07 | −0.001 | 0.998 | 0.80125 | unknown |

That which is claimed is:

1. A variant of Halobacterium sp NCR-1 strain comprising increased gene transcripts of rfa3, rfa8 and ral with at least a 1.9 to 4 fold increase of such genes transcripts relative to a wild type Halobacterium sp. NRC-1 strain.

2. The variant according to claim 1 comprising about 3 fold increase of rfa 3, about 2 fold increase of rfa8 and about 2 fold increase of ral gene transcript relative to a wild-type Halobacterium sp. NRC-1 strain.

3. The variant according to claim 1 comprising about 5 fold increase of rfa3, about 3.5 fold increase of rfa8 and about 3 fold increase of ral gene transcript relative to a wild-type Halobacterium sp. NRC-1 strain.

4. The variant according to claim 2, wherein the variant strain lacks gas vesicle production capability relative to the wild-type Halobacterium sp. NRC-1 strain.

5. The variant according to claim 1, wherein the exhibits resistance to ionizing radiation at a dose range from about 15 to about 23 kGy.

6. The variant according to claim 1, wherein the variant Halobacterium sp NRC-1 strains exhibit an LD₅₀ from about 11 kGy to about 25kGy.

7. A method of isolating variants of halophile Halobacterium sp. NRC-1 that exhibit resistance to extreme levels of radiation, the method comprising:
a) providing a culture medium comprising a NRC-1 halobacterium sp;
b) irradiating the culture with at least a dose rate of 10 kGy/s to 30 kGy/s
c) isolating the viable halobacterium strains;
d) repeating steps b and c at least one additional time; and
e) selecting and isolating variants of Halobacterium displaying increased gene transcripts of rfa3, rfa8 and ral with at least a 1.9 to 4 fold increase relative to the wild-type and/or non-irradiated Halobacterium sp. NRC-1 strain.

8. The method according to claim 7, wherein the dose rate is from about 16 kGy to 23 kGy.

9. The method according to claim 7, wherein the steps of b and c are repeated at least three additional times to isolate organisms with increased radiation resistance.

10. The method according to claim 7, wherein the radiation is preferably delivered by a source that provides a pulsed electron beam having the ability to deliver 18-20 MeV electrons.

11. The method according to claim 10, wherein the pulsed electron beam includes a pulse width of 1 to 3 ms, with a repetition rate of from about 40 to 80 HZ.

12. The method according to claim 7, wherein the halobacterium sp NRC-1 is in a growing phase during irradiation.

13. The method according to claim 7, wherein a culture of Halobacterium sp. NRC-1 strains is first radiated during a growth cycle with a dose of about 20 kGy, then viable bacteria is isolated and radiated with a dose of 23 kGy.

14. The method according to claim 7, wherein a culture of Halobacterium sp. NRC-1 strains is first radiated during a growth cycle with a dose of about 16 kGy, then viable bacteria is isolated and radiated with a dose of 23 kGy.

15. The method according to claim 7, wherein an electron beam irradiates the system.

16. A method for decontaminating a waste site comprising radioactive materials, the method comprising;
introducing a variant mutant Halobacterium sp NRC-1 strain according to claim 1 into a waste site; and
providing sufficient nutrients for maintaining the culture for decontaminating waste products in the waste site.

17. A biological indicator to ensure the quality control of radiation sterilization in the food industry, hospital settings or space travel, wherein the biological indicator is a mutant Halobacterium sp NRC-1 strain isolated according to claim 1, after exposing the Halobacterium sp NRC-1 strain four cycles of radiation.

18. A halophile Halobacterium sp. NRC-1 that exhibit resistance to extreme levels of radiation produced by the following steps:
a) providing a culture medium comprising a NRC-1 halobacterium sp;
b) irradiating the culture with at least a dose rate of 10 kGy/s to 30 kGy/s
c) isolating the viable halobacterium strains;
d) repeating steps b and c at least three additional time; and
e) selecting and isolating variants of Halobacterium that display increased gene transcripts of rfa3, rfa8 and ral with at least a 1.9 to 4 fold increase relative to the wild-type and/or non-irradiated Halobacterium sp. NRC-1 strain.


SEQUENCE LISTING (8 sequences)

SEQ ID NO: 1 - DNA, 1422 bp, Halobacterium sp. NRC-1
atgacggcgtcctcgccgaaccgcgcccgcacctcgctggcggcgtacgcgagcgccggccccgtgtccttgcccgccggctccacgaggacggtggcctcggggacctcgtcgcggacgcggtccgcgtacgcctcgcgggtgaccgccagcaggtcgtcggcgaaggccgcgcggtctgcggtccggcggagcaggctgcggtcgtcgtcgccgagcgcgagcagctgcttggggcggtggctgcggctcgcgggatacagccgggtgccggtgccgccggcgagcaggacggcagcgacagtgtgtgtcacggcgcgctcacccggagggtggagtgcatgccggcgggttcgacgccgcggtccaaatgcctgtctccggggggcgagcgaccgcgttcaccccgggtgcggagatttaaagggggttgggagcaaacaccgatacgtctcccatcgaaacacacgcggagacggatacacgaccatgagcgacctttcagacacggctcaggaggtccacgacgacctctcggagcacgtcgacgcgaccgtcgacgacgtggaagcacggctggagaaactggtttctgagtacaaagtcccggtcgacgaggcccgccgcagcgtcgagaaccactacctcgacgaggccggcctcgaccgcgacgacctcgccggcggcggcggcaacgacaccgtcgaagtcgaggacgtcgacgaggccgagcagtgggtggacatcacggcgaaagtcgtggaactgtgggagccccgttcggactccgtcgggcaggtcggcctgctgggcgacccgaccggcacgatcaagttcacgaagtgggcgaagtcggacctcgaaaccctcgacgaggggcaagtctatcgcctcgggaacgtcgtcaccgacgagtaccagggccgatactcggtgaagctgaacaagacaacgtccatcgaggcggtcgacgaggagttcgaggtcggcgacaacgaggacgaggtcgcgggcgcgctcgtggacgtacagtccggcagcgggctgatcaagcgctgccccgaggaggactgcacgcgcgtcctccagaacggccgttgcaacgagcacggcgaaggcgacggggagttcgacctccgcatcaagggggtgctcgacgacggcaccgacgtccacgaggtcatcttcgacgaggaggccacggaagccctcactggcatcggcctggaggaggccaagcagatggccaaagacgccctcgacacgaccgtcgttgccgacgagatgcgcgccacgatcctcgggaagtactatcgggtgacggggccgacgttcggtcggtacgtgctcgccaacgacaccgagttgcttaccacccggccggacactgaatccgtgctgatccgcgcgaggtcgatctga

SEQ ID NO: 2 - DNA, 573 bp, Halobacterium sp. NRC-1
atgagcggcgcacccacccgcgaagtcgcacgccgcgtcttcgcggccgagttcaacgacgccacgttcacgttcaaggagtccgacgacgagcgcgcgccgctgtacgcgttgctcccgaccggcgagcgcgcgaaccgcgtgttcgtcaccgggacgctcaccgagaccgaggacatcggcgaggacgcggagtactggcggggccgcgtcgtcgacccgacggggacgtttttcgtctacgcgggccagtaccagccggaagcggcggcggcactgcgggacgccgagacgccgacgtacgtcgccgtggtcgggaagccacggacctacgagaccgacgacgggtccgtgaacgtctccctgcggccggagtcgatcacgatggtcgaggagagcgttcgtgaccgctgggtggtcgaggccgccgagcgcacgctcgaccgcatcgaggccttcaacgaggagggcaacgagtacgccgagcgcgcccgcgccgagtacggcgcggatacctcggcgtaccggcaggcggtcgtcgacgcgctggcggagttcgacgcgccggagtga

SEQ ID NO: 3 - DNA, 405 bp, Halobacterium sp. NRC-1
atggggaacaagaacaagaccatctcgttccgggtgaacgaggaggcgtttgacgcgctcagcgagatcgccgaggagcgcaacatctccctgtcggcggtgttccgtgattacgttgactcgcttgtgacccacgacgggaaggtccgggtcgtgcccgaggacagcacgagagcgcgagacggcgacgacgacgacttcccgccgaccgtcgaggtgtcgaagagcttcgtgcgcgaacacgagcgcctcgaactggaagccgaacacctccgcgagcagctggacgagcagaaggcgtacatcgaccacctccggagtcagaccgacagcgaagacgagatcggtgacgtggtgaagttggaggaactggatgacgacgacgaaccctaccagctcggatag

SEQ ID NO: 4 - protein, 473 aa, Halobacterium sp. NRC-1
MTASSPNRARTSLAAYASAGPVSLPAGSTRTVASGTSSRTRSAYASRVTASRSSAKAARSAVRRSRLRSSSPSASSCLGRWLRLAGYSRVPVPPASRTAATVCVTARSPGGWSACRRVRRRGPNACLRGASDRVHPGCGDLKGVGSKHRYVSHRNTRGDGYTTMSDLSDTAQEVHDDLSEHVDATVDDVEARLEKLVSEYKVPVDEARRSVENHYLDEAGLDRDDLAGGGGNDTVEVEDVDEAEQWVDITAKVVELWEPRSDSVGQVGLLGDPTGTIKFTKWAKSDLETLDEGQVYRLGNVVTDEYQGRYSVKLNKTTSIEAVDEEFEVGDNEDEVAGALVDVQSGSGLIKRCPEEDCTRVLQNGRCNEHGEGDGEFDLRIKGVLDDGTDVHEVIFDEEATEALTGIGLEEAKQMAKDALDTTVVADEMRATILGKYYRVTGPTFGRYVLANDTELLTTRPDTESVLIRARSI

SEQ ID NO: 5 - protein, 190 aa, Halobacterium sp. NRC-1
MSGAPTREVARRVFAAEFNDATFTFKESDDERAPLYALLPTGERANRVFVTGTLTETEDIGEDAEYWRGRVVDPTGTFFVYAGQYQPEAAAALRDAETPTYVAVVGKPRTYETDDGSVNVSLRPESITMVEESVRDRWVVEAAERTLDRIEAFNEEGNEYAERARAEYGADTSAYRQAVVDALAEFDAPE

SEQ ID NO: 6 - protein, 134 aa, Halobacterium sp. NRC-1
MGNKNKTISFRVNEEAFDALSEIAEERNISLSAVFRDYVDSLVTHDGKVRVVPEDSTRARDGDDDDFPPTVEVSKSFVREHERLELEAEHLREQLDEQKAYIDHLRSQTDSEDEIGDVVKLEELDDDDEPYQLG

SEQ ID NO: 7 - DNA, 51 bp, Artificial Sequence
  Synthetic construct
gcgagcgaccgcgttcaccccgggtgcggagatttaaagggggttgggagc

SEQ ID NO: 8 - DNA, 53 bp, Artificial Sequence
  Synthetic construct
aacaccgatacgtctcccatcgaaacacacgcggagacggatacacgaccatg